SEQUENCE LISTING (117 sequences)

SEQ ID NO: 1 - protein, 24 aa, Unknown
  source: /note=“Description of Unknown Chloroplast localization signal sequence”
VCSLARNLCFSLFSTHKSGTGSSG

SEQ ID NO: 2 - protein, 9 aa, Unknown
  source: /note=“Description of Unknown Peroxisome localization signal sequence”
RLRIIGGHL

SEQ ID NO: 3 - protein, 54 aa, Artificial Sequence
  source: /note=“Description of Artificial Sequence Synthetic polypeptide”
MEVCSLARNLCFSLFSTHKSGTGSSGDSSSSGVSTKPQDRLRIIGGHLNVAAEA

SEQ ID NO: 4 - protein, 55 aa, Artificial Sequence
  source: /note=“Description of Artificial Sequence Synthetic polypeptide”
MDSSSSPVSTKPQDRLRIIGGHLNVAAEAMEVCSLARNLCFSLFSTHKSGTGSSG

SEQ ID NO: 5 - protein, 9 aa, Artificial Sequence
  source: /note=“Description of Artificial Sequence Synthetic peptide”
RLXXXXXHL

SEQ ID NO: 6 - protein, 58 aa, Unknown
  source: /note=“Description of Unknown chloroplast localization signal sequence”
MASSVISSAAVATRTNVTQAGSMIAPFTGLKSAATFPVSRKQNLDITSIASNGGRVRC

SEQ ID NO: 7 - protein, 61 aa, Artificial Sequence
  source: /note=“Description of Artificial Sequence Synthetic polypeptide”
MASSVISSAAVATRTNVTQAGSMIAPFTGLKSAATFPVSRLRVLSAHLITSIASNGGRVRC

SEQ ID NO: 8 - DNA, 6 bp, Artificial Sequence
  source: /note=“Description of Artificial Sequence Synthetic oligonucleotide”
gtatgt

SEQ ID NO: 9 - DNA, 6 bp, Artificial Sequence
  source: /note=“Description of Artificial Sequence Synthetic oligonucleotide”
gtatac

SEQ ID NO: 10 - DNA, 6 bp, Artificial Sequence
  source: /note=“Description of Artificial Sequence Synthetic oligonucleotide”
ttccag

SEQ ID NO: 11 - DNA, 6 bp, Artificial Sequence
  source: /note=“Description of Artificial Sequence Synthetic oligonucleotide”
gtatat

SEQ ID NO: 12 - DNA, 6 bp, Artificial Sequence
  source: /note=“Description of Artificial Sequence Synthetic oligonucleotide”
tgtaag

SEQ ID NO: 13 - DNA, 6 bp, Artificial Sequence
  source: /note=“Description of Artificial Sequence Synthetic oligonucleotide”
ttgcag

SEQ ID NO: 14 - DNA, 72 bp, Artificial Sequence
  source: /note=“Description of Artificial Sequence Synthetic oligonucleotide”
gtatgttctcttgccaggaatctctgcttcagtttattctcaacacataagagtggaactggctcgagcggc

SEQ ID NO: 15 - DNA, 177 bp, Artificial Sequence
  source: /note=“Description of Artificial Sequence Synthetic polynucleotide”
atggcttcctctgttatttcctctgccgctgttgctacacgcaccaatgttacacaagctggcagcatgattgcacctttcactggtctcaaatctgctgctactttccctgtttcaaggaagcaaaaccttgacatcacttccattgctagcaatggtggaagagttaggtgcatg

SEQ ID NO: 16 - DNA, 27 bp, Artificial Sequence
  source: /note=“Description of Artificial Sequence Synthetic oligonucleotide”
cgtctgagaatcattggagggcatttg

SEQ ID NO: 17 - DNA, 27 bp, Artificial Sequence
  source: /note=“Description of Artificial Sequence Synthetic oligonucleotide”
aggcttagagttctttctgctcatttg

SEQ ID NO: 18 - DNA, 437 bp, Artificial Sequence
  source: /note=“Description of Artificial Sequence Synthetic polynucleotide”
atggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcgacagctctagctctcctgtttcaacaaaacctcaaggtatattgatgatttaccaaatcttttccttgtcaaagttttgtgtttgactgtgtgggtttgaacctgttaggattcagtatgatatcaagtatgtgtcttttggaatacaaggatttacccttatggctatctttgttatctgtgtgaccttttctactttctcgctttgtaagatcgtctgagaatcattggagggcatttgaatgttgcagctgaagcaatg

SEQ ID NO: 19 - DNA, 440 bp, Artificial Sequence
  source: /note=“Description of Artificial Sequence Synthetic polynucleotide”
atggacagctctagctctcctgtttcaacaaaacctcaaggtatattgatgatttaccaaatcttttccttgtcaaagttttgtgtttgactgtgtgggtttgaacctgttaggattcagtatgatatcaagtatgtgtcttttggaatacaaggatttacccttatggctatctttgttatctgtgtgaccttttctactttctcgctttgtaagatcgtctgagaatcattggagggcatttgaatgttgcagctgaagcaatggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcatg

SEQ ID NO: 20 - DNA, 186 bp, Artificial Sequence
  source: /note=“Description of Artificial Sequence Synthetic polynucleotide”
atggcttcctctgttatttcctctgccgctgttgctacacgcaccaatgttacacaagctggcagcatgattgcacctttcactggtctcaaatctgctgctactttccctgtttcaaggcttagagttctttctgctcatttgatcacttccattgctagcaatggtggaagagttaggtgcatg

SEQ ID NO: 21 - protein, 37 aa, Artificial Sequence
  source: /note=“Description of Artificial Sequence Synthetic polypeptide”
MESGTGSSGDSSSSGVSTKPQDRLRIIGGHLNVAAEA

SEQ ID NO: 22 - protein, 41 aa, Artificial Sequence
  source: /note=“Description of Artificial Sequence Synthetic polypeptide”
MEVCSLARNLCFSLFSTHKSGTGSSGDSSSSGVSTKPQAEA

SEQ ID NO: 23 - protein, 24 aa, Artificial Sequence
  source: /note=“Description of Artificial Sequence Synthetic peptide”
MESGTGSSGDSSSSGVSTKPQAEA

SEQ ID NO: 24 - protein, 42 aa, Artificial Sequence
  source: /note=“Description of Artificial Sequence Synthetic polypeptide”
MDSSSSPVSTKPQAEAMEVCSLARNLCFSLFSTHKSGTGSSG

SEQ ID NO: 25 - protein, 38 aa, Artificial Sequence
  source: /note=“Description of Artificial Sequence Synthetic polypeptide”
MDSSSSPVSTKPQDRLRIIGGHLNVAAEAMESGTGSSG

SEQ ID NO: 26 - protein, 25 aa, Artificial Sequence
  source: /note=“Description of Artificial Sequence Synthetic peptide”
MDSSSSPVSTKPQAEAMESGTGSSG

SEQ ID NO: 27 - protein, 9 aa, Artificial Sequence
  source: /note=“Description of Artificial Sequence Synthetic peptide”
RLRVLSAHL

SEQ ID NO: 28 - DNA, 1934 bp, Artificial Sequence
  source: /note=“Description of Artificial Sequence Synthetic polynucleotide”
atggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcgacagctctagctctcctgtttcaacaaaacctcaaggtatattgatgatttaccaaatcttttccttgtcaaagttttgtgtttgactgtgtgggtttgaacctgttaggattcagtatgatatcaagtatgtgtcttttggaatacaaggatttacccttatggctatctttgttatctgtgtgaccttttctactttctcgctttgtaagatcgtctgagaatcattggagggcatttgaatgttgcagctgaagcaatgtctattctttatgaagagagactcgatggagctttaccagatgttgatagaacctcagtgctcatggcattaagggaacatgttcctggacttgaaattcttcacacagatgaagagattatcccatatgaatgtgatggtttgtctgcttacagaactaggcctcttttggttgtgctcccaaagcagatggaacaggttacagctattcttgcagtgtgccatagattgagggttcctgttgtgacaagaggagctggtaccggactttcaggaggtgcactcccattagaaaagggtgttctcttagtgatggctaggttcaaagagatattggatattaatcctgtgggaagaagggctagagttcaaccaggtgtgaggaatctcgcaattagtcaggctgttgcacctcacaacctttattacgctcctgatccatcttcacaaatcgcatgttctataggtggtaatgtggctgaaaacgcaggaggtgttcattgccttaagtacggattgactgtgcacaaccttttgaaaatcgaagttcagactcttgatggagaggctcttacattgggtagtgatgcattggattctcctggttttgatctcttagctctcttcacaggttctgaaggaatgttaggtgttactacagaggttaccgttaaacttttgccaaaacctccagttgctagagtgctcttagcatcttttgattcagtggaaaaagctggacttgcagttggagatataattgctaacggaattattcctggaggtctcgaaatgatggataacttatctataagagctgctgaagatttcattcatgctggatatccagttgatgctgaggcaatacttttgtgtgaacttgatggtgttgagtcagatgtgcaagaagattgcgagagagttaatgatattctcttaaaggctggagcaactgatgtgaggttggctcaggatgaagcagagagagttaggttttgggctggaagaaaaaacgctttccctgctgttggtaggatctcaccagattattactgtatggatggtacaatacctagaagggctctcccaggagttttagagggtattgcaagacttagtcaacagtacgatttgagggttgctaatgtgtttcatgcaggagatggaaacatgcaccctctcatcttatttgatgctaatgagccaggagagttcgctagagcagaagagcttggaggaaagattcttgaactttgtgttgaagtgggaggtagtatctctggtgaacatggtattggaagagagaaaatcaatcaaatgtgcgctcagttcaactctgatgaaatcaccacttttcatgctgttaaggctgcattcgatcctgatggacttttgaatcctggaaagaatataccaacattgcacagatgcgctgagttcggagcaatgcacgttcaccacggacaccttccttttcctgagttggagagattctga

SEQ ID NO: 29 - DNA, 2540 bp, Artificial Sequence
  source: /note=“Description of Artificial Sequence Synthetic polynucleotide”
atggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcgacagctctagctctcctgtttcaacaaaacctcaaggtatattgatgatttaccaaatcttttccttgtcaaagttttgtgtttgactgtgtgggtttgaacctgttaggattcagtatgatatcaagtatgtgtcttttggaatacaaggatttacccttatggctatctttgttatctgtgtgaccttttctactttctcgctttgtaagatcgtctgagaatcattggagggcatttgaatgttgcagctgaagcaatgctcagagaatgcgattattctcaggctcttttggagcaagtgaatcaggcaatttcagataagactcctcttgttatccaaggttctaactcaaaggcttttcttggtagaccagtgactggacagacacttgatgttagatgtcataggggtatcgtgaactacgatcctactgaattggttataacagctagagtgggaaccccacttgttactattgaagctgcattggagtctgctggtcaaatgctcccatgtgagcctccacactacggagaagaggcaacttggggtggtatggttgcttgcggacttgcaggtcctagaaggccatggagtggttctgttagagattttgtgttgggaacaaggattatcaccggagctggaaagcatctcagattcggaggtgaagttatgaaaaatgtggcaggttatgatctctcaaggttaatggttggaagttacggttgtcttggagtgttgacagaaatttctatgaaggttcttcctagaccaagggcttcacttagtttgagaagggaaatatctttgcaagaggctatgtcagaaattgcagagtggcaactccagcctttaccaattagtggattgtgctattttgataacgctctctggatcagattagaaggaggagagggttcagtgaaagctgcaagggaactcttaggaggtgaagaggttgctggacagttctggcaacagcttagagagcaacagttgcctttcttttctcttccaggtacattgtggaggataagtcttccttctgatgctccaatgatggatctccctggagaacaattaatcgattggggaggtgctcttagatggttgaagtcaacagcagaggataatcagatccatagaatagctaggaacgcaggaggtcacgctaccagattttcagcaggagatggaggtttcgctcctctcagtgcaccactttttagataccaccaacagttgaagcagcagttagatccttgtggtgtgttcaatcctggaagaatgtacgctgagttgtgaatgctcagagaatgcgattattctcaggctcttttggagcaagtgaatcaggcaatttcagataagactcctcttgttatccaaggttctaactcaaaggcttttcttggtagaccagtgactggacagacacttgatgttagatgtcataggggtatcgtgaactacgatcctactgaattggttataacagctagagtgggaaccccacttgttactattgaagctgcattggagtctgctggtcaaatgctcccatgtgagcctccacactacggagaagaggcaacttggggtggtatggttgcttgcggacttgcaggtcctagaaggccatggagtggttctgttagagattttgtgttgggaacaaggattatcaccggagctggaaagcatctcagattcggaggtgaagttatgaaaaatgtggcaggttatgatctctcaaggttaatggttggaagttacggttgtcttggagtgttgacagaaatttctatgaaggttcttcctagaccaagggcttcacttagtttgagaagggaaatatctttgcaagaggctatgtcagaaattgcagagtggcaactccagcctttaccaattagtggattgtgctattttgataacgctctctggatcagattagaaggaggagagggttcagtgaaagctgcaagggaactcttaggaggtgaagaggttgctggacagttctggcaacagcttagagagcaacagttgcctttcttttctcttccaggtacattgtggaggataagtcttccttctgatgctccaatgatggatctccctggagaacaattaatcgattggggaggtgctcttagatggttgaagtcaacagcagaggataatcagatccatagaatagctaggaacgcaggaggtcacgctaccagattttcagcaggagatggaggtttcgctcctctcagtgcaccactttttagataccaccaacagttgaagcagcagttagatccttgtggtgtgttcaatcctggaagaatgtacgctgagttgtga

SEQ ID NO: 30 - DNA, 1658 bp, Artificial Sequence
  source: /note=“Description of Artificial Sequence Synthetic polynucleotide”
atggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcgacagctctagctctcctgtttcaacaaaacctcaaggtatattgatgatttaccaaatcttttccttgtcaaagttttgtgtttgactgtgtgggtttgaacctgttaggattcagtatgatatcaagtatgtgtcttttggaatacaaggatttacccttatggctatctttgttatctgtgtgaccttttctactttctcgctttgtaagatcgtctgagaatcattggagggcatttgaatgttgcagctgaagcaatgcaaactcagcttacagaagagatgagacaaaatgctagggcactcgaagctgattctatcttaagagcatgtgttcattgcggattctgtaccgctacttgccctacttatcaacttttgggagatgagcttgatggaccaagaggtagaatatacctcattaagcaagttttagaaggaaacgaggtgaccttgaaaactcaggaacatcttgatagatgcttgacatgtaggaattgcgagactacatgtccatcaggagttaggtatcacaacctcttagatatcggtagagatatagttgaacagaaggtgaaaagacctcttccagaaagaatactcagggagggattaagacaagttgtgcctaggccagctgtgtttagagcattgactcaagttggtcttgtgttgaggcctttccttccagaacaggttagagcaaagttgcctgctgaaacagtgaaggctaaaccaagacctccacttaggcataaaagaagggttctcatgttagagggatgtgctcagcctactttgtctccaaatacaaacgctgcaaccgctagagttcttgataggttgggtatttcagtgatgcctgcaaatgaggctggatgttgcggtgctgttgattaccacctcaacgcacaagagaagggattagctagagcaaggaataacatagatgcttggtggccagcaattgaagctggtgcagaggctatccttcaaactgcttcaggatgcggtgcatttgttaaggaatatggacagatgcttaaaaatgatgcattgtacgctgataaggcaagacaagtgagtgaacttgctgttgatttggtggagcttttgagagaagagcctcttgaaaaacttgctataagaggagataagaaattggcatttcattgtccatgcacacttcaacacgctcagaagttgaacggagaagttgagaaagtgctcttaagactcggtttcacattaaccgatgttcctgatagtcatctctgttgcggatctgctggtacttatgcattaacacaccctgatcttgctagacagttgagggataataagatgaacgctctcgaaagtggaaaacctgagatgattgttaccgctaatatcggttgtcaaactcatttggcatctgctggtaggacctctgtgaggcactggattgagatcgtggaacaggctcttgagaaggagtga

SEQ ID NO: 31 - DNA, 1688 bp, Artificial Sequence
  source: /note=“Description of Artificial Sequence Synthetic polynucleotide”
atggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcgacagctctagctctcctgtttcaacaaaacctcaaggtatattgatgatttaccaaatcttttccttgtcaaagttttgtgtttgactgtgtgggtttgaacctgttaggattcagtatgatatcaagtatgtgtcttttggaatacaaggatttacccttatggctatctttgttatctgtgtgaccttttctactttctcgctttgtaagatcgtctgagaatcattggagggcatttgaatgttgcagctgaagcaatgcaaactcagcttacagaagagatgagacaaaatgctagggcactcgaagctgattctatcttaagagcatgtgttcattgcggattctgtaccgctacttgccctacttatcaacttttgggagatgagcttgatggaccaagaggtagaatatacctcattaagcaagttttagaaggaaacgaggtgaccttgaaaactcaggaacatcttgatagatgcttgacatgtaggaattgcgagactacatgtccatcaggagttaggtatcacaacctcttagatatcggtagagatatagttgaacagaaggtgaaaagacctcttccagaaagaatactcagggagggattaagacaagttgtgcctaggccagctgtgtttagagcattgactcaagttggtcttgtgttgaggcctttccttccagaacaggttagagcaaagttgcctgctgaaacagtgaaggctaaaccaagacctccacttaggcataaaagaagggttctcatgttagagggatgtgctcagcctactttgtctccaaatacaaacgctgcaaccgctagagttcttgataggttgggtatttcagtgatgcctgcaaatgaggctggatgttgcggtgctgttgattaccacctcaacgcacaagagaagggattagctagagcaaggaataacatagatgcttggtggccagcaattgaagctggtgcagaggctatccttcaaactgcttcaggatgcggtgcatttgttaaggaatatggacagatgcttaaaaatgatgcattgtacgctgataaggcaagacaagtgagtgaacttgctgttgatttggtggagcttttgagagaagagcctcttgaaaaacttgctataagaggagataagaaattggcatttcattgtccatgcacacttcaacacgctcagaagttgaacggagaagttgagaaagtgctcttaagactcggtttcacattaaccgatgttcctgatagtcatctctgttgcggatctgctggtacttatgcattaacacaccctgatcttgctagacagttgagggataataagatgaacgctctcgaaagtggaaaacctgagatgattgttaccgctaatatcggttgtcaaactcatttggcatctgctggtaggacctctgtgaggcactggattgagatcgtggaacaggctcttgagaaggaggaacaaaaactcatctcagaagaggatctttga

SEQ ID NO: 32 - DNA, 1154 bp, Artificial Sequence
  source: /note=“Description of Artificial Sequence Synthetic polynucleotide”
atggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcgacagctctagctctcctgtttcaacaaaacctcaaggtatattgatgatttaccaaatcttttccttgtcaaagttttgtgtttgactgtgtgggtttgaacctgttaggattcagtatgatatcaagtatgtgtcttttggaatacaaggatttacccttatggctatctttgttatctgtgtgaccttttctactttctcgctttgtaagatcgtctgagaatcattggagggcatttgaatgttgcagctgaagcaatggcgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccttggccaacacttgtcactactttctcttatggtgttcaatgcttttcaagatacccagatcatatgaagcggcacgacttcttcaagagcgccatgcctgagggatacgtgcaggagaggaccatctctttcaaggacgacgggaactacaagacacgtgctgaagtcaagtttgagggagacaccctcgtcaacaggatcgagcttaagggaattgatttcaaggaggacggaaacatcctcggccacaagttggaatacaactacaactcccacaacgtatacatcacggcagacaaacaaaagaatggaatcaaagctaacttcaaaattagacacaacattgaagatggaagcgttcaactagcagaccattatcaacaaaatactcctattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 33 - DNA, 1937 bp, Artificial Sequence
  source: /note=“Description of Artificial Sequence Synthetic polynucleotide”
atggacagctctagctctcctgtttcaacaaaacctcaaggtatattgatgatttaccaaatcttttccttgtcaaagttttgtgtttgactgtgtgggtttgaacctgttaggattcagtatgatatcaagtatgtgtcttttggaatacaaggatttacccttatggctatctttgttatctgtgtgaccttttctactttctcgctttgtaagatcgtctgagaatcattggagggcatttgaatgttgcagctgaagcaatggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcatgtctattctttatgaagagagactcgatggagctttaccagatgttgatagaacctcagtgctcatggcattaagggaacatgttcctggacttgaaattcttcacacagatgaagagattatcccatatgaatgtgatggtttgtctgcttacagaactaggcctcttttggttgtgctcccaaagcagatggaacaggttacagctattcttgcagtgtgccatagattgagggttcctgttgtgacaagaggagctggtaccggactttcaggaggtgcactcccattagaaaagggtgttctcttagtgatggctaggttcaaagagatattggatattaatcctgtgggaagaagggctagagttcaaccaggtgtgaggaatctcgcaattagtcaggctgttgcacctcacaacctttattacgctcctgatccatcttcacaaatcgcatgttctataggtggtaatgtggctgaaaacgcaggaggtgttcattgccttaagtacggattgactgtgcacaaccttttgaaaatcgaagttcagactcttgatggagaggctcttacattgggtagtgatgcattggattctcctggttttgatctcttagctctcttcacaggttctgaaggaatgttaggtgttactacagaggttaccgttaaacttttgccaaaacctccagttgctagagtgctcttagcatcttttgattcagtggaaaaagctggacttgcagttggagatataattgctaacggaattattcctggaggtctcgaaatgatggataacttatctataagagctgctgaagatttcattcatgctggatatccagttgatgctgaggcaatacttttgtgtgaacttgatggtgttgagtcagatgtgcaagaagattgcgagagagttaatgatattctcttaaaggctggagcaactgatgtgaggttggctcaggatgaagcagagagagttaggttttgggctggaagaaaaaacgctttccctgctgttggtaggatctcaccagattattactgtatggatggtacaatacctagaagggctctcccaggagttttagagggtattgcaagacttagtcaacagtacgatttgagggttgctaatgtgtttcatgcaggagatggaaacatgcaccctctcatcttatttgatgctaatgagccaggagagttcgctagagcagaagagcttggaggaaagattcttgaactttgtgttgaagtgggaggtagtatctctggtgaacatggtattggaagagagaaaatcaatcaaatgtgcgctcagttcaactctgatgaaatcaccacttttcatgctgttaaggctgcattcgatcctgatggacttttgaatcctggaaagaatataccaacattgcacagatgcgctgagttcggagcaatgcacgttcaccacggacaccttccttttcctgagttggagagattctga

SEQ ID NO: 34 - DNA, 2543 bp, Artificial Sequence
  source: /note=“Description of Artificial Sequence Synthetic polynucleotide”
atggacagctctagctctcctgtttcaacaaaacctcaaggtatattgatgatttaccaaatcttttccttgtcaaagttttgtgtttgactgtgtgggtttgaacctgttaggattcagtatgatatcaagtatgtgtcttttggaatacaaggatttacccttatggctatctttgttatctgtgtgaccttttctactttctcgctttgtaagatcgtctgagaatcattggagggcatttgaatgttgcagctgaagcaatggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcatgctcagagaatgcgattattctcaggctcttttggagcaagtgaatcaggcaatttcagataagactcctcttgttatccaaggttctaactcaaaggcttttcttggtagaccagtgactggacagacacttgatgttagatgtcataggggtatcgtgaactacgatcctactgaattggttataacagctagagtgggaaccccacttgttactattgaagctgcattggagtctgctggtcaaatgctcccatgtgagcctccacactacggagaagaggcaacttggggtggtatggttgcttgcggacttgcaggtcctagaaggccatggagtggttctgttagagattttgtgttgggaacaaggattatcaccggagctggaaagcatctcagattcggaggtgaagttatgaaaaatgtggcaggttatgatctctcaaggttaatggttggaagttacggttgtcttggagtgttgacagaaatttctatgaaggttcttcctagaccaagggcttcacttagtttgagaagggaaatatctttgcaagaggctatgtcagaaattgcagagtggcaactccagcctttaccaattagtggattgtgctattttgataacgctctctggatcagattagaaggaggagagggttcagtgaaagctgcaagggaactcttaggaggtgaagaggttgctggacagttctggcaacagcttagagagcaacagttgcctttcttttctcttccaggtacattgtggaggataagtcttccttctgatgctccaatgatggatctccctggagaacaattaatcgattggggaggtgctcttagatggttgaagtcaacagcagaggataatcagatccatagaatagctaggaacgcaggaggtcacgctaccagattttcagcaggagatggaggtttcgctcctctcagtgcaccactttttagataccaccaacagttgaagcagcagttagatccttgtggtgtgttcaatcctggaagaatgtacgctgagttgtgaatgctcagagaatgcgattattctcaggctcttttggagcaagtgaatcaggcaatttcagataagactcctcttgttatccaaggttctaactcaaaggcttttcttggtagaccagtgactggacagacacttgatgttagatgtcataggggtatcgtgaactacgatcctactgaattggttataacagctagagtgggaaccccacttgttactattgaagctgcattggagtctgctggtcaaatgctcccatgtgagcctccacactacggagaagaggcaacttggggtggtatggttgcttgcggacttgcaggtcctagaaggccatggagtggttctgttagagattttgtgttgggaacaaggattatcaccggagctggaaagcatctcagattcggaggtgaagttatgaaaaatgtggcaggttatgatctctcaaggttaatggttggaagttacggttgtcttggagtgttgacagaaatttctatgaaggttcttcctagaccaagggcttcacttagtttgagaagggaaatatctttgcaagaggctatgtcagaaattgcagagtggcaactccagcctttaccaattagtggattgtgctattttgataacgctctctggatcagattagaaggaggagagggttcagtgaaagctgcaagggaactcttaggaggtgaagaggttgctggacagttctggcaacagcttagagagcaacagttgcctttcttttctcttccaggtacattgtggaggataagtcttccttctgatgctccaatgatggatctccctggagaacaattaatcgattggggaggtgctcttagatggttgaagtcaacagcagaggataatcagatccatagaatagctaggaacgcaggaggtcacgctaccagattttcagcaggagatggaggtttcgctcctctcagtgcaccactttttagataccaccaacagttgaagcagcagttagatccttgtggtgtgttcaatcctggaagaatgtacgctgagttgtga

SEQ ID NO: 35 - DNA, 1661 bp, Artificial Sequence
  source: /note=“Description of Artificial Sequence Synthetic polynucleotide”
atggacagctctagctctcctgtttcaacaaaacctcaaggtatattgatgatttaccaaatcttttccttgtcaaagttttgtgtttgactgtgtgggtttgaacctgttaggattcagtatgatatcaagtatgtgtcttttggaatacaaggatttacccttatggctatctttgttatctgtgtgaccttttctactttctcgctttgtaagatcgtctgagaatcattggagggcatttgaatgttgcagctgaagcaatggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcatgcaaactcagcttacagaagagatgagacaaaatgctagggcactcgaagctgattctatcttaagagcatgtgttcattgcggattctgtaccgctacttgccctacttatcaacttttgggagatgagcttgatggaccaagaggtagaatatacctcattaagcaagttttagaaggaaacgaggtgaccttgaaaactcaggaacatcttgatagatgcttgacatgtaggaattgcgagactacatgtccatcaggagttaggtatcacaacctcttagatatcggtagagatatagttgaacagaaggtgaaaagacctcttccagaaagaatactcagggagggattaagacaagttgtgcctaggccagctgtgtttagagcattgactcaagttggtcttgtgttgaggcctttccttccagaacaggttagagcaaagttgcctgctgaaacagtgaaggctaaaccaagacctccacttaggcataaaagaagggttctcatgttagagggatgtgctcagcctactttgtctccaaatacaaacgctgcaaccgctagagttcttgataggttgggtatttcagtgatgcctgcaaatgaggctggatgttgcggtgctgttgattaccacctcaacgcacaagagaagggattagctagagcaaggaataacatagatgcttggtggccagcaattgaagctggtgcagaggctatccttcaaactgcttcaggatgcggtgcatttgttaaggaatatggacagatgcttaaaaatgatgcattgtacgctgataaggcaagacaagtgagtgaacttgctgttgatttggtggagcttttgagagaagagcctcttgaaaaacttgctataagaggagataagaaattggcatttcattgtccatgcacacttcaacacgctcagaagttgaacggagaagttgagaaagtgctcttaagactcggtttcacattaaccgatgttcctgatagtcatctctgttgcggatctgctggtacttatgcattaacacaccctgatcttgctagacagttgagggataataagatgaacgctctcgaaagtggaaaacctgagatgattgttaccgctaatatcggttgtcaaactcatttggcatctgctggtaggacctctgtgaggcactggattgagatcgtggaacaggctcttgagaaggagtga

SEQ ID NO: 36 - DNA, 1691 bp, Artificial Sequence
  source: /note=“Description of Artificial Sequence Synthetic polynucleotide”
atggacagctctagctctcctgtttcaacaaaacctcaaggtatattgatgatttaccaaatcttttccttgtcaaagttttgtgtttgactgtgtgggtttgaacctgttaggattcagtatgatatcaagtatgtgtcttttggaatacaaggatttacccttatggctatctttgttatctgtgtgaccttttctactttctcgctttgtaagatcgtctgagaatcattggagggcatttgaatgttgcagctgaagcaatggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcatgcaaactcagcttacagaagagatgagacaaaatgctagggcactcgaagctgattctatcttaagagcatgtgttcattgcggattctgtaccgctacttgccctacttatcaacttttgggagatgagcttgatggaccaagaggtagaatatacctcattaagcaagttttagaaggaaacgaggtgaccttgaaaactcaggaacatcttgatagatgcttgacatgtaggaattgcgagactacatgtccatcaggagttaggtatcacaacctcttagatatcggtagagatatagttgaacagaaggtgaaaagacctcttccagaaagaatactcagggagggattaagacaagttgtgcctaggccagctgtgtttagagcattgactcaagttggtcttgtgttgaggcctttccttccagaacaggttagagcaaagttgcctgctgaaacagtgaaggctaaaccaagacctccacttaggcataaaagaagggttctcatgttagagggatgtgctcagcctactttgtctccaaatacaaacgctgcaaccgctagagttcttgataggttgggtatttcagtgatgcctgcaaatgaggctggatgttgcggtgctgttgattaccacctcaacgcacaagagaagggattagctagagcaaggaataacatagatgcttggtggccagcaattgaagctggtgcagaggctatccttcaaactgcttcaggatgcggtgcatttgttaaggaatatggacagatgcttaaaaatgatgcattgtacgctgataaggcaagacaagtgagtgaacttgctgttgatttggtggagcttttgagagaagagcctcttgaaaaacttgctataagaggagataagaaattggcatttcattgtccatgcacacttcaacacgctcagaagttgaacggagaagttgagaaagtgctcttaagactcggtttcacattaaccgatgttcctgatagtcatctctgttgcggatctgctggtacttatgcattaacacaccctgatcttgctagacagttgagggataataagatgaacgctctcgaaagtggaaaacctgagatgattgttaccgctaatatcggttgtcaaactcatttggcatctgctggtaggacctctgtgaggcactggattgagatcgtggaacaggctcttgagaaggaggaacaaaaactcatctcagaagaggatctttga

SEQ ID NO: 37 - DNA, 1157 bp, Artificial Sequence
  source: /note=“Description of Artificial Sequence Synthetic polynucleotide”
atggacagctctagctctcctgtttcaacaaaacctcaaggtatattgatgatttaccaaatcttttccttgtcaaagttttgtgtttgactgtgtgggtttgaacctgttaggattcagtatgatatcaagtatgtgtcttttggaatacaaggatttacccttatggctatctttgttatctgtgtgaccttttctactttctcgctttgtaagatcgtctgagaatcattggagggcatttgaatgttgcagctgaagcaatggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcatggcgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccttggccaacacttgtcactactttctcttatggtgttcaatgcttttcaagatacccagatcatatgaagcggcacgacttcttcaagagcgccatgcctgagggatacgtgcaggagaggaccatctctttcaaggacgacgggaactacaagacacgtgctgaagtcaagtttgagggagacaccctcgtcaacaggatcgagcttaagggaattgatttcaaggaggacggaaacatcctcggccacaagttggaatacaactacaactcccacaacgtatacatcacggcagacaaacaaaagaatggaatcaaagctaacttcaaaattagacacaacattgaagatggaagcgttcaactagcagaccattatcaacaaaatactcctattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 38 - DNA, 1683 bp, Artificial Sequence
  source: /note=“Description of Artificial Sequence Synthetic polynucleotide”
atggcttcctctgttatttcctctgccgctgttgctacacgcaccaatgttacacaagctggcagcatgattgcacctttcactggtctcaaatctgctgctactttccctgtttcaaggcttagagttctttctgctcatttgatcacttccattgctagcaatggtggaagagttaggtgcatgtctattctttatgaagagagactcgatggagctttaccagatgttgatagaacctcagtgctcatggcattaagggaacatgttcctggacttgaaattcttcacacagatgaagagattatcccatatgaatgtgatggtttgtctgcttacagaactaggcctcttttggttgtgctcccaaagcagatggaacaggttacagctattcttgcagtgtgccatagattgagggttcctgttgtgacaagaggagctggtaccggactttcaggaggtgcactcccattagaaaagggtgttctcttagtgatggctaggttcaaagagatattggatattaatcctgtgggaagaagggctagagttcaaccaggtgtgaggaatctcgcaattagtcaggctgttgcacctcacaacctttattacgctcctgatccatcttcacaaatcgcatgttctataggtggtaatgtggctgaaaacgcaggaggtgttcattgccttaagtacggattgactgtgcacaaccttttgaaaatcgaagttcagactcttgatggagaggctcttacattgggtagtgatgcattggattctcctggttttgatctcttagctctcttcacaggttctgaaggaatgttaggtgttactacagaggttaccgttaaacttttgccaaaacctccagttgctagagtgctcttagcatcttttgattcagtggaaaaagctggacttgcagttggagatataattgctaacggaattattcctggaggtctcgaaatgatggataacttatctataagagctgctgaagatttcattcatgctggatatccagttgatgctgaggcaatacttttgtgtgaacttgatggtgttgagtcagatgtgcaagaagattgcgagagagttaatgatattctcttaaaggctggagcaactgatgtgaggttggctcaggatgaagcagagagagttaggttttgggctggaagaaaaaacgctttccctgctgttggtaggatctcaccagattattactgtatggatggtacaatacctagaagggctctcccaggagttttagagggtattgcaagacttagtcaacagtacgatttgagggttgctaatgtgtttcatgcaggagatggaaacatgcaccctctcatcttatttgatgctaatgagccaggagagttcgctagagcagaagagcttggaggaaagattcttgaactttgtgttgaagtgggaggtagtatctctggtgaacatggtattggaagagagaaaatcaatcaaatgtgcgctcagttcaactctgatgaaatcaccacttttcatgctgttaaggctgcattcgatcctgatggacttttgaatcctggaaagaatataccaacattgcacagatgcgctgagttcggagcaatgcacgttcaccacggacaccttccttttcctgagttggagagattctga

SEQ ID NO: 39 - DNA, 2289 bp, Artificial Sequence
  source: /note=“Description of Artificial Sequence Synthetic polynucleotide”
atggcttcctctgttatttcctctgccgctgttgctacacgcaccaatgttacacaagctggcagcatgattgcacctttcactggtctcaaatctgctgctactttccctgtttcaaggcttagagttctttctgctcatttgatcacttccattgctagcaatggtggaagagttaggtgcatgctcagagaatgcgattattctcaggctcttttggagcaagtgaatcaggcaatttcagataagactcctcttgttatccaaggttctaactcaaaggcttttcttggtagaccagtgactggacagacacttgatgttagatgtcataggggtatcgtgaactacgatcctactgaattggttataacagctagagtgggaaccccacttgttactattgaagctgcattggagtctgctggtcaaatgctcccatgtgagcctccacactacggagaagaggcaacttggggtggtatggttgcttgcggacttgcaggtcctagaaggccatggagtggttctgttagagattttgtgttgggaacaaggattatcaccggagctggaaagcatctcagattcggaggtgaagttatgaaaaatgtggcaggttatgatctctcaaggttaatggttggaagttacggttgtcttggagtgttgacagaaatttctatgaaggttcttcctagaccaagggcttcacttagtttgagaagggaaatatctttgcaagaggctatgtcagaaattgcagagtggcaactccagcctttaccaattagtggattgtgctattttgataacgctctctggatcagattagaaggaggagagggttcagtgaaagctgcaagggaactcttaggaggtgaagaggttgctggacagttctggcaacagcttagagagcaacagttgcctttcttttctcttccaggtacattgtggaggataagtcttccttctgatgctccaatgatggatctccctggagaacaattaatcgattggggaggtgctcttagatggttgaagtcaacagcagaggataatcagatccatagaatagctaggaacgcaggaggtcacgctaccagattttcagcaggagatggaggtttcgctcctctcagtgcaccactttttagataccaccaacagttgaagcagcagttagatccttgtggtgtgttcaatcctggaagaatgtacgctgagttgtgaatgctcagagaatgcgattattctcaggctcttttggagcaagtgaatcaggcaatttcagataagactcctcttgttatccaaggttctaactcaaaggcttttcttggtagaccagtgactggacagacacttgatgttagatgtcataggggtatcgtgaactacgatcctactgaattggttataacagctagagtgggaaccccacttgttactattgaagctgcattggagtctgctggtcaaatgctcccatgtgagcctccacactacggagaagaggcaacttggggtggtatggttgcttgcggacttgcaggtcctagaaggccatggagtggttctgttagagattttgtgttgggaacaaggattatcaccggagctggaaagcatctcagattcggaggtgaagttatgaaaaatgtggcaggttatgatctctcaaggttaatggttggaagttacggttgtcttggagtgttgacagaaatttctatgaaggttcttcctagaccaagggcttcacttagtttgagaagggaaatatctttgcaagaggctatgtcagaaattgcagagtggcaactccagcctttaccaattagtggattgtgctattttgataacgctctctggatcagattagaaggaggagagggttcagtgaaagctgcaagggaactcttaggaggtgaagaggttgctggacagttctggcaacagcttagagagcaacagttgcctttcttttctcttccaggtacattgtggaggataagtcttccttctgatgctccaatgatggatctccctggagaacaattaatcgattggggaggtgctcttagatggttgaagtcaacagcagaggataatcagatccatagaatagctaggaacgcaggaggtcacgctaccagattttcagcaggagatggaggtttcgctcctctcagtgcaccactttttagataccaccaacagttgaagcagcagttagatccttgtggtgtgttcaatcctggaagaatgtacgctgagttgtga

SEQ ID NO: 40 - DNA, 1407 bp, Artificial Sequence
  source: /note=“Description of Artificial Sequence Synthetic polynucleotide”
atggcttcctctgttatttcctctgccgctgttgctacacgcaccaatgttacacaagctggcagcatgattgcacctttcactggtctcaaatctgctgctactttccctgtttcaaggcttagagttctttctgctcatttgatcacttccattgctagcaatggtggaagagttaggtgcatgcaaactcagcttacagaagagatgagacaaaatgctagggcactcgaagctgattctatcttaagagcatgtgttcattgcggattctgtaccgctacttgccctacttatcaacttttgggagatgagcttgatggaccaagaggtagaatatacctcattaagcaagttttagaaggaaacgaggtgaccttgaaaactcaggaacatcttgatagatgcttgacatgtaggaattgcgagactacatgtccatcaggagttaggtatcacaacctcttagatatcggtagagatatagttgaacagaaggtgaaaagacctcttccagaaagaatactcagggagggattaagacaagttgtgcctaggccagctgtgtttagagcattgactcaagttggtcttgtgttgaggcctttccttccagaacaggttagagcaaagttgcctgctgaaacagtgaaggctaaaccaagacctccacttaggcataaaagaagggttctcatgttagagggatgtgctcagcctactttgtctccaaatacaaacgctgcaaccgctagagttcttgataggttgggtatttcagtgatgcctgcaaatgaggctggatgttgcggtgctgttgattaccacctcaacgcacaagagaagggattagctagagcaaggaataacatagatgcttggtggccagcaattgaagctggtgcagaggctatccttcaaactgcttcaggatgcggtgcatttgttaaggaatatggacagatgcttaaaaatgatgcattgtacgctgataaggcaagacaagtgagtgaacttgctgttgatttggtggagcttttgagagaagagcctcttgaaaaacttgctataagaggagataagaaattggcatttcattgtccatgcacacttcaacacgctcagaagttgaacggagaagttgagaaagtgctcttaagactcggtttcacattaaccgatgttcctgatagtcatctctgttgcggatctgctggtacttatgcattaacacaccctgatcttgctagacagttgagggataataagatgaacgctctcgaaagtggaaaacctgagatgattgttaccgctaatatcggttgtcaaactcatttggcatctgctggtaggacctctgtgaggcactggattgagatcgtggaacaggctcttgagaaggagtga

SEQ ID NO: 41 - DNA, 1437 bp, Artificial Sequence
  source: /note=“Description of Artificial Sequence Synthetic polynucleotide”
atggcttcctctgttatttcctctgccgctgttgctacacgcaccaatgttacacaagctggcagcatgattgcacctttcactggtctcaaatctgctgctactttccctgtttcaaggcttagagttctttctgctcatttgatcacttccattgctagcaatggtggaagagttaggtgcatgcaaactcagcttacagaagagatgagacaaaatgctagggcactcgaagctgattctatcttaagagcatgtgttcattgcggattctgtaccgctacttgccctacttatcaacttttgggagatgagcttgatggaccaagaggtagaatatacctcattaagcaagttttagaaggaaacgaggtgaccttgaaaactcaggaacatcttgatagatgcttgacatgtaggaattgcgagactacatgtccatcaggagttaggtatcacaacctcttagatatcggtagagatatagttgaacagaaggtgaaaagacctcttccagaaagaatactcagggagggattaagacaagttgtgcctaggccagctgtgtttagagcattgactcaagttggtcttgtgttgaggcctttccttccagaacaggttagagcaaagttgcctgctgaaacagtgaaggctaaaccaagacctccacttaggcataaaagaagggttctcatgttagagggatgtgctcagcctactttgtctccaaatacaaacgctgcaaccgctagagttcttgataggttgggtatttcagtgatgcctgcaaatgaggctggatgttgcggtgctgttgattaccacctcaacgcacaagagaagggattagctagagcaaggaataacatagatgcttggtggccagcaattgaagctggtgcagaggctatccttcaaactgcttcaggatgcggtgcatttgttaaggaatatggacagatgcttaaaaatgatgcattgtacgctgataaggcaagacaagtgagtgaacttgctgttgatttggtggagcttttgagagaagagcctcttgaaaaacttgctataagaggagataagaaattggcatttcattgtccatgcacacttcaacacgctcagaagttgaacggagaagttgagaaagtgctcttaagactcggtttcacattaaccgatgttcctgatagtcatctctgttgcggatctgctggtacttatgcattaacacaccctgatcttgctagacagttgagggataataagatgaacgctctcgaaagtggaaaacctgagatgattgttaccgctaatatcggttgtcaaactcatttggcatctgctggtaggacctctgtgaggcactggattgagatcgtggaacaggctcttgagaaggaggaacaaaaactcatctcagaagaggatctttga

SEQ ID NO: 42 - DNA, 903 bp, Artificial Sequence
  source: /note=“Description of Artificial Sequence Synthetic polynucleotide”
atggcttcctctgttatttcctctgccgctgttgctacacgcaccaatgttacacaagctggcagcatgattgcacctttcactggtctcaaatctgctgctactttccctgtttcaaggcttagagttctttctgctcatttgatcacttccattgctagcaatggtggaagagttaggtgcatggcgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccttggccaacacttgtcactactttctcttatggtgttcaatgcttttcaagatacccagatcatatgaagcggcacgacttcttcaagagcgccatgcctgagggatacgtgcaggagaggaccatctctttcaaggacgacgggaactacaagacacgtgctgaagtcaagtttgagggagacaccctcgtcaacaggatcgagcttaagggaattgatttcaaggaggacggaaacatcctcggccacaagttggaatacaactacaactcccacaacgtatacatcacggcagacaaacaaaagaatggaatcaaagctaacttcaaaattagacacaacattgaagatggaagcgttcaactagcagaccattatcaacaaaatactcctattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 43 - protein, 523 aa, Artificial Sequence
  source: /note=“Description of Artificial Sequence Synthetic polypeptide”
MESGTGSSGDSSSSPVSTKPQAEAMSILYEERLDGALPDVDRTSVLMALREHVPGLEILHTDEEIIPYECDGLSAYRTRPLLVVLPKQMEQVTAILAVCHRLRVPVVTRGAGTGLSGGALPLEKGVLLVMARFKEILDINPVGRRARVQPGVRNLAISQAVAPHNLYYAPDPSSQIACSIGGNVAENAGGVHCLKYGLTVHNLLKIEVQTLDGEALTLGSDALDSPGFDLLALFTGSEGMLGVTTEVTVKLLPKPPVARVLLASFDSVEKAGLAVGDIIANGIIPGGLEMMDNLSIRAAEDFIHAGYPVDAEAILLCELDGVESDVQEDCERVNDILLKAGATDVRLAQDEAERVRFWAGRKNAFPAVGRISPDYYCMDGTIPRRALPGVLEGIARLSQQYDLRVANVFHAGDGNMHPLILFDANEPGEFARAEELGGKILELCVEVGGSISGEHGIGREKINQMCAQFNSDEITTFHAVKAAFDPDGLLNPGKNIPTLHRCAEFGAMHVHHGHLPFPELERF

SEQ ID NO: 44 - protein, 374 aa, Artificial Sequence
  source: /note=“Description of Artificial Sequence Synthetic polypeptide”
MESGTGSSGDSSSSPVSTKPQAEAMLRECDYSQALLEQVNQAISDKTPLVIQGSNSKAFLGRPVTGQTLDVRCHRGIVNYDPTELVITARVGTPLVTIEAALESAGQMLPCEPPHYGEEATWGGMVACGLAGPRRPWSGSVRDFVLGTRIITGAGKHLRFGGEVMKNVAGYDLSRLMVGSYGCLGVLTEISMKVLPRPRASLSLRREISLQEAMSEIAEWQLQPLPISGLCYFDNALWIRLEGGEGSVKAARELLGGEEVAGQFWQQLREQQLPFFSLPGTLWRISLPSDAPMMDLPGEQLIDWGGALRWLKSTAEDNQIHRIARNAGGHATRFSAGDGGFAPLSAPLFRYHQQLKQQLDPCGVFNPGRMYAEL

SEQ ID NO: 45 - protein, 431 aa, Artificial Sequence
  source: /note=“Description of Artificial Sequence Synthetic polypeptide”
MESGTGSSGDSSSSPVSTKPQAEAMQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKE

SEQ ID NO: 46 - protein, 441 aa, Artificial Sequence
  source: /note=“Description of Artificial Sequence Synthetic polypeptide”
MESGTGSSGDSSSSPVSTKPQAEAMQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKEEQKLISEEDL

SEQ ID NO: 47 - protein, 263 aa, Artificial Sequence
  source: /note=“Description of Artificial Sequence Synthetic polypeptide”
MESGTGSSGDSSSSPVSTKPQAEAMASKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 48 - protein, 540 aa, Artificial Sequence
  source: /note=“Description of Artificial Sequence Synthetic polypeptide”
MEVCSLARNLCFSLFSTHKSGTGSSGDSSSSPVSTKPQAEAMSILYEERLDGALPDVDRTSVLMALREHVPGLEILHTDEEIIPYECDGLSAYRTRPLLVVLPKQMEQVTAILAVCHRLRVPVVTRGAGTGLSGGALPLEKGVLLVMARFKEILDINPVGRRARVQPGVRNLAISQAVAPHNLYYAPDPSSQIACSIGGNVAENAGGVHCLKYGLTVHNLLKIEVQTLDGEALTLGSDALDSPGFDLLALFTGSEGMLGVTTEVTVKLLPKPPVARVLLASFDSVEKAGLAVGDIIANGIIPGGLEMMDNLSIRAAEDFIHAGYPVDAEAILLCELDGVESDVQEDCERVNDILLKAGATDVRLAQDEAERVRFWAGRKNAFPAVGRISPDYYCMDGTIPRRALPGVLEGIARLSQQYDLRVANVFHAGDGNMHPLILFDANEPGEFARAEELGGKILELCVEVGGSISGEHGIGREKINQMCAQFNSDEITTFHAVKAAFDPDGLLNPGKNIPTLHRCAEFGAMHVHHGHLPFPELERF

SEQ ID NO: 49 - protein, 391 aa, Artificial Sequence
  source: /note=“Description of Artificial Sequence Synthetic polypeptide”
MEVCSLARNLCFSLFSTHKSGTGSSGDSSSSPVSTKPQAEAMLRECDYSQALLEQVNQAISDKTPLVIQGSNSKAFLGRPVTGQTLDVRCHRGIVNYDPTELVITARVGTPLVTIEAALESAGQMLPCEPPHYGEEATWGGMVACGLAGPRRPWSGSVRDFVLGTRIITGAGKHLRFGGEVMKNVAGYDLSRLMVGSYGCLGVLTEISMKVLPRPRASLSLRREISLQEAMSEIAEWQLQPLPISGLCYFDNALWIRLEGGEGSVKAARELLGGEEVAGQFWQQLREQQLPFFSLPGTLWRISLPSDAPMMDLPGEQLIDWGGALRWLKSTAEDNQIHRIARNAGGHATRFSAGDGGFAPLSAPLFRYHQQLKQQLDPCGVFNPGRMYAEL

SEQ ID NO: 50 - protein, 448 aa, Artificial Sequence
  source: /note=“Description of Artificial Sequence Synthetic polypeptide”
MEVCSLARNLCFSLFSTHKSGTGSSGDSSSSPVSTKPQAEAMQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKE

SEQ ID NO: 51 - protein, 458 aa, Artificial Sequence
  source: /note=“Description of Artificial Sequence Synthetic polypeptide”
MEVCSLARNLCFSLFSTHKSGTGSSGDSSSSPVSTKPQAEAMQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKEEQKLISEEDL

SEQ ID NO: 52 - protein, 280 aa, Artificial Sequence
  source: /note=“Description of Artificial Sequence Synthetic polypeptide”
MEVCSLARNLCFSLFSTHKSGTGSSGDSSSSPVSTKPQAEAMASKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 53 - protein, 536 aa, Artificial Sequence
  source: /note=“Description of Artificial Sequence Synthetic polypeptide”
MESGTGSSGDSSSSPVSTKPQDRLRIIGGHLNVAAEAMSILYEERLDGALPDVDRTSVLMALREHVPGLEILHTDEEIIPYECDGLSAYRTRPLLVVLPKQMEQVTAILAVCHRLRVPVVTRGAGTGLSGGALPLEKGVLLVMARFKEILDINPVGRRARVQPGVRNLAISQAVAPHNLYYAPDPSSQIACSIGGNVAENAGGVHCLKYGLTVHNLLKIEVQTLDGEALTLGSDALDSPGFDLLALFTGSEGMLGVTTEVTVKLLPKPPVARVLLASFDSVEKAGLAVGDIIANGIIPGGLEMMDNLSIRAAEDFIHAGYPVDAEAILLCELDGVESDVQEDCERVNDILLKAGATDVRLAQDEAERVRFWAGRKNAFPAVGRISPDYYCMDGTIPRRALPGVLEGIARLSQQYDLRVANVFHAGDGNMHPLILFDANEPGEFARAEELGGKILELCVEVGGSISGEHGIGREKINQMCAQFNSDEITTFHAVKAAFDPDGLLNPGKNIPTLHRCAEFGAMHVHHGHLPFPELERF

SEQ ID NO: 54 - protein, 387 aa, Artificial Sequence
  source: /note=“Description of Artificial Sequence Synthetic polypeptide”
MESGTGSSGDSSSSPVSTKPQDRLRIIGGHLNVAAEAMLRECDYSQALLEQVNQAISDKTPLVIQGSNSKAFLGRPVTGQTLDVRCHRGIVNYDPTELVITARVGTPLVTIEAALESAGQMLPCEPPHYGEEATWGGMVACGLAGPRRPWSGSVRDFVLGTRIITGAGKHLRFGGEVMKNVAGYDLSRLMVGSYGCLGVLTEISMKVLPRPRASLSLRREISLQEAMSEIAEWQLQPLPISGLCYFDNALWIRLEGGEGSVKAARELLGGEEVAGQFWQQLREQQLPFFSLPGTLWRISLPSDAPMMDLPGEQLIDWGGALRWLKSTAEDNQIHRIARNAGGHATRFSAGDGGFAPLSAPLFRYHQQLKQQLDPCGVFNPGRMYAEL

SEQ ID NO: 55 - protein, 444 aa, Artificial Sequence
  source: /note=“Description of Artificial Sequence Synthetic polypeptide”
MESGTGSSGDSSSSPVSTKPQDRLRIIGGHLNVAAEAMQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKE

SEQ ID NO: 56 - protein, 454 aa, Artificial Sequence
  source: /note=“Description of Artificial Sequence Synthetic polypeptide”
MESGTGSSGDSSSSPVSTKPQDRLRIIGGHLNVAAEAMQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKEEQKLISEEDL

SEQ ID NO: 57 - protein, 276 aa, Artificial Sequence
  source: /note=“Description of Artificial Sequence Synthetic polypeptide”
MESGTGSSGDSSSSPVSTKPQDRLRIIGGHLNVAAEAMASKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 58 - protein, 553 aa, Artificial Sequence
  source: /note=“Description of Artificial Sequence Synthetic polypeptide”
MEVCSLARNLCFSLFSTHKSGTGSSGDSSSSPVSTKPQDRLRIIGGHLNVAAEAMSILYEERLDGALPDVDRTSVLMALREHVPGLEILHTDEEIIPYECDGLSAYRTRPLLVVLPKQMEQVTAILAVCHRLRVPVVTRGAGTGLSGGALPLEKGVLLVMARFKEILDINPVGRRARVQPGVRNLAISQAVAPHNLYYAPDPSSQIACSIGGNVAENAGGVHCLKYGLTVHNLLKIEVQTLDGEALTLGSDALDSPGFDLLALFTGSEGMLGVTTEVTVKLLPKPPVARVLLASFDSVEKAGLAVGDIIANGIIPGGLEMMDNLSIRAAEDFIHAGYPVDAEAILLCELDGVESDVQEDCERVNDILLKAGATDVRLAQDEAERVRFWAGRKNAFPAVGRISPDYYCMDGTIPRRALPGVLEGIARLSQQYDLRVANVFHAGDGNMHPLILFDANEPGEFARAEELGGKILELCVEVGGSISGEHGIGREKINQMCAQFNSDEITTFHAVKAAFDPDGLLNPGKNIPTLHRCAEFGAMHVHHGHLPFPELERF

SEQ ID NO: 59 - protein, 404 aa, Artificial Sequence
  source: /note=“Description of Artificial Sequence Synthetic polypeptide”
MEVCSLARNLCFSLFSTHKSGTGSSGDSSSSPVSTKPQDRLRIIGGHLNVAAEAMLRECDYSQALLEQVNQAISDKTPLVIQGSNSKAFLGRPVTGQTLDVRCHRGIVNYDPTELVITARVGTPLVTIEAALESAGQMLPCEPPHYGEEATWGGMVACGLAGPRRPWSGSVRDFVLGTRIITGAGKHLRFGGEVMKNVAGYDLSRLMVGSYGCLGVLTEISMKVLPRPRASLSLRREISLQEAMSEIAEWQLQPLPISGLCYFDNALWIRLEGGEGSVKAARELLGGEEVAGQFWQQLREQQLPFFSLPGTLWRISLPSDAPMMDLPGEQLIDWGGALRWLKSTAEDNQIHRIARNAGGHATRFSAGDGGFAPLSAPLFRYHQQLKQQLDPCGVFNPGRMYAEL

SEQ ID NO: 60 - protein, 461 aa, Artificial Sequence
  source: /note=“Description of Artificial Sequence Synthetic polypeptide”
MEVCSLARNLCFSLFSTHKSGTGSSGDSSSSPVSTKPQDRLRIIGGHLNVAAEAMQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKE

SEQ ID NO: 61 - protein, 471 aa, Artificial Sequence
  source: /note=“Description of Artificial Sequence Synthetic polypeptide”
MEVCSLARNLCFSLFSTHKSGTGSSGDSSSSPVSTKPQDRLRIIGGHLNVAAEAMQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKEEQKLISEEDL

SEQ ID NO: 62 - protein, 293 aa, Artificial Sequence
  source: /note=“Description of Artificial Sequence Synthetic polypeptide”
MEVCSLARNLCFSLFSTHKSGTGSSGDSSSSPVSTKPQDRLRIIGGHLNVAAEAMASKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 63 - protein, 524 aa, Artificial Sequence
  source: /note=“Description of Artificial Sequence Synthetic polypeptide”
MDSSSSPVSTKPQAEAMESGTGSSGMSILYEERLDGALPDVDRTSVLMALREHVPGLEILHTDEEIIPYECDGLSAYRTRPLLVVLPKQMEQVTAILAVCHRLRVPVVTRGAGTGLSGGALPLEKGVLLVMARFKEILDINPVGRRARVQPGVRNLAISQAVAPHNLYYAPDPSSQIACSIGGNVAENAGGVHCLKYGLTVHNLLKIEVQTLDGEALTLGSDALDSPGFDLLALFTGSEGMLGVTTEVTVKLLPKPPVARVLLASFDSVEKAGLAVGDIIANGIIPGGLEMMDNLSIRAAEDFIHAGYPVDAEAILLCELDGVESDVQEDCERVNDILLKAGATDVRLAQDEAERVRFWAGRKNAFPAVGRISPDYYCMDGTIPRRALPGVLEGIARLSQQYDLRVANVFHAGDGNMHPLILFDANEPGEFARAEELGGKILELCVEVGGSISGEHGIGREKINQMCAQFNSDEITTFHAVKAAFDPDGLLNPGKNIPTLHRCAEFGAMHVHHGHLPFPELERF

SEQ ID NO: 64 - protein, 375 aa, Artificial Sequence
  source: /note=“Description of Artificial Sequence Synthetic polypeptide”
MDSSSSPVSTKPQAEAMESGTGSSGMLRECDYSQALLEQVNQAISDKTPLVIQGSNSKAFLGRPVTGQTLDVRCHRGIVNYDPTELVITARVGTPLVTIEAALESAGQMLPCEPPHYGEEATWGGMVACGLAGPRRPWSGSVRDFVLGTRIITGAGKHLRFGGEVMKNVAGYDLSRLMVGSYGCLGVLTEISMKVLPRPRASLSLRREISLQEAMSEIAEWQLQPLPISGLCYFDNALWIRLEGGEGSVKAARELLGGEEVAGQFWQQLREQQLPFFSLPGTLWRISLPSDAPMMDLPGEQLIDWGGALRWLKSTAEDNQIHRIARNAGGHATRFSAGDGGFAPLSAPLFRYHQQLKQQLDPCGVFNPGRMYAEL

SEQ ID NO: 65 - protein, 432 aa, Artificial Sequence
  source: /note=“Description of Artificial Sequence Synthetic polypeptide”
MDSSSSPVSTKPQAEAMESGTGSSGMQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKE

SEQ ID NO: 66 - protein, 442 aa, Artificial Sequence
  source: /note=“Description of Artificial Sequence Synthetic polypeptide”
MDSSSSPVSTKPQAEAMESGTGSSGMQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKEEQKLISEEDL

SEQ ID NO: 67 - protein, 264 aa, Artificial Sequence
  source: /note=“Description of Artificial Sequence Synthetic polypeptide”
MDSSSSPVSTKPQAEAMESGTGSSGMASKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 68 - protein, 537 aa, Artificial Sequence
  source: /note=“Description of Artificial Sequence Synthetic polypeptide”
MDSSSSPVSTKPQDRLRIIGGHLNVAAEAMESGTGSSGMSILYEERLDGALPDVDRTSVLMALREHVPGLEILHTDEEIIPYECDGLSAYRTRPLLVVLPKQMEQVTAILAVCHRLRVPVVTRGAGTGLSGGALPLEKGVLLVMARFKEILDINPVGRRARVQPGVRNLAISQAVAPHNLYYAPDPSSQIACSIGGNVAENAGGVHCLKYGLTVHNLLKIEVQTLDGEALTLGSDALDSPGFDLLALFTGSEGMLGVTTEVTVKLLPKPPVARVLLASFDSVEKAGLAVGDIIANGIIPGGLEMMDNLSIRAAEDFIHAGYPVDAEAILLCELDGVESDVQEDCERVNDILLKAGATDVRLAQDEAERVRFWAGRKNAFPAVGRISPDYYCMDGTIPRRALPGVLEGIARLSQQYDLRVANVFHAGDGNMHPLILFDANEPGEFARAEELGGKILELCVEVGGSISGEHGIGREKINQMCAQFNSDEITTFHAVKAAFDPDGLLNPGKNIPTLHRCAEFGAMHVHHGHLPFPELERF

SEQ ID NO: 69 - protein, 388 aa, Artificial Sequence
  source: /note=“Description of Artificial Sequence Synthetic polypeptide”
MDSSSSPVSTKPQDRLRIIGGHLNVAAEAMESGTGSSGMLRECDYSQALLEQVNQAISDKTPLVIQGSNSKAFLGRPVTGQTLDVRCHRGIVNYDPTELVITARVGTPLVTIEAALESAGQMLPCEPPHYGEEATWGGMVACGLAGPRRPWSGSVRDFVLGTRIITGAGKHLRFGGEVMKNVAGYDLSRLMVGSYGCLGVLTEISMKVLPRPRASLSLRREISLQEAMSEIAEWQLQPLPISGLCYFDNALWIRLEGGEGSVKAARELLGGEEVAGQFWQQLREQQLPFFSLPGTLWRISLPSDAPMMDLPGEQLIDWGGALRWLKSTAEDNQIHRIARNAGGHATRFSAGDGGFAPLSAPLFRYHQQLKQQLDPCGVFNPGRMYAEL

SEQ ID NO: 70 - protein, 445 aa, Artificial Sequence
  source: /note=“Description of Artificial Sequence Synthetic polypeptide”
MDSSSSPVSTKPQDRLRIIGGHLNVAAEAMESGTGSSGMQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKE

SEQ ID NO: 71 - protein, 455 aa, Artificial Sequence
  source: /note=“Description of Artificial Sequence Synthetic polypeptide”
MDSSSSPVSTKPQDRLRIIGGHLNVAAEAMESGTGSSGMQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKEEQKLISEEDL

SEQ ID NO: 72 - protein, 277 aa, Artificial Sequence
  source: /note=“Description of Artificial Sequence Synthetic polypeptide”
MDSSSSPVSTKPQDRLRIIGGHLNVAAEAMESGTGSSGMASKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 73 - protein, 541 aa, Artificial Sequence
  source: /note=“Description of Artificial Sequence Synthetic polypeptide”
MDSSSSPVSTKPQAEAMEVCSLARNLCFSLFSTHKSGTGSSGMSILYEERLDGALPDVDRTSVLMALREHVPGLEILHTDEEIIPYECDGLSAYRTRPLLVVLPKQMEQVTAILAVCHRLRVPVVTRGAGTGLSGGALPLEKGVLLVMARFKEILDINPVGRRARVQPGVRNLAISQAVAPHNLYYAPDPSSQIACSIGGNVAENAGGVHCLKYGLTVHNLLKIEVQTLDGEALTLGSDALDSPGFDLLALFTGSEGMLGVTTEVTVKLLPKPPVARVLLASFDSVEKAGLAVGDIIANGIIPGGLEMMDNLSIRAAEDFIHAGYPVDAEAILLCELDGVESDVQEDCERVNDILLKAGATDVRLAQDEAERVRFWAGRKNAFPAVGRISPDYYCMDGTIPRRALPGVLEGIARLSQQYDLRVANVFHAGDGNMHPLILFDANEPGEFARAEELGGKILELCVEVGGSISGEHGIGREKINQMCAQFNSDEITTFHAVKAAFDPDGLLNPGKNIPTLHRCAEFGAMHVHHGHLPFPELERF

SEQ ID NO: 74 - protein, 392 aa, Artificial Sequence
  source: /note=“Description of Artificial Sequence Synthetic polypeptide”
MDSSSSPVSTKPQAEAMEVCSLARNLCFSLFSTHKSGTGSSGMLRECDYSQALLEQVNQAISDKTPLVIQGSNSKAFLGRPVTGQTLDVRCHRGIVNYDPTELVITARVGTPLVTIEAALESAGQMLPCEPPHYGEEATWGGMVACGLAGPRRPWSGSVRDFVLGTRIITGAGKHLRFGGEVMKNVAGYDLSRLMVGSYGCLGVLTEISMKVLPRPRASLSLRREISLQEAMSEIAEWQLQPLPISGLCYFDNALWIRLEGGEGSVKAARELLGGEEVAGQFWQQLREQQLPFFSLPGTLWRISLPSDAPMMDLPGEQLIDWGGALRWLKSTAEDNQIHRIARNAGGHATRFSAGDGGFAPLSAPLFRYHQQLKQQLDPCGVFNPGRMYAEL

SEQ ID NO: 75 - protein, 449 aa, Artificial Sequence
  source: /note=“Description of Artificial Sequence Synthetic polypeptide”
MDSSSSPVSTKPQAEAMEVCSLARNLCFSLFSTHKSGTGSSGMQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKE

SEQ ID NO: 76 - protein, 459 aa, Artificial Sequence
  source: /note=“Description of Artificial Sequence Synthetic polypeptide”
MDSSSSPVSTKPQAEAMEVCSLARNLCFSLFSTHKSGTGSSGMQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKEEQKLISEEDL

SEQ ID NO: 77 - protein, 281 aa, Artificial Sequence
  source: /note=“Description of Artificial Sequence Synthetic polypeptide”
MDSSSSPVSTKPQAEAMEVCSLARNLCFSLFSTHKSGTGSSGMASKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 78 - protein, 554 aa, Artificial Sequence
  source: /note=“Description of Artificial Sequence Synthetic polypeptide”
MDSSSSPVSTKPQDRLRIIGGHLNVAAEAMEVCSLARNLCFSLFSTHKSGTGSSGMSILYEERLDGALPDVDRTSVLMALREHVPGLEILHTDEEIIPYECDGLSAYRTRPLLVVLPKQMEQVTAILAVCHRLRVPVVTRGAGTGLSGGALPLEKGVLLVMARFKEILDINPVGRRARVQPGVRNLAISQAVAPHNLYYAPDPSSQIACSIGGNVAENAGGVHCLKYGLTVHNLLKIEVQTLDGEALTLGSDALDSPGFDLLALFTGSEGMLGVTTEVTVKLLPKPPVARVLLASFDSVEKAGLAVGDIIANGIIPGGLEMMDNLSIRAAEDFIHAGYPVDAEAILLCELDGVESDVQEDCERVNDILLKAGATDVRLAQDEAERVRFWAGRKNAFPAVGRISPDYYCMDGTIPRRALPGVLEGIARLSQQYDLRVANVFHAGDGNMHPLILFDANEPGEFARAEELGGKILELCVEVGGSISGEHGIGREKINQMCAQFNSDEITTFHAVKAAFDPDGLLNPGKNIPTLHRCAEFGAMHVHHGHLPFPELERF

SEQ ID NO: 79 - protein, 405 aa, Artificial Sequence
  source: /note=“Description of Artificial Sequence Synthetic polypeptide”
MDSSSSPVSTKPQDRLRIIGGHLNVAAEAMEVCSLARNLCFSLFSTHKSGTGSSGMLRECDYSQALLEQVNQAISDKTPLVIQGSNSKAFLGRPVTGQTLDVRCHRGIVNYDPTELVITARVGTPLVTIEAALESAGQMLPCEPPHYGEEATWGGMVACGLAGPRRPWSGSVRDFVLGTRIITGAGKHLRFGGEVMKNVAGYDLSRLMVGSYGCLGVLTEISMKVLPRPRASLSLRREISLQEAMSEIAEWQLQPLPISGLCYFDNALWIRLEGGEGSVKAARELLGGEEVAGQFWQQLREQQLPFFSLPGTLWRISLPSDAPMMDLPGEQLIDWGGALRWLKSTAEDNQIHRIARNAGGHATRFSAGDGGFAPLSAPLFRYHQQLKQQLDPCGVFNPGRMYAEL

SEQ ID NO: 80 - protein, 462 aa, Artificial Sequence
  source: /note=“Description of Artificial Sequence Synthetic polypeptide”
MDSSSSPVSTKPQDRLRIIGGHLNVAAEAMEVCSLARNLCFSLFSTHKSGTGSSGMQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKE

SEQ ID NO: 81 - protein, 472 aa, Artificial Sequence
  source: /note=“Description of Artificial Sequence Synthetic polypeptide”
MDSSSSPVSTKPQDRLRIIGGHLNVAAEAMEVCSLARNLCFSLFSTHKSGTGSSGMQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKEEQKLISEEDL

SEQ ID NO: 82 - protein, 294 aa, Artificial Sequence
  source: /note=“Description of Artificial Sequence Synthetic polypeptide”
MDSSSSPVSTKPQDRLRIIGGHLNVAAEAMEVCSLARNLCFSLFSTHKSGTGSSGMASKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 83 - protein, 560 aa, Artificial Sequence
  source: /note=“Description of Artificial Sequence Synthetic polypeptide”
MASSVISSAAVATRTNVTQAGSMIAPFTGLKSAATFPVSRLRVLSAHLITSIASNGGRVRCMSILYEERLDGALPDVDRTSVLMALREHVPGLEILHTDEEIIPYECDGLSAYRTRPLLVVLPKQMEQVTAILAVCHRLRVPVVTRGAGTGLSGGALPLEKGVLLVMARFKEILDINPVGRRARVQPGVRNLAISQAVAPHNLYYAPDPSSQIACSIGGNVAENAGGVHCLKYGLTVHNLLKIEVQTLDGEALTLGSDALDSPGFDLLALFTGSEGMLGVTTEVTVKLLPKPPVARVLLASFDSVEKAGLAVGDIIANGIIPGGLEMMDNLSIRAAEDFIHAGYPVDAEAILLCELDGVESDVQEDCERVNDILLKAGATDVRLAQDEAERVRFWAGRKNAFPAVGRISPDYYCMDGTIPRRALPGVLEGIARLSQQYDLRVANVFHAGDGNMHPLILFDANEPGEFARAEELGGKILELCVEVGGSISGEHGIGREKINQMCAQFNSDEITTFHAVKAAFDPDGLLNPGKNIPTLHRCAEFGAMHVHHGHLPFPELERF

SEQ ID NO: 84 - protein, 411 aa, Artificial Sequence
  source: /note=“Description of Artificial Sequence Synthetic polypeptide”
MASSVISSAAVATRTNVTQAGSMIAPFTGLKSAATFPVSRLRVLSAHLITSIASNGGRVRCMLRECDYSQALLEQVNQAISDKTPLVIQGSNSKAFLGRPVTGQTLDVRCHRGIVNYDPTELVITARVGTPLVTIEAALESAGQMLPCEPPHYGEEATWGGMVACGLAGPRRPWSGSVRDFVLGTRIITGAGKHLRFGGEVMKNVAGYDLSRLMVGSYGCLGVLTEISMKVLPRPRASLSLRREISLQEAMSEIAEWQLQPLPISGLCYFDNALWIRLEGGEGSVKAARELLGGEEVAGQFWQQLREQQLPFFSLPGTLWRISLPSDAPMMDLPGEQLIDWGGALRWLKSTAEDNQIHRIARNAGGHATRFSAGDGGFAPLSAPLFRYHQQLKQQLDPCGVFNPGRMYAEL

SEQ ID NO: 85 - protein, 468 aa, Artificial Sequence
  source: /note=“Description of Artificial Sequence Synthetic polypeptide”
MASSVISSAAVATRTNVTQAGSMIAPFTGLKSAATFPVSRLRVLSAHLITSIASNGGRVRCMQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKE

SEQ ID NO: 86 - protein, 478 aa, Artificial Sequence
  source: /note=“Description of Artificial Sequence Synthetic polypeptide”
MASSVISSAAVATRTNVTQAGSMIAPFTGLKSAATFPVSRLRVLSAHLITSIASNGGRVRCMQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKEEQKLISEEDL

SEQ ID NO: 87 - protein, 300 aa, Artificial Sequence
  source: /note=“Description of Artificial Sequence Synthetic polypeptide”
MASSVISSAAVATRTNVTQAGSMIAPFTGLKSAATFPVSRLRVLSAHLITSIASNGGRVRCMASKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 88 - DNA, 5388 bp, Artificial Sequence
  source: /note=“Description of Artificial Sequence Synthetic polynucleotide”
ggcccccagcaggaggcccgcacgacgggctattagctcagtggtagagcgcgcccctgataattgcgtcgttgtgcctgggctgtgagggctctcagccacatggatagttcaatgtgctcatcagcgcctgaccctgagatgtggatcatccaaggcacattagcatggcgtactcctcctgttcgaaccggggtttgaaaccaaacttctcctcaggaggatagatggggcgattcaggtgagatccaatgtagatccaactttctattcactcgtgggatccgggcggtccggaggggaccactatggctcctctcttctcgagaatccatacatcccttatcagtgtatggacagctatctctcgagcgcaggtttaggttcggcctcaatgggaaaataaaatggagcacctaacaacgtatcttcacagaccaagaactacgagatcacccctttcattctggggtgacggagggatcgtaccgttcgagcctttttttcatgttatctatctcttgactcgaaatgggagcaggtttgaaaaaggatcttagagtgtctagggttaggccagtagggtctcttaacgccctcttttttcttctcatcgaagttatttcacaaatacttcctatggtaaggaagagggggggaacaagcacacttggagagcgcagtacaacggagagttgtatgctgcgttcgggaaggatgaatcgctcccgaaaaggaatctattgattctctcccaattggttggaccataggtgcgatgatttacttcacgggcgaggtctctggttcaaatccaggatggcccagctgcggctccctcgctgtgatcgaataagaatggataagaggctcgtgggattgacgtgagggggtaggggtagctatatttctgggagcgaactccatgcgaatatgaagcgcatggatacaagttatgacttggaatgaaagacaattccgaatcgaattcgcatgcctgcaggtcgactctagaggatccccgggtaccgagctcgaattaggaggaattaataatgattgaacaagatggattgcacgcaggttctccggccgcttgggtggagaggctattcggctatgactgggcacaacagacaatcggctgctctgatgccgccgtgttccggctgtcagcgcaggggaggccggttctttttgtcaagaccgacctgtccggtgccctgaatgaacttcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggcgcgccttcgttagtgttagtctagaactagtttagtaaaaaacgagcaatataagccttctttaaataagaaagagggcttatattactcgtttttttctataaaaatgagcaaatttttatagagtatcatattttactttatttattatattaataataaataataataataaataataaaaaattactatatattttttattagaaaaaaaataaggtggaatttgctacctttttttattttttattgaaatttgtatttttttttttttttagacaatacaaaaaagaatagatagtagcgtaggggctccacttggctcgggggatatagctcagttggtagagctccgctcttgcaattgggtcgttgcgattacgggttgggtgtctaattgtccaggcggtaatgatagtatcttgtacctgaaccggtggctcactttttctaagtaatggggaaaaggaccgaaacatgccactgaaagactctactgagacaaagatgggctgtcaagaacgtagaggaggtaggatggtcagttggtcagatctagtatggatcgtacatggacggtagttggagtcggcggctctcctagggttccctcgtctgggattgatccctggggaagaggatcaagttggcccttgcgaacagcttgatgcactatctcccttcaaccctttgagcgaaatgcggcaaaaggaaggaaaatccatggaccgaccccatcgtctccaccccgtaggaactacgagatcaccccaaggacgccttcggtatccaggggtcgcggaccgaccatagaaccctgttcaataagtggaatgcattagctgtccgctcgcaggttgggcagtaagggtcggagaagggcaatcactcattcttaaaaccagcattcgaaagagttggggcggaaaagggggggaaagctctccgttcctggttctcctgtagctggatcctctagaaccacaagaatccttagttggaatgggattccagctcatcaccttttgagattttgagaagagttgctctttggagagcacagtacgatgaaagttgtaagctgtgttcgggggggagttcttgtctatcgttggcctctatggtagaatcagtcaggggcctgataggcggtggtttaccctgtggcggatgtcagcggttcgagtccgcttatctccaactcgtgaacttagccgatacaaagctatatgatagcacccaatttttccgattcggcacactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 89 - DNA, 18922 bp, Artificial Sequence
  source: /note=“Description of Artificial Sequence Synthetic polynucleotide”
ggcccccagcaggaggcccgcacgacgggctattagctcagtggtagagcgcgcccctgataattgcgtcgttgtgcctgggctgtgagggctctcagccacatggatagttcaatgtgctcatcagcgcctgaccctgagatgtggatcatccaaggcacattagcatggcgtactcctcctgttcgaaccggggtttgaaaccaaacttctcctcaggaggatagatggggcgattcaggtgagatccaatgtagatccaactttctattcactcgtgggatccgggcggtccggaggggaccactatggctcctctcttctcgagaatccatacatcccttatcagtgtatggacagctatctctcgagcgcaggtttaggttcggcctcaatgggaaaataaaatggagcacctaacaacgtatcttcacagaccaagaactacgagatcacccctttcattctggggtgacggagggatcgtaccgttcgagcctttttttcatgttatctatctcttgactcgaaatgggagcaggtttgaaaaaggatcttagagtgtctagggttaggccagtagggtctcttaacgccctcttttttcttctcatcgaagttatttcacaaatacttcctatggtaaggaagagggggggaacaagcacacttggagagcgcagtacaacggagagttgtatgctgcgttcgggaaggatgaatcgctcccgaaaaggaatctattgattctctcccaattggttggaccataggtgcgatgatttacttcacgggcgaggtctctggttcaaatccaggatggcccagctgcggctccctcgctgtgatcgaataagaatggataagaggctcgtgggattgacgtgagggggtaggggtagctatatttctgggagcgaactccatgcgaatatgaagcgcatggatacaagttatgacttggaatgaaagacaattccgaatcgaattcagatctaggaggattatattatgggcgagcagaagctgattagcgaggaagacctaagcggcacaggtcgtctggcaggcaaaattgcactgatcacgggcggtgcgggcaacatcggtagcgaactgacccgtcgctttctggccgaaggtgcaacggtgattatctctggccgtaatcgcgccaaactgaccgcactggcggaacgtatgcaggccgaagcaggtgtgcctgcgaaacgcattgatctggaagttatggatggtagtgatccggtggcggttcgtgctggcattgaagcaatcgtggcgcgccacggtcagattgatatcctggttaacaatgcaggcagcgcaggagcacagcgtcgcctggcagaaattccgctgaccgaagcagaactgggtccgggtgcagaagaaacgctgcacgccagtatcgcaaacctgctgggcatgggttggcatctgatgcgtattgcagcaccgcacatgccggttggcagcgcagtgatcaatgttagcaccattttctctcgtgcggaatattacggtcgcattccgtatgtgacgccgaaagcagcgctgaacgccctgtctcagctggcagcacgcgaactgggagcacgtggtatccgcgttaataccatttttccgggtccgatcgaaagtgatcgtattcgcacggtgttccagcgtatggatcagctgaaaggccgcccggaaggtgataccgcccatcactttctgaacacgatgcgtctgtgccgcgcgaatgatcagggagccctggaacgtcgctttccgagcgtgggtgatgttgcggatgcagccgttttcctggcatctgcggaaagtgcagcgctgtctggtgaaaccatcgaagtgacgcatggcatggaactgccagcgtgtagcgaaacctctctgctggcccgcaccgatctgcgtactattgatgccagcggtcgtaccacgctgatctgcgcaggtgatcagattgaagaagttatggcgctgaccggaatgcttcgtacttgtggtagcgaagtgattatcggctttcgctctgccgcagcgctggcacagttcgaacaggcagtgaacgaaagccgtcgcctggcaggtgcagattttaccccgccgatcgcactgccgctggacccgcgtgatccggcgacgattgatgccgttttcgattggggcgcgggtgaaaacaccggcggtatccatgccgcagtgattctgccagcaacgagccacgaaccggcaccgtgcgtgatcgaagttgatgatgagcgcgttctgaatttcctggcggatgaaattacaggtacgattgtgatcgcaagtcgtctggcgcgctattggcagagccagcgtctgaccccaggtgcgcgtgcccgtggtccgcgcgttatctttctgagtaacggcgcggatcagaacggcaatgtgtatggtcgtattcagagcgcggccattggtcagctgatccgtgtttggcgtcatgaagccgaactggattaccagcgcgcaagcgcagcaggtgatcacgtgctgccgccggtttgggcgaaccagattgtgcgctttgccaatcgttctctggaaggtctggaatttgcttgcgcgtggaccgcgcagctgctgcatagtcagcgtcacattaatgaaatcacgctgaacattccggccaatatctctgcaaccacgggagcacgcagtgcaagcgttggttgggcggaaagtctgatcggcctgcatctgggtaaagtggcactgattaccggcggtagcgcgggtattggcggtcagatcggtcgtctgctggcactgtctggtgcccgcgttatgctggcagcacgtgatcgccataaactggaacagatgcaggccatgatccagagcgaactggcagaagtgggctataccgatgtggaagatcgtgttcacattgccccaggttgtgatgtgagttctgaagcacagctggcggatctggttgaacgcaccctgtctgcgtttggcacggtggattatctgatcaacaatgcaggcattgccggtgtggaagaaatggttatcgatatgccggttgaaggttggcgtcataccctgttcgccaacctgattagtaattacagcctgatgcgcaaactggcaccgctgatgaaaaaacagggctctggttatatcctgaacgtgagtagctactttggcggtgaaaaagatgcggccattccgtatccgaatcgtgcggattacgccgttagtaaagcgggccagcgtgcgatggcagaagtgtttgcccgcttcctgggtccagaaattcagatcaatgcgattgcaccgggtccggtggaaggtgatcgtctgcgtggtacaggtgaacgtccgggtctgtttgcacgtcgcgcacgtctgatcctggaaaacaaacgcctgaatgaactgcacgcagctctgattgccgcagcgcgcaccgatgaacgtagtatgcacgaactggtggaactgctgctgccgaacgatgttgcagcactggaacagaatccggcagcaccgaccgcactgcgtgaactggcacgtcgcttccgcagtgaaggtgatccggcagcgtctagtagctctgccctgctgaaccgtagcatcgccgcaaaactgctggcgcgcctgcataatggcggttatgttctgccagcagatatttttgcgaacctgccgaatccgccggacccgtttttcacccgtgcgcagatcgatcgtgaagcccgcaaagtgcgtgatggcattatgggtatgctgtacctgcaacgtatgccgaccgaatttgatgtggcaatggcgacggtttattacctggcggatcgcaacgtttctggcgaaacctttcacccgagtggcggtctgcgctatgaacgtaccccgacgggcggtgaactgttcggtctgccgtctccggaacgtctggcggaactggtgggtagtaccgtttacctgattggcgaacatctgacggaacacctgaatctgctggcccgcgcatatctggaacgctacggagcccgtcaggtggttatgatcgtggaaaccgaaacgggtgcggaaaccatgcgtcgcctgctgcatgatcacgtggaagcaggtcgcctgatgacgattgttgcgggcgatcagattgaagcggccatcgatcaggcgattacccgctatggtcgtccgggtccggtggtttgcaccccgtttcgcccgctgccgacggtgccgctggttggccgtaaagattctgattggagtaccgtgctgagcgaagccgaatttgcagaactgtgtgaacatcagctgacccatcacttccgcgttgcccgtaaaattgcactgagtgatggtgcaagcctggcactggtgaccccggaaaccacggcaacgagcaccacggaacagtttgcgctggccaacttcatcaaaaccacgctgcacgcattcaccgcgacgattggtgttgaatctgaacgcaccgcgcagcgtattctgatcaatcaggtggatctgactcgtcgcgcacgcgcggaagaaccgcgtgatccgcatgaacgccagcaggaactggaacgttttattgaagcagtgctgctggttaccgcaccgctgccgccggaagcagatactcgttacgcaggtcgtatccaccgtggtcgcgcgattaccgtgtaaggatctaggaggattatattatgatcgataccgcaccgctggcaccgccgcgtgctccgcgcagcaatccgattcgtgatcgcgtggattgggaagcgcagcgtgcagcagcactggccgatccgggtgcatttcatggtgcgatcgcccgtaccgttattcactggtatgatccgcagcatcactgctggattcgcttcaacgaaagctctcagcgttgggaaggtctggatgcagcaacgggtgctccggttacagtggattatcctgccgattaccagccgtggcagcaggcatttgatgatagtgaagcgccgttttatcgctggttcagcggcggtctgacgaacgcatgttttaatgaagttgatcgtcacgtgacaatgggttacggcgatgaagtggcgtattacttcgaaggtgatcgctgggataatagcctgaacaatggccgtggcggtccggtggttcaggaaacgattacccgtcgccgtctgctggttgaagtggttaaagcagcgcaggttctgcgcgatctgggcctgaaaaaaggtgatcgtatcgcgctgaacatgccgaatatcatgccgcagatttattacaccgaagccgcaaaacgcctgggtattctgtatacgccggtgtttggcggtttcagtgataaaaccctgagcgatcgcatccataatgcaggtgcgcgtgtggttattacctctgatggcgcgtatcgtaacgcccaggtggttccgtataaagaagcctacacggatcaggcactggataaatacatcccggtggaaaccgcccaggcaattgttgcacagacgctggcaaccctgccgctgaccgaaagtcagcgccagacgattatcaccgaagtggaagcagcactggcaggtgaaattacggttgaacgttctgatgttatgcgcggtgtgggcagtgcgctggccaaactgcgcgatctggatgccagtgtgcaggcaaaagttcgtaccgtgctggcacaggcgctggttgaaagcccgccgcgcgtggaagcagtggttgtggttcgtcatacgggtcaggaaatcctgtggaatgaaggccgtgatcgctggagccacgatctgctggatgcagcactggcgaaaattctggctaacgcacgcgccgcaggttttgatgttcactctgaaaacgatctgctgaatctgccggatgatcagctgatccgtgctctgtatgcgagtattccgtgcgaaccagttgatgccgaatatccgatgtttattatctacacgagcggttctaccggcaaaccgaaaggtgttattcatgttcacggcggttacgtggcgggcgtggttcataccctgcgcgttagtttcgatgccgaaccgggcgatacgatttatgtgatcgcagatccgggctggatcacaggtcagagctacatgctgacggcaaccatggcaggtcgtctgactggtgtgattgccgaaggttctccgctgtttccgagtgcgggccgctatgcctctattatcgaacgttacggtgttcagatttttaaagcgggcgttacgttcctgaaaaccgtgatgagtaacccgcagaatgttgaagatgtgcgcctgtatgatatgcacagtctgcgtgtggcaaccttttgtgcagagccggttagcccggcagtgcagcagttcggtatgcagatcatgacgccgcagtatattaatagctactgggcgacggaacatggcggtattgtgtggacccacttttatggcaaccaggatttcccgctgcgtccagatgcacatacgtacccgctgccgtgggttatgggtgatgtttgggtggcagaaaccgatgaatctggcaccacgcgctatcgcgtggcggatttcgatgaaaaaggtgaaatcgttatcaccgcaccgtatccgtacctgacgcgaaccctgtggggtgatgtgccgggttttgaagcgtatctgcgtggtgaaatcccgctgcgtgcatggaaaggtgatgcagaacgtttcgttaaaacctactggcgtcgtggtccgaatggcgaatggggttatatccagggcgattttgcgattaaatacccggatggtagtttcacgctgcatggccgcagcgatgatgttattaatgtgtccggccaccgtatgggtacggaagaaatcgaaggtgccattctgcgtgatcgccagatcaccccggattctccggtgggtaactgcattgtggttggcgcgccgcatcgtgaaaaaggcctgaccccggttgcatttatccagccagcaccgggtcgtcacctgacgggtgcagatcgccgtcgcctggatgaactggtgcgtaccgaaaaaggtgcagttagcgtgccggaagattatattgaagttagtgcgtttccggaaacccgcagcggtaaatacatgcgtcgcttcctgcgtaatatgatgctggatgaaccgctgggcgataccacgaccctgcgcaacccggaagtgctggaagaaatcgcggccaaaattgccgaatggaaacgtcgccagcgcatggcagaagaacagcagattatcgaacgttatcgctactttcgtattgaatatcatccgccgaccgcaagtgcaggtaaactggcagtggttacggttaccaatccgccggtgaacgccctgaatgaacgtgctctggatgaactgaacaccatcgtggatcacctggcgcgtcgccaggatgttgcagcgattgtgtttacgggtcagggtgctcgcagcttcgtggccggtgcggatatccgtcagctgctggaagaaattcataccgttgaagaagccatggcactgccgaacaatgcgcacctggcctttcgcaaaattgaacgtatgaacaaaccgtgcattgccgcaatcaatggtgtggcactgggcggtggcctggaatttgcgatggcctgtcattatcgcgttgccgatgtgtacgcagaatttggtcagccggaaatcaacctgcgtctgctgccgggttatggtggtacgcagcgtctgccgcgtctgctgtacaaacgcaacaatggtacaggcctgctgcgtgcgctggaaatgattctgggtggccgcagcgtgccagcagatgaagcactggaactgggtctgattgatgcaatcgcgaccggcgatcaggatagtctgagcctggcctgcgcactggcgcgtgcggcaatcggtgcagatggtcagctgattgaaagcgcagcggtgacccaggcctttcgtcatcgccacgaacagctggatgaatggcgtaaaccggacccgcgcttcgcggatgatgaactgcgctctattatcgcccatccgcgtatcgaacgcattatccgtcaggcgcataccgttggtcgtgatgcagcagtgcaccgtgcactggatgcaattcgttatggcattatccatggttttgaagccggcctggaacacgaagcaaaactgttcgccgaagcagtggttgatccgaatggtggcaaacgcggcatccgtgaatttctggatcgtcagtctgcaccgctgccgacacgtcgcccgctgattaccccggaacaggaacagctgctgcgtgatcagaaagaactgctgccggtgggtagtccgtttttccctggcgttgatcgcatcccgaaatggcagtatgcgcaggccgtgattcgtgatcccgatactggtgcagcagcacatggcgatccgatcgttgcggaaaaacagattatcgttccggtggaacgtccgcgtgcgaaccaggcactgatttacgttctggcgagcgaagtgaactttaatgatatttgggccatcacaggtattccggtgagccgcttcgatgaacatgatcgtgattggcacgtgacgggttctggtggcatcggcctgattgttgcgctgggcgaagaagcccgtcgcgaaggtcgtctgaaagttggcgatctggtggcgatctatagcggccagtctgatctgctgagcccgctgatgggtctggacccgatggcagccgattttgtgattcagggtaatgataccccggatggctctcatcagcagttcatgctggcacaggcaccgcagtgcctgccgatcccgacggatatgagcattgaagcagcgggttcttatatcctgaacctgggcaccatttaccgcgcactgtttacgaccctgcaaattaaagcgggtcgtacgattttcatcgaaggtgcagcaacgggtacaggtctggatgcagcacgcagcgcagcacgtaatggtctgcgcgttatcggcatggtgagtagctctagtcgcgcgtctaccctgctggcagcaggagcacatggtgcaattaaccgcaaagacccggaagtggccgattgttttacgcgagttccggaagatccgagcgcatgggcagcatgggaagcagccggtcagccgctgctggcaatgttccgtgcccagaatgatggtcgtctggccgattatgtggttagccacgcaggcgaaaccgcgtttccgcgctctttccagctgctgggtgaaccgcgtgatggtcatatcccgacgctgaccttttatggtgcgacgagtggctaccactttaccttcctgggcaaaccgggttctgccagtccgaccgaaatgctgcgtcgcgcaaacctgcgtgccggtgaagcagttctgatttattacggtgtgggcagcgatgatctggttgataccggaggcctggaagcgatcgaagccgcacgtcagatgggagcccgcattgtggttgtgacggtgtctgatgcccagcgcgaatttgttctgagtctgggtttcggtgcagcactgcgtggtgttgtgagcctggcggaactgaaacgtcgctttggcgatgaatttgaatggccgcgtaccatgccgccgctgccgaatgcacgtcaggacccgcagggcctgaaagaagcggtgcgtcgctttaacgatctggttttcaaaccgctgggtagcgcagttggcgtgtttctgcgctctgcggataacccgcgtggttatccggatctgattatcgaacgcgcagcgcatgatgccctggcagtgagtgccatgctgattaaaccgtttaccggccgtatcgtttatttcgaagatattggtggccgtcgctacagctttttcgcaccgcagatttgggtgcgtcagcgtcgcatttatatgccgacggcccagatttttggtacacacctgtctaacgcatacgaaattctgcgtctgaatgatgaaatcagtgcaggcctgctgacgattaccgaaccggcggttgtgccgtgggatgaactgccggaagcgcatcaggccatgtgggaaaaccgccacactgccgcaacctatgttgtgaatcatgcgctgccgcgcctgggtctgaaaaaccgtgatgaactgtacgaagcatggaccgcaggcgaacgtgaacaaaaactcatctcagaagaggatctgtaaggatctaagaggagaaagtgctatgcgtaaactggcccataacttttataaaccgctggcaattggagcaccggaaccgatccgcgaactgccggttcgtccggaacgcgtggttcatttctttccgccgcacgtggaaaaaattcgtgctcgcatcccggaagttgctaaacaggttgatgtcctgtgcggcaacctggaagatgcaattccgatggacgctaaagaagcggcccgtaatggttttatcgaagtcgtgaaagcaaccgatttcggcgacacggctctgtgggtgcgcgttaacgcgctgaacagcccgtgggtgctggatgacattgccgaaatcgttgcagctgtcggtaacaaactggatgtcattatgatcccgaaagtggaaggcccgtgggatattcactttgttgaccagtacctggcgctgctggaagcccgtcatcaaatcaaaaaaccgattctgatccacgcgctgctggaaaccgcccagggtatggtgaatctggaagaaattgcgggtgccagcccgcgtatgcacggtttctctctgggtccggcggatctggcagcctcgcgtggtatgaaaaccacgcgcgttggcggtggccacccgttttatggtgtcctggccgatccgcaggaaggccaagcagaacgtccgttctatcagcaggatctgtggcattacaccattgcgcgtatggttgacgtggcagttgctcacggtctgcgtgccttttacggtccgttcggcgatatcaaagacgaagcagcttgcgaagcacagtttcgcaatgctttcctgctgggttgtacgggcgcatggagtctggctccgaaccaaattccgatcgcaaaacgtgtcttttccccggatgtgaatgaagttctgttcgcgaaacgcattctggaagccatgccggatggttctggcgtggcgatgatcgacggtaaaatgcaggatgacgcgacgtggaaacaagccaaagtcattgtggatctggcgcgtatgatcgccaaaaaagatccggacctggcgcaggcctatggcctgtagttcacacaggaaaccacatgaaaggtattctgcatggtctgcgtgtggtggaaggttcggcttttgtcgctgccccgctgggtggtatgacgctggctcaactgggtgcagatgttatccgctttgacccgattggcggtggcctggattataaacgttggccggtcaccctggacggcaaacatagtctgttctgggctggtctgaacaaaggcaaacgctccattgcgatcgatattcgccatccgcgtggtcaggaactgctgacccaactgatctgcgctccgggtgaacacgcaggcctgtttattacgaatttcccggctcgtggttggctgtcatacgatgaactgaaacgtcaccgcgcggacctgatcatggttaatctggtcggtcgtcgcgatggtggctcggaagtggactacaccgttaatccgcagctgggtctgccgtttatgacgggtccggtgaccacgccggatgtggttaaccatgttctgcctgcctgggacatcgtcacaggtcagatgattgcactgggcctgctggcggcagaacgtcaccgtcgcctgacgggtgaaggccaactggtgaaaatcgctctgaaagatgttggtctggcgatgattggtcatctgggcatgatcgccgaagtgatgattaacgataccgaccgtccgcgtcagggcaattatctgtacggtgcatttggccgcgatttcgaaaccctggacggtaaacgtgttatggtcgtgggcctgacggatctgcaatggaaagccctgggtaaagcaaccggcctgacggacgcatttaacgctctgggtgcgcgtctgggcctgaatatggatgaagaaggtgaccgtttccgcgcgcgtcatgaaattgcagctctgctggaaccgtggtttcacgctcgtaccctggcggaagtgcgtcgcatcttcgaacagcatcgtgtcacctgggctccgtatcgcacggtgcgtgaagcgattgcccaggacccggactgtagcaccgataatccgatgtttgctatggttgaacaaccgggtatcggcagctacctgatgccgggctctccgctggatttcaccgcagtcccgcgtctgccggtgcagccagcaccgcgtctgggtgaacacacggatgaaattctgctggaagttctgggcctgagtgaagccgaagttggtcgtctgcatgatgaaggtattgttgctggcccggatcgtgctgcctgaattaaagaggagaaatagcaatgtcctcggcggattggatggcttggattggtcgcacggaacaggtggaagatgatatttgtctggcacaggctattgcagcggctgctaccctggaaccgccgagcggagcaccgacggctgattctccgctgccgccgctgtggcattggttttatttcctgccgcgtgccccgcagagtcaactgagctctgacggtcacccgcagcgcggcggttttattccgccgatcccgtacccgcgtcgcatgtttgcgggtgcccgtattcgcttccatcacccgctgcgtatcggtcagccagcacgtcgcgaaggtgtgattcgtaacatcacccaaaaaagtggccgctccggtccgctggcattcgttacggtcggctatcagatttaccaacatgaaatgctgtgcattgaagaagaacaggatatcgtttatcgtgaaccgggtgctccggtcccggcaccgaccccggtcgaactgccgccggttcacgatgcgattacccgtacagtggttcctgacccgcgtctgctgtttcgcttctccgcactgacgtttaacgctcatcgtatccactatgatcgcccttacgcgcagcatgaagaaggctaccctggtctggtcgttcacggtccgctggttgcggttctgctgatggaactggcgcgtcatcacaccagccgcccgattgttggtttttcattccgttcgcaagcgccgctgtttgacctggcaccgttccgtctgctggcacgtccgaatggtgatcgcatcgacctggaagcacaaggcccggatggcgcaacggcactgtcggcaacggtggaactgggtggttaaaggagggcatctatgtccgcaaaaacgaatccgggcaacttctttgaagatttccgtctgggccaaaccattgtccacgctacgccgcgcaccattaccgaaggcgatgtggccctgtataccagcctgtacggttctcgttttgcactgaccagctctacgccgttcgctcagtcactgggcctggaacgtgctccgattgactcgctgctggtgtttcatatcgttttcggcaaaaccgttccggatattagtctgaacgcgatcgccaatctgggttatgcgggcggtcgttttggtgccgtggtttacccaggtgacaccctgtcaaccacgtcgaaagtgattggcctgcgccagaacaaagatggcaaaacgggtgtcgtgtatgttcactctgtcggtgtgaatcaatgggacgaagttgtcctggaatacatccgttgggttatggtccgtaaacgcgatccgaacgcaccggctccggaaaccgtggttccggatctgccggacagcgtgccggttaccgatctgacggtcccgtataccgtgagtgcggccaactacaatctggcgcatgccggttccaattatctgtgggatgactacgaagtgggcgaaaaaattgatcatgtggacggtgtgaccatcgaagaagcagaacacatgcaggctacccgtctgtatcaaaacacggcccgcgttcattttaatctgcacgtcgaacgtgaaggccgcttcggtcgtcgcattgtttacggcggtcatattatcagcctggcacgtagtctgtcctttaacggcctggcaaatgctctgagtattgcagctatcaactccggccgccacaccaatccgagcttcgcaggtgacacgatttatgcttggtctgaaatcctggcgaaaatggccattccgggtcgtaccgatatcggagcactgcgtgttcgtaccgtcgcaacgaaagatcgtccgtgccacgacttcccgtatcgcgatgcggaaggtaactatgacccggctgttgtgctggattttgattacaccgtgctgatgccgcgtcgtggcgaacaaaaactcatctcagaagaggatctgaatagcgccgtcgactaagcttgcatgcctgcaggtcgactctagaggatccccgggtaccgagctcgaattaggaggaattaataatgattgaacaagatggattgcacgcaggttctccggccgcttgggtggagaggctattcggctatgactgggcacaacagacaatcggctgctctgatgccgccgtgttccggctgtcagcgcaggggaggccggttctttttgtcaagaccgacctgtccggtgccctgaatgaacttcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggcgcgccttcgttagtgttagtctagaactagtttagtaaaaaacgagcaatataagccttctttaaataagaaagagggcttatattactcgtttttttctataaaaatgagcaaatttttatagagtatcatattttactttatttattatattaataataaataataataataaataataaaaaattactatatattttttattagaaaaaaaataaggtggaatttgctacctttttttattttttattgaaatttgtatttttttttttttttagacaatacaaaaaagaatagatagtagcgtaggggctccacttggctcgggggatatagctcagttggtagagctccgctcttgcaattgggtcgttgcgattacgggttgggtgtctaattgtccaggcggtaatgatagtatcttgtacctgaaccggtggctcactttttctaagtaatggggaaaaggaccgaaacatgccactgaaagactctactgagacaaagatgggctgtcaagaacgtagaggaggtaggatggtcagttggtcagatctagtatggatcgtacatggacggtagttggagtcggcggctctcctagggttccctcgtctgggattgatccctggggaagaggatcaagttggcccttgcgaacagcttgatgcactatctcccttcaaccctttgagcgaaatgcggcaaaaggaaggaaaatccatggaccgaccccatcgtctccaccccgtaggaactacgagatcaccccaaggacgccttcggtatccaggggtcgcggaccgaccatagaaccctgttcaataagtggaatgcattagctgtccgctcgcaggttgggcagtaagggtcggagaagggcaatcactcattcttaaaaccagcattcgaaagagttggggcggaaaagggggggaaagctctccgttcctggttctcctgtagctggatcctctagaaccacaagaatccttagttggaatgggattccagctcatcaccttttgagattttgagaagagttgctctttggagagcacagtacgatgaaagttgtaagctgtgttcgggggggagttcttgtctatcgttggcctctatggtagaatcagtcaggggcctgataggcggtggtttaccctgtggcggatgtcagcggttcgagtccgcttatctccaactcgtgaacttagccgatacaaagctatatgatagcacccaatttttccgattcggcacactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 90 - DNA, 14617 bp, Artificial Sequence
  source: /note=“Description of Artificial Sequence Synthetic polynucleotide”
ggcccccagcaggaggcccgcacgacgggctattagctcagtggtagagcgcgcccctgataattgcgtcgttgtgcctgggctgtgagggctctcagccacatggatagttcaatgtgctcatcagcgcctgaccctgagatgtggatcatccaaggcacattagcatggcgtactcctcctgttcgaaccggggtttgaaaccaaacttctcctcaggaggatagatggggcgattcaggtgagatccaatgtagatccaactttctattcactcgtgggatccgggcggtccggaggggaccactatggctcctctcttctcgagaatccatacatcccttatcagtgtatggacagctatctctcgagcgcaggtttaggttcggcctcaatgggaaaataaaatggagcacctaacaacgtatcttcacagaccaagaactacgagatcacccctttcattctggggtgacggagggatcgtaccgttcgagcctttttttcatgttatctatctcttgactcgaaatgggagcaggtttgaaaaaggatcttagagtgtctagggttaggccagtagggtctcttaacgccctcttttttcttctcatcgaagttatttcacaaatacttcctatggtaaggaagagggggggaacaagcacacttggagagcgcagtacaacggagagttgtatgctgcgttcgggaaggatgaatcgctcccgaaaaggaatctattgattctctcccaattggttggaccataggtgcgatgatttacttcacgggcgaggtctctggttcaaatccaggatggcccagctgcggctccctcgctgtgatcgaataagaatggataagaggctcgtgggattgacgtgagggggtaggggtagctatatttctgggagcgaactccatgcgaatatgaagcgcatggatacaagttatgacttggaatgaaagacaattccgaatcgaattcagatctaggaggattatattatgggcgagcagaagctgattagcgaggaagacctaagcggcacaggtcgtctggcaggcaaaattgcactgatcacgggcggtgcgggcaacatcggtagcgaactgacccgtcgctttctggccgaaggtgcaacggtgattatctctggccgtaatcgcgccaaactgaccgcactggcggaacgtatgcaggccgaagcaggtgtgcctgcgaaacgcattgatctggaagttatggatggtagtgatccggtggcggttcgtgctggcattgaagcaatcgtggcgcgccacggtcagattgatatcctggttaacaatgcaggcagcgcaggagcacagcgtcgcctggcagaaattccgctgaccgaagcagaactgggtccgggtgcagaagaaacgctgcacgccagtatcgcaaacctgctgggcatgggttggcatctgatgcgtattgcagcaccgcacatgccggttggcagcgcagtgatcaatgttagcaccattttctctcgtgcggaatattacggtcgcattccgtatgtgacgccgaaagcagcgctgaacgccctgtctcagctggcagcacgcgaactgggagcacgtggtatccgcgttaataccatttttccgggtccgatcgaaagtgatcgtattcgcacggtgttccagcgtatggatcagctgaaaggccgcccggaaggtgataccgcccatcactttctgaacacgatgcgtctgtgccgcgcgaatgatcagggagccctggaacgtcgctttccgagcgtgggtgatgttgcggatgcagccgttttcctggcatctgcggaaagtgcagcgctgtctggtgaaaccatcgaagtgacgcatggcatggaactgccagcgtgtagcgaaacctctctgctggcccgcaccgatctgcgtactattgatgccagcggtcgtaccacgctgatctgcgcaggtgatcagattgaagaagttatggcgctgaccggaatgcttcgtacttgtggtagcgaagtgattatcggctttcgctctgccgcagcgctggcacagttcgaacaggcagtgaacgaaagccgtcgcctggcaggtgcagattttaccccgccgatcgcactgccgctggacccgcgtgatccggcgacgattgatgccgttttcgattggggcgcgggtgaaaacaccggcggtatccatgccgcagtgattctgccagcaacgagccacgaaccggcaccgtgcgtgatcgaagttgatgatgagcgcgttctgaatttcctggcggatgaaattacaggtacgattgtgatcgcaagtcgtctggcgcgctattggcagagccagcgtctgaccccaggtgcgcgtgcccgtggtccgcgcgttatctttctgagtaacggcgcggatcagaacggcaatgtgtatggtcgtattcagagcgcggccattggtcagctgatccgtgtttggcgtcatgaagccgaactggattaccagcgcgcaagcgcagcaggtgatcacgtgctgccgccggtttgggcgaaccagattgtgcgctttgccaatcgttctctggaaggtctggaatttgcttgcgcgtggaccgcgcagctgctgcatagtcagcgtcacattaatgaaatcacgctgaacattccggccaatatctctgcaaccacgggagcacgcagtgcaagcgttggttgggcggaaagtctgatcggcctgcatctgggtaaagtggcactgattaccggcggtagcgcgggtattggcggtcagatcggtcgtctgctggcactgtctggtgcccgcgttatgctggcagcacgtgatcgccataaactggaacagatgcaggccatgatccagagcgaactggcagaagtgggctataccgatgtggaagatcgtgttcacattgccccaggttgtgatgtgagttctgaagcacagctggcggatctggttgaacgcaccctgtctgcgtttggcacggtggattatctgatcaacaatgcaggcattgccggtgtggaagaaatggttatcgatatgccggttgaaggttggcgtcataccctgttcgccaacctgattagtaattacagcctgatgcgcaaactggcaccgctgatgaaaaaacagggctctggttatatcctgaacgtgagtagctactttggcggtgaaaaagatgcggccattccgtatccgaatcgtgcggattacgccgttagtaaagcgggccagcgtgcgatggcagaagtgtttgcccgcttcctgggtccagaaattcagatcaatgcgattgcaccgggtccggtggaaggtgatcgtctgcgtggtacaggtgaacgtccgggtctgtttgcacgtcgcgcacgtctgatcctggaaaacaaacgcctgaatgaactgcacgcagctctgattgccgcagcgcgcaccgatgaacgtagtatgcacgaactggtggaactgctgctgccgaacgatgttgcagcactggaacagaatccggcagcaccgaccgcactgcgtgaactggcacgtcgcttccgcagtgaaggtgatccggcagcgtctagtagctctgccctgctgaaccgtagcatcgccgcaaaactgctggcgcgcctgcataatggcggttatgttctgccagcagatatttttgcgaacctgccgaatccgccggacccgtttttcacccgtgcgcagatcgatcgtgaagcccgcaaagtgcgtgatggcattatgggtatgctgtacctgcaacgtatgccgaccgaatttgatgtggcaatggcgacggtttattacctggcggatcgcaacgtttctggcgaaacctttcacccgagtggcggtctgcgctatgaacgtaccccgacgggcggtgaactgttcggtctgccgtctccggaacgtctggcggaactggtgggtagtaccgtttacctgattggcgaacatctgacggaacacctgaatctgctggcccgcgcatatctggaacgctacggagcccgtcaggtggttatgatcgtggaaaccgaaacgggtgcggaaaccatgcgtcgcctgctgcatgatcacgtggaagcaggtcgcctgatgacgattgttgcgggcgatcagattgaagcggccatcgatcaggcgattacccgctatggtcgtccgggtccggtggtttgcaccccgtttcgcccgctgccgacggtgccgctggttggccgtaaagattctgattggagtaccgtgctgagcgaagccgaatttgcagaactgtgtgaacatcagctgacccatcacttccgcgttgcccgtaaaattgcactgagtgatggtgcaagcctggcactggtgaccccggaaaccacggcaacgagcaccacggaacagtttgcgctggccaacttcatcaaaaccacgctgcacgcattcaccgcgacgattggtgttgaatctgaacgcaccgcgcagcgtattctgatcaatcaggtggatctgactcgtcgcgcacgcgcggaagaaccgcgtgatccgcatgaacgccagcaggaactggaacgttttattgaagcagtgctgctggttaccgcaccgctgccgccggaagcagatactcgttacgcaggtcgtatccaccgtggtcgcgcgattaccgtgtaaggatctaggaggattatattatgatcgataccgcaccgctggcaccgccgcgtgctccgcgcagcaatccgattcgtgatcgcgtggattgggaagcgcagcgtgcagcagcactggccgatccgggtgcatttcatggtgcgatcgcccgtaccgttattcactggtatgatccgcagcatcactgctggattcgcttcaacgaaagctctcagcgttgggaaggtctggatgcagcaacgggtgctccggttacagtggattatcctgccgattaccagccgtggcagcaggcatttgatgatagtgaagcgccgttttatcgctggttcagcggcggtctgacgaacgcatgttttaatgaagttgatcgtcacgtgacaatgggttacggcgatgaagtggcgtattacttcgaaggtgatcgctgggataatagcctgaacaatggccgtggcggtccggtggttcaggaaacgattacccgtcgccgtctgctggttgaagtggttaaagcagcgcaggttctgcgcgatctgggcctgaaaaaaggtgatcgtatcgcgctgaacatgccgaatatcatgccgcagatttattacaccgaagccgcaaaacgcctgggtattctgtatacgccggtgtttggcggtttcagtgataaaaccctgagcgatcgcatccataatgcaggtgcgcgtgtggttattacctctgatggcgcgtatcgtaacgcccaggtggttccgtataaagaagcctacacggatcaggcactggataaatacatcccggtggaaaccgcccaggcaattgttgcacagacgctggcaaccctgccgctgaccgaaagtcagcgccagacgattatcaccgaagtggaagcagcactggcaggtgaaattacggttgaacgttctgatgttatgcgcggtgtgggcagtgcgctggccaaactgcgcgatctggatgccagtgtgcaggcaaaagttcgtaccgtgctggcacaggcgctggttgaaagcccgccgcgcgtggaagcagtggttgtggttcgtcatacgggtcaggaaatcctgtggaatgaaggccgtgatcgctggagccacgatctgctggatgcagcactggcgaaaattctggctaacgcacgcgccgcaggttttgatgttcactctgaaaacgatctgctgaatctgccggatgatcagctgatccgtgctctgtatgcgagtattccgtgcgaaccagttgatgccgaatatccgatgtttattatctacacgagcggttctaccggcaaaccgaaaggtgttattcatgttcacggcggttacgtggcgggcgtggttcataccctgcgcgttagtttcgatgccgaaccgggcgatacgatttatgtgatcgcagatccgggctggatcacaggtcagagctacatgctgacggcaaccatggcaggtcgtctgactggtgtgattgccgaaggttctccgctgtttccgagtgcgggccgctatgcctctattatcgaacgttacggtgttcagatttttaaagcgggcgttacgttcctgaaaaccgtgatgagtaacccgcagaatgttgaagatgtgcgcctgtatgatatgcacagtctgcgtgtggcaaccttttgtgcagagccggttagcccggcagtgcagcagttcggtatgcagatcatgacgccgcagtatattaatagctactgggcgacggaacatggcggtattgtgtggacccacttttatggcaaccaggatttcccgctgcgtccagatgcacatacgtacccgctgccgtgggttatgggtgatgtttgggtggcagaaaccgatgaatctggcaccacgcgctatcgcgtggcggatttcgatgaaaaaggtgaaatcgttatcaccgcaccgtatccgtacctgacgcgaaccctgtggggtgatgtgccgggttttgaagcgtatctgcgtggtgaaatcccgctgcgtgcatggaaaggtgatgcagaacgtttcgttaaaacctactggcgtcgtggtccgaatggcgaatggggttatatccagggcgattttgcgattaaatacccggatggtagtttcacgctgcatggccgcagcgatgatgttattaatgtgtccggccaccgtatgggtacggaagaaatcgaaggtgccattctgcgtgatcgccagatcaccccggattctccggtgggtaactgcattgtggttggcgcgccgcatcgtgaaaaaggcctgaccccggttgcatttatccagccagcaccgggtcgtcacctgacgggtgcagatcgccgtcgcctggatgaactggtgcgtaccgaaaaaggtgcagttagcgtgccggaagattatattgaagttagtgcgtttccggaaacccgcagcggtaaatacatgcgtcgcttcctgcgtaatatgatgctggatgaaccgctgggcgataccacgaccctgcgcaacccggaagtgctggaagaaatcgcggccaaaattgccgaatggaaacgtcgccagcgcatggcagaagaacagcagattatcgaacgttatcgctactttcgtattgaatatcatccgccgaccgcaagtgcaggtaaactggcagtggttacggttaccaatccgccggtgaacgccctgaatgaacgtgctctggatgaactgaacaccatcgtggatcacctggcgcgtcgccaggatgttgcagcgattgtgtttacgggtcagggtgctcgcagcttcgtggccggtgcggatatccgtcagctgctggaagaaattcataccgttgaagaagccatggcactgccgaacaatgcgcacctggcctttcgcaaaattgaacgtatgaacaaaccgtgcattgccgcaatcaatggtgtggcactgggcggtggcctggaatttgcgatggcctgtcattatcgcgttgccgatgtgtacgcagaatttggtcagccggaaatcaacctgcgtctgctgccgggttatggtggtacgcagcgtctgccgcgtctgctgtacaaacgcaacaatggtacaggcctgctgcgtgcgctggaaatgattctgggtggccgcagcgtgccagcagatgaagcactggaactgggtctgattgatgcaatcgcgaccggcgatcaggatagtctgagcctggcctgcgcactggcgcgtgcggcaatcggtgcagatggtcagctgattgaaagcgcagcggtgacccaggcctttcgtcatcgccacgaacagctggatgaatggcgtaaaccggacccgcgcttcgcggatgatgaactgcgctctattatcgcccatccgcgtatcgaacgcattatccgtcaggcgcataccgttggtcgtgatgcagcagtgcaccgtgcactggatgcaattcgttatggcattatccatggttttgaagccggcctggaacacgaagcaaaactgttcgccgaagcagtggttgatccgaatggtggcaaacgcggcatccgtgaatttctggatcgtcagtctgcaccgctgccgacacgtcgcccgctgattaccccggaacaggaacagctgctgcgtgatcagaaagaactgctgccggtgggtagtccgtttttccctggcgttgatcgcatcccgaaatggcagtatgcgcaggccgtgattcgtgatcccgatactggtgcagcagcacatggcgatccgatcgttgcggaaaaacagattatcgttccggtggaacgtccgcgtgcgaaccaggcactgatttacgttctggcgagcgaagtgaactttaatgatatttgggccatcacaggtattccggtgagccgcttcgatgaacatgatcgtgattggcacgtgacgggttctggtggcatcggcctgattgttgcgctgggcgaagaagcccgtcgcgaaggtcgtctgaaagttggcgatctggtggcgatctatagcggccagtctgatctgctgagcccgctgatgggtctggacccgatggcagccgattttgtgattcagggtaatgataccccggatggctctcatcagcagttcatgctggcacaggcaccgcagtgcctgccgatcccgacggatatgagcattgaagcagcgggttcttatatcctgaacctgggcaccatttaccgcgcactgtttacgaccctgcaaattaaagcgggtcgtacgattttcatcgaaggtgcagcaacgggtacaggtctggatgcagcacgcagcgcagcacgtaatggtctgcgcgttatcggcatggtgagtagctctagtcgcgcgtctaccctgctggcagcaggagcacatggtgcaattaaccgcaaagacccggaagtggccgattgttttacgcgagttccggaagatccgagcgcatgggcagcatgggaagcagccggtcagccgctgctggcaatgttccgtgcccagaatgatggtcgtctggccgattatgtggttagccacgcaggcgaaaccgcgtttccgcgctctttccagctgctgggtgaaccgcgtgatggtcatatcccgacgctgaccttttatggtgcgacgagtggctaccactttaccttcctgggcaaaccgggttctgccagtccgaccgaaatgctgcgtcgcgcaaacctgcgtgccggtgaagcagttctgatttattacggtgtgggcagcgatgatctggttgataccggaggcctggaagcgatcgaagccgcacgtcagatgggagcccgcattgtggttgtgacggtgtctgatgcccagcgcgaatttgttctgagtctgggtttcggtgcagcactgcgtggtgttgtgagcctggcggaactgaaacgtcgctttggcgatgaatttgaatggccgcgtaccatgccgccgctgccgaatgcacgtcaggacccgcagggcctgaaagaagcggtgcgtcgctttaacgatctggttttcaaaccgctgggtagcgcagttggcgtgtttctgcgctctgcggataacccgcgtggttatccggatctgattatcgaacgcgcagcgcatgatgccctggcagtgagtgccatgctgattaaaccgtttaccggccgtatcgtttatttcgaagatattggtggccgtcgctacagctttttcgcaccgcagatttgggtgcgtcagcgtcgcatttatatgccgacggcccagatttttggtacacacctgtctaacgcatacgaaattctgcgtctgaatgatgaaatcagtgcaggcctgctgacgattaccgaaccggcggttgtgccgtgggatgaactgccggaagcgcatcaggccatgtgggaaaaccgccacactgccgcaacctatgttgtgaatcatgcgctgccgcgcctgggtctgaaaaaccgtgatgaactgtacgaagcatggaccgcaggcgaacgtgaacaaaaactcatctcagaagaggatctgtaaggatccctcgactctagaggatccccgggtaccgagctcgaattaggaggaattaataatgattgaacaagatggattgcacgcaggttctccggccgcttgggtggagaggctattcggctatgactgggcacaacagacaatcggctgctctgatgccgccgtgttccggctgtcagcgcaggggaggccggttctttttgtcaagaccgacctgtccggtgccctgaatgaacttcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggcgcgccttcgttagtgttagtctagaactagtttagtaaaaaacgagcaatataagccttctttaaataagaaagagggcttatattactcgtttttttctataaaaatgagcaaatttttatagagtatcatattttactttatttattatattaataataaataataataataaataataaaaaattactatatattttttattagaaaaaaaataaggtggaatttgctacctttttttattttttattgaaatttgtatttttttttttttttagacaatacaaaaaagaatagatagtagcgtaggggctccacttggctcgggggatatagctcagttggtagagctccgctcttgcaattgggtcgttgcgattacgggttgggtgtctaattgtccaggcggtaatgatagtatcttgtacctgaaccggtggctcactttttctaagtaatggggaaaaggaccgaaacatgccactgaaagactctactgagacaaagatgggctgtcaagaacgtagaggaggtaggatggtcagttggtcagatctagtatggatcgtacatggacggtagttggagtcggcggctctcctagggttccctcgtctgggattgatccctggggaagaggatcaagttggcccttgcgaacagcttgatgcactatctcccttcaaccctttgagcgaaatgcggcaaaaggaaggaaaatccatggaccgaccccatcgtctccaccccgtaggaactacgagatcaccccaaggacgccttcggtatccaggggtcgcggaccgaccatagaaccctgttcaataagtggaatgcattagctgtccgctcgcaggttgggcagtaagggtcggagaagggcaatcactcattcttaaaaccagcattcgaaagagttggggcggaaaagggggggaaagctctccgttcctggttctcctgtagctggatcctctagaaccacaagaatccttagttggaatgggattccagctcatcaccttttgagattttgagaagagttgctctttggagagcacagtacgatgaaagttgtaagctgtgttcgggggggagttcttgtctatcgttggcctctatggtagaatcagtcaggggcctgataggcggtggtttaccctgtggcggatgtcagcggttcgagtccgcttatctccaactcgtgaacttagccgatacaaagctatatgatagcacccaatttttccgattcggcacactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 91 - DNA, 9190 bp, Artificial Sequence
  source: /note=“Description of Artificial Sequence Synthetic polynucleotide”
ggcccccagcaggaggcccgcacgacgggctattagctcagtggtagagcgcgcccctgataattgcgtcgttgtgcctgggctgtgagggctctcagccacatggatagttcaatgtgctcatcagcgcctgaccctgagatgtggatcatccaaggcacattagcatggcgtactcctcctgttcgaaccggggtttgaaaccaaacttctcctcaggaggatagatggggcgattcaggtgagatccaatgtagatccaactttctattcactcgtgggatccgggcggtccggaggggaccactatggctcctctcttctcgagaatccatacatcccttatcagtgtatggacagctatctctcgagcgcaggtttaggttcggcctcaatgggaaaataaaatggagcacctaacaacgtatcttcacagaccaagaactacgagatcacccctttcattctggggtgacggagggatcgtaccgttcgagcctttttttcatgttatctatctcttgactcgaaatgggagcaggtttgaaaaaggatcttagagtgtctagggttaggccagtagggtctcttaacgccctcttttttcttctcatcgaagttatttcacaaatacttcctatggtaaggaagagggggggaacaagcacacttggagagcgcagtacaacggagagttgtatgctgcgttcgggaaggatgaatcgctcccgaaaaggaatctattgattctctcccaattggttggaccataggtgcgatgatttacttcacgggcgaggtctctggttcaaatccaggatggcccagctgcggctccctcgctgtgatcgaataagaatggataagaggctcgtgggattgacgtgagggggtaggggtagctatatttctgggagcgaactccatgcgaatatgaagcgcatggatacaagttatgacttggaatgaaagacaattccgaatcgaattcagatctaagaggagaaagtgctatgagcatcttgtacgaagagcgtcttgatggcgctttacccgatgtcgaccgcacatcggtactgatggcactgcgtgagcatgtccctggacttgagatcctgcataccgatgaggagatcattccttacgagtgtgacgggttgagcgcgtatcgcacgcgtccattactggttgttctgcctaagcaaatggaacaggtgacagcgattctggctgtctgccatcgcctgcgtgtaccggtggtgacccgtggtgcaggcaccgggctttctggtggcgcgctgccgctggaaaaaggtgtgttgttggtgatggcgcgctttaaagagatcctcgacattaaccccgttggtcgccgcgcgcgcgtgcagccaggcgtgcgtaacctggcgatctcccaggccgttgcaccgcataatctctactacgcaccggacccttcctcacaaatcgcctgttccattggcggcaatgtggctgaaaatgccggcggcgtccactgcctgaaatatggtctgaccgtacataacctgctgaaaattgaagtgcaaacgctggacggcgaggcactgacgcttggatcggacgcgctggattcacctggttttgacctgctggcgctgttcaccggatcggaaggtatgctcggcgtgaccaccgaagtgacggtaaaactgctgccgaagccgcccgtggcgcgggttctgttagccagctttgactcggtagaaaaagccggacttgcggttggtgacatcatcgccaatggcattatccccggcgggctggagatgatggataacctgtcgatccgcgcggcggaagattttattcatgccggttatcccgtcgacgccgaagcgattttgttatgcgagctggacggcgtggagtctgacgtacaggaagactgcgagcgggttaacgacatcttgttgaaagcgggcgcgactgacgtccgtctggcacaggacgaagcagagcgcgtacgtttctgggccggtcgcaaaaatgcgttcccggcggtaggacgtatctccccggattactactgcatggatggcaccatcccgcgtcgcgccctgcctggcgtactggaaggcattgcccgtttatcgcagcaatatgatttacgtgttgccaacgtctttcatgccggagatggcaacatgcacccgttaatccttttcgatgccaacgaacccggtgaatttgcccgcgcggaagagctgggcgggaagatcctcgaactctgcgttgaagttggcggcagcatcagtggcgaacatggcatcgggcgagaaaaaatcaatcaaatgtgcgcccagttcaacagcgatgaaatcacgaccttccatgcggtcaaggcggcgtttgaccccgatggtttgctgaaccctgggaaaaacattcccacgctacaccgctgtgctgaatttggtgccatgcatgtgcatcacggtcatttacctttccctgaactggagcgtttctgatgctacgcgagtgtgattacagccaggcgctgctggagcaggtgaatcaggcgattagcgataaaacgccgctggtgattcagggcagcaatagcaaagcctttttaggtcgccctgtcaccgggcaaacgctggatgttcgttgtcatcgcggcattgttaattacgacccgaccgagctggtgataaccgcgcgtgtcggaacgccgctggtgacaattgaagcggcgctggaaagcgcggggcaaatgctcccctgtgagccgccgcattatggtgaagaagccacctggggcgggatggtcgcctgcgggctggcggggccgcgtcgcccgtggagcggttcggtccgcgattttgtcctcggcacgcgcatcattaccggcgctggaaaacatctgcgttttggtggcgaagtgatgaaaaacgttgccggatacgatctctcacggttaatggtcggaagctacggttgtcttggcgtgctcactgaaatctcaatgaaagtgttaccgcgaccgcgcgcctccctgagcctgcgtcgggaaatcagcctgcaagaagccatgagtgaaatcgccgagtggcaactccagccattacccattagtggcttatgttacttcgacaatgcgttgtggatccgccttgagggcggcgaaggatcggtaaaagcagcgcgtgaactgctgggtggcgaagaggttgccggtcagttctggcagcaattgcgtgaacaacaactgccgttcttctcgttaccaggtaccttatggcgcatttcattacccagtgatgcgccgatgatggatttacccggcgagcaactgatcgactggggcggggcgttacgctggctgaaatcgacagccgaggacaatcaaatccatcgcatcgcccgcaacgctggcggtcatgcgacccgctttagtgccggagatggtggctttgccccgctatcggctcctttattccgctatcaccagcagcttaaacagcagctcgacccttgcggcgtgtttaaccccggtcgcatgtacgcggaactttgaggagcaggctatgcaaacccaattaactgaagagatgcggcagaacgcgcgcgcgctggaagccgacagcatcctgcgcgcctgtgttcactgcggattttgtaccgcaacctgcccaacctatcagcttctgggcgatgaactggacgggccgcgcgggcgcatctatctgattaaacaggtgctggaaggcaacgaagtcacgcttaaaacacaggagcatctcgatcgctgcctcacttgccgtaattgtgaaaccacctgtccttctggtgtgcgctatcacaatttgctggatatcgggcgtgatattgtcgagcagaaagtgaaacgcccactgccggagcgaatactgcgcgaaggattgcgccaggtagtgccgcgtccggcggtcttccgtgcgctgacgcaggtagggctggtgctgcgaccgtttttaccggaacaggtcagagcaaaactgcctgctgaaacggtgaaagctaaaccgcgtccgccgctgcgccataagcgtcgggttttaatgttggaaggctgcgcccagcctacgctttcgcccaacaccaacgcggcaactgcgcgagtgctggatcgtctggggatcagcgtcatgccagctaacgaagcaggctgttgtggcgcggtggactatcatcttaatgcgcaggagaaagggctggcacgggcgcgcaataatattgatgcctggtggcccgcgattgaagcaggtgccgaggcaattttgcaaaccgccagcggctgcggcgcgtttgtcaaagagtatgggcagatgctgaaaaacgatgcgttatatgccgataaagcacgtcaggtcagtgaactggcggtcgatttagtcgaacttctgcgcgaggaaccgctggaaaaactggcaattcgcggcgataaaaagctggccttccactgtccgtgtaccctacaacatgcgcaaaagctgaacggcgaagtggaaaaagtgttgcttcgtcttggatttaccttaacggacgttcccgacagccatctgtgctgcggttcagcgggaacatatgcgttaacgcatcccgatctggcacgccagctgcgggataacaaaatgaatgcgctggaaagcggcaaaccggaaatgatcgtcaccgccaacattggttgccagacgcatctggcgagcgccggtcgtacctctgtgcgtcactggattgaaattgtagaacaagcccttgaaaaggaataaggatccctcgactctagaggatccccgggtaccgagctcgaattaggaggaattaataatgattgaacaagatggattgcacgcaggttctccggccgcttgggtggagaggctattcggctatgactgggcacaacagacaatcggctgctctgatgccgccgtgttccggctgtcagcgcaggggaggccggttctttttgtcaagaccgacctgtccggtgccctgaatgaacttcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggcgcgccttcgttagtgttagtctagaactagtttagtaaaaaacgagcaatataagccttctttaaataagaaagagggcttatattactcgtttttttctataaaaatgagcaaatttttatagagtatcatattttactttatttattatattaataataaataataataataaataataaaaaattactatatattttttattagaaaaaaaataaggtggaatttgctacctttttttattttttattgaaatttgtatttttttttttttttagacaatacaaaaaagaatagatagtagcgtaggggctccacttggctcgggggatatagctcagttggtagagctccgctcttgcaattgggtcgttgcgattacgggttgggtgtctaattgtccaggcggtaatgatagtatcttgtacctgaaccggtggctcactttttctaagtaatggggaaaaggaccgaaacatgccactgaaagactctactgagacaaagatgggctgtcaagaacgtagaggaggtaggatggtcagttggtcagatctagtatggatcgtacatggacggtagttggagtcggcggctctcctagggttccctcgtctgggattgatccctggggaagaggatcaagttggcccttgcgaacagcttgatgcactatctcccttcaaccctttgagcgaaatgcggcaaaaggaaggaaaatccatggaccgaccccatcgtctccaccccgtaggaactacgagatcaccccaaggacgccttcggtatccaggggtcgcggaccgaccatagaaccctgttcaataagtggaatgcattagctgtccgctcgcaggttgggcagtaagggtcggagaagggcaatcactcattcttaaaaccagcattcgaaagagttggggcggaaaagggggggaaagctctccgttcctggttctcctgtagctggatcctctagaaccacaagaatccttagttggaatgggattccagctcatcaccttttgagattttgagaagagttgctctttggagagcacagtacgatgaaagttgtaagctgtgttcgggggggagttcttgtctatcgttggcctctatggtagaatcagtcaggggcctgataggcggtggtttaccctgtggcggatgtcagcggttcgagtccgcttatctccaactcgtgaacttagccgatacaaagctatatgatagcacccaatttttccgattcggcacactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 92 - DNA, 6126 bp, Artificial Sequence
  source: /note=“Description of Artificial Sequence Synthetic polynucleotide”
ggcccccagcaggaggcccgcacgacgggctattagctcagtggtagagcgcgcccctgataattgcgtcgttgtgcctgggctgtgagggctctcagccacatggatagttcaatgtgctcatcagcgcctgaccctgagatgtggatcatccaaggcacattagcatggcgtactcctcctgttcgaaccggggtttgaaaccaaacttctcctcaggaggatagatggggcgattcaggtgagatccaatgtagatccaactttctattcactcgtgggatccgggcggtccggaggggaccactatggctcctctcttctcgagaatccatacatcccttatcagtgtatggacagctatctctcgagcgcaggtttaggttcggcctcaatgggaaaataaaatggagcacctaacaacgtatcttcacagaccaagaactacgagatcacccctttcattctggggtgacggagggatcgtaccgttcgagcctttttttcatgttatctatctcttgactcgaaatgggagcaggtttgaaaaaggatcttagagtgtctagggttaggccagtagggtctcttaacgccctcttttttcttctcatcgaagttatttcacaaatacttcctatggtaaggaagagggggggaacaagcacacttggagagcgcagtacaacggagagttgtatgctgcgttcgggaaggatgaatcgctcccgaaaaggaatctattgattctctcccaattggttggaccataggtgcgatgatttacttcacgggcgaggtctctggttcaaatccaggatggcccagctgcggctccctcgctgtgatcgaataagaatggataagaggctcgtgggattgacgtgagggggtaggggtagctatatttctgggagcgaactccatgcgaatatgaagcgcatggatacaagttatgacttggaatgaaagacaattccgaatcgaattcagatctagttgtagggagggatttatggcgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccttggccaacacttgtcactactttctcttatggtgttcaatgcttttcaagatacccagatcatatgaagcggcacgacttcttcaagagcgccatgcctgagggatacgtgcaggagaggaccatctctttcaaggacgacgggaactacaagacacgtgctgaagtcaagtttgagggagacaccctcgtcaacaggatcgagcttaagggaattgatttcaaggaggacggaaacatcctcggccacaagttggaatacaactacaactcccacaacgtatacatcacggcagacaaacaaaagaatggaatcaaagctaacttcaaaattagacacaacattgaagatggaagcgttcaactagcagaccattatcaacaaaatactcctattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataaggatccctcgactctagaggatccccgggtaccgagctcgaattaggaggaattaataatgattgaacaagatggattgcacgcaggttctccggccgcttgggtggagaggctattcggctatgactgggcacaacagacaatcggctgctctgatgccgccgtgttccggctgtcagcgcaggggaggccggttctttttgtcaagaccgacctgtccggtgccctgaatgaacttcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggcgcgccttcgttagtgttagtctagaactagtttagtaaaaaacgagcaatataagccttctttaaataagaaagagggcttatattactcgtttttttctataaaaatgagcaaatttttatagagtatcatattttactttatttattatattaataataaataataataataaataataaaaaattactatatattttttattagaaaaaaaataaggtggaatttgctacctttttttattttttattgaaatttgtatttttttttttttttagacaatacaaaaaagaatagatagtagcgtaggggctccacttggctcgggggatatagctcagttggtagagctccgctcttgcaattgggtcgttgcgattacgggttgggtgtctaattgtccaggcggtaatgatagtatcttgtacctgaaccggtggctcactttttctaagtaatggggaaaaggaccgaaacatgccactgaaagactctactgagacaaagatgggctgtcaagaacgtagaggaggtaggatggtcagttggtcagatctagtatggatcgtacatggacggtagttggagtcggcggctctcctagggttccctcgtctgggattgatccctggggaagaggatcaagttggcccttgcgaacagcttgatgcactatctcccttcaaccctttgagcgaaatgcggcaaaaggaaggaaaatccatggaccgaccccatcgtctccaccccgtaggaactacgagatcaccccaaggacgccttcggtatccaggggtcgcggaccgaccatagaaccctgttcaataagtggaatgcattagctgtccgctcgcaggttgggcagtaagggtcggagaagggcaatcactcattcttaaaaccagcattcgaaagagttggggcggaaaagggggggaaagctctccgttcctggttctcctgtagctggatcctctagaaccacaagaatccttagttggaatgggattccagctcatcaccttttgagattttgagaagagttgctctttggagagcacagtacgatgaaagttgtaagctgtgttcgggggggagttcttgtctatcgttggcctctatggtagaatcagtcaggggcctgataggcggtggtttaccctgtggcggatgtcagcggttcgagtccgcttatctccaactcgtgaacttagccgatacaaagctatatgatagcacccaatttttccgattcggcacactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 93 - DNA, 18922 bp, Artificial Sequence; identical to SEQ ID NO: 89
  source: /note=“Description of Artificial Sequence Synthetic polynucleotide”

SEQ ID NO: 94 - DNA, 18425 bp, Artificial Sequence
  source: /note=“Description of Artificial Sequence Synthetic polynucleotide”
ggcccccagcaggaggcccgcacgacgggctattagctcagtggtagagcgcgcccctgataattgcgtcgttgtgcctgggctgtgagggctctcagccacatggatagttcaatgtgctcatcagcgcctgaccctgagatgtggatcatccaaggcacattagcatggcgtactcctcctgttcgaaccggggtttgaaaccaaacttctcctcaggaggatagatggggcgattcaggtgagatccaatgtagatccaactttctattcactcgtgggatccgggcggtccggaggggaccactatggctcctctcttctcgagaatccatacatcccttatcagtgtatggacagctatctctcgagcgcaggtttaggttcggcctcaatgggaaaataaaatggagcacctaacaacgtatcttcacagaccaagaactacgagatcacccctttcattctggggtgacggagggatcgtaccgttcgagcctttttttcatgttatctatctcttgactcgaaatgggagcaggtttgaaaaaggatcttagagtgtctagggttaggccagtagggtctcttaacgccctcttttttcttctcatcgaagttatttcacaaatacttcctatggtaaggaagagggggggaacaagcacacttggagagcgcagtacaacggagagttgtatgctgcgttcgggaaggatgaatcgctcccgaaaaggaatctattgattctctcccaattggttggaccataggtgcgatgatttacttcacgggcgaggtctctggttcaaatccaggatggcccagctgcggctccctcgctgtgatcgaataagaatggataagaggctcgtgggattgacgtgagggggtaggggtagctatatttctgggagcgaactccatgcgaatatgaagcgcatggatacaagttatgacttggaatgaaagacaattccgaatcgaattcagatctaggaggattatattatgggcgagcagaagctgattagcgaggaagacctaagcggcacaggtcgtctggcaggcaaaattgcactgatcacgggcggtgcgggcaacatcggtagcgaactgacccgtcgctttctggccgaaggtgcaacggtgattatctctggccgtaatcgcgccaaactgaccgcactggcggaacgtatgcaggccgaagcaggtgtgcctgcgaaacgcattgatctggaagttatggatggtagtgatccggtggcggttcgtgctggcattgaagcaatcgtggcgcgccacggtcagattgatatcctggttaacaatgcaggcagcgcaggagcacagcgtcgcctggcagaaattccgctgaccgaagcagaactgggtccgggtgcagaagaaacgctgcacgccagtatcgcaaacctgctgggcatgggttggcatctgatgcgtattgcagcaccgcacatgccggttggcagcgcagtgatcaatgttagcaccattttctctcgtgcggaatattacggtcgcattccgtatgtgacgccgaaagcagcgctgaacgccctgtctcagctggcagcacgcgaactgggagcacgtggtatccgcgttaataccatttttccgggtccgatcgaaagtgatcgtattcgcacggtgttccagcgtatggatcagctgaaaggccgcccggaaggtgataccgcccatcactttctgaacacgatgcgtctgtgccgcgcgaatgatcagggagccctggaacgtcgctttccgagcgtgggtgatgttgcggatgcagccgttttcctggcatctgcggaaagtgcagcgctgtctggtgaaaccatcgaagtgacgcatggcatggaactgccagcgtgtagcgaaacctctctgctggcccgcaccgatctgcgtactattgatgccagcggtcgtaccacgctgatctgcgcaggtgatcagattgaagaagttatggcgctgaccggaatgcttcgtacttgtggtagcgaagtgattatcggctttcgctctgccgcagcgctggcacagttcgaacaggcagtgaacgaaagccgtcgcctggcaggtgcagattttaccccgccgatcgcactgccgctggacccgcgtgatccggcgacgattgatgccgttttcgattggggcgcgggtgaaaacaccggcggtatccatgccgcagtgattctgccagcaacgagccacgaaccggcaccgtgcgtgatcgaagttgatgatgagcgcgttctgaatttcctggcggatgaaattacaggtacgattgtgatcgcaagtcgtctggcgcgctattggcagagccagcgtctgaccccaggtgcgcgtgcccgtggtccgcgcgttatctttctgagtaacggcgcggatcagaacggcaatgtgtatggtcgtattcagagcgcggccattggtcagctgatccgtgtttggcgtcatgaagccgaactggattaccagcgcgcaagcgcagcaggtgatcacgtgctgccgccggtttgggcgaaccagattgtgcgctttgccaatcgttctctggaaggtctggaatttgcttgcgcgtggaccgcgcagctgctgcatagtcagcgtcacattaatgaaatcacgctgaacattccggccaatatctctgcaaccacgggagcacgcagtgcaagcgttggttgggcggaaagtctgatcggcctgcatctgggtaaagtggcactgattaccggcggtagcgcgggtattggcggtcagatcggtcgtctgctggcactgtctggtgcccgcgttatgctggcagcacgtgatcgccataaactggaacagatgcaggccatgatccagagcgaactggcagaagtgggctataccgatgtggaagatcgtgttcacattgccccaggttgtgatgtgagttctgaagcacagctggcggatctggttgaacgcaccctgtctgcgtttggcacggtggattatctgatcaacaatgcaggcattgccggtgtggaagaaatggttatcgatatgccggttgaaggttggcgtcataccctgttcgccaacctgattagtaattacagcctgatgcgcaaactggcaccgctgatgaaaaaacagggctctggttatatcctgaacgtgagtagctactttggcggtgaaaaagatgcggccattccgtatccgaatcgtgcggattacgccgttagtaaagcgggccagcgtgcgatggcagaagtgtttgcccgcttcctgggtccagaaattcagatcaatgcgattgcaccgggtccggtggaaggtgatcgtctgcgtggtacaggtgaacgtccgggtctgtttgcacgtcgcgcacgtctgatcctggaaaacaaacgcctgaatgaactgcacgcagctctgattgccgcagcgcgcaccgatgaacgtagtatgcacgaactggtggaactgctgctgccgaacgatgttgcagcactggaacagaatccggcagcaccgaccgcactgcgtgaactggcacgtcgcttccgcagtgaaggtgatccggcagcgtctagtagctctgccctgctgaaccgtagcatcgccgcaaaactgctggcgcgcctgcataatggcggttatgttctgccagcagatatttttgcgaacctgccgaatccgccggacccgtttttcacccgtgcgcagatcgatcgtgaagcccgcaaagtgcgtgatggcattatgggtatgctgtacctgcaacgtatgccgaccgaatttgatgtggcaatggcgacggtttattacctggcggatcgcaacgtttctggcgaaacctttcacccgagtggcggtctgcgctatgaacgtaccccgacgggcggtgaactgttcggtctgccgtctccggaacgtctggcggaactggtgggtagtaccgtttacctgattggcgaacatctgacggaacacctgaatctgctggcccgcgcatatctggaacgctacggagcccgtcaggtggttatgatcgtggaaaccgaaacgggtgcggaaaccatgcgtcgcctgctgcatgatcacgtggaagcaggtcgcctgatgacgattgttgcgggcgatcagattgaagcggccatcgatcaggcgattacccgctatggtcgtccgggtccggtggtttgcaccccgtttcgcccgctgccgacggtgccgctggttggccgtaaagattctgattggagtaccgtgctgagcgaagccgaatttgcagaactgtgtgaacatcagctgacccatcacttccgcgttgcccgtaaaattgcactgagtgatggtgcaagcctggcactggtgaccccggaaaccacggcaacgagcaccacggaacagtttgcgctggccaacttcatcaaaaccacgctgcacgcattcaccgcgacgattggtgttgaatctgaacgcaccgcgcagcgtattctgatcaatcaggtggatctgactcgtcgcgcacgcgcggaagaaccgcgtgatccgcatgaacgccagcaggaactggaacgttttattgaagcagtgctgctggttaccgcaccgctgccgccggaagcagatactcgttacgcaggtcgtatccaccgtggtcgcgcgattaccgtgtaaggatctaggaggattatattatgatcgataccgcaccgctggcaccgccgcgtgctccgcgcagcaatccgattcgtgatcgcgtggattgggaagcgcagcgtgcagcagcactggccgatccgggtgcatttcatggtgcgatcgcccgtaccgttattcactggtatgatccgcagcatcactgctggattcgcttcaacgaaagctctcagcgttgggaaggtctggatgcagcaacgggtgctccggttacagtggattatcctgccgattaccagccgtggcagcaggcatttgatgatagtgaagcgccgttttatcgctggttcagcggcggtctgacgaacgcatgttttaatgaagttgatcgtcacgtgacaatgggttacggcgatgaagtggcgtattacttcgaaggtgatcgctgggataatagcctgaacaatggccgtggcggtccggtggttcaggaaacgattacccgtcgccgtctgctggttgaagtggttaaagcagcgcaggttctgcgcgatctgggcctgaaaaaaggtgatcgtatcgcgctgaacatgccgaatatcatgccgcagatttattacaccgaagccgcaaaacgcctgggtattctgtatacgccggtgtttggcggtttcagtgataaaaccctgagcgatcgcatccataatgcaggtgcgcgtgtggttattacctctgatggcgcgtatcgtaacgcccaggtggttccgtataaagaagcctacacggatcaggcactggataaatacatcccggtggaaaccgcccaggcaattgttgcacagacgctggcaaccctgccgctgaccgaaagtcagcgccagacgattatcaccgaagtggaagcagcactggcaggtgaaattacggttgaacgttctgatgttatgcgcggtgtgggcagtgcgctggccaaactgcgcgatctggatgccagtgtgcaggcaaaagttcgtaccgtgctggcacaggcgctggttgaaagcccgccgcgcgtggaagcagtggttgtggttcgtcatacgggtcaggaaatcctgtggaatgaaggccgtgatcgctggagccacgatctgctggatgcagcactggcgaaaattctggctaacgcacgcgccgcaggttttgatgttcactctgaaaacgatctgctgaatctgccggatgatcagctgatccgtgctctgtatgcgagtattccgtgcgaaccagttgatgccgaatatccgatgtttattatctacacgagcggttctaccggcaaaccgaaaggtgttattcatgttcacggcggttacgtggcgggcgtggttcataccctgcgcgttagtttcgatgccgaaccgggcgatacgatttatgtgatcgcagatccgggctggatcacaggtcagagctacatgctgacggcaaccatggcaggtcgtctgactggtgtgattgccgaaggttctccgctgtttccgagtgcgggccgctatgcctctattatcgaacgttacggtgttcagatttttaaagcgggcgttacgttcctgaaaaccgtgatgagtaacccgcagaatgttgaagatgtgcgcctgtatgatatgcacagtctgcgtgtggcaaccttttgtgcagagccggttagcccggcagtgcagcagttcggtatgcagatcatgacgccgcagtatattaatagctactgggcgacggaacatggcggtattgtgtggacccacttttatggcaaccaggatttcccgctgcgtccagatgcacatacgtacccgctgccgtgggttatgggtgatgtttgggtggcagaaaccgatgaatctggcaccacgcgctatcgcgtggcggatttcgatgaaaaaggtgaaatcgttatcaccgcaccgtatccgtacctgacgcgaaccctgtggggtgatgtgccgggttttgaagcgtatctgcgtggtgaaatcccgctgcgtgcatggaaaggtgatgcagaacgtttcgttaaaacctactggcgtcgtggtccgaatggcgaatggggttatatccagggcgattttgcgattaaatacccggatggtagtttcacgctgcatggccgcagcgatgatgttattaatgtgtccggccaccgtatgggtacggaagaaatcgaaggtgccattctgcgtgatcgccagatcaccccggattctccggtgggtaactgcattgtggttggcgcgccgcatcgtgaaaaaggcctgaccccggttgcatttatccagccagcaccgggtcgtcacctgacgggtgcagatcgccgtcgcctggatgaactggtgcgtaccgaaaaaggtgcagttagcgtgccggaagattatattgaagttagtgcgtttccggaaacccgcagcggtaaatacatgcgtcgcttcctgcgtaatatgatgctggatgaaccgctgggcgataccacgaccctgcgcaacccggaagtgctggaagaaatcgcggccaaaattgccgaatggaaacgtcgccagcgcatggcagaagaacagcagattatcgaacgttatcgctactttcgtattgaatatcatccgccgaccgcaagtgcaggtaaactggcagtggttacggttaccaatccgccggtgaacgccctgaatgaacgtgctctggatgaactgaacaccatcgtggatcacctggcgcgtcgccaggatgttgcagcgattgtgtttacgggtcagggtgctcgcagcttcgtggccggtgcggatatccgtcagctgctggaagaaattcataccgttgaagaagccatggcactgccgaacaatgcgcacctggcctttcgcaaaattgaacgtatgaacaaaccgtgcattgccgcaatcaatggtgtggcactgggcggtggcctggaatttgcgatggcctgtcattatcgcgttgccgatgtgtacgcagaatttggtcagccggaaatcaacctgcgtctgctgccgggttatggtggtacgcagcgtctgccgcgtctgctgtacaaacgcaacaatggtacaggcctgctgcgtgcgctggaaatgattctgggtggccgcagcgtgccagcagatgaagcactggaactgggtctgattgatgcaatcgcgaccggcgatcaggatagtctgagcctggcctgcgcactggcgcgtgcggcaatcggtgcagatggtcagctgattgaaagcgcagcggtgacccaggcctttcgtcatcgccacgaacagctggatgaatggcgtaaaccggacccgcgcttcgcggatgatgaactgcgctctattatcgcccatccgcgtatcgaacgcattatccgtcaggcgcataccgttggtcgtgatgcagcagtgcaccgtgcactggatgcaattcgttatggcattatccatggttttgaagccggcctggaacacgaagcaaaactgttcgccgaagcagtggttgatccgaatggtggcaaacgcggcatccgtgaatttctggatcgtcagtctgcaccgctgccgacacgtcgcccgctgattaccccggaacaggaacagctgctgcgtgatcagaaagaactgctgccggtgggtagtccgtttttccctggcgttgatcgcatcccgaaatggcagtatgcgcaggccgtgattcgtgatcccgatactggtgcagcagcacatggcgatccgatcgttgcggaaaaacagattatcgttccggtggaacgtccgcgtgcgaaccaggcactgatttacgttctggcgagcgaagtgaactttaatgatatttgggccatcacaggtattccggtgagccgcttcgatgaacatgatcgtgattggcacgtgacgggttctggtggcatcggcctgattgttgcgctgggcgaagaagcccgtcgcgaaggtcgtctgaaagttggcgatctggtggcgatctatagcggccagtctgatctgctgagcccgctgatgggtctggacccgatggcagccgattttgtgattcagggtaatgataccccggatggctctcatcagcagttcatgctggcacaggcaccgcagtgcctgccgatcccgacggatatgagcattgaagcagcgggttcttatatcctgaacctgggcaccatttaccgcgcactgtttacgaccctgcaaattaaagcgggtcgtacgattttcatcgaaggtgcagcaacgggtacaggtctggatgcagcacgcagcgcagcacgtaatggtctgcgcgttatcggcatggtgagtagctctagtcgcgcgtctaccctgctggcagcaggagcacatggtgcaattaaccgcaaagacccggaagtggccgattgttttacgcgagttccggaagatccgagcgcatgggcagcatgggaagcagccggtcagccgctgctggcaatgttccgtgcccagaatgatggtcgtctggccgattatgtggttagccacgcaggcgaaaccgcgtttccgcgctctttccagctgctgggtgaaccgcgtgatggtcatatcccgacgctgaccttttatggtgcgacgagtggctaccactttaccttcctgggcaaaccgggttctgccagtccgaccgaaatgctgcgtcgcgcaaacctgcgtgccggtgaagcagttctgatttattacggtgtgggcagcgatgatctggttgataccggaggcctggaagcgatcgaagccgcacgtcagatgggagcccgcattgtggttgtgacggtgtctgatgcccagcgcgaatttgttctgagtctgggtttcggtgcagcactgcgtggtgttgtgagcctggcggaactgaaacgtcgctttggcgatgaatttgaatggccgcgtaccatgccgccgctgccgaatgcacgtcaggacccgcagggcctgaaagaagcggtgcgtcgctttaacgatctggttttcaaaccgctgggtagcgcagttggcgtgtttctgcgctctgcggataacccgcgtggttatccggatctgattatcgaacgcgcagcgcatgatgccctggcagtgagtgccatgctgattaaaccgtttaccggccgtatcgtttatttcgaagatattggtggccgtcgctacagctttttcgcaccgcagatttgggtgcgtcagcgtcgcatttatatgccgacggcccagatttttggtacacacctgtctaacgcatacgaaattctgcgtctgaatgatgaaatcagtgcaggcctgctgacgattaccgaaccggcggttgtgccgtgggatgaactgccggaagcgcatcaggccatgtgggaaaaccgccacactgccgcaacctatgttgtgaatcatgcgctgccgcgcctgggtctgaaaaaccgtgatgaactgtacgaagcatggaccgcaggcgaacgtgaacaaaaactcatctcagaagaggatctgtaaggatctaagaggagaaagtgctatgagcatcttgtacgaagagcgtcttgatggcgctttacccgatgtcgaccgcacatcggtactgatggcactgcgtgagcatgtccctggacttgagatcctgcataccgatgaggagatcattccttacgagtgtgacgggttgagcgcgtatcgcacgcgtccattactggttgttctgcctaagcaaatggaacaggtgacagcgattctggctgtctgccatcgcctgcgtgtaccggtggtgacccgtggtgcaggcaccgggctttctggtggcgcgctgccgctggaaaaaggtgtgttgttggtgatggcgcgctttaaagagatcctcgacattaaccccgttggtcgccgcgcgcgcgtgcagccaggcgtgcgtaacctggcgatctcccaggccgttgcaccgcataatctctactacgcaccggacccttcctcacaaatcgcctgttccattggcggcaatgtggctgaaaatgccggcggcgtccactgcctgaaatatggtctgaccgtacataacctgctgaaaattgaagtgcaaacgctggacggcgaggcactgacgcttggatcggacgcgctggattcacctggttttgacctgctggcgctgttcaccggatcggaaggtatgctcggcgtgaccaccgaagtgacggtaaaactgctgccgaagccgcccgtggcgcgggttctgttagccagctttgactcggtagaaaaagccggacttgcggttggtgacatcatcgccaatggcattatccccggcgggctggagatgatggataacctgtcgatccgcgcggcggaagattttattcatgccggttatcccgtcgacgccgaagcgattttgttatgcgagctggacggcgtggagtctgacgtacaggaagactgcgagcgggttaacgacatcttgttgaaagcgggcgcgactgacgtccgtctggcacaggacgaagcagagcgcgtacgtttctgggccggtcgcaaaaatgcgttcccggcggtaggacgtatctccccggattactactgcatggatggcaccatcccgcgtcgcgccctgcctggcgtactggaaggcattgcccgtttatcgcagcaatatgatttacgtgttgccaacgtctttcatgccggagatggcaacatgcacccgttaatccttttcgatgccaacgaacccggtgaatttgcccgcgcggaagagctgggcgggaagatcctcgaactctgcgttgaagttggcggcagcatcagtggcgaacatggcatcgggcgagaaaaaatcaatcaaatgtgcgcccagttcaacagcgatgaaatcacgaccttccatgcggtcaaggcggcgtttgaccccgatggtttgctgaaccctgggaaaaacattcccacgctacaccgctgtgctgaatttggtgccatgcatgtgcatcacggtcatttacctttccctgaactggagcgtttctgatgctacgcgagtgtgattacagccaggcgctgctggagcaggtgaatcaggcgattagcgataaaacgccgctggtgattcagggcagcaatagcaaagcctttttaggtcgccctgtcaccgggcaaacgctggatgttcgttgtcatcgcggcattgttaattacgacccgaccgagctggtgataaccgcgcgtgtcggaacgccgctggtgacaattgaagcggcgctggaaagcgcggggcaaatgctcccctgtgagccgccgcattatggtgaagaagccacctggggcgggatggtcgcctgcgggctggcggggccgcgtcgcccgtggagcggttcggtccgcgattttgtcctcggcacgcgcatcattaccggcgctggaaaacatctgcgttttggtggcgaagtgatgaaaaacgttgccggatacgatctctcacggttaatggtcggaagctacggttgtcttggcgtgctcactgaaatctcaatgaaagtgttaccgcgaccgcgcgcctccctgagcctgcgtcgggaaatcagcctgcaagaagccatgagtgaaatcgccgagtggcaactccagccattacccattagtggcttatgttacttcgacaatgcgttgtggatccgccttgagggcggcgaaggatcggtaaaagcagcgcgtgaactgctgggtggcgaagaggttgccggtcagttctggcagcaattgcgtgaacaacaactgccgttcttctcgttaccaggtaccttatggcgcatttcattacccagtgatgcgccgatgatggatttacccggcgagcaactgatcgactggggcggggcgttacgctggctgaaatcgacagccgaggacaatcaaatccatcgcatcgcccgcaacgctggcggtcatgcgacccgctttagtgccggagatggtggctttgccccgctatcggctcctttattccgctatcaccagcagcttaaacagcagctcgacccttgcggcgtgtttaaccccggtcgcatgtacgcggaactttgaggagcaggctatgcaaacccaattaactgaagagatgcggcagaacgcgcgcgcgctggaagccgacagcatcctgcgcgcctgtgttcactgcggattttgtaccgcaacctgcccaacctatcagcttctgggcgatgaactggacgggccgcgcgggcgcatctatctgattaaacaggtgctggaaggcaacgaagtcacgcttaaaacacaggagcatctcgatcgctgcctcacttgccgtaattgtgaaaccacctgtccttctggtgtgcgctatcacaatttgctggatatcgggcgtgatattgtcgagcagaaagtgaaacgcccactgccggagcgaatactgcgcgaaggattgcgccaggtagtgccgcgtccggcggtcttccgtgcgctgacgcaggtagggctggtgctgcgaccgtttttaccggaacaggtcagagcaaaactgcctgctgaaacggtgaaagctaaaccgcgtccgccgctgcgccataagcgtcgggttttaatgttggaaggctgcgcccagcctacgctttcgcccaacaccaacgcggcaactgcgcgagtgctggatcgtctggggatcagcgtcatgccagctaacgaagcaggctgttgtggcgcggtggactatcatcttaatgcgcaggagaaagggctggcacgggcgcgcaataatattgatgcctggtggcccgcgattgaagcaggtgccgaggcaattttgcaaaccgccagcggctgcggcgcgtttgtcaaagagtatgggcagatgctgaaaaacgatgcgttatatgccgataaagcacgtcaggtcagtgaactggcggtcgatttagtcgaacttctgcgcgaggaaccgctggaaaaactggcaattcgcggcgataaaaagctggccttccactgtccgtgtaccctacaacatgcgcaaaagctgaacggcgaagtggaaaaagtgttgcttcgtcttggatttaccttaacggacgttcccgacagccatctgtgctgcggttcagcgggaacatatgcgttaacgcatcccgatctggcacgccagctgcgggataacaaaatgaatgcgctggaaagcggcaaaccggaaatgatcgtcaccgccaacattggttgccagacgcatctggcgagcgccggtcgtacctctgtgcgtcactggattgaaattgtagaacaagcccttgaaaaggaataaggatccctcgactctagaggatccccgggtaccgagctcgaattaggaggaattaataatgattgaacaagatggattgcacgcaggttctccggccgcttgggtggagaggctattcggctatgactgggcacaacagacaatcggctgctctgatgccgccgtgttccggctgtcagcgcaggggaggccggttctttttgtcaagaccgacctgtccggtgccctgaatgaacttcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggcgcgccttcgttagtgttagtctagaactagtttagtaaaaaacgagcaatataagccttctttaaataagaaagagggcttatattactcgtttttttctataaaaatgagcaaatttttatagagtatcatattttactttatttattatattaataataaataataataataaataataaaaaattactatatattttttattagaaaaaaaataaggtggaatttgctacctttttttattttttattgaaatttgtatttttttttttttttagacaatacaaaaaagaatagatagtagcgtaggggctccacttggctcgggggatatagctcagttggtagagctccgctcttgcaattgggtcgttgcgattacgggttgggtgtctaattgtccaggcggtaatgatagtatcttgtacctgaaccggtggctcactttttctaagtaatggggaaaaggaccgaaacatgccactgaaagactctactgagacaaagatgggctgtcaagaacgtagaggaggtaggatggtcagttggtcagatctagtatggatcgtacatggacggtagttggagtcggcggctctcctagggttccctcgtctgggattgatccctggggaagaggatcaagttggcccttgcgaacagcttgatgcactatctcccttcaaccctttgagcgaaatgcggcaaaaggaaggaaaatccatggaccgaccccatcgtctccaccccgtaggaactacgagatcaccccaaggacgccttcggtatccaggggtcgcggaccgaccatagaaccctgttcaataagtggaatgcattagctgtccgctcgcaggttgggcagtaagggtcggagaagggcaatcactcattcttaaaaccagcattcgaaagagttggggcggaaaagggggggaaagctctccgttcctggttctcctgtagctggatcctctagaaccacaagaatccttagttggaatgggattccagctcatcaccttttgagattttgagaagagttgctctttggagagcacagtacgatgaaagttgtaagctgtgttcgggggggagttcttgtctatcgttggcctctatggtagaatcagtcaggggcctgataggcggtggtttaccctgtggcggatgtcagcggttcgagtccgcttatctccaactcgtgaacttagccgatacaaagctatatgatagcacccaatttttccgattcggcacactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 95 - DNA, 13513 bp, Artificial Sequence
  source: /note=“Description of Artificial Sequence Synthetic polynucleotide”
ggcccccagcaggaggcccgcacgacgggctattagctcagtggtagagcgcgcccctgataattgcgtcgttgtgcctgggctgtgagggctctcagccacatggatagttcaatgtgctcatcagcgcctgaccctgagatgtggatcatccaaggcacattagcatggcgtactcctcctgttcgaaccggggtttgaaaccaaacttctcctcaggaggatagatggggcgattcaggtgagatccaatgtagatccaactttctattcactcgtgggatccgggcggtccggaggggaccactatggctcctctcttctcgagaatccatacatcccttatcagtgtatggacagctatctctcgagcgcaggtttaggttcggcctcaatgggaaaataaaatggagcacctaacaacgtatcttcacagaccaagaactacgagatcacccctttcattctggggtgacggagggatcgtaccgttcgagcctttttttcatgttatctatctcttgactcgaaatgggagcaggtttgaaaaaggatcttagagtgtctagggttaggccagtagggtctcttaacgccctcttttttcttctcatcgaagttatttcacaaatacttcctatggtaaggaagagggggggaacaagcacacttggagagcgcagtacaacggagagttgtatgctgcgttcgggaaggatgaatcgctcccgaaaaggaatctattgattctctcccaattggttggaccataggtgcgatgatttacttcacgggcgaggtctctggttcaaatccaggatggcccagctgcggctccctcgctgtgatcgaataagaatggataagaggctcgtgggattgacgtgagggggtaggggtagctatatttctgggagcgaactccatgcgaatatgaagcgcatggatacaagttatgacttggaatgaaagacaattccgaatcgaattcagatctaagaggagaaagtgctatgcgtaaactggcccataacttttataaaccgctggcaattggagcaccggaaccgatccgcgaactgccggttcgtccggaacgcgtggttcatttctttccgccgcacgtggaaaaaattcgtgctcgcatcccggaagttgctaaacaggttgatgtcctgtgcggcaacctggaagatgcaattccgatggacgctaaagaagcggcccgtaatggttttatcgaagtcgtgaaagcaaccgatttcggcgacacggctctgtgggtgcgcgttaacgcgctgaacagcccgtgggtgctggatgacattgccgaaatcgttgcagctgtcggtaacaaactggatgtcattatgatcccgaaagtggaaggcccgtgggatattcactttgttgaccagtacctggcgctgctggaagcccgtcatcaaatcaaaaaaccgattctgatccacgcgctgctggaaaccgcccagggtatggtgaatctggaagaaattgcgggtgccagcccgcgtatgcacggtttctctctgggtccggcggatctggcagcctcgcgtggtatgaaaaccacgcgcgttggcggtggccacccgttttatggtgtcctggccgatccgcaggaaggccaagcagaacgtccgttctatcagcaggatctgtggcattacaccattgcgcgtatggttgacgtggcagttgctcacggtctgcgtgccttttacggtccgttcggcgatatcaaagacgaagcagcttgcgaagcacagtttcgcaatgctttcctgctgggttgtacgggcgcatggagtctggctccgaaccaaattccgatcgcaaaacgtgtcttttccccggatgtgaatgaagttctgttcgcgaaacgcattctggaagccatgccggatggttctggcgtggcgatgatcgacggtaaaatgcaggatgacgcgacgtggaaacaagccaaagtcattgtggatctggcgcgtatgatcgccaaaaaagatccggacctggcgcaggcctatggcctgtagttcacacaggaaaccacatgaaaggtattctgcatggtctgcgtgtggtggaaggttcggcttttgtcgctgccccgctgggtggtatgacgctggctcaactgggtgcagatgttatccgctttgacccgattggcggtggcctggattataaacgttggccggtcaccctggacggcaaacatagtctgttctgggctggtctgaacaaaggcaaacgctccattgcgatcgatattcgccatccgcgtggtcaggaactgctgacccaactgatctgcgctccgggtgaacacgcaggcctgtttattacgaatttcccggctcgtggttggctgtcatacgatgaactgaaacgtcaccgcgcggacctgatcatggttaatctggtcggtcgtcgcgatggtggctcggaagtggactacaccgttaatccgcagctgggtctgccgtttatgacgggtccggtgaccacgccggatgtggttaaccatgttctgcctgcctgggacatcgtcacaggtcagatgattgcactgggcctgctggcggcagaacgtcaccgtcgcctgacgggtgaaggccaactggtgaaaatcgctctgaaagatgttggtctggcgatgattggtcatctgggcatgatcgccgaagtgatgattaacgataccgaccgtccgcgtcagggcaattatctgtacggtgcatttggccgcgatttcgaaaccctggacggtaaacgtgttatggtcgtgggcctgacggatctgcaatggaaagccctgggtaaagcaaccggcctgacggacgcatttaacgctctgggtgcgcgtctgggcctgaatatggatgaagaaggtgaccgtttccgcgcgcgtcatgaaattgcagctctgctggaaccgtggtttcacgctcgtaccctggcggaagtgcgtcgcatcttcgaacagcatcgtgtcacctgggctccgtatcgcacggtgcgtgaagcgattgcccaggacccggactgtagcaccgataatccgatgtttgctatggttgaacaaccgggtatcggcagctacctgatgccgggctctccgctggatttcaccgcagtcccgcgtctgccggtgcagccagcaccgcgtctgggtgaacacacggatgaaattctgctggaagttctgggcctgagtgaagccgaagttggtcgtctgcatgatgaaggtattgttgctggcccggatcgtgctgcctgaattaaagaggagaaatagcaatgtcctcggcggattggatggcttggattggtcgcacggaacaggtggaagatgatatttgtctggcacaggctattgcagcggctgctaccctggaaccgccgagcggagcaccgacggctgattctccgctgccgccgctgtggcattggttttatttcctgccgcgtgccccgcagagtcaactgagctctgacggtcacccgcagcgcggcggttttattccgccgatcccgtacccgcgtcgcatgtttgcgggtgcccgtattcgcttccatcacccgctgcgtatcggtcagccagcacgtcgcgaaggtgtgattcgtaacatcacccaaaaaagtggccgctccggtccgctggcattcgttacggtcggctatcagatttaccaacatgaaatgctgtgcattgaagaagaacaggatatcgtttatcgtgaaccgggtgctccggtcccggcaccgaccccggtcgaactgccgccggttcacgatgcgattacccgtacagtggttcctgacccgcgtctgctgtttcgcttctccgcactgacgtttaacgctcatcgtatccactatgatcgcccttacgcgcagcatgaagaaggctaccctggtctggtcgttcacggtccgctggttgcggttctgctgatggaactggcgcgtcatcacaccagccgcccgattgttggtttttcattccgttcgcaagcgccgctgtttgacctggcaccgttccgtctgctggcacgtccgaatggtgatcgcatcgacctggaagcacaaggcccggatggcgcaacggcactgtcggcaacggtggaactgggtggttaaaggagggcatctatgtccgcaaaaacgaatccgggcaacttctttgaagatttccgtctgggccaaaccattgtccacgctacgccgcgcaccattaccgaaggcgatgtggccctgtataccagcctgtacggttctcgttttgcactgaccagctctacgccgttcgctcagtcactgggcctggaacgtgctccgattgactcgctgctggtgtttcatatcgttttcggcaaaaccgttccggatattagtctgaacgcgatcgccaatctgggttatgcgggcggtcgttttggtgccgtggtttacccaggtgacaccctgtcaaccacgtcgaaagtgattggcctgcgccagaacaaagatggcaaaacgggtgtcgtgtatgttcactctgtcggtgtgaatcaatgggacgaagttgtcctggaatacatccgttgggttatggtccgtaaacgcgatccgaacgcaccggctccggaaaccgtggttccggatctgccggacagcgtgccggttaccgatctgacggtcccgtataccgtgagtgcggccaactacaatctggcgcatgccggttccaattatctgtgggatgactacgaagtgggcgaaaaaattgatcatgtggacggtgtgaccatcgaagaagcagaacacatgcaggctacccgtctgtatcaaaacacggcccgcgttcattttaatctgcacgtcgaacgtgaaggccgcttcggtcgtcgcattgtttacggcggtcatattatcagcctggcacgtagtctgtcctttaacggcctggcaaatgctctgagtattgcagctatcaactccggccgccacaccaatccgagcttcgcaggtgacacgatttatgcttggtctgaaatcctggcgaaaatggccattccgggtcgtaccgatatcggagcactgcgtgttcgtaccgtcgcaacgaaagatcgtccgtgccacgacttcccgtatcgcgatgcggaaggtaactatgacccggctgttgtgctggattttgattacaccgtgctgatgccgcgtcgtggcgaacaaaaactcatctcagaagaggatctgaatagcgccgtcgactaagcttgcatgcctgcaggtcgactctagaggatctaagaggagaaagtgctatgagcatcttgtacgaagagcgtcttgatggcgctttacccgatgtcgaccgcacatcggtactgatggcactgcgtgagcatgtccctggacttgagatcctgcataccgatgaggagatcattccttacgagtgtgacgggttgagcgcgtatcgcacgcgtccattactggttgttctgcctaagcaaatggaacaggtgacagcgattctggctgtctgccatcgcctgcgtgtaccggtggtgacccgtggtgcaggcaccgggctttctggtggcgcgctgccgctggaaaaaggtgtgttgttggtgatggcgcgctttaaagagatcctcgacattaaccccgttggtcgccgcgcgcgcgtgcagccaggcgtgcgtaacctggcgatctcccaggccgttgcaccgcataatctctactacgcaccggacccttcctcacaaatcgcctgttccattggcggcaatgtggctgaaaatgccggcggcgtccactgcctgaaatatggtctgaccgtacataacctgctgaaaattgaagtgcaaacgctggacggcgaggcactgacgcttggatcggacgcgctggattcacctggttttgacctgctggcgctgttcaccggatcggaaggtatgctcggcgtgaccaccgaagtgacggtaaaactgctgccgaagccgcccgtggcgcgggttctgttagccagctttgactcggtagaaaaagccggacttgcggttggtgacatcatcgccaatggcattatccccggcgggctggagatgatggataacctgtcgatccgcgcggcggaagattttattcatgccggttatcccgtcgacgccgaagcgattttgttatgcgagctggacggcgtggagtctgacgtacaggaagactgcgagcgggttaacgacatcttgttgaaagcgggcgcgactgacgtccgtctggcacaggacgaagcagagcgcgtacgtttctgggccggtcgcaaaaatgcgttcccggcggtaggacgtatctccccggattactactgcatggatggcaccatcccgcgtcgcgccctgcctggcgtactggaaggcattgcccgtttatcgcagcaatatgatttacgtgttgccaacgtctttcatgccggagatggcaacatgcacccgttaatccttttcgatgccaacgaacccggtgaatttgcccgcgcggaagagctgggcgggaagatcctcgaactctgcgttgaagttggcggcagcatcagtggcgaacatggcatcgggcgagaaaaaatcaatcaaatgtgcgcccagttcaacagcgatgaaatcacgaccttccatgcggtcaaggcggcgtttgaccccgatggtttgctgaaccctgggaaaaacattcccacgctacaccgctgtgctgaatttggtgccatgcatgtgcatcacggtcatttacctttccctgaactggagcgtttctgatgctacgcgagtgtgattacagccaggcgctgctggagcaggtgaatcaggcgattagcgataaaacgccgctggtgattcagggcagcaatagcaaagcctttttaggtcgccctgtcaccgggcaaacgctggatgttcgttgtcatcgcggcattgttaattacgacccgaccgagctggtgataaccgcgcgtgtcggaacgccgctggtgacaattgaagcggcgctggaaagcgcggggcaaatgctcccctgtgagccgccgcattatggtgaagaagccacctggggcgggatggtcgcctgcgggctggcggggccgcgtcgcccgtggagcggttcggtccgcgattttgtcctcggcacgcgcatcattaccggcgctggaaaacatctgcgttttggtggcgaagtgatgaaaaacgttgccggatacgatctctcacggttaatggtcggaagctacggttgtcttggcgtgctcactgaaatctcaatgaaagtgttaccgcgaccgcgcgcctccctgagcctgcgtcgggaaatcagcctgcaagaagccatgagtgaaatcgccgagtggcaactccagccattacccattagtggcttatgttacttcgacaatgcgttgtggatccgccttgagggcggcgaaggatcggtaaaagcagcgcgtgaactgctgggtggcgaagaggttgccggtcagttctggcagcaattgcgtgaacaacaactgccgttcttctcgttaccaggtaccttatggcgcatttcattacccagtgatgcgccgatgatggatttacccggcgagcaactgatcgactggggcggggcgttacgctggctgaaatcgacagccgaggacaatcaaatccatcgcatcgcccgcaacgctggcggtcatgcgacccgctttagtgccggagatggtggctttgccccgctatcggctcctttattccgctatcaccagcagcttaaacagcagctcgacccttgcggcgtgtttaaccccggtcgcatgtacgcggaactttgaggagcaggctatgcaaacccaattaactgaagagatgcggcagaacgcgcgcgcgctggaagccgacagcatcctgcgcgcctgtgttcactgcggattttgtaccgcaacctgcccaacctatcagcttctgggcgatgaactggacgggccgcgcgggcgcatctatctgattaaacaggtgctggaaggcaacgaagtcacgcttaaaacacaggagcatctcgatcgctgcctcacttgccgtaattgtgaaaccacctgtccttctggtgtgcgctatcacaatttgctggatatcgggcgtgatattgtcgagcagaaagtgaaacgcccactgccggagcgaatactgcgcgaaggattgcgccaggtagtgccgcgtccggcggtcttccgtgcgctgacgcaggtagggctggtgctgcgaccgtttttaccggaacaggtcagagcaaaactgcctgctgaaacggtgaaagctaaaccgcgtccgccgctgcgccataagcgtcgggttttaatgttggaaggctgcgcccagcctacgctttcgcccaacaccaacgcggcaactgcgcgagtgctggatcgtctggggatcagcgtcatgccagctaacgaagcaggctgttgtggcgcggtggactatcatcttaatgcgcaggagaaagggctggcacgggcgcgcaataatattgatgcctggtggcccgcgattgaagcaggtgccgaggcaattttgcaaaccgccagcggctgcggcgcgtttgtcaaagagtatgggcagatgctgaaaaacgatgcgttatatgccgataaagcacgtcaggtcagtgaactggcggtcgatttagtcgaacttctgcgcgaggaaccgctggaaaaactggcaattcgcggcgataaaaagctggccttccactgtccgtgtaccctacaacatgcgcaaaagctgaacggcgaagtggaaaaagtgttgcttcgtcttggatttaccttaacggacgttcccgacagccatctgtgctgcggttcagcgggaacatatgcgttaacgcatcccgatctggcacgccagctgcgggataacaaaatgaatgcgctggaaagcggcaaaccggaaatgatcgtcaccgccaacattggttgccagacgcatctggcgagcgccggtcgtacctctgtgcgtcactggattgaaattgtagaacaagcccttgaaaaggaataaggatccctcgactctagaggatccccgggtaccgagctcgaattaggaggaattaataatgattgaacaagatggattgcacgcaggttctccggccgcttgggtggagaggctattcggctatgactgggcacaacagacaatcggctgctctgatgccgccgtgttccggctgtcagcgcaggggaggccggttctttttgtcaagaccgacctgtccggtgccctgaatgaacttcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggcgcgccttcgttagtgttagtctagaactagtttagtaaaaaacgagcaatataagccttctttaaataagaaagagggcttatattactcgtttttttctataaaaatgagcaaatttttatagagtatcatattttactttatttattatattaataataaataataataataaataataaaaaattactatatattttttattagaaaaaaaataaggtggaatttgctacctttttttattttttattgaaatttgtatttttttttttttttagacaatacaaaaaagaatagatagtagcgtaggggctccacttggctcgggggatatagctcagttggtagagctccgctcttgcaattgggtcgttgcgattacgggttgggtgtctaattgtccaggcggtaatgatagtatcttgtacctgaaccggtggctcactttttctaagtaatggggaaaaggaccgaaacatgccactgaaagactctactgagacaaagatgggctgtcaagaacgtagaggaggtaggatggtcagttggtcagatctagtatggatcgtacatggacggtagttggagtcggcggctctcctagggttccctcgtctgggattgatccctggggaagaggatcaagttggcccttgcgaacagcttgatgcactatctcccttcaaccctttgagcgaaatgcggcaaaaggaaggaaaatccatggaccgaccccatcgtctccaccccgtaggaactacgagatcaccccaaggacgccttcggtatccaggggtcgcggaccgaccatagaaccctgttcaataagtggaatgcattagctgtccgctcgcaggttgggcagtaagggtcggagaagggcaatcactcattcttaaaaccagcattcgaaagagttggggcggaaaagggggggaaagctctccgttcctggttctcctgtagctggatcctctagaaccacaagaatccttagttggaatgggattccagctcatcaccttttgagattttgagaagagttgctctttggagagcacagtacgatgaaagttgtaagctgtgttcgggggggagttcttgtctatcgttggcctctatggtagaatcagtcaggggcctgataggcggtggtttaccctgtggcggatgtcagcggttcgagtccgcttatctccaactcgtgaacttagccgatacaaagctatatgatagcacccaatttttccgattcggcacactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 96 - DNA, 22748 bp, Artificial Sequence
  source: /note=“Description of Artificial Sequence Synthetic polynucleotide”
ggcccccagcaggaggcccgcacgacgggctattagctcagtggtagagcgcgcccctgataattgcgtcgttgtgcctgggctgtgagggctctcagccacatggatagttcaatgtgctcatcagcgcctgaccctgagatgtggatcatccaaggcacattagcatggcgtactcctcctgttcgaaccggggtttgaaaccaaacttctcctcaggaggatagatggggcgattcaggtgagatccaatgtagatccaactttctattcactcgtgggatccgggcggtccggaggggaccactatggctcctctcttctcgagaatccatacatcccttatcagtgtatggacagctatctctcgagcgcaggtttaggttcggcctcaatgggaaaataaaatggagcacctaacaacgtatcttcacagaccaagaactacgagatcacccctttcattctggggtgacggagggatcgtaccgttcgagcctttttttcatgttatctatctcttgactcgaaatgggagcaggtttgaaaaaggatcttagagtgtctagggttaggccagtagggtctcttaacgccctcttttttcttctcatcgaagttatttcacaaatacttcctatggtaaggaagagggggggaacaagcacacttggagagcgcagtacaacggagagttgtatgctgcgttcgggaaggatgaatcgctcccgaaaaggaatctattgattctctcccaattggttggaccataggtgcgatgatttacttcacgggcgaggtctctggttcaaatccaggatggcccagctgcggctccctcgctgtgatcgaataagaatggataagaggctcgtgggattgacgtgagggggtaggggtagctatatttctgggagcgaactccatgcgaatatgaagcgcatggatacaagttatgacttggaatgaaagacaattccgaatcgaattcagatctaggaggattatattatgggcgagcagaagctgattagcgaggaagacctaagcggcacaggtcgtctggcaggcaaaattgcactgatcacgggcggtgcgggcaacatcggtagcgaactgacccgtcgctttctggccgaaggtgcaacggtgattatctctggccgtaatcgcgccaaactgaccgcactggcggaacgtatgcaggccgaagcaggtgtgcctgcgaaacgcattgatctggaagttatggatggtagtgatccggtggcggttcgtgctggcattgaagcaatcgtggcgcgccacggtcagattgatatcctggttaacaatgcaggcagcgcaggagcacagcgtcgcctggcagaaattccgctgaccgaagcagaactgggtccgggtgcagaagaaacgctgcacgccagtatcgcaaacctgctgggcatgggttggcatctgatgcgtattgcagcaccgcacatgccggttggcagcgcagtgatcaatgttagcaccattttctctcgtgcggaatattacggtcgcattccgtatgtgacgccgaaagcagcgctgaacgccctgtctcagctggcagcacgcgaactgggagcacgtggtatccgcgttaataccatttttccgggtccgatcgaaagtgatcgtattcgcacggtgttccagcgtatggatcagctgaaaggccgcccggaaggtgataccgcccatcactttctgaacacgatgcgtctgtgccgcgcgaatgatcagggagccctggaacgtcgctttccgagcgtgggtgatgttgcggatgcagccgttttcctggcatctgcggaaagtgcagcgctgtctggtgaaaccatcgaagtgacgcatggcatggaactgccagcgtgtagcgaaacctctctgctggcccgcaccgatctgcgtactattgatgccagcggtcgtaccacgctgatctgcgcaggtgatcagattgaagaagttatggcgctgaccggaatgcttcgtacttgtggtagcgaagtgattatcggctttcgctctgccgcagcgctggcacagttcgaacaggcagtgaacgaaagccgtcgcctggcaggtgcagattttaccccgccgatcgcactgccgctggacccgcgtgatccggcgacgattgatgccgttttcgattggggcgcgggtgaaaacaccggcggtatccatgccgcagtgattctgccagcaacgagccacgaaccggcaccgtgcgtgatcgaagttgatgatgagcgcgttctgaatttcctggcggatgaaattacaggtacgattgtgatcgcaagtcgtctggcgcgctattggcagagccagcgtctgaccccaggtgcgcgtgcccgtggtccgcgcgttatctttctgagtaacggcgcggatcagaacggcaatgtgtatggtcgtattcagagcgcggccattggtcagctgatccgtgtttggcgtcatgaagccgaactggattaccagcgcgcaagcgcagcaggtgatcacgtgctgccgccggtttgggcgaaccagattgtgcgctttgccaatcgttctctggaaggtctggaatttgcttgcgcgtggaccgcgcagctgctgcatagtcagcgtcacattaatgaaatcacgctgaacattccggccaatatctctgcaaccacgggagcacgcagtgcaagcgttggttgggcggaaagtctgatcggcctgcatctgggtaaagtggcactgattaccggcggtagcgcgggtattggcggtcagatcggtcgtctgctggcactgtctggtgcccgcgttatgctggcagcacgtgatcgccataaactggaacagatgcaggccatgatccagagcgaactggcagaagtgggctataccgatgtggaagatcgtgttcacattgccccaggttgtgatgtgagttctgaagcacagctggcggatctggttgaacgcaccctgtctgcgtttggcacggtggattatctgatcaacaatgcaggcattgccggtgtggaagaaatggttatcgatatgccggttgaaggttggcgtcataccctgttcgccaacctgattagtaattacagcctgatgcgcaaactggcaccgctgatgaaaaaacagggctctggttatatcctgaacgtgagtagctactttggcggtgaaaaagatgcggccattccgtatccgaatcgtgcggattacgccgttagtaaagcgggccagcgtgcgatggcagaagtgtttgcccgcttcctgggtccagaaattcagatcaatgcgattgcaccgggtccggtggaaggtgatcgtctgcgtggtacaggtgaacgtccgggtctgtttgcacgtcgcgcacgtctgatcctggaaaacaaacgcctgaatgaactgcacgcagctctgattgccgcagcgcgcaccgatgaacgtagtatgcacgaactggtggaactgctgctgccgaacgatgttgcagcactggaacagaatccggcagcaccgaccgcactgcgtgaactggcacgtcgcttccgcagtgaaggtgatccggcagcgtctagtagctctgccctgctgaaccgtagcatcgccgcaaaactgctggcgcgcctgcataatggcggttatgttctgccagcagatatttttgcgaacctgccgaatccgccggacccgtttttcacccgtgcgcagatcgatcgtgaagcccgcaaagtgcgtgatggcattatgggtatgctgtacctgcaacgtatgccgaccgaatttgatgtggcaatggcgacggtttattacctggcggatcgcaacgtttctggcgaaacctttcacccgagtggcggtctgcgctatgaacgtaccccgacgggcggtgaactgttcggtctgccgtctccggaacgtctggcggaactggtgggtagtaccgtttacctgattggcgaacatctgacggaacacctgaatctgctggcccgcgcatatctggaacgctacggagcccgtcaggtggttatgatcgtggaaaccgaaacgggtgcggaaaccatgcgtcgcctgctgcatgatcacgtggaagcaggtcgcctgatgacgattgttgcgggcgatcagattgaagcggccatcgatcaggcgattacccgctatggtcgtccgggtccggtggtttgcaccccgtttcgcccgctgccgacggtgccgctggttggccgtaaagattctgattggagtaccgtgctgagcgaagccgaatttgcagaactgtgtgaacatcagctgacccatcacttccgcgttgcccgtaaaattgcactgagtgatggtgcaagcctggcactggtgaccccggaaaccacggcaacgagcaccacggaacagtttgcgctggccaacttcatcaaaaccacgctgcacgcattcaccgcgacgattggtgttgaatctgaacgcaccgcgcagcgtattctgatcaatcaggtggatctgactcgtcgcgcacgcgcggaagaaccgcgtgatccgcatgaacgccagcaggaactggaacgttttattgaagcagtgctgctggttaccgcaccgctgccgccggaagcagatactcgttacgcaggtcgtatccaccgtggtcgcgcgattaccgtgtaaggatctaggaggattatattatgatcgataccgcaccgctggcaccgccgcgtgctccgcgcagcaatccgattcgtgatcgcgtggattgggaagcgcagcgtgcagcagcactggccgatccgggtgcatttcatggtgcgatcgcccgtaccgttattcactggtatgatccgcagcatcactgctggattcgcttcaacgaaagctctcagcgttgggaaggtctggatgcagcaacgggtgctccggttacagtggattatcctgccgattaccagccgtggcagcaggcatttgatgatagtgaagcgccgttttatcgctggttcagcggcggtctgacgaacgcatgttttaatgaagttgatcgtcacgtgacaatgggttacggcgatgaagtggcgtattacttcgaaggtgatcgctgggataatagcctgaacaatggccgtggcggtccggtggttcaggaaacgattacccgtcgccgtctgctggttgaagtggttaaagcagcgcaggttctgcgcgatctgggcctgaaaaaaggtgatcgtatcgcgctgaacatgccgaatatcatgccgcagatttattacaccgaagccgcaaaacgcctgggtattctgtatacgccggtgtttggcggtttcagtgataaaaccctgagcgatcgcatccataatgcaggtgcgcgtgtggttattacctctgatggcgcgtatcgtaacgcccaggtggttccgtataaagaagcctacacggatcaggcactggataaatacatcccggtggaaaccgcccaggcaattgttgcacagacgctggcaaccctgccgctgaccgaaagtcagcgccagacgattatcaccgaagtggaagcagcactggcaggtgaaattacggttgaacgttctgatgttatgcgcggtgtgggcagtgcgctggccaaactgcgcgatctggatgccagtgtgcaggcaaaagttcgtaccgtgctggcacaggcgctggttgaaagcccgccgcgcgtggaagcagtggttgtggttcgtcatacgggtcaggaaatcctgtggaatgaaggccgtgatcgctggagccacgatctgctggatgcagcactggcgaaaattctggctaacgcacgcgccgcaggttttgatgttcactctgaaaacgatctgctgaatctgccggatgatcagctgatccgtgctctgtatgcgagtattccgtgcgaaccagttgatgccgaatatccgatgtttattatctacacgagcggttctaccggcaaaccgaaaggtgttattcatgttcacggcggttacgtggcgggcgtggttcataccctgcgcgttagtttcgatgccgaaccgggcgatacgatttatgtgatcgcagatccgggctggatcacaggtcagagctacatgctgacggcaaccatggcaggtcgtctgactggtgtgattgccgaaggttctccgctgtttccgagtgcgggccgctatgcctctattatcgaacgttacggtgttcagatttttaaagcgggcgttacgttcctgaaaaccgtgatgagtaacccgcagaatgttgaagatgtgcgcctgtatgatatgcacagtctgcgtgtggcaaccttttgtgcagagccggttagcccggcagtgcagcagttcggtatgcagatcatgacgccgcagtatattaatagctactgggcgacggaacatggcggtattgtgtggacccacttttatggcaaccaggatttcccgctgcgtccagatgcacatacgtacccgctgccgtgggttatgggtgatgtttgggtggcagaaaccgatgaatctggcaccacgcgctatcgcgtggcggatttcgatgaaaaaggtgaaatcgttatcaccgcaccgtatccgtacctgacgcgaaccctgtggggtgatgtgccgggttttgaagcgtatctgcgtggtgaaatcccgctgcgtgcatggaaaggtgatgcagaacgtttcgttaaaacctactggcgtcgtggtccgaatggcgaatggggttatatccagggcgattttgcgattaaatacccggatggtagtttcacgctgcatggccgcagcgatgatgttattaatgtgtccggccaccgtatgggtacggaagaaatcgaaggtgccattctgcgtgatcgccagatcaccccggattctccggtgggtaactgcattgtggttggcgcgccgcatcgtgaaaaaggcctgaccccggttgcatttatccagccagcaccgggtcgtcacctgacgggtgcagatcgccgtcgcctggatgaactggtgcgtaccgaaaaaggtgcagttagcgtgccggaagattatattgaagttagtgcgtttccggaaacccgcagcggtaaatacatgcgtcgcttcctgcgtaatatgatgctggatgaaccgctgggcgataccacgaccctgcgcaacccggaagtgctggaagaaatcgcggccaaaattgccgaatggaaacgtcgccagcgcatggcagaagaacagcagattatcgaacgttatcgctactttcgtattgaatatcatccgccgaccgcaagtgcaggtaaactggcagtggttacggttaccaatccgccggtgaacgccctgaatgaacgtgctctggatgaactgaacaccatcgtggatcacctggcgcgtcgccaggatgttgcagcgattgtgtttacgggtcagggtgctcgcagcttcgtggccggtgcggatatccgtcagctgctggaagaaattcataccgttgaagaagccatggcactgccgaacaatgcgcacctggcctttcgcaaaattgaacgtatgaacaaaccgtgcattgccgcaatcaatggtgtggcactgggcggtggcctggaatttgcgatggcctgtcattatcgcgttgccgatgtgtacgcagaatttggtcagccggaaatcaacctgcgtctgctgccgggttatggtggtacgcagcgtctgccgcgtctgctgtacaaacgcaacaatggtacaggcctgctgcgtgcgctggaaatgattctgggtggccgcagcgtgccagcagatgaagcactggaactgggtctgattgatgcaatcgcgaccggcgatcaggatagtctgagcctggcctgcgcactggcgcgtgcggcaatcggtgcagatggtcagctgattgaaagcgcagcggtgacccaggcctttcgtcatcgccacgaacagctggatgaatggcgtaaaccggacccgcgcttcgcggatgatgaactgcgctctattatcgcccatccgcgtatcgaacgcattatccgtcaggcgcataccgttggtcgtgatgcagcagtgcaccgtgcactggatgcaattcgttatggcattatccatggttttgaagccggcctggaacacgaagcaaaactgttcgccgaagcagtggttgatccgaatggtggcaaacgcggcatccgtgaatttctggatcgtcagtctgcaccgctgccgacacgtcgcccgctgattaccccggaacaggaacagctgctgcgtgatcagaaagaactgctgccggtgggtagtccgtttttccctggcgttgatcgcatcccgaaatggcagtatgcgcaggccgtgattcgtgatcccgatactggtgcagcagcacatggcgatccgatcgttgcggaaaaacagattatcgttccggtggaacgtccgcgtgcgaaccaggcactgatttacgttctggcgagcgaagtgaactttaatgatatttgggccatcacaggtattccggtgagccgcttcgatgaacatgatcgtgattggcacgtgacgggttctggtggcatcggcctgattgttgcgctgggcgaagaagcccgtcgcgaaggtcgtctgaaagttggcgatctggtggcgatctatagcggccagtctgatctgctgagcccgctgatgggtctggacccgatggcagccgattttgtgattcagggtaatgataccccggatggctctcatcagcagttcatgctggcacaggcaccgcagtgcctgccgatcccgacggatatgagcattgaagcagcgggttcttatatcctgaacctgggcaccatttaccgcgcactgtttacgaccctgcaaattaaagcgggtcgtacgattttcatcgaaggtgcagcaacgggtacaggtctggatgcagcacgcagcgcagcacgtaatggtctgcgcgttatcggcatggtgagtagctctagtcgcgcgtctaccctgctggcagcaggagcacatggtgcaattaaccgcaaagacccggaagtggccgattgttttacgcgagttccggaagatccgagcgcatgggcagcatgggaagcagccggtcagccgctgctggcaatgttccgtgcccagaatgatggtcgtctggccgattatgtggttagccacgcaggcgaaaccgcgtttccgcgctctttccagctgctgggtgaaccgcgtgatggtcatatcccgacgctgaccttttatggtgcgacgagtggctaccactttaccttcctgggcaaaccgggttctgccagtccgaccgaaatgctgcgtcgcgcaaacctgcgtgccggtgaagcagttctgatttattacggtgtgggcagcgatgatctggttgataccggaggcctggaagcgatcgaagccgcacgtcagatgggagcccgcattgtggttgtgacggtgtctgatgcccagcgcgaatttgttctgagtctgggtttcggtgcagcactgcgtggtgttgtgagcctggcggaactgaaacgtcgctttggcgatgaatttgaatggccgcgtaccatgccgccgctgccgaatgcacgtcaggacccgcagggcctgaaagaagcggtgcgtcgctttaacgatctggttttcaaaccgctgggtagcgcagttggcgtgtttctgcgctctgcggataacccgcgtggttatccggatctgattatcgaacgcgcagcgcatgatgccctggcagtgagtgccatgctgattaaaccgtttaccggccgtatcgtttatttcgaagatattggtggccgtcgctacagctttttcgcaccgcagatttgggtgcgtcagcgtcgcatttatatgccgacggcccagatttttggtacacacctgtctaacgcatacgaaattctgcgtctgaatgatgaaatcagtgcaggcctgctgacgattaccgaaccggcggttgtgccgtgggatgaactgccggaagcgcatcaggccatgtgggaaaaccgccacactgccgcaacctatgttgtgaatcatgcgctgccgcgcctgggtctgaaaaaccgtgatgaactgtacgaagcatggaccgcaggcgaacgtgaacaaaaactcatctcagaagaggatctgtaaggatctaagaggagaaagtgctatgcgtaaactggcccataacttttataaaccgctggcaattggagcaccggaaccgatccgcgaactgccggttcgtccggaacgcgtggttcatttctttccgccgcacgtggaaaaaattcgtgctcgcatcccggaagttgctaaacaggttgatgtcctgtgcggcaacctggaagatgcaattccgatggacgctaaagaagcggcccgtaatggttttatcgaagtcgtgaaagcaaccgatttcggcgacacggctctgtgggtgcgcgttaacgcgctgaacagcccgtgggtgctggatgacattgccgaaatcgttgcagctgtcggtaacaaactggatgtcattatgatcccgaaagtggaaggcccgtgggatattcactttgttgaccagtacctggcgctgctggaagcccgtcatcaaatcaaaaaaccgattctgatccacgcgctgctggaaaccgcccagggtatggtgaatctggaagaaattgcgggtgccagcccgcgtatgcacggtttctctctgggtccggcggatctggcagcctcgcgtggtatgaaaaccacgcgcgttggcggtggccacccgttttatggtgtcctggccgatccgcaggaaggccaagcagaacgtccgttctatcagcaggatctgtggcattacaccattgcgcgtatggttgacgtggcagttgctcacggtctgcgtgccttttacggtccgttcggcgatatcaaagacgaagcagcttgcgaagcacagtttcgcaatgctttcctgctgggttgtacgggcgcatggagtctggctccgaaccaaattccgatcgcaaaacgtgtcttttccccggatgtgaatgaagttctgttcgcgaaacgcattctggaagccatgccggatggttctggcgtggcgatgatcgacggtaaaatgcaggatgacgcgacgtggaaacaagccaaagtcattgtggatctggcgcgtatgatcgccaaaaaagatccggacctggcgcaggcctatggcctgtagttcacacaggaaaccacatgaaaggtattctgcatggtctgcgtgtggtggaaggttcggcttttgtcgctgccccgctgggtggtatgacgctggctcaactgggtgcagatgttatccgctttgacccgattggcggtggcctggattataaacgttggccggtcaccctggacggcaaacatagtctgttctgggctggtctgaacaaaggcaaacgctccattgcgatcgatattcgccatccgcgtggtcaggaactgctgacccaactgatctgcgctccgggtgaacacgcaggcctgtttattacgaatttcccggctcgtggttggctgtcatacgatgaactgaaacgtcaccgcgcggacctgatcatggttaatctggtcggtcgtcgcgatggtggctcggaagtggactacaccgttaatccgcagctgggtctgccgtttatgacgggtccggtgaccacgccggatgtggttaaccatgttctgcctgcctgggacatcgtcacaggtcagatgattgcactgggcctgctggcggcagaacgtcaccgtcgcctgacgggtgaaggccaactggtgaaaatcgctctgaaagatgttggtctggcgatgattggtcatctgggcatgatcgccgaagtgatgattaacgataccgaccgtccgcgtcagggcaattatctgtacggtgcatttggccgcgatttcgaaaccctggacggtaaacgtgttatggtcgtgggcctgacggatctgcaatggaaagccctgggtaaagcaaccggcctgacggacgcatttaacgctctgggtgcgcgtctgggcctgaatatggatgaagaaggtgaccgtttccgcgcgcgtcatgaaattgcagctctgctggaaccgtggtttcacgctcgtaccctggcggaagtgcgtcgcatcttcgaacagcatcgtgtcacctgggctccgtatcgcacggtgcgtgaagcgattgcccaggacccggactgtagcaccgataatccgatgtttgctatggttgaacaaccgggtatcggcagctacctgatgccgggctctccgctggatttcaccgcagtcccgcgtctgccggtgcagccagcaccgcgtctgggtgaacacacggatgaaattctgctggaagttctgggcctgagtgaagccgaagttggtcgtctgcatgatgaaggtattgttgctggcccggatcgtgctgcctgaattaaagaggagaaatagcaatgtcctcggcggattggatggcttggattggtcgcacggaacaggtggaagatgatatttgtctggcacaggctattgcagcggctgctaccctggaaccgccgagcggagcaccgacggctgattctccgctgccgccgctgtggcattggttttatttcctgccgcgtgccccgcagagtcaactgagctctgacggtcacccgcagcgcggcggttttattccgccgatcccgtacccgcgtcgcatgtttgcgggtgcccgtattcgcttccatcacccgctgcgtatcggtcagccagcacgtcgcgaaggtgtgattcgtaacatcacccaaaaaagtggccgctccggtccgctggcattcgttacggtcggctatcagatttaccaacatgaaatgctgtgcattgaagaagaacaggatatcgtttatcgtgaaccgggtgctccggtcccggcaccgaccccggtcgaactgccgccggttcacgatgcgattacccgtacagtggttcctgacccgcgtctgctgtttcgcttctccgcactgacgtttaacgctcatcgtatccactatgatcgcccttacgcgcagcatgaagaaggctaccctggtctggtcgttcacggtccgctggttgcggttctgctgatggaactggcgcgtcatcacaccagccgcccgattgttggtttttcattccgttcgcaagcgccgctgtttgacctggcaccgttccgtctgctggcacgtccgaatggtgatcgcatcgacctggaagcacaaggcccggatggcgcaacggcactgtcggcaacggtggaactgggtggttaaaggagggcatctatgtccgcaaaaacgaatccgggcaacttctttgaagatttccgtctgggccaaaccattgtccacgctacgccgcgcaccattaccgaaggcgatgtggccctgtataccagcctgtacggttctcgttttgcactgaccagctctacgccgttcgctcagtcactgggcctggaacgtgctccgattgactcgctgctggtgtttcatatcgttttcggcaaaaccgttccggatattagtctgaacgcgatcgccaatctgggttatgcgggcggtcgttttggtgccgtggtttacccaggtgacaccctgtcaaccacgtcgaaagtgattggcctgcgccagaacaaagatggcaaaacgggtgtcgtgtatgttcactctgtcggtgtgaatcaatgggacgaagttgtcctggaatacatccgttgggttatggtccgtaaacgcgatccgaacgcaccggctccggaaaccgtggttccggatctgccggacagcgtgccggttaccgatctgacggtcccgtataccgtgagtgcggccaactacaatctggcgcatgccggttccaattatctgtgggatgactacgaagtgggcgaaaaaattgatcatgtggacggtgtgaccatcgaagaagcagaacacatgcaggctacccgtctgtatcaaaacacggcccgcgttcattttaatctgcacgtcgaacgtgaaggccgcttcggtcgtcgcattgtttacggcggtcatattatcagcctggcacgtagtctgtcctttaacggcctggcaaatgctctgagtattgcagctatcaactccggccgccacaccaatccgagcttcgcaggtgacacgatttatgcttggtctgaaatcctggcgaaaatggccattccgggtcgtaccgatatcggagcactgcgtgttcgtaccgtcgcaacgaaagatcgtccgtgccacgacttcccgtatcgcgatgcggaaggtaactatgacccggctgttgtgctggattttgattacaccgtgctgatgccgcgtcgtggcgaacaaaaactcatctcagaagaggatctgaatagcgccgtcgactaagcttgcatgcctgcaggtcgactctagaggatctaagaggagaaagtgctatgagcatcttgtacgaagagcgtcttgatggcgctttacccgatgtcgaccgcacatcggtactgatggcactgcgtgagcatgtccctggacttgagatcctgcataccgatgaggagatcattccttacgagtgtgacgggttgagcgcgtatcgcacgcgtccattactggttgttctgcctaagcaaatggaacaggtgacagcgattctggctgtctgccatcgcctgcgtgtaccggtggtgacccgtggtgcaggcaccgggctttctggtggcgcgctgccgctggaaaaaggtgtgttgttggtgatggcgcgctttaaagagatcctcgacattaaccccgttggtcgccgcgcgcgcgtgcagccaggcgtgcgtaacctggcgatctcccaggccgttgcaccgcataatctctactacgcaccggacccttcctcacaaatcgcctgttccattggcggcaatgtggctgaaaatgccggcggcgtccactgcctgaaatatggtctgaccgtacataacctgctgaaaattgaagtgcaaacgctggacggcgaggcactgacgcttggatcggacgcgctggattcacctggttttgacctgctggcgctgttcaccggatcggaaggtatgctcggcgtgaccaccgaagtgacggtaaaactgctgccgaagccgcccgtggcgcgggttctgttagccagctttgactcggtagaaaaagccggacttgcggttggtgacatcatcgccaatggcattatccccggcgggctggagatgatggataacctgtcgatccgcgcggcggaagattttattcatgccggttatcccgtcgacgccgaagcgattttgttatgcgagctggacggcgtggagtctgacgtacaggaagactgcgagcgggttaacgacatcttgttgaaagcgggcgcgactgacgtccgtctggcacaggacgaagcagagcgcgtacgtttctgggccggtcgcaaaaatgcgttcccggcggtaggacgtatctccccggattactactgcatggatggcaccatcccgcgtcgcgccctgcctggcgtactggaaggcattgcccgtttatcgcagcaatatgatttacgtgttgccaacgtctttcatgccggagatggcaacatgcacccgttaatccttttcgatgccaacgaacccggtgaatttgcccgcgcggaagagctgggcgggaagatcctcgaactctgcgttgaagttggcggcagcatcagtggcgaacatggcatcgggcgagaaaaaatcaatcaaatgtgcgcccagttcaacagcgatgaaatcacgaccttccatgcggtcaaggcggcgtttgaccccgatggtttgctgaaccctgggaaaaacattcccacgctacaccgctgtgctgaatttggtgccatgcatgtgcatcacggtcatttacctttccctgaactggagcgtttctgatgctacgcgagtgtgattacagccaggcgctgctggagcaggtgaatcaggcgattagcgataaaacgccgctggtgattcagggcagcaatagcaaagcctttttaggtcgccctgtcaccgggcaaacgctggatgttcgttgtcatcgcggcattgttaattacgacccgaccgagctggtgataaccgcgcgtgtcggaacgccgctggtgacaattgaagcggcgctggaaagcgcggggcaaatgctcccctgtgagccgccgcattatggtgaagaagccacctggggcgggatggtcgcctgcgggctggcggggccgcgtcgcccgtggagcggttcggtccgcgattttgtcctcggcacgcgcatcattaccggcgctggaaaacatctgcgttttggtggcgaagtgatgaaaaacgttgccggatacgatctctcacggttaatggtcggaagctacggttgtcttggcgtgctcactgaaatctcaatgaaagtgttaccgcgaccgcgcgcctccctgagcctgcgtcgggaaatcagcctgcaagaagccatgagtgaaatcgccgagtggcaactccagccattacccattagtggcttatgttacttcgacaatgcgttgtggatccgccttgagggcggcgaaggatcggtaaaagcagcgcgtgaactgctgggtggcgaagaggttgccggtcagttctggcagcaattgcgtgaacaacaactgccgttcttctcgttaccaggtaccttatggcgcatttcattacccagtgatgcgccgatgatggatttacccggcgagcaactgatcgactggggcggggcgttacgctggctgaaatcgacagccgaggacaatcaaatccatcgcatcgcccgcaacgctggcggtcatgcgacccgctttagtgccggagatggtggctttgccccgctatcggctcctttattccgctatcaccagcagcttaaacagcagctcgacccttgcggcgtgtttaaccccggtcgcatgtacgcggaactttgaggagcaggctatgcaaacccaattaactgaagagatgcggcagaacgcgcgcgcgctggaagccgacagcatcctgcgcgcctgtgttcactgcggattttgtaccgcaacctgcccaacctatcagcttctgggcgatgaactggacgggccgcgcgggcgcatctatctgattaaacaggtgctggaaggcaacgaagtcacgcttaaaacacaggagcatctcgatcgctgcctcacttgccgtaattgtgaaaccacctgtccttctggtgtgcgctatcacaatttgctggatatcgggcgtgatattgtcgagcagaaagtgaaacgcccactgccggagcgaatactgcgcgaaggattgcgccaggtagtgccgcgtccggcggtcttccgtgcgctgacgcaggtagggctggtgctgcgaccgtttttaccggaacaggtcagagcaaaactgcctgctgaaacggtgaaagctaaaccgcgtccgccgctgcgccataagcgtcgggttttaatgttggaaggctgcgcccagcctacgctttcgcccaacaccaacgcggcaactgcgcgagtgctggatcgtctggggatcagcgtcatgccagctaacgaagcaggctgttgtggcgcggtggactatcatcttaatgcgcaggagaaagggctggcacgggcgcgcaataatattgatgcctggtggcccgcgattgaagcaggtgccgaggcaattttgcaaaccgccagcggctgcggcgcgtttgtcaaagagtatgggcagatgctgaaaaacgatgcgttatatgccgataaagcacgtcaggtcagtgaactggcggtcgatttagtcgaacttctgcgcgaggaaccgctggaaaaactggcaattcgcggcgataaaaagctggccttccactgtccgtgtaccctacaacatgcgcaaaagctgaacggcgaagtggaaaaagtgttgcttcgtcttggatttaccttaacggacgttcccgacagccatctgtgctgcggttcagcgggaacatatgcgttaacgcatcccgatctggcacgccagctgcgggataacaaaatgaatgcgctggaaagcggcaaaccggaaatgatcgtcaccgccaacattggttgccagacgcatctggcgagcgccggtcgtacctctgtgcgtcactggattgaaattgtagaacaagcccttgaaaaggaataaggatccctcgactctagaggatccccgggtaccgagctcgaattaggaggaattaataatgattgaacaagatggattgcacgcaggttctccggccgcttgggtggagaggctattcggctatgactgggcacaacagacaatcggctgctctgatgccgccgtgttccggctgtcagcgcaggggaggccggttctttttgtcaagaccgacctgtccggtgccctgaatgaacttcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggcgcgccttcgttagtgttagtctagaactagtttagtaaaaaacgagcaatataagccttctttaaataagaaagagggcttatattactcgtttttttctataaaaatgagcaaatttttatagagtatcatattttactttatttattatattaataataaataataataataaataataaaaaattactatatattttttattagaaaaaaaataaggtggaatttgctacctttttttattttttattgaaatttgtatttttttttttttttagacaatacaaaaaagaatagatagtagcgtaggggctccacttggctcgggggatatagctcagttggtagagctccgctcttgcaattgggtcgttgcgattacgggttgggtgtctaattgtccaggcggtaatgatagtatcttgtacctgaaccggtggctcactttttctaagtaatggggaaaaggaccgaaacatgccactgaaagactctactgagacaaagatgggctgtcaagaacgtagaggaggtaggatggtcagttggtcagatctagtatggatcgtacatggacggtagttggagtcggcggctctcctagggttccctcgtctgggattgatccctggggaagaggatcaagttggcccttgcgaacagcttgatgcactatctcccttcaaccctttgagcgaaatgcggcaaaaggaaggaaaatccatggaccgaccccatcgtctccaccccgtaggaactacgagatcaccccaaggacgccttcggtatccaggggtcgcggaccgaccatagaaccctgttcaataagtggaatgcattagctgtccgctcgcaggttgggcagtaagggtcggagaagggcaatcactcattcttaaaaccagcattcgaaagagttggggcggaaaagggggggaaagctctccgttcctggttctcctgtagctggatcctctagaaccacaagaatccttagttggaatgggattccagctcatcaccttttgagattttgagaagagttgctctttggagagcacagtacgatgaaagttgtaagctgtgttcgggggggagttcttgtctatcgttggcctctatggtagaatcagtcaggggcctgataggcggtggtttaccctgtggcggatgtcagcggttcgagtccgcttatctccaactcgtgaacttagccgatacaaagctatatgatagcacccaatttttccgattcggcacactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 97 - DNA, 8700 bp, Artificial Sequence
  source: /note=“Description of Artificial Sequence Synthetic polynucleotide”
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggatctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcggaaagcttgaattcgcggccgcttctagaggggatcttctgcaagcatctctatttcctgaaggtctaacctcgaagatttaagatttaattacgtttataattacaaaattgattctagtatctttaatttaatgcttatacattattaattaatttagtactttcaatttgttttcagaaattattttactattttttataaaataaaagggagaaaatggctatttaaatactagcctattttatttcaattttagcttaaaatcagccccaattagccccaatttcaaattcaaatggtccagcccaattcctaaataacccacccctaacccgcccggtttccccttttgatccatgcagtcaacgcccagaatttccctatataattttttaattcccaaacacccctaactctatcccatttctcaccaaccgccacatagatctatcctcttatctctcaaactctctcgaaccttcccctaaccctagcagcctctcatcatcctcacctcaaaacccaccggatactagtagcggccgctgcagagagctcatggcgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccttggccaacacttgtcactactttctcttatggtgttcaatgcttttcaagatacccagatcatatgaagcggcacgacttcttcaagagcgccatgcctgagggatacgtgcaggagaggaccatctctttcaaggacgacgggaactacaagacacgtgctgaagtcaagtttgagggagacaccctcgtcaacaggatcgagcttaagggaattgatttcaaggaggacggaaacatcctcggccacaagttggaatacaactacaactcccacaacgtatacatcacggcagacaaacaaaagaatggaatcaaagctaacttcaaaattagacacaacattgaagatggaagcgttcaactagcagaccattatcaacaaaatactcctattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataaggtaccaaaagctagtgatatccctgtgtgaaattgttatccgctacgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcccatgggaagttcctattccgaagttcctattctctgaaaagtataggaacttcagcgatcgcagacgtcaacgtggatacttggcagtggttacttggcttttcctttattttcttttggacggaagcggtggttactttgtcacacatttaaaaaaacacgtgtttctcacttttttctattcccgtcacaaacaattttaagaaagatccatctatcgtgatctttctatcaaacaaaagaaaaaaggtcttcatagtaacgctacaacatcaaatatgtggttgctctgacatcagtcgggaaaataaggatatggcggcattggccacatctattggggtcccaacttcctttcacaaaaaaattaaattgggtgtcccaacttttatctttgatatagtgacatgagtatcgggagcattggacaatggataaaatgagaactaaaaaaattctggttaatttttgatcattgttatttaaaaggttattttatctataatctacccatattgatcagttttatttaaatttgtttagctaccgctccacgagagagatcctcatcttaaaaatggaatatggaaattacacacgaccccaaaagtatattttttctctggagaatgctatttagagctttgactatatggtctgaattagaaagacgggaaataaaatctgctaagtgatataagctctaagtaggcgatgtgtgatggagaacaccttttctttaacagtcttcatgttttacagattcgcgaacttcgaatatccctatacggtctgtctaaccctcgtgtgtcttttgagtccaagataaaggccattattgagtaacatagacatgctggaatccaaccattgaagtcacaactgtccatgtagattctttggagaatctgaaaagtcttaataaaggtggtgtttcaaagaaaacaaaacaaatgagttaagaaaaaaaaatatcatgtagtggtcgagtattatgttatttattgtgtagctaccaatctttattctttaaatctgacataaaatgctacaaactttttacctcgtctatagccccaaaaaacctaaccacggttctaaaaccacacacagtgattttggttgacgacaatgcctctccttcctcaaaacgatttatttacattttttaaatcaaatgttacattttataccataattaagtctttttacagaatacttagatggaagagatgtataaaaaaggaggaaattgtaaaaaacatatttcgatcaattaaaccaggattcataaaaatataagtatatatataaatgatgtttcgtttagcgatgaacttcactcatatgataatacttaacaatataagtacataaaaaataaaataaaattaattgtttacgaaaagtctacaaatactgcatgtataattaatgttctctttatttatttatttataccttaccaagatatatctataaccgcatagaaatagaaggcgaagagataatttccaaaaacaagaaaaacctctaagctcaaaagggccggccatgattgaacaagatggattgcacgcaggttctccggccgcttgggtggagaggctattcggctatgactgggcacaacagacaatcggctgctctgatgccgccgtgttccggctgtcagcgcaggggaggccggttctttttgtcaagaccgacctgtccggtgccctgaatgaacttcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggcgcgcc

SEQ ID NO: 98 - DNA, 11728 bp, Artificial Sequence
  source: /note=“Description of Artificial Sequence Synthetic polynucleotide”
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggatctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcgggaattcgcggccgcttctagagtcgattaaaaatcccaattatatttggtctaatttagtttggtattgagtaaaacaaattcgaaccaaaccaaaatataaatatatagtttttatatatatgcctttaagactttttatagaattttctttaaaaaatatctagtaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtgcggccgcagatctagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgctcgagtcaacacaacatatacaaaacaaacgaatctcaagcaatcaagcattctacttctattgcagcaatttaaatcatttcttttaaagcaaaagcaattttctgaaaattttcaccatttacgaacgatagccatggctagttcagtgatcagttcagcagcagtggcaacaaggactaacgttacccaggcaggtagtatgatcgcaccattcacaggtctcaaatcagctgcaaccttcccagttagtagaaagcaaaatcttgatattacttctatcgcttctaacggaggtagagtgaggtgtatggcatcaaaaggtgaagagttgtttactggagttgtgcctatacttgttgaattggatggagatgtgaacggacataagttctctgtttcaggagaaggagagggagatgctacatacggaaaacttaccctcaagtttatttgtactacaggaaaacttcctgttccatggcctactttggtgaccactttttcttatggtgttcaatgcttctcaagatacccagatcatatgaagaggcacgatttctttaaaagtgctatgcctgaaggttatgtgcaggagagaacaatctcttttaaggatgatggaaactacaaaactagggcagaggttaagttcgagggagatacacttgtgaatagaatcgaattgaagggaatagatttcaaagaggatggtaacattctcggacataagttagaatacaactacaactcacacaatgtttacattacagctgataagcaaaagaacggaattaaggcaaacttcaagataaggcataacatagaggatggatctgttcagcttgctgatcactatcaacagaatacaccaattggagatggaccagtgcttttgcctgataaccattacctctcaacccagagtgcactctctaaagatcctaatgagaagagagatcacatggttcttttggagttcgttactgctgctggaatcacacacggtatggatgaattgtacaagtgaggtaccaaaagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaccggcatgcaagctgataagctttcgattaaaaatcccaattatatttggtctaatttagtttggtattgagtaaaacaaattcgaaccaaaccaaaatataaatatatagtttttatatatatgcctttaagactttttatagaattttctttaaaaaatatctagtaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagttactagtagcggccgctgcaggctagtgatatccctgtgtgaaattgttatccgctacgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcccatgggaagttcctattccgaagttcctattctctgaaaagtataggaacttcagcgatcgcagacgtcaacgtggatacttggcagtggttacttggcttttcctttattttcttttggacggaagcggtggttactttgtcacacatttaaaaaaacacgtgtttctcacttttttctattcccgtcacaaacaattttaagaaagatccatctatcgtgatctttctatcaaacaaaagaaaaaaggtcttcatagtaacgctacaacatcaaatatgtggttgctctgacatcagtcgggaaaataaggatatggcggcattggccacatctattggggtcccaacttcctttcacaaaaaaattaaattgggtgtcccaacttttatctttgatatagtgacatgagtatcgggagcattggacaatggataaaatgagaactaaaaaaattctggttaatttttgatcattgttatttaaaaggttattttatctataatctacccatattgatcagttttatttaaatttgtttagctaccgctccacgagagagatcctcatcttaaaaatggaatatggaaattacacacgaccccaaaagtatattttttctctggagaatgctatttagagctttgactatatggtctgaattagaaagacgggaaataaaatctgctaagtgatataagctctaagtaggcgatgtgtgatggagaacaccttttctttaacagtcttcatgttttacagattcgcgaacttcgaatatccctatacggtctgtctaaccctcgtgtgtcttttgagtccaagataaaggccattattgagtaacatagacatgctggaatccaaccattgaagtcacaactgtccatgtagattctttggagaatctgaaaagtcttaataaaggtggtgtttcaaagaaaacaaaacaaatgagttaagaaaaaaaaatatcatgtagtggtcgagtattatgttatttattgtgtagctaccaatctttattctttaaatctgacataaaatgctacaaactttttacctcgtctatagccccaaaaaacctaaccacggttctaaaaccacacacagtgattttggttgacgacaatgcctctccttcctcaaaacgatttatttacattttttaaatcaaatgttacattttataccataattaagtctttttacagaatacttagatggaagagatgtataaaaaaggaggaaattgtaaaaaacatatttcgatcaattaaaccaggattcataaaaatataagtatatatataaatgatgtttcgtttagcgatgaacttcactcatatgataatacttaacaatataagtacataaaaaataaaataaaattaattgtttacgaaaagtctacaaatactgcatgtataattaatgttctctttatttatttatttataccttaccaagatatatctataaccgcatagaaatagaaggcgaagagataatttccaaaaacaagaaaaacctctaagctcaaaagggccggccatgtctccggagaggagaccagttgagattaggccagctacagcagctgatatggccgctgtttgtgacatcgttaaccattacattgagacttctacagtgaactttaggacagagccacaaacaccacaagagtggattgatgatcttgagaggttgcaagatagatacccttggttggttgctgaggttgagggtgttgtggctggtattgcttacgctggaccttggaaggctaggaacgcttacgattggacagttgagagtactgtttacgtgtcacataggcatcaaaggttgggcctcggatctacattgtacacacatttgcttaagtctatggaggcgcaaggttttaagtctgtggttgctgttattggccttccaaacgatccatctgttaggttgcatgaggctttgggatacacagccaggggtacattgcgcgcagctggatacaagcatggtggatggcatgatgttggtttttggcaaagggattttgagttgccagctcctccaaggccagttagaccagttacccagatctgaggcgcgcc

SEQ ID NO: 99 - DNA, 9128 bp, Artificial Sequence
  source: /note=“Description of Artificial Sequence Synthetic polynucleotide”
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggatctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcggaaagcttgaattcgcggccgcttctagaggggatcttctgcaagcatctctatttcctgaaggtctaacctcgaagatttaagatttaattacgtttataattacaaaattgattctagtatctttaatttaatgcttatacattattaattaatttagtactttcaatttgttttcagaaattattttactattttttataaaataaaagggagaaaatggctatttaaatactagcctattttatttcaattttagcttaaaatcagccccaattagccccaatttcaaattcaaatggtccagcccaattcctaaataacccacccctaacccgcccggtttccccttttgatccatgcagtcaacgcccagaatttccctatataattttttaattcccaaacacccctaactctatcccatttctcaccaaccgccacatagatctatcctcttatctctcaaactctctcgaaccttcccctaaccctagcagcctctcatcatcctcacctcaaaacccaccggatactagtagcggccgctgcagaatggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcgacagctctagctctcctgtttcaacaaaacctcaaggtatattgatgatttaccaaatcttttccttgtcaaagttttgtgtttgactgtgtgggtttgaacctgttaggattcagtatgatatcaagtatgtgtcttttggaatacaaggatttacccttatggctatctttgttatctgtgtgaccttttctactttctcgctttgtaagatcgtctgagaatcattggagggcatttgaatgttgcagctgaagcaatggcgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccttggccaacacttgtcactactttctcttatggtgttcaatgcttttcaagatacccagatcatatgaagcggcacgacttcttcaagagcgccatgcctgagggatacgtgcaggagaggaccatctctttcaaggacgacgggaactacaagacacgtgctgaagtcaagtttgagggagacaccctcgtcaacaggatcgagcttaagggaattgatttcaaggaggacggaaacatcctcggccacaagttggaatacaactacaactcccacaacgtatacatcacggcagacaaacaaaagaatggaatcaaagctaacttcaaaattagacacaacattgaagatggaagcgttcaactagcagaccattatcaacaaaatactcctattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataaggtaccaaaagctagtgatatccctgtgtgaaattgttatccgctacgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcccatgggaagttcctattccgaagttcctattctctgaaaagtataggaacttcagcgatcgcagacgtcaacgtggatacttggcagtggttacttggcttttcctttattttcttttggacggaagcggtggttactttgtcacacatttaaaaaaacacgtgtttctcacttttttctattcccgtcacaaacaattttaagaaagatccatctatcgtgatctttctatcaaacaaaagaaaaaaggtcttcatagtaacgctacaacatcaaatatgtggttgctctgacatcagtcgggaaaataaggatatggcggcattggccacatctattggggtcccaacttcctttcacaaaaaaattaaattgggtgtcccaacttttatctttgatatagtgacatgagtatcgggagcattggacaatggataaaatgagaactaaaaaaattctggttaatttttgatcattgttatttaaaaggttattttatctataatctacccatattgatcagttttatttaaatttgtttagctaccgctccacgagagagatcctcatcttaaaaatggaatatggaaattacacacgaccccaaaagtatattttttctctggagaatgctatttagagctttgactatatggtctgaattagaaagacgggaaataaaatctgctaagtgatataagctctaagtaggcgatgtgtgatggagaacaccttttctttaacagtcttcatgttttacagattcgcgaacttcgaatatccctatacggtctgtctaaccctcgtgtgtcttttgagtccaagataaaggccattattgagtaacatagacatgctggaatccaaccattgaagtcacaactgtccatgtagattctttggagaatctgaaaagtcttaataaaggtggtgtttcaaagaaaacaaaacaaatgagttaagaaaaaaaaatatcatgtagtggtcgagtattatgttatttattgtgtagctaccaatctttattctttaaatctgacataaaatgctacaaactttttacctcgtctatagccccaaaaaacctaaccacggttctaaaaccacacacagtgattttggttgacgacaatgcctctccttcctcaaaacgatttatttacattttttaaatcaaatgttacattttataccataattaagtctttttacagaatacttagatggaagagatgtataaaaaaggaggaaattgtaaaaaacatatttcgatcaattaaaccaggattcataaaaatataagtatatatataaatgatgtttcgtttagcgatgaacttcactcatatgataatacttaacaatataagtacataaaaaataaaataaaattaattgtttacgaaaagtctacaaatactgcatgtataattaatgttctctttatttatttatttataccttaccaagatatatctataaccgcatagaaatagaaggcgaagagataatttccaaaaacaagaaaaacctctaagctcaaaagggccggccatgattgaacaagatggattgcacgcaggttctccggccgcttgggtggagaggctattcggctatgactgggcacaacagacaatcggctgctctgatgccgccgtgttccggctgtcagcgcaggggaggccggttctttttgtcaagaccgacctgtccggtgccctgaatgaacttcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggcgcgcc

SEQ ID NO: 100 - DNA, 9131 bp, Artificial Sequence
  source: /note=“Description of Artificial Sequence Synthetic polynucleotide”
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggatctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcggaaagcttgaattcgcggccgcttctagaggggatcttctgcaagcatctctatttcctgaaggtctaacctcgaagatttaagatttaattacgtttataattacaaaattgattctagtatctttaatttaatgcttatacattattaattaatttagtactttcaatttgttttcagaaattattttactattttttataaaataaaagggagaaaatggctatttaaatactagcctattttatttcaattttagcttaaaatcagccccaattagccccaatttcaaattcaaatggtccagcccaattcctaaataacccacccctaacccgcccggtttccccttttgatccatgcagtcaacgcccagaatttccctatataattttttaattcccaaacacccctaactctatcccatttctcaccaaccgccacatagatctatcctcttatctctcaaactctctcgaaccttcccctaaccctagcagcctctcatcatcctcacctcaaaacccaccggatactagtagcggccgctgcagaatggacagctctagctctcctgtttcaacaaaacctcaaggtatattgatgatttaccaaatcttttccttgtcaaagttttgtgtttgactgtgtgggtttgaacctgttaggattcagtatgatatcaagtatgtgtcttttggaatacaaggatttacccttatggctatctttgttatctgtgtgaccttttctactttctcgctttgtaagatcgtctgagaatcattggagggcatttgaatgttgcagctgaagcaatggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcatggcgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccttggccaacacttgtcactactttctcttatggtgttcaatgcttttcaagatacccagatcatatgaagcggcacgacttcttcaagagcgccatgcctgagggatacgtgcaggagaggaccatctctttcaaggacgacgggaactacaagacacgtgctgaagtcaagtttgagggagacaccctcgtcaacaggatcgagcttaagggaattgatttcaaggaggacggaaacatcctcggccacaagttggaatacaactacaactcccacaacgtatacatcacggcagacaaacaaaagaatggaatcaaagctaacttcaaaattagacacaacattgaagatggaagcgttcaactagcagaccattatcaacaaaatactcctattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataaggtaccaaaagctagtgatatccctgtgtgaaattgttatccgctacgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcccatgggaagttcctattccgaagttcctattctctgaaaagtataggaacttcagcgatcgcagacgtcaacgtggatacttggcagtggttacttggcttttcctttattttcttttggacggaagcggtggttactttgtcacacatttaaaaaaacacgtgtttctcacttttttctattcccgtcacaaacaattttaagaaagatccatctatcgtgatctttctatcaaacaaaagaaaaaaggtcttcatagtaacgctacaacatcaaatatgtggttgctctgacatcagtcgggaaaataaggatatggcggcattggccacatctattggggtcccaacttcctttcacaaaaaaattaaattgggtgtcccaacttttatctttgatatagtgacatgagtatcgggagcattggacaatggataaaatgagaactaaaaaaattctggttaatttttgatcattgttatttaaaaggttattttatctataatctacccatattgatcagttttatttaaatttgtttagctaccgctccacgagagagatcctcatcttaaaaatggaatatggaaattacacacgaccccaaaagtatattttttctctggagaatgctatttagagctttgactatatggtctgaattagaaagacgggaaataaaatctgctaagtgatataagctctaagtaggcgatgtgtgatggagaacaccttttctttaacagtcttcatgttttacagattcgcgaacttcgaatatccctatacggtctgtctaaccctcgtgtgtcttttgagtccaagataaaggccattattgagtaacatagacatgctggaatccaaccattgaagtcacaactgtccatgtagattctttggagaatctgaaaagtcttaataaaggtggtgtttcaaagaaaacaaaacaaatgagttaagaaaaaaaaatatcatgtagtggtcgagtattatgttatttattgtgtagctaccaatctttattctttaaatctgacataaaatgctacaaactttttacctcgtctatagccccaaaaaacctaaccacggttctaaaaccacacacagtgattttggttgacgacaatgcctctccttcctcaaaacgatttatttacattttttaaatcaaatgttacattttataccataattaagtctttttacagaatacttagatggaagagatgtataaaaaaggaggaaattgtaaaaaacatatttcgatcaattaaaccaggattcataaaaatataagtatatatataaatgatgtttcgtttagcgatgaacttcactcatatgataatacttaacaatataagtacataaaaaataaaataaaattaattgtttacgaaaagtctacaaatactgcatgtataattaatgttctctttatttatttatttataccttaccaagatatatctataaccgcatagaaatagaaggcgaagagataatttccaaaaacaagaaaaacctctaagctcaaaagggccggccatgattgaacaagatggattgcacgcaggttctccggccgcttgggtggagaggctattcggctatgactgggcacaacagacaatcggctgctctgatgccgccgtgttccggctgtcagcgcaggggaggccggttctttttgtcaagaccgacctgtccggtgccctgaatgaacttcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggcgcgcc

SEQ ID NO: 101 - DNA, 8877 bp, Artificial Sequence
  source: /note=“Description of Artificial Sequence Synthetic polynucleotide”
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggatctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcggaaagcttgaattcgcggccgcttctagaggggatcttctgcaagcatctctatttcctgaaggtctaacctcgaagatttaagatttaattacgtttataattacaaaattgattctagtatctttaatttaatgcttatacattattaattaatttagtactttcaatttgttttcagaaattattttactattttttataaaataaaagggagaaaatggctatttaaatactagcctattttatttcaattttagcttaaaatcagccccaattagccccaatttcaaattcaaatggtccagcccaattcctaaataacccacccctaacccgcccggtttccccttttgatccatgcagtcaacgcccagaatttccctatataattttttaattcccaaacacccctaactctatcccatttctcaccaaccgccacatagatctatcctcttatctctcaaactctctcgaaccttcccctaaccctagcagcctctcatcatcctcacctcaaaacccaccggatactagtagcggccgctgcagaatggcttcctctgttatttcctctgccgctgttgctacacgcaccaatgttacacaagctggcagcatgattgcacctttcactggtctcaaatctgctgctactttccctgtttcaaggcttagagttctttctgctcatttgatcacttccattgctagcaatggtggaagagttaggtgcatggcgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccttggccaacacttgtcactactttctcttatggtgttcaatgcttttcaagatacccagatcatatgaagcggcacgacttcttcaagagcgccatgcctgagggatacgtgcaggagaggaccatctctttcaaggacgacgggaactacaagacacgtgctgaagtcaagtttgagggagacaccctcgtcaacaggatcgagcttaagggaattgatttcaaggaggacggaaacatcctcggccacaagttggaatacaactacaactcccacaacgtatacatcacggcagacaaacaaaagaatggaatcaaagctaacttcaaaattagacacaacattgaagatggaagcgttcaactagcagaccattatcaacaaaatactcctattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataaggtaccaaaagctagtgatatccctgtgtgaaattgttatccgctacgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcccatgggaagttcctattccgaagttcctattctctgaaaagtataggaacttcagcgatcgcagacgtcaacgtggatacttggcagtggttacttggcttttcctttattttcttttggacggaagcggtggttactttgtcacacatttaaaaaaacacgtgtttctcacttttttctattcccgtcacaaacaattttaagaaagatccatctatcgtgatctttctatcaaacaaaagaaaaaaggtcttcatagtaacgctacaacatcaaatatgtggttgctctgacatcagtcgggaaaataaggatatggcggcattggccacatctattggggtcccaacttcctttcacaaaaaaattaaattgggtgtcccaacttttatctttgatatagtgacatgagtatcgggagcattggacaatggataaaatgagaactaaaaaaattctggttaatttttgatcattgttatttaaaaggttattttatctataatctacccatattgatcagttttatttaaatttgtttagctaccgctccacgagagagatcctcatcttaaaaatggaatatggaaattacacacgaccccaaaagtatattttttctctggagaatgctatttagagctttgactatatggtctgaattagaaagacgggaaataaaatctgctaagtgatataagctctaagtaggcgatgtgtgatggagaacaccttttctttaacagtcttcatgttttacagattcgcgaacttcgaatatccctatacggtctgtctaaccctcgtgtgtcttttgagtccaagataaaggccattattgagtaacatagacatgctggaatccaaccattgaagtcacaactgtccatgtagattctttggagaatctgaaaagtcttaataaaggtggtgtttcaaagaaaacaaaacaaatgagttaagaaaaaaaaatatcatgtagtggtcgagtattatgttatttattgtgtagctaccaatctttattctttaaatctgacataaaatgctacaaactttttacctcgtctatagccccaaaaaacctaaccacggttctaaaaccacacacagtgattttggttgacgacaatgcctctccttcctcaaaacgatttatttacattttttaaatcaaatgttacattttataccataattaagtctttttacagaatacttagatggaagagatgtataaaaaaggaggaaattgtaaaaaacatatttcgatcaattaaaccaggattcataaaaatataagtatatatataaatgatgtttcgtttagcgatgaacttcactcatatgataatacttaacaatataagtacataaaaaataaaataaaattaattgtttacgaaaagtctacaaatactgcatgtataattaatgttctctttatttatttatttataccttaccaagatatatctataaccgcatagaaatagaaggcgaagagataatttccaaaaacaagaaaaacctctaagctcaaaagggccggccatgattgaacaagatggattgcacgcaggttctccggccgcttgggtggagaggctattcggctatgactgggcacaacagacaatcggctgctctgatgccgccgtgttccggctgtcagcgcaggggaggccggttctttttgtcaagaccgacctgtccggtgccctgaatgaacttcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggcgcgcc

SEQ ID NO: 102 - DNA, 17662 bp, Artificial Sequence
  source: /note=“Description of Artificial Sequence Synthetic polynucleotide”
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggatctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcgggaattcgcggccgcttctagagtcgattaaaaatcccaattatatttggtctaatttagtttggtattgagtaaaacaaattcgaaccaaaccaaaatataaatatatagtttttatatatatgcctttaagactttttatagaattttctttaaaaaatatctagtaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtgcggccgcagatctagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgctcgagtcaacacaacatatacaaaacaaacgaatctcaagcaatcaagcattctacttctattgcagcaatttaaatcatttcttttaaagcaaaagcaattttctgaaaattttcaccatttacgaacgatagccatggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcatgtctattctttatgaagagagactcgatggagctttaccagatgttgatagaacctcagtgctcatggcattaagggaacatgttcctggacttgaaattcttcacacagatgaagagattatcccatatgaatgtgatggtttgtctgcttacagaactaggcctcttttggttgtgctcccaaagcagatggaacaggttacagctattcttgcagtgtgccatagattgagggttcctgttgtgacaagaggagctggtaccggactttcaggaggtgcactcccattagaaaagggtgttctcttagtgatggctaggttcaaagagatattggatattaatcctgtgggaagaagggctagagttcaaccaggtgtgaggaatctcgcaattagtcaggctgttgcacctcacaacctttattacgctcctgatccatcttcacaaatcgcatgttctataggtggtaatgtggctgaaaacgcaggaggtgttcattgccttaagtacggattgactgtgcacaaccttttgaaaatcgaagttcagactcttgatggagaggctcttacattgggtagtgatgcattggattctcctggttttgatctcttagctctcttcacaggttctgaaggaatgttaggtgttactacagaggttaccgttaaacttttgccaaaacctccagttgctagagtgctcttagcatcttttgattcagtggaaaaagctggacttgcagttggagatataattgctaacggaattattcctggaggtctcgaaatgatggataacttatctataagagctgctgaagatttcattcatgctggatatccagttgatgctgaggcaatacttttgtgtgaacttgatggtgttgagtcagatgtgcaagaagattgcgagagagttaatgatattctcttaaaggctggagcaactgatgtgaggttggctcaggatgaagcagagagagttaggttttgggctggaagaaaaaacgctttccctgctgttggtaggatctcaccagattattactgtatggatggtacaatacctagaagggctctcccaggagttttagagggtattgcaagacttagtcaacagtacgatttgagggttgctaatgtgtttcatgcaggagatggaaacatgcaccctctcatcttatttgatgctaatgagccaggagagttcgctagagcagaagagcttggaggaaagattcttgaactttgtgttgaagtgggaggtagtatctctggtgaacatggtattggaagagagaaaatcaatcaaatgtgcgctcagttcaactctgatgaaatcaccacttttcatgctgttaaggctgcattcgatcctgatggacttttgaatcctggaaagaatataccaacattgcacagatgcgctgagttcggagcaatgcacgttcaccacggacaccttccttttcctgagttggagagattctgactagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaccggcatgcaagctgataagcttagatctagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgctcgagtcaacacaacatatacaaaacaaacgaatctcaagcaatcaagcattctacttctattgcagcaatttaaatcatttcttttaaagcaaaagcaattttctgaaaattttcaccatttacgaacgatagccatggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcatgctcagagaatgcgattattctcaggctcttttggagcaagtgaatcaggcaatttcagataagactcctcttgttatccaaggttctaactcaaaggcttttcttggtagaccagtgactggacagacacttgatgttagatgtcataggggtatcgtgaactacgatcctactgaattggttataacagctagagtgggaaccccacttgttactattgaagctgcattggagtctgctggtcaaatgctcccatgtgagcctccacactacggagaagaggcaacttggggtggtatggttgcttgcggacttgcaggtcctagaaggccatggagtggttctgttagagattttgtgttgggaacaaggattatcaccggagctggaaagcatctcagattcggaggtgaagttatgaaaaatgtggcaggttatgatctctcaaggttaatggttggaagttacggttgtcttggagtgttgacagaaatttctatgaaggttcttcctagaccaagggcttcacttagtttgagaagggaaatatctttgcaagaggctatgtcagaaattgcagagtggcaactccagcctttaccaattagtggattgtgctattttgataacgctctctggatcagattagaaggaggagagggttcagtgaaagctgcaagggaactcttaggaggtgaagaggttgctggacagttctggcaacagcttagagagcaacagttgcctttcttttctcttccaggtacattgtggaggataagtcttccttctgatgctccaatgatggatctccctggagaacaattaatcgattggggaggtgctcttagatggttgaagtcaacagcagaggataatcagatccatagaatagctaggaacgcaggaggtcacgctaccagattttcagcaggagatggaggtttcgctcctctcagtgcaccactttttagataccaccaacagttgaagcagcagttagatccttgtggtgtgttcaatcctggaagaatgtacgctgagttgtgactagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaccggcatgcaagctgatgagctcagatctagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgctcgagtcaacacaacatatacaaaacaaacgaatctcaagcaatcaagcattctacttctattgcagcaatttaaatcatttcttttaaagcaaaagcaattttctgaaaattttcaccatttacgaacgatagccatggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcatgcaaactcagcttacagaagagatgagacaaaatgctagggcactcgaagctgattctatcttaagagcatgtgttcattgcggattctgtaccgctacttgccctacttatcaacttttgggagatgagcttgatggaccaagaggtagaatatacctcattaagcaagttttagaaggaaacgaggtgaccttgaaaactcaggaacatcttgatagatgcttgacatgtaggaattgcgagactacatgtccatcaggagttaggtatcacaacctcttagatatcggtagagatatagttgaacagaaggtgaaaagacctcttccagaaagaatactcagggagggattaagacaagttgtgcctaggccagctgtgtttagagcattgactcaagttggtcttgtgttgaggcctttccttccagaacaggttagagcaaagttgcctgctgaaacagtgaaggctaaaccaagacctccacttaggcataaaagaagggttctcatgttagagggatgtgctcagcctactttgtctccaaatacaaacgctgcaaccgctagagttcttgataggttgggtatttcagtgatgcctgcaaatgaggctggatgttgcggtgctgttgattaccacctcaacgcacaagagaagggattagctagagcaaggaataacatagatgcttggtggccagcaattgaagctggtgcagaggctatccttcaaactgcttcaggatgcggtgcatttgttaaggaatatggacagatgcttaaaaatgatgcattgtacgctgataaggcaagacaagtgagtgaacttgctgttgatttggtggagcttttgagagaagagcctcttgaaaaacttgctataagaggagataagaaattggcatttcattgtccatgcacacttcaacacgctcagaagttgaacggagaagttgagaaagtgctcttaagactcggtttcacattaaccgatgttcctgatagtcatctctgttgcggatctgctggtacttatgcattaacacaccctgatcttgctagacagttgagggataataagatgaacgctctcgaaagtggaaaacctgagatgattgttaccgctaatatcggttgtcaaactcatttggcatctgctggtaggacctctgtgaggcactggattgagatcgtggaacaggctcttgagaaggagtgactagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaccggcatgcaagctgatgcggccgctcgattaaaaatcccaattatatttggtctaatttagtttggtattgagtaaaacaaattcgaaccaaaccaaaatataaatatatagtttttatatatatgcctttaagactttttatagaattttctttaaaaaatatctagtaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagttactagtagcggccgctgcaggctagtgatatccctgtgtgaaattgttatccgctacgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcccatgggaagttcctattccgaagttcctattctctgaaaagtataggaacttcagcgatcgcagacgtcaacgtggatacttggcagtggttacttggcttttcctttattttcttttggacggaagcggtggttactttgtcacacatttaaaaaaacacgtgtttctcacttttttctattcccgtcacaaacaattttaagaaagatccatctatcgtgatctttctatcaaacaaaagaaaaaaggtcttcatagtaacgctacaacatcaaatatgtggttgctctgacatcagtcgggaaaataaggatatggcggcattggccacatctattggggtcccaacttcctttcacaaaaaaattaaattgggtgtcccaacttttatctttgatatagtgacatgagtatcgggagcattggacaatggataaaatgagaactaaaaaaattctggttaatttttgatcattgttatttaaaaggttattttatctataatctacccatattgatcagttttatttaaatttgtttagctaccgctccacgagagagatcctcatcttaaaaatggaatatggaaattacacacgaccccaaaagtatattttttctctggagaatgctatttagagctttgactatatggtctgaattagaaagacgggaaataaaatctgctaagtgatataagctctaagtaggcgatgtgtgatggagaacaccttttctttaacagtcttcatgttttacagattcgcgaacttcgaatatccctatacggtctgtctaaccctcgtgtgtcttttgagtccaagataaaggccattattgagtaacatagacatgctggaatccaaccattgaagtcacaactgtccatgtagattctttggagaatctgaaaagtcttaataaaggtggtgtttcaaagaaaacaaaacaaatgagttaagaaaaaaaaatatcatgtagtggtcgagtattatgttatttattgtgtagctaccaatctttattctttaaatctgacataaaatgctacaaactttttacctcgtctatagccccaaaaaacctaaccacggttctaaaaccacacacagtgattttggttgacgacaatgcctctccttcctcaaaacgatttatttacattttttaaatcaaatgttacattttataccataattaagtctttttacagaatacttagatggaagagatgtataaaaaaggaggaaattgtaaaaaacatatttcgatcaattaaaccaggattcataaaaatataagtatatatataaatgatgtttcgtttagcgatgaacttcactcatatgataatacttaacaatataagtacataaaaaataaaataaaattaattgtttacgaaaagtctacaaatactgcatgtataattaatgttctctttatttatttatttataccttaccaagatatatctataaccgcatagaaatagaaggcgaagagataatttccaaaaacaagaaaaacctctaagctcaaaagggccggccatgtctccggagaggagaccagttgagattaggccagctacagcagctgatatggccgctgtttgtgacatcgttaaccattacattgagacttctacagtgaactttaggacagagccacaaacaccacaagagtggattgatgatcttgagaggttgcaagatagatacccttggttggttgctgaggttgagggtgttgtggctggtattgcttacgctggaccttggaaggctaggaacgcttacgattggacagttgagagtactgtttacgtgtcacataggcatcaaaggttgggcctcggatctacattgtacacacatttgcttaagtctatggaggcgcaaggttttaagtctgtggttgctgttattggccttccaaacgatccatctgttaggttgcatgaggctttgggatacacagccaggggtacattgcgcgcagctggatacaagcatggtggatggcatgatgttggtttttggcaaagggattttgagttgccagctcctccaaggccagttagaccagttacccagatctgaggcgcgcc

SEQ ID NO: 103 - DNA, 18442 bp, Artificial Sequence
  source: /note=“Description of Artificial Sequence Synthetic polynucleotide”
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggatctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcgggaattcgcggccgcttctagagtcgattaaaaatcccaattatatttggtctaatttagtttggtattgagtaaaacaaattcgaaccaaaccaaaatataaatatatagtttttatatatatgcctttaagactttttatagaattttctttaaaaaatatctagtaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtgcggccgcagatctagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgctcgagtcaacacaacatatacaaaacaaacgaatctcaagcaatcaagcattctacttctattgcagcaatttaaatcatttcttttaaagcaaaagcaattttctgaaaattttcaccatttacgaacgatagccatggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcgacagctctagctctcctgtttcaacaaaacctcaaggtatattgatgatttaccaaatcttttccttgtcaaagttttgtgtttgactgtgtgggtttgaacctgttaggattcagtatgatatcaagtatgtgtcttttggaatacaaggatttacccttatggctatctttgttatctgtgtgaccttttctactttctcgctttgtaagatcgtctgagaatcattggagggcatttgaatgttgcagctgaagcaatgtctattctttatgaagagagactcgatggagctttaccagatgttgatagaacctcagtgctcatggcattaagggaacatgttcctggacttgaaattcttcacacagatgaagagattatcccatatgaatgtgatggtttgtctgcttacagaactaggcctcttttggttgtgctcccaaagcagatggaacaggttacagctattcttgcagtgtgccatagattgagggttcctgttgtgacaagaggagctggtaccggactttcaggaggtgcactcccattagaaaagggtgttctcttagtgatggctaggttcaaagagatattggatattaatcctgtgggaagaagggctagagttcaaccaggtgtgaggaatctcgcaattagtcaggctgttgcacctcacaacctttattacgctcctgatccatcttcacaaatcgcatgttctataggtggtaatgtggctgaaaacgcaggaggtgttcattgccttaagtacggattgactgtgcacaaccttttgaaaatcgaagttcagactcttgatggagaggctcttacattgggtagtgatgcattggattctcctggttttgatctcttagctctcttcacaggttctgaaggaatgttaggtgttactacagaggttaccgttaaacttttgccaaaacctccagttgctagagtgctcttagcatcttttgattcagtggaaaaagctggacttgcagttggagatataattgctaacggaattattcctggaggtctcgaaatgatggataacttatctataagagctgctgaagatttcattcatgctggatatccagttgatgctgaggcaatacttttgtgtgaacttgatggtgttgagtcagatgtgcaagaagattgcgagagagttaatgatattctcttaaaggctggagcaactgatgtgaggttggctcaggatgaagcagagagagttaggttttgggctggaagaaaaaacgctttccctgctgttggtaggatctcaccagattattactgtatggatggtacaatacctagaagggctctcccaggagttttagagggtattgcaagacttagtcaacagtacgatttgagggttgctaatgtgtttcatgcaggagatggaaacatgcaccctctcatcttatttgatgctaatgagccaggagagttcgctagagcagaagagcttggaggaaagattcttgaactttgtgttgaagtgggaggtagtatctctggtgaacatggtattggaagagagaaaatcaatcaaatgtgcgctcagttcaactctgatgaaatcaccacttttcatgctgttaaggctgcattcgatcctgatggacttttgaatcctggaaagaatataccaacattgcacagatgcgctgagttcggagcaatgcacgttcaccacggacaccttccttttcctgagttggagagattctgactagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaccggcatgcaagctgataagcttagatctagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgctcgagtcaacacaacatatacaaaacaaacgaatctcaagcaatcaagcattctacttctattgcagcaatttaaatcatttcttttaaagcaaaagcaattttctgaaaattttcaccatttacgaacgatagccatggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcgacagctctagctctcctgtttcaacaaaacctcaaggtatattgatgatttaccaaatcttttccttgtcaaagttttgtgtttgactgtgtgggtttgaacctgttaggattcagtatgatatcaagtatgtgtcttttggaatacaaggatttacccttatggctatctttgttatctgtgtgaccttttctactttctcgctttgtaagatcgtctgagaatcattggagggcatttgaatgttgcagctgaagcaatgctcagagaatgcgattattctcaggctcttttggagcaagtgaatcaggcaatttcagataagactcctcttgttatccaaggttctaactcaaaggcttttcttggtagaccagtgactggacagacacttgatgttagatgtcataggggtatcgtgaactacgatcctactgaattggttataacagctagagtgggaaccccacttgttactattgaagctgcattggagtctgctggtcaaatgctcccatgtgagcctccacactacggagaagaggcaacttggggtggtatggttgcttgcggacttgcaggtcctagaaggccatggagtggttctgttagagattttgtgttgggaacaaggattatcaccggagctggaaagcatctcagattcggaggtgaagttatgaaaaatgtggcaggttatgatctctcaaggttaatggttggaagttacggttgtcttggagtgttgacagaaatttctatgaaggttcttcctagaccaagggcttcacttagtttgagaagggaaatatctttgcaagaggctatgtcagaaattgcagagtggcaactccagcctttaccaattagtggattgtgctattttgataacgctctctggatcagattagaaggaggagagggttcagtgaaagctgcaagggaactcttaggaggtgaagaggttgctggacagttctggcaacagcttagagagcaacagttgcctttcttttctcttccaggtacattgtggaggataagtcttccttctgatgctccaatgatggatctccctggagaacaattaatcgattggggaggtgctcttagatggttgaagtcaacagcagaggataatcagatccatagaatagctaggaacgcaggaggtcacgctaccagattttcagcaggagatggaggtttcgctcctctcagtgcaccactttttagataccaccaacagttgaagcagcagttagatccttgtggtgtgttcaatcctggaagaatgtacgctgagttgtgactagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaccggcatgcaagctgatgagctcagatctagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgctcgagtcaacacaacatatacaaaacaaacgaatctcaagcaatcaagcattctacttctattgcagcaatttaaatcatttcttttaaagcaaaagcaattttctgaaaattttcaccatttacgaacgatagccatggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcgacagctctagctctcctgtttcaacaaaacctcaaggtatattgatgatttaccaaatcttttccttgtcaaagttttgtgtttgactgtgtgggtttgaacctgttaggattcagtatgatatcaagtatgtgtcttttggaatacaaggatttacccttatggctatctttgttatctgtgtgaccttttctactttctcgctttgtaagatcgtctgagaatcattggagggcatttgaatgttgcagctgaagcaatgcaaactcagcttacagaagagatgagacaaaatgctagggcactcgaagctgattctatcttaagagcatgtgttcattgcggattctgtaccgctacttgccctacttatcaacttttgggagatgagcttgatggaccaagaggtagaatatacctcattaagcaagttttagaaggaaacgaggtgaccttgaaaactcaggaacatcttgatagatgcttgacatgtaggaattgcgagactacatgtccatcaggagttaggtatcacaacctcttagatatcggtagagatatagttgaacagaaggtgaaaagacctcttccagaaagaatactcagggagggattaagacaagttgtgcctaggccagctgtgtttagagcattgactcaagttggtcttgtgttgaggcctttccttccagaacaggttagagcaaagttgcctgctgaaacagtgaaggctaaaccaagacctccacttaggcataaaagaagggttctcatgttagagggatgtgctcagcctactttgtctccaaatacaaacgctgcaaccgctagagttcttgataggttgggtatttcagtgatgcctgcaaatgaggctggatgttgcggtgctgttgattaccacctcaacgcacaagagaagggattagctagagcaaggaataacatagatgcttggtggccagcaattgaagctggtgcagaggctatccttcaaactgcttcaggatgcggtgcatttgttaaggaatatggacagatgcttaaaaatgatgcattgtacgctgataaggcaagacaagtgagtgaacttgctgttgatttggtggagcttttgagagaagagcctcttgaaaaacttgctataagaggagataagaaattggcatttcattgtccatgcacacttcaacacgctcagaagttgaacggagaagttgagaaagtgctcttaagactcggtttcacattaaccgatgttcctgatagtcatctctgttgcggatctgctggtacttatgcattaacacaccctgatcttgctagacagttgagggataataagatgaacgctctcgaaagtggaaaacctgagatgattgttaccgctaatatcggttgtcaaactcatttggcatctgctggtaggacctctgtgaggcactggattgagatcgtggaacaggctcttgagaaggagtgactagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaccggcatgcaagctgatgcggccgctcgattaaaaatcccaattatatttggtctaatttagtttggtattgagtaaaacaaattcgaaccaaaccaaaatataaatatatagtttttatatatatgcctttaagactttttatagaattttctttaaaaaatatctagtaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagttactagtagcggccgctgcaggctagtgatatccctgtgtgaaattgttatccgctacgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcccatgggaagttcctattccgaagttcctattctctgaaaagtataggaacttcagcgatcgcagacgtcaacgtggatacttggcagtggttacttggcttttcctttattttcttttggacggaagcggtggttactttgtcacacatttaaaaaaacacgtgtttctcacttttttctattcccgtcacaaacaattttaagaaagatccatctatcgtgatctttctatcaaacaaaagaaaaaaggtcttcatagtaacgctacaacatcaaatatgtggttgctctgacatcagtcgggaaaataaggatatggcggcattggccacatctattggggtcccaacttcctttcacaaaaaaattaaattgggtgtcccaacttttatctttgatatagtgacatgagtatcgggagcattggacaatggataaaatgagaactaaaaaaattctggttaatttttgatcattgttatttaaaaggttattttatctataatctacccatattgatcagttttatttaaatttgtttagctaccgctccacgagagagatcctcatcttaaaaatggaatatggaaattacacacgaccccaaaagtatattttttctctggagaatgctatttagagctttgactatatggtctgaattagaaagacgggaaataaaatctgctaagtgatataagctctaagtaggcgatgtgtgatggagaacaccttttctttaacagtcttcatgttttacagattcgcgaacttcgaatatccctatacggtctgtctaaccctcgtgtgtcttttgagtccaagataaaggccattattgagtaacatagacatgctggaatccaaccattgaagtcacaactgtccatgtagattctttggagaatctgaaaagtcttaataaaggtggtgtttcaaagaaaacaaaacaaatgagttaagaaaaaaaaatatcatgtagtggtcgagtattatgttatttattgtgtagctaccaatctttattctttaaatctgacataaaatgctacaaactttttacctcgtctatagccccaaaaaacctaaccacggttctaaaaccacacacagtgattttggttgacgacaatgcctctccttcctcaaaacgatttatttacattttttaaatcaaatgttacattttataccataattaagtctttttacagaatacttagatggaagagatgtataaaaaaggaggaaattgtaaaaaacatatttcgatcaattaaaccaggattcataaaaatataagtatatatataaatgatgtttcgtttagcgatgaacttcactcatatgataatacttaacaatataagtacataaaaaataaaataaaattaattgtttacgaaaagtctacaaatactgcatgtataattaatgttctctttatttatttatttataccttaccaagatatatctataaccgcatagaaatagaaggcgaagagataatttccaaaaacaagaaaaacctctaagctcaaaagggccggccatgtctccggagaggagaccagttgagattaggccagctacagcagctgatatggccgctgtttgtgacatcgttaaccattacattgagacttctacagtgaactttaggacagagccacaaacaccacaagagtggattgatgatcttgagaggttgcaagatagatacccttggttggttgctgaggttgagggtgttgtggctggtattgcttacgctggaccttggaaggctaggaacgcttacgattggacagttgagagtactgtttacgtgtcacataggcatcaaaggttgggcctcggatctacattgtacacacatttgcttaagtctatggaggcgcaaggttttaagtctgtggttgctgttattggccttccaaacgatccatctgttaggttgcatgaggctttgggatacacagccaggggtacattgcgcgcagctggatacaagcatggtggatggcatgatgttggtttttggcaaagggattttgagttgccagctcctccaaggccagttagaccagttacccagatctgaggcgcgcc

SEQ ID NO: 104 - DNA, 18451 bp, Artificial Sequence
  source: /note=“Description of Artificial Sequence Synthetic polynucleotide”
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggatctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcgggaattcgcggccgcttctagagtcgattaaaaatcccaattatatttggtctaatttagtttggtattgagtaaaacaaattcgaaccaaaccaaaatataaatatatagtttttatatatatgcctttaagactttttatagaattttctttaaaaaatatctagtaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtgcggccgcagatctagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgctcgagtcaacacaacatatacaaaacaaacgaatctcaagcaatcaagcattctacttctattgcagcaatttaaatcatttcttttaaagcaaaagcaattttctgaaaattttcaccatttacgaacgatagccatggacagctctagctctcctgtttcaacaaaacctcaaggtatattgatgatttaccaaatcttttccttgtcaaagttttgtgtttgactgtgtgggtttgaacctgttaggattcagtatgatatcaagtatgtgtcttttggaatacaaggatttacccttatggctatctttgttatctgtgtgaccttttctactttctcgctttgtaagatcgtctgagaatcattggagggcatttgaatgttgcagctgaagcaatggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcatgtctattctttatgaagagagactcgatggagctttaccagatgttgatagaacctcagtgctcatggcattaagggaacatgttcctggacttgaaattcttcacacagatgaagagattatcccatatgaatgtgatggtttgtctgcttacagaactaggcctcttttggttgtgctcccaaagcagatggaacaggttacagctattcttgcagtgtgccatagattgagggttcctgttgtgacaagaggagctggtaccggactttcaggaggtgcactcccattagaaaagggtgttctcttagtgatggctaggttcaaagagatattggatattaatcctgtgggaagaagggctagagttcaaccaggtgtgaggaatctcgcaattagtcaggctgttgcacctcacaacctttattacgctcctgatccatcttcacaaatcgcatgttctataggtggtaatgtggctgaaaacgcaggaggtgttcattgccttaagtacggattgactgtgcacaaccttttgaaaatcgaagttcagactcttgatggagaggctcttacattgggtagtgatgcattggattctcctggttttgatctcttagctctcttcacaggttctgaaggaatgttaggtgttactacagaggttaccgttaaacttttgccaaaacctccagttgctagagtgctcttagcatcttttgattcagtggaaaaagctggacttgcagttggagatataattgctaacggaattattcctggaggtctcgaaatgatggataacttatctataagagctgctgaagatttcattcatgctggatatccagttgatgctgaggcaatacttttgtgtgaacttgatggtgttgagtcagatgtgcaagaagattgcgagagagttaatgatattctcttaaaggctggagcaactgatgtgaggttggctcaggatgaagcagagagagttaggttttgggctggaagaaaaaacgctttccctgctgttggtaggatctcaccagattattactgtatggatggtacaatacctagaagggctctcccaggagttttagagggtattgcaagacttagtcaacagtacgatttgagggttgctaatgtgtttcatgcaggagatggaaacatgcaccctctcatcttatttgatgctaatgagccaggagagttcgctagagcagaagagcttggaggaaagattcttgaactttgtgttgaagtgggaggtagtatctctggtgaacatggtattggaagagagaaaatcaatcaaatgtgcgctcagttcaactctgatgaaatcaccacttttcatgctgttaaggctgcattcgatcctgatggacttttgaatcctggaaagaatataccaacattgcacagatgcgctgagttcggagcaatgcacgttcaccacggacaccttccttttcctgagttggagagattctgactagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaccggcatgcaagctgataagcttagatctagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgctcgagtcaacacaacatatacaaaacaaacgaatctcaagcaatcaagcattctacttctattgcagcaatttaaatcatttcttttaaagcaaaagcaattttctgaaaattttcaccatttacgaacgatagccatggacagctctagctctcctgtttcaacaaaacctcaaggtatattgatgatttaccaaatcttttccttgtcaaagttttgtgtttgactgtgtgggtttgaacctgttaggattcagtatgatatcaagtatgtgtcttttggaatacaaggatttacccttatggctatctttgttatctgtgtgaccttttctactttctcgctttgtaagatcgtctgagaatcattggagggcatttgaatgttgcagctgaagcaatggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcatgctcagagaatgcgattattctcaggctcttttggagcaagtgaatcaggcaatttcagataagactcctcttgttatccaaggttctaactcaaaggcttttcttggtagaccagtgactggacagacacttgatgttagatgtcataggggtatcgtgaactacgatcctactgaattggttataacagctagagtgggaaccccacttgttactattgaagctgcattggagtctgctggtcaaatgctcccatgtgagcctccacactacggagaagaggcaacttggggtggtatggttgcttgcggacttgcaggtcctagaaggccatggagtggttctgttagagattttgtgttgggaacaaggattatcaccggagctggaaagcatctcagattcggaggtgaagttatgaaaaatgtggcaggttatgatctctcaaggttaatggttggaagttacggttgtcttggagtgttgacagaaatttctatgaaggttcttcctagaccaagggcttcacttagtttgagaagggaaatatctttgcaagaggctatgtcagaaattgcagagtggcaactccagcctttaccaattagtggattgtgctattttgataacgctctctggatcagattagaaggaggagagggttcagtgaaagctgcaagggaactcttaggaggtgaagaggttgctggacagttctggcaacagcttagagagcaacagttgcctttcttttctcttccaggtacattgtggaggataagtcttccttctgatgctccaatgatggatctccctggagaacaattaatcgattggggaggtgctcttagatggttgaagtcaacagcagaggataatcagatccatagaatagctaggaacgcaggaggtcacgctaccagattttcagcaggagatggaggtttcgctcctctcagtgcaccactttttagataccaccaacagttgaagcagcagttagatccttgtggtgtgttcaatcctggaagaatgtacgctgagttgtgactagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaccggcatgcaagctgatgagctcagatctagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgctcgagtcaacacaacatatacaaaacaaacgaatctcaagcaatcaagcattctacttctattgcagcaatttaaatcatttcttttaaagcaaaagcaattttctgaaaattttcaccatttacgaacgatagccatggacagctctagctctcctgtttcaacaaaacctcaaggtatattgatgatttaccaaatcttttccttgtcaaagttttgtgtttgactgtgtgggtttgaacctgttaggattcagtatgatatcaagtatgtgtcttttggaatacaaggatttacccttatggctatctttgttatctgtgtgaccttttctactttctcgctttgtaagatcgtctgagaatcattggagggcatttgaatgttgcagctgaagcaatggaggtatgttctcttgccaggaatctctgcttcagtttattctcaacacataaggtatacaaatgggttatttggtgtttctctgtgttgtgtgactgattttgtgcttatagacgatttttaatatgttgatggtgttagcaattccagagtggaactggctcgagcggcatgcaaactcagcttacagaagagatgagacaaaatgctagggcactcgaagctgattctatcttaagagcatgtgttcattgcggattctgtaccgctacttgccctacttatcaacttttgggagatgagcttgatggaccaagaggtagaatatacctcattaagcaagttttagaaggaaacgaggtgaccttgaaaactcaggaacatcttgatagatgcttgacatgtaggaattgcgagactacatgtccatcaggagttaggtatcacaacctcttagatatcggtagagatatagttgaacagaaggtgaaaagacctcttccagaaagaatactcagggagggattaagacaagttgtgcctaggccagctgtgtttagagcattgactcaagttggtcttgtgttgaggcctttccttccagaacaggttagagcaaagttgcctgctgaaacagtgaaggctaaaccaagacctccacttaggcataaaagaagggttctcatgttagagggatgtgctcagcctactttgtctccaaatacaaacgctgcaaccgctagagttcttgataggttgggtatttcagtgatgcctgcaaatgaggctggatgttgcggtgctgttgattaccacctcaacgcacaagagaagggattagctagagcaaggaataacatagatgcttggtggccagcaattgaagctggtgcagaggctatccttcaaactgcttcaggatgcggtgcatttgttaaggaatatggacagatgcttaaaaatgatgcattgtacgctgataaggcaagacaagtgagtgaacttgctgttgatttggtggagcttttgagagaagagcctcttgaaaaacttgctataagaggagataagaaattggcatttcattgtccatgcacacttcaacacgctcagaagttgaacggagaagttgagaaagtgctcttaagactcggtttcacattaaccgatgttcctgatagtcatctctgttgcggatctgctggtacttatgcattaacacaccctgatcttgctagacagttgagggataataagatgaacgctctcgaaagtggaaaacctgagatgattgttaccgctaatatcggttgtcaaactcatttggcatctgctggtaggacctctgtgaggcactggattgagatcgtggaacaggctcttgagaaggagtgactagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaccggcatgcaagctgatgcggccgctcgattaaaaatcccaattatatttggtctaatttagtttggtattgagtaaaacaaattcgaaccaaaccaaaatataaatatatagtttttatatatatgcctttaagactttttatagaattttctttaaaaaatatctagtaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagttactagtagcggccgctgcaggctagtgatatccctgtgtgaaattgttatccgctacgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcccatgggaagttcctattccgaagttcctattctctgaaaagtataggaacttcagcgatcgcagacgtcaacgtggatacttggcagtggttacttggcttttcctttattttcttttggacggaagcggtggttactttgtcacacatttaaaaaaacacgtgtttctcacttttttctattcccgtcacaaacaattttaagaaagatccatctatcgtgatctttctatcaaacaaaagaaaaaaggtcttcatagtaacgctacaacatcaaatatgtggttgctctgacatcagtcgggaaaataaggatatggcggcattggccacatctattggggtcccaacttcctttcacaaaaaaattaaattgggtgtcccaacttttatctttgatatagtgacatgagtatcgggagcattggacaatggataaaatgagaactaaaaaaattctggttaatttttgatcattgttatttaaaaggttattttatctataatctacccatattgatcagttttatttaaatttgtttagctaccgctccacgagagagatcctcatcttaaaaatggaatatggaaattacacacgaccccaaaagtatattttttctctggagaatgctatttagagctttgactatatggtctgaattagaaagacgggaaataaaatctgctaagtgatataagctctaagtaggcgatgtgtgatggagaacaccttttctttaacagtcttcatgttttacagattcgcgaacttcgaatatccctatacggtctgtctaaccctcgtgtgtcttttgagtccaagataaaggccattattgagtaacatagacatgctggaatccaaccattgaagtcacaactgtccatgtagattctttggagaatctgaaaagtcttaataaaggtggtgtttcaaagaaaacaaaacaaatgagttaagaaaaaaaaatatcatgtagtggtcgagtattatgttatttattgtgtagctaccaatctttattctttaaatctgacataaaatgctacaaactttttacctcgtctatagccccaaaaaacctaaccacggttctaaaaccacacacagtgattttggttgacgacaatgcctctccttcctcaaaacgatttatttacattttttaaatcaaatgttacattttataccataattaagtctttttacagaatacttagatggaagagatgtataaaaaaggaggaaattgtaaaaaacatatttcgatcaattaaaccaggattcataaaaatataagtatatatataaatgatgtttcgtttagcgatgaacttcactcatatgataatacttaacaatataagtacataaaaaataaaataaaattaattgtttacgaaaagtctacaaatactgcatgtataattaatgttctctttatttatttatttataccttaccaagatatatctataaccgcatagaaatagaaggcgaagagataatttccaaaaacaagaaaaacctctaagctcaaaagggccggccatgtctccggagaggagaccagttgagattaggccagctacagcagctgatatggccgctgtttgtgacatcgttaaccattacattgagacttctacagtgaactttaggacagagccacaaacaccacaagagtggattgatgatcttgagaggttgcaagatagatacccttggttggttgctgaggttgagggtgttgtggctggtattgcttacgctggaccttggaaggctaggaacgcttacgattggacagttgagagtactgtttacgtgtcacataggcatcaaaggttgggcctcggatctacattgtacacacatttgcttaagtctatggaggcgcaaggttttaagtctgtggttgctgttattggccttccaaacgatccatctgttaggttgcatgaggctttgggatacacagccaggggtacattgcgcgcagctggatacaagcatggtggatggcatgatgttggtttttggcaaagggattttgagttgccagctcctccaaggccagttagaccagttacccagatctgaggcgcgcc

SEQ ID NO: 105 - DNA, 17689 bp, Artificial Sequence
  source: /note=“Description of Artificial Sequence Synthetic polynucleotide”
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggatctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcgggaattcgcggccgcttctagagtcgattaaaaatcccaattatatttggtctaatttagtttggtattgagtaaaacaaattcgaaccaaaccaaaatataaatatatagtttttatatatatgcctttaagactttttatagaattttctttaaaaaatatctagtaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtgcggccgcagatctagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgctcgagtcaacacaacatatacaaaacaaacgaatctcaagcaatcaagcattctacttctattgcagcaatttaaatcatttcttttaaagcaaaagcaattttctgaaaattttcaccatttacgaacgatagccatggcttcctctgttatttcctctgccgctgttgctacacgcaccaatgttacacaagctggcagcatgattgcacctttcactggtctcaaatctgctgctactttccctgtttcaaggcttagagttctttctgctcatttgatcacttccattgctagcaatggtggaagagttaggtgcatgtctattctttatgaagagagactcgatggagctttaccagatgttgatagaacctcagtgctcatggcattaagggaacatgttcctggacttgaaattcttcacacagatgaagagattatcccatatgaatgtgatggtttgtctgcttacagaactaggcctcttttggttgtgctcccaaagcagatggaacaggttacagctattcttgcagtgtgccatagattgagggttcctgttgtgacaagaggagctggtaccggactttcaggaggtgcactcccattagaaaagggtgttctcttagtgatggctaggttcaaagagatattggatattaatcctgtgggaagaagggctagagttcaaccaggtgtgaggaatctcgcaattagtcaggctgttgcacctcacaacctttattacgctcctgatccatcttcacaaatcgcatgttctataggtggtaatgtggctgaaaacgcaggaggtgttcattgccttaagtacggattgactgtgcacaaccttttgaaaatcgaagttcagactcttgatggagaggctcttacattgggtagtgatgcattggattctcctggttttgatctcttagctctcttcacaggttctgaaggaatgttaggtgttactacagaggttaccgttaaacttttgccaaaacctccagttgctagagtgctcttagcatcttttgattcagtggaaaaagctggacttgcagttggagatataattgctaacggaattattcctggaggtctcgaaatgatggataacttatctataagagctgctgaagatttcattcatgctggatatccagttgatgctgaggcaatacttttgtgtgaacttgatggtgttgagtcagatgtgcaagaagattgcgagagagttaatgatattctcttaaaggctggagcaactgatgtgaggttggctcaggatgaagcagagagagttaggttttgggctggaagaaaaaacgctttccctgctgttggtaggatctcaccagattattactgtatggatggtacaatacctagaagggctctcccaggagttttagagggtattgcaagacttagtcaacagtacgatttgagggttgctaatgtgtttcatgcaggagatggaaacatgcaccctctcatcttatttgatgctaatgagccaggagagttcgctagagcagaagagcttggaggaaagattcttgaactttgtgttgaagtgggaggtagtatctctggtgaacatggtattggaagagagaaaatcaatcaaatgtgcgctcagttcaactctgatgaaatcaccacttttcatgctgttaaggctgcattcgatcctgatggacttttgaatcctggaaagaatataccaacattgcacagatgcgctgagttcggagcaatgcacgttcaccacggacaccttccttttcctgagttggagagattctgactagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaccggcatgcaagctgataagcttagatctagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgctcgagtcaacacaacatatacaaaacaaacgaatctcaagcaatcaagcattctacttctattgcagcaatttaaatcatttcttttaaagcaaaagcaattttctgaaaattttcaccatttacgaacgatagccatggcttcctctgttatttcctctgccgctgttgctacacgcaccaatgttacacaagctggcagcatgattgcacctttcactggtctcaaatctgctgctactttccctgtttcaaggcttagagttctttctgctcatttgatcacttccattgctagcaatggtggaagagttaggtgcatgctcagagaatgcgattattctcaggctcttttggagcaagtgaatcaggcaatttcagataagactcctcttgttatccaaggttctaactcaaaggcttttcttggtagaccagtgactggacagacacttgatgttagatgtcataggggtatcgtgaactacgatcctactgaattggttataacagctagagtgggaaccccacttgttactattgaagctgcattggagtctgctggtcaaatgctcccatgtgagcctccacactacggagaagaggcaacttggggtggtatggttgcttgcggacttgcaggtcctagaaggccatggagtggttctgttagagattttgtgttgggaacaaggattatcaccggagctggaaagcatctcagattcggaggtgaagttatgaaaaatgtggcaggttatgatctctcaaggttaatggttggaagttacggttgtcttggagtgttgacagaaatttctatgaaggttcttcctagaccaagggcttcacttagtttgagaagggaaatatctttgcaagaggctatgtcagaaattgcagagtggcaactccagcctttaccaattagtggattgtgctattttgataacgctctctggatcagattagaaggaggagagggttcagtgaaagctgcaagggaactcttaggaggtgaagaggttgctggacagttctggcaacagcttagagagcaacagttgcctttcttttctcttccaggtacattgtggaggataagtcttccttctgatgctccaatgatggatctccctggagaacaattaatcgattggggaggtgctcttagatggttgaagtcaacagcagaggataatcagatccatagaatagctaggaacgcaggaggtcacgctaccagattttcagcaggagatggaggtttcgctcctctcagtgcaccactttttagataccaccaacagttgaagcagcagttagatccttgtggtgtgttcaatcctggaagaatgtacgctgagttgtgactagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaccggcatgcaagctgatgagctcagatctagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgctcgagtcaacacaacatatacaaaacaaacgaatctcaagcaatcaagcattctacttctattgcagcaatttaaatcatttcttttaaagcaaaagcaattttctgaaaattttcaccatttacgaacgatagccatggcttcctctgttatttcctctgccgctgttgctacacgcaccaatgttacacaagctggcagcatgattgcacctttcactggtctcaaatctgctgctactttccctgtttcaaggcttagagttctttctgctcatttgatcacttccattgctagcaatggtggaagagttaggtgcatgcaaactcagcttacagaagagatgagacaaaatgctagggcactcgaagctgattctatcttaagagcatgtgttcattgcggattctgtaccgctacttgccctacttatcaacttttgggagatgagcttgatggaccaagaggtagaatatacctcattaagcaagttttagaaggaaacgaggtgaccttgaaaactcaggaacatcttgatagatgcttgacatgtaggaattgcgagactacatgtccatcaggagttaggtatcacaacctcttagatatcggtagagatatagttgaacagaaggtgaaaagacctcttccagaaagaatactcagggagggattaagacaagttgtgcctaggccagctgtgtttagagcattgactcaagttggtcttgtgttgaggcctttccttccagaacaggttagagcaaagttgcctgctgaaacagtgaaggctaaaccaagacctccacttaggcataaaagaagggttctcatgttagagggatgtgctcagcctactttgtctccaaatacaaacgctgcaaccgctagagttcttgataggttgggtatttcagtgatgcctgcaaatgaggctggatgttgcggtgctgttgattaccacctcaacgcacaagagaagggattagctagagcaaggaataacatagatgcttggtggccagcaattgaagctggtgcagaggctatccttcaaactgcttcaggatgcggtgcatttgttaaggaatatggacagatgcttaaaaatgatgcattgtacgctgataaggcaagacaagtgagtgaacttgctgttgatttggtggagcttttgagagaagagcctcttgaaaaacttgctataagaggagataagaaattggcatttcattgtccatgcacacttcaacacgctcagaagttgaacggagaagttgagaaagtgctcttaagactcggtttcacattaaccgatgttcctgatagtcatctctgttgcggatctgctggtacttatgcattaacacaccctgatcttgctagacagttgagggataataagatgaacgctctcgaaagtggaaaacctgagatgattgttaccgctaatatcggttgtcaaactcatttggcatctgctggtaggacctctgtgaggcactggattgagatcgtggaacaggctcttgagaaggagtgactagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaccggcatgcaagctgatgcggccgctcgattaaaaatcccaattatatttggtctaatttagtttggtattgagtaaaacaaattcgaaccaaaccaaaatataaatatatagtttttatatatatgcctttaagactttttatagaattttctttaaaaaatatctagtaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagttactagtagcggccgctgcaggctagtgatatccctgtgtgaaattgttatccgctacgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcccatgggaagttcctattccgaagttcctattctctgaaaagtataggaacttcagcgatcgcagacgtcaacgtggatacttggcagtggttacttggcttttcctttattttcttttggacggaagcggtggttactttgtcacacatttaaaaaaacacgtgtttctcacttttttctattcccgtcacaaacaattttaagaaagatccatctatcgtgatctttctatcaaacaaaagaaaaaaggtcttcatagtaacgctacaacatcaaatatgtggttgctctgacatcagtcgggaaaataaggatatggcggcattggccacatctattggggtcccaacttcctttcacaaaaaaattaaattgggtgtcccaacttttatctttgatatagtgacatgagtatcgggagcattggacaatggataaaatgagaactaaaaaaattctggttaatttttgatcattgttatttaaaaggttattttatctataatctacccatattgatcagttttatttaaatttgtttagctaccgctccacgagagagatcctcatcttaaaaatggaatatggaaattacacacgaccccaaaagtatattttttctctggagaatgctatttagagctttgactatatggtctgaattagaaagacgggaaataaaatctgctaagtgatataagctctaagtaggcgatgtgtgatggagaacaccttttctttaacagtcttcatgttttacagattcgcgaacttcgaatatccctatacggtctgtctaaccctcgtgtgtcttttgagtccaagataaaggccattattgagtaacatagacatgctggaatccaaccattgaagtcacaactgtccatgtagattctttggagaatctgaaaagtcttaataaaggtggtgtttcaaagaaaacaaaacaaatgagttaagaaaaaaaaatatcatgtagtggtcgagtattatgttatttattgtgtagctaccaatctttattctttaaatctgacataaaatgctacaaactttttacctcgtctatagccccaaaaaacctaaccacggttctaaaaccacacacagtgattttggttgacgacaatgcctctccttcctcaaaacgatttatttacattttttaaatcaaatgttacattttataccataattaagtctttttacagaatacttagatggaagagatgtataaaaaaggaggaaattgtaaaaaacatatttcgatcaattaaaccaggattcataaaaatataagtatatatataaatgatgtttcgtttagcgatgaacttcactcatatgataatacttaacaatataagtacataaaaaataaaataaaattaattgtttacgaaaagtctacaaatactgcatgtataattaatgttctctttatttatttatttataccttaccaagatatatctataaccgcatagaaatagaaggcgaagagataatttccaaaaacaagaaaaacctctaagctcaaaagggccggccatgtctccggagaggagaccagttgagattaggccagctacagcagctgatatggccgctgtttgtgacatcgttaaccattacattgagacttctacagtgaactttaggacagagccacaaacaccacaagagtggattgatgatcttgagaggttgcaagatagatacccttggttggttgctgaggttgagggtgttgtggctggtattgcttacgctggaccttggaaggctaggaacgcttacgattggacagttgagagtactgtttacgtgtcacataggcatcaaaggttgggcctcggatctacattgtacacacatttgcttaagtctatggaggcgcaaggttttaagtctgtggttgctgttattggccttccaaacgatccatctgttaggttgcatgaggctttgggatacacagccaggggtacattgcgcgcagctggatacaagcatggtggatggcatgatgttggtttttggcaaagggattttgagttgccagctcctccaaggccagttagaccagttacccagatctgaggcgcgcc

SEQ ID NO: 106 - protein, 25 aa, Unknown
  source: /note=“Description of Unknown Mitochondrion localization signal sequence”
MLSLRQSIRFFKPATRTLCSSRYLL

SEQ ID NO: 107 - protein, 29 aa, Unknown
  source: /note=“Description of Unknown Secretory pathway localization signal sequence”
MMSFVSLLLVGILFWATEAEQLTKCEVFQ

SEQ ID NO: 108 - protein, 13 aa, Unknown
  source: /note=“Description of Unknown Endoplasmic reticulum retention localization signal sequence”
MTGASRRSARGRI

SEQ ID NO: 109 - protein, 36 aa, Unknown
  source: /note=“Description of Unknown Vacuole secretion localization signal sequence”
MKAFTLALFLALSLYLLPNPAHSRFNPIRLPTTHPA

SEQ ID NO: 110 - DNA, 437 bp, Artificial Sequence; identical to SEQ ID NO: 18
  source: /note=“Description of Artificial Sequence Synthetic polynucleotide”

SEQ ID NO: 111 - protein, 55 aa, Artificial Sequence
  source: /note=“Description of Artificial Sequence Synthetic polypeptide”
MEVCSLARNLCFSLFSTHKSGTGSSGDSSSSPVSTKPQDRLRIIGGHLNVAAEAM

SEQ ID NO: 112 - DNA, 440 bp, Artificial Sequence; identical to SEQ ID NO: 19
  source: /note=“Description of Artificial Sequence Synthetic polynucleotide”

SEQ ID NO: 113 - protein, 56 aa, Artificial Sequence
  source: /note=“Description of Artificial Sequence Synthetic polypeptide”
MDSSSSPVSTKPQDRLRIIGGHLNVAAEAMEVCSLARNLCFSLFSTHKSGTGSSGM

SEQ ID NO: 114 - DNA, 177 bp, Artificial Sequence; identical to SEQ ID NO: 15
  source: /note=“Description of Artificial Sequence Synthetic polynucleotide”

SEQ ID NO: 115 - protein, 59 aa, Artificial Sequence
  source: /note=“Description of Artificial Sequence Synthetic polypeptide”
MASSVISSAAVATRTNVTQAGSMIAPFTGLKSAATFPVSRKQNLDITSIASNGGRVRCM

SEQ ID NO: 116 - DNA, 186 bp, Artificial Sequence; identical to SEQ ID NO: 20
  source: /note=“Description of Artificial Sequence Synthetic polynucleotide”

SEQ ID NO: 117 - protein, 62 aa, Artificial Sequence
  source: /note=“Description of Artificial Sequence Synthetic polypeptide”
MASSVISSAAVATRTNVTQAGSMIAPFTGLKSAATFPVSRLRVLSAHLITSIASNGGRVRCM

(19) United States
(12) Patent Application Publication
(10) Pub. No.: US 2015/0275207 A1
(43) Pub. Date: Oct. 1, 2015

(54) COMPOSITIONS AND METHODS FOR MODULATING POLYPEPTIDE LOCALIZATION IN PLANTS

(71) Applicant: PRESIDENT AND FELLOWS OF HARVARD COLLEGE, Cambridge, MA (US)

(72) Inventors: Jeffrey C. Way, Cambridge, MA (US); Matthew Mattozzi, Boston, MA (US); Mathias J. Voges, Stanford, CA (US)

(73) Assignee: PRESIDENT AND FELLOWS OF HARVARD COLLEGE, Cambridge, MA (US)

(21) Appl. No.: 14/430,994

(22) PCT Filed: Sep. 4, 2013
(86) PCT No.: PCT/US2013/057974

§ 371 Date: Mar. 25, 2015

Related U.S. Application Data
(60) Provisional application No. 61/708,909, filed on Oct. 2, 2012.

(51) Int. Cl. C12N 15/113

(52) U.S. Cl. CPC C12N 15/113

(57) ABSTRACT

Described herein are engineered multiple localization tags which, when translated and processed into peptides, will direct operably linked polypeptides to multiple subcellular locations.

### CROSS-REFERENCE TO RELATED APPLICATIONS

This application claims benefit under 35 U.S.C. §119(e) of U.S. Provisional Application No. 61/708,909 filed Oct. 2, 2012, the content of which is incorporated herein by reference in its entirety.

### GOVERNMENT SUPPORT

This invention was made with federal funding under Cooperative Agreement DE-000079 with the Department of Energy Advanced Research Projects Agency. The U.S. government has certain rights in the invention.

### SEQUENCE LISTING

The instant application contains a Sequence Listing which has been submitted in ASCII format via EFS-Web and is hereby incorporated by reference in its entirety. Said ASCII copy, created on Sep. 4, 2013, is named 002806-075472-PCT_SL.txt and is 555,556 bytes in size.

### TECHNICAL FIELD

The technology described herein relates to methods and compostions for modulating the localization of polypeptides within a plant cell.

### BACKGROUND

In engineering cells and/or organisms, it can be desirable to target particular polypeptides to specific subcellular locations. Current technologies allow polypeptides to be directed to one specific location by adding a single localization signal to either the N- or C-terminus.

However, the design of cells and/or organisms with re-engineered biosynthetic and/or metabolic pathways often requires that polypeptides be present in multiple subcellular locations. For example, when creating plants with a re-engineered photorespiration pathway, the plants will optimally have certain polypeptides concentrated in both the chloroplasts and the peroxisomes (Kebeish, R. et al. (2007) Nature Biotechnology 25, 593-9; Maier, A., et al. (2012) Frontiers in Plant Science 3, 38). One approach to target proteins to more than one location involves using multiple copies of the relevant transgene, each having a different localization signal. Using this approach requires multiple transformation events, which is time-consuming and results in a cell with multiple insertion events. This makes it increasingly difficult to ensure that each copy performs as intended (Que, Q., et al. (2010) GM crops 1, 220-9; Dafny-Yelin, M. & Tzfira, T. (2007) Plant Physiology 145, 1118-28).

Although in some instances polypeptides can be directed to two subcellular locations by adding a second localization signal to the second terminus of the polypeptide (Hyunjong, B., et al. (2006) Journal of Experimental Botany 57, 161-9), this approach is limited by the possible combinations that can be made from available, compatible N- and C-terminal extensions. Additionally, not all polypeptides will retain their activity when localization signals are added to both termini—e.g. some polypeptides will lose activity if sequence is appended to a certain terminus.

### SUMMARY

Described herein are compositions and methods relating to localization signals that permit a polypeptide to be directed to at least two (e.g. two, three, four, or more) subcellular locations using a tag located on a single terminus of the polypeptide. The technology described herein reduces the amount of cloning and the size of DNA constructs required to target a polypeptide to multiple locations in a cell and/or organism.

In one aspect, described herein is an engineered multiple localization tag comprising a nucleic acid sequence encoding at least two localization signal sequences; wherein each of the localization signal sequences will direct localization of a polypeptide encoded by an operably linked sequence to a different set of subcellular compartments. In some embodiments, the localization signal sequences are not separated by an exon. In some embodiments, the localization signal sequences are separated by an exon of no more than 300 bases. In some embodiments, the exon can comprise glycine and serine residues.

In some embodiments, the tag can further comprise a set of compatible splicing sequences; wherein the set comprises two alternative splice donor sequences and one splice acceptor sequence; wherein the two alternative splice donor sequences flank one localization signal sequence; and the splice acceptor sequence is located 3′ of both splice donor sequences of the set. In some embodiments, the set of splicing sequences can be located 5′ of a second localization signal. In some embodiments, the set of splicing sequences can be located 3′ of a second localization signal.

In some embodiments, the tag can further comprise a set of compatible splicing sequences; wherein the set comprises two alternative splice acceptor sequences and one splice donor sequence; wherein the two alternative splice acceptor sequences flank a localization signal sequence; and the splice donor sequence is located 5′ of both splice acceptor sequences of the set. In some embodiments, the set of splicing sequences can be located 3′ of a second localization signal. In some embodiments, the set of splicing sequences can be located 5′ of a second localization signal.

In some embodiments, a pair of alternative splice sites can comprise a weak and a strong splice site. In some embodiments, the weak splice site can be located 5′ of the flanked localization signal and the strong splice site can be located 3′ of the flanked localization signal. In some embodiments, a set of compatible splicing sites can comprise the weak splice donor site of SEQ ID NO: 8; the strong splice donor site of SEQ ID NO: 9, and the splice acceptor site of SEQ ID NO: 10. In some embodiments, a set of compatible splicing sites can comprise the splice donor site of SEQ ID NO: 11, the weak splice acceptor site of SEQ ID NO: 12; and the strong splice acceptor site of SEQ ID NO: 13.

In some embodiments, each of the localization signals is selected from the group consisting of a chloroplast localization signal; a peroxisome localization signal; a mitochondrion localization signal; a secretory pathway localization signal; an endoplasmic reticulum localization signal; and a vacuole secretion localization signal. In some embodiments, the chloroplast localization signal can comprise a nucleic acid sequence encoding CTPa (SEQ ID NO:1) or a polypeptide having at least 90% identity to CTPa. In some embodiments, the chloroplast localization signal can comprise the nucleic acid sequence of SEQ ID NO: 14 or a sequence having at least 90% identity to SEQ ID NO: 14. In some embodiments, the chloroplast localization signal can comprise a nucleic acid sequence encoding CTPb (SEQ ID NO: 6) or a polypeptide having at least 90% identity to CTPb. In some embodiments, the chloroplast localization signal can comprise the nucleic acid sequence of SEQ ID NO: 15 or a sequence having at least 90% identity to SEQ ID NO: 15. In some embodiments, the peroxisome localization signal can comprise a nucleic acid sequence encoding PTS2 (SEQ ID NO: 2) or a polypeptide having at least 90% identity to PTS2. In some embodiments, the peroxisome localization signal can comprise the nucleic acid sequence of SEQ ID NO: 16 or a polypeptide having at least 90% identity to SEQ ID NO: 16. In some embodiments, the peroxisome localization signal can comprise SEQ ID NO: 5. In some embodiments, the peroxisome localization signal can comprise the nucleic acid sequence of SEQ ID NO: 17 or a sequence having at least 90% identity to SEQ ID NO: 17.

In some embodiments, the tag can comprise a nucleic acid sequence encoding a polypeptide of any of SEQ ID NOs: 3 and 21-23 or a polypeptide having at least 90% identity to any of SEQ ID NOs: 3 and 21-23. In some embodiments, the tag can comprise a nucleic acid sequence of SEQ ID NO: 18 or a sequence having at least 90% identity to SEQ ID NO: 18.

In some embodiments, the tag can comprise the sequence of any of SEQ ID NOs: 4 and 24-26 or a sequence having at least 90% identity to any of SEQ ID NOs: 4 and 24-26. In some embodiments, the tag can comprise the nucleic acid sequence of SEQ ID NO: 19 or a sequence having at least 90% identity to SEQ ID NO: 19.

In some embodiments, a first localization signal is comprised within a second localization signal. In some embodiments, the first localization signal is substituted for the amino acids equivalent to residues 37 to 46 of SEQ ID NO: 6. In some embodiments, the tag can comprise the sequence of SEQ ID NO:7 or a sequence having at least 90% identity to SEQ ID NO:7. In some embodiments, the tag can comprise the nucleic acid sequence of SEQ ID NO: 20 or a sequence having at least 90% identity to SEQ ID NO: 20.

In one aspect, described herein is a vector comprising an engineered multiple localization tag described herein. In some embodiments, the entirety of the engineered multiple localization tag can be located on one flank of a cloning site or an operably linked sequence encoding a peptide. In some embodiments, the engineered multiple localization tag can be located 5′ of an operably linked sequence encoding a polypeptide.

In one aspect, described herein is an engineered cell or organism comprising an engineered multiple localization tag as described herein or a vector comprising an engineered multiple localization tag as described herein. In one aspect, described herein is a nucleic acid molecule having the sequence of, or encoding the polypeptide having the sequence of, any of SEQ ID NO: 28-87, or a sequence having at least 90% identity thereto. In one aspect, described herein is a vector comprising a nucleic acid molecule having the sequence of, or encoding the polypeptide having the sequence of, any of SEQ ID NO: 28-87, or a sequence having at least 90% identity thereto. In one aspect, described herein is an engineered cell or organism comprising (a) a nucleic acid molecule having the sequence of, or encoding the polypeptide having the sequence of, any of SEQ ID NO: 28-87, or a sequence having at least 90% identity thereto or (b) a vector comprising a nucleic acid molecule having the sequence of, or encoding the polypeptide having the sequence of, any of SEQ ID NO: 28-87, or a sequence having at least 90% identity thereto.

### BRIEF DESCRIPTION OF THE DRAWINGS

FIGS. 1A-1D depict the design of alternatively spliced elements TriTag-1 and TriTag-2. FIGS. 1A and 1B depict schematic splice diagrams of TriTag-1 (FIG. 1A) and TriTag-2 (FIG. 1B), showing non-targeting sequences (shaded), chloroplast targeting sequences (Chl;), peroxisome targeting sequences (Per), and the enhanced GFP coding sequence used in transient expression experiments (eGFP). FIGS. 1C and 1D depict the design of TriTag-1 (SEQ ID NOS 110 (DNA) and 111 (protein); FIG. 1C) and TriTag-2 (SEQ ID NOS 112 (DNA) and 113 (protein); FIG. 1D) sequences. The ATG codon at the end corresponds to the first residue of the GFP open reading frame. Alternatively spliced targeting regions are underlined. Donor and acceptor dimers are underlined. The DNA sequences shown in unshaded boxes with solid lines derive from the PIMT2 5′ coding region (Dinkins et al. 2008) and include sequences encoding the chloroplast targeting sequence (amino acids shown in white boxes with solid lines). The DNA sequences shown in white boxes with dashed lines derive from the TTL 5′ coding region (Reumann et al. 2007) and include sequences encoding the peroxisome targeting sequence (amino acids shown in white boxes with dashed lines).

FIGS. 2A-2D depict a comparison of chloroplast transit peptide (CTPb) with the peroxisome target signal (PTS2)-embedded element TriTag-3. FIGS. 2A-2B depict diagrams of CTPb (FIG. 2A) and TriTag-3 (FIG. 2B), showing chloroplast targeting sequences (Chl), peroxisome targeting sequences (Per), flexible regions (shaded region), and the enhanced GFP coding sequence used in transient expression experiments (eGFP). FIGS. 2C-2D depict CTPb (SEQ ID NOS 114 (DNA) and 115 (protein); FIG. 2C) and TriTag-3 (SEQ ID NOS 116 (DNA) and 117 (protein); FIG. 2D) sequences. The ATG codon at the end corresponds to the first residue of the GFP open reading frame. The DNA sequences shown in white boxes with solid lines derive from the rbcS1 5′ coding region (Kebeish et al. 2007) and encode a chloroplast targeting sequence (white boxes with solid lines). The DNA sequences shown in white boxes with dashed lines (FIG. 2D) code for a consensus PTS2 signal (white boxes with dashed lines). The PTS2 sequence is embedded within a flexible region of CTPb (shaded region).

FIG. 3 depicts a table and schematic of compartments of a typical tobacco leaf epidermal cell. The relative sizes and locations within the cell, and the relative expression levels observed via confocal microscopy are indicated.

FIG. 4 depicts a schematic visualization of plant cell compartments and the proposed effect of the 3-HOP engineering approach to enhance carbon fixation and reduce carbon loss from photorespiration in C₃ plants. Bold arrows indicate reactions catalyzed by heterologous enzymes, dotted arrows indicate natively occurring reactions. GOX, glycolate oxidase.

FIG. 5 depicts a schematic of expression of E. coli glycolate dehydrogenase within the chloroplast, peroxisomes, and cytoplasm leading to an increased production of reducing equivalents and bypass the peroxide-producing oxidation reaction native to the peroxisomes. Bold arrows indicate reactions catalyzed by heterologous enzymes, dotted arrows indicate natively occurring reactions. Native conversion of glyoxylate to P-glycerate has been observed in literature (Kebeish, et al. 2007).

FIG. 6 depicts a schematic illustration of ‘payload’ integration into the plastome by homologous recombination. Note that transformants will either retain their original left and right arm sequences or replace these with the left and right arm sequences of the vector—given that the latter transformants are viable. Image redrawn from (Day and Goldschmidt-Clermont 2011).

FIG. 7 depicts a schematic vector map of pMV02 plastome integration vector with annotation of the reactions as in Zarzycki et al 2008 PNAS. 2, malonyl-CoA reductase; 3, propionyl-CoA synthase; 10, (S)-malyl-CoA/β-methylmalyl-CoA/(S)-citramalyl-CoA lyase; 11, mesaconyl-C1-CoA hydratase (β-methylmalyl-CoA dehydratase); 12, mesaconyl-CoA C1:C4 CoA transferase; 13, mesaconyl-C4-CoA hydratase; glcDEF, E. coli glycolate dehydrogenase; neo, neomycin phosphotransferase II; psbA-TT, photosystem II terminator; trniltrnA, tRNA-isoleucine/tRNA-alanine; AmpR, β-lactamase; ori, pMB1 origin of replication.

FIG. 8 depicts a schematic of TriTag1. Splice variant βγ-χω expresses a fused protein of interest with a CTP (chloroplast transit peptide) directing it towards the chloroplast. Splice variant αγ-χψ expresses the fused protein of interest with a PTS2 directing it to the peroxisome. Splice variant αγ-χω expresses the fused protein of interest without a transit peptide, which will localize in the cytoplasm. Splice variant βγ-χψ expresses the fused protein of interest with a CTP along with PTS2; i.e. an ambiguous signal.

FIG. 9 depicts a schematic of TriTag-2, composed of module 2 followed by module 1, in frame. This combination affords functional splice variants expressing transit peptides with either/and PTS2 or/and CTP or/and no defined targeting signal (cytoplasmic localization). Splice variant αγ-χψ expresses the fused protein of interest with a CTP directing it towards the chloroplast. Splice variant βγ-χω expresses the fused protein of interest with a PTS2 directing it to the peroxisome. Splice variant αγ-χω expresses the fused protein of interest without a transit peptide, which will localize in the cytoplasm. Splice variant βγ-χψ expresses the fused protein of interest with a CTP along with PTS2; i.e. an ambiguous signal.

FIG. 10 depicts a schematic illustration of TriTag3. Illustration of a PTS2 signal superimposed onto the Solanum tuberosum potato rbcS1 chloroplast peptide. The conserved PTS2 amino acid sequence was placed closer to the C-terminal end of the CTP peptide, as this region is expected to play a smaller role in chloroplast uptake than the region closer to the N-terminus.

FIG. 11 depicts a schematic of Tic-Toc chloroplast protein uptake mechanism. High protein expression levels and limited availability of ATP for protein import can result in a bottleneck at equilibrium (1), causing the retention of the preprotein and, in the case of the GFP fusions described here, fluorescence indicative of cytoplasmic GFP (Image: Jarvis P 2008 New Phytol 179:257).

FIG. 12 depicts a vector map illustration of plasmids constructed for the delivery of E. coli GDH subunits into the genome of Arabidopsis thaliana by agrobacterium tumeficiens (floral dip method). Nuclear scaffold, RB7 nucleotides region to minimize the probability of silencing (Halweg, Thompson and Spiker 2005); CaMV 35S-P, Cauliflower Mosaic Virus 25S “long” promoter as described in (Horstmann, et al. 2004); 5′UTR, 5′ untranslated region from Tobacco Etch Virus; Targeting peptide, rbcS1 chloroplast transit peptide, TriTag1, TriTag2 or TriTag3; Terminator, nopaline synthase terminator (NOS); PAT; phosphinothricin acetyltransferase, glufosinate (Finale Herbicide) resistance marker; KanR, neomycin phosphotransferase II; ori, origin of replication for E. coli and A. tumeficiencs; glcD/glcE/glcF, E. coli GDH subunits codon optimized for genomic A. thaliana expression.

FIGS. 13A-13B demonstrate some embodiments of the engineered multiple localization tags described herein. FIG. 13A depicts a schematic of the general structure of an embodiment of a DNA construct mediating localization of a protein of interest to multiple compartments, including DNA elements encoding localization sequences that may localize a protein to the nucleus, cytoplasm, endoplasmic reticulum, plastid, peroxisome, mitochondria, and/or other cellular compartments. Three tags are shown, but more or fewer tags may be used depending on the needs of the user. Alternative splicing is used to generate mRNAs encoding one or more localization sequence N-terminal to the ORF of interest, which encodes the protein of interest. Short sequences comprising donor and acceptor sites and a small number of amino acids (typically less than 50 amino acids), are optionally used to allow for efficient splicing of the mRNA. FIG. 13B depicts schematics of representative possible spliced mRNAs generated from the DNA construct depicted in FIG. 13A.

### DETAILED DESCRIPTION

Described herein are methods and compositions for directing polypeptides to specific subcellular locations. As described herein, the inventors have discovered methods to engineer a single transgene which is translated into one or more polypeptide isoforms that are targeted to multiple subcellular locations, e.g. organelles and/or the cytoplasm, something which was previously accomplished by utilizing multiple transgenes, each with a unique sequence targeting it to a single subcellular location.

In one aspect, described herein is an engineered multiple localization tag. As used herein the term “engineered multiple localization tag” or “EML tag” refers to a nucleic acid sequence comprising at least two localization signal sequences, e.g. two localization signal sequences, three localization signal sequences, four localization signal sequences, or more localization signal sequences. In some embodiments, the term “EML tag” can also refer to the one or more polypeptide isoforms encoded by an EML tag nucleic acid sequence. In an EML tag, each of the at least two localization signal sequences can, individually, direct the localization of an operably linked polypeptide (referred to herein as a “cargo polypeptide”) to a different set of subcellular locations. The sets of subcellular locations can overlap, but are not identical. The cargo polypeptide can be any polypeptide, e.g. an enzyme, a scaffold protein, a polypeptide native to the cell in which it is present while operably linked to the EML tag, and/or polypeptide heterologous to the cell in which it is present while operably linked to the EML tag.

As used herein, a “localization signal sequence” refers to a nucleic acid sequence (or a peptide encoded by that nucleic acid sequence) that when translated as part of a larger polypeptide comprising a cargo polypeptide, will localize the cargo polypeptide to a specific subcellular location, typically a particular organelle and/or a plasma membrane. As used herein, a cargo polypeptide is “localized” to a particular subcellular location by a localization signal and/or EML tag if, when transcribed with that operably linked signal or tag, its concentration at that subcellular location is at least 10% greater than without the operably linked signal or tag, e.g. at least 10%, at least 20%, at least 30%, at least 50%, at least 75%, at least 100%, at least 200%, or at least 500% or greater than without the operably linked signal or tag. The concentration can be the absolute concentration, e.g. the μg/mL of the polypeptide found in, e.g., chloroplasts, or the relative concentration, e.g. the % of the polypeptide which is found in the chloroplasts relative to the rest of the cell. As used herein, a “subcellular compartment” or “subcellular location” refers to a discreet location within a cell. Non-limiting examples can include organelles, chloroplasts, mitochondria, endosomes, peroxisomes, nucleus, ER, Golgi, lysosomes, and plasma membranes (including organelle and cellular membranes).

Localization signals that traffic their cargo polypeptides to specific subcellular locations are known in the art, e.g. signals that traffic to the nucleus, ER, Golgi, endosomes, lysosomes, peroxisomes, chloroplasts, mitochondria, and/or plasma membrane. Examples of localization signals are known in the art, e.g. in the SPdb (Signal Peptide Database) (Choo et al. BMC Bioinformatics 2005; 6:249; which is incorporated by reference herein in its entirety), which is freely available on the world wide web at http://proline.bic.nus.edu.sg/spdb/index.html. Bioinformatics tools for predicting localization signals are known in the art (see, e.g., Alexandersson et al. Frontiers in Plant Sci 2013 4:9; which is incorporated by reference herein in its entirety), e.g. SignalP (described, e.g., in Petersen et al Nature Methods 2011 8:785; which is incorporated by reference herein in its entirety). In some embodiments, a localization signal can be selected from the group consisting of a chloroplast localization signal and a peroxisome localization signal. In some embodiments, a localization signal can be selected from the group consisting of a chloroplast localization signal (e.g. SEQ ID NO: 1 or 6); a peroxisome localization signal (e.g. SEQ ID NO: 2); a mitochondrion localization signal (e.g. H₂N-MLSLRQSIRFFKPATRTLCSSRYLL, SEQ ID NO: 106); a secretory pathway localization signal (e.g. H₂N-MMSFVSLLLVGILFWATEAEQLTKCEVFQ; SEQ ID NO: 107); an endoplasmic reticulum retention localization signal (e.g. H₂N-MTGASRRSARGRI; SEQ ID NO: 108); and a vacuole secretion localization signal (e.g. H₂N-MKAFTLALFLALSLYLLPNPAHSRFNPIRLPTTHPA; SEQ ID NO: 109). Other examples of localization signals are known in the art and can be predicted, e.g. using Signal P (see, e.g. Petersen et al Nature Methods 2011 8:785; which is incorporated by reference herein in its entirety.

In some embodiments, a choloroplast localization signal can comprise a nucleic acid sequence encoding CTPa (e.g. a nucleic acid sequence encoding SEQ ID NO:1) or encoding a polypeptide that promotes or mediates chloroplast localization and has at least 80% identity to CTPa. e.g., at least 80% identity, at least 90% identity, at least 95% identity, or at least 98% identity. In some embodiments, a choloroplast localization signal can comprise the nucleic acid sequence of SEQ ID NO: 14 or a nucleic acid having at least 80% identity to the sequence of SEQ ID NO: 14. e.g., at least 80%, at least 90% identity, at least 95% identity, or at least 98% identity. In some embodiments, a choloroplast localization signal can comprise a nucleic acid sequence encoding CTPb (e.g. a nucleic acid sequence encoding SEQ ID NO:6) or encoding a polypeptide having at least 80% identity to CTPb. e.g., at least 80% identity, at least 90% identity, at least 95% identity, or at least 98% identity. In some embodiments, a choloroplast localization signal can comprise the nucleic acid sequence of SEQ ID NO: 15 or a nucleic acid having at least 80% identity to the sequence of SEQ ID NO: 15, e.g., at least 80% identity, at least 90% identity, at least 95% identity, or at least 98% identity.

In some embodiments, a peroxisome localization signal can comprise a nucleic acid sequence encoding PTS2, (e.g. a nucleic acid sequence encoding SEQ ID NO:2) or encoding a polypeptide having at least 80% identity to PTS2. e.g., at least 80% identity, at least 90% identity, at least 95% identity, or at least 98% identity. In some embodiments, a peroxisome localization signal can comprise the nucleic acid sequence of SEQ ID NO: 16 or a nucleic acid having at least 80% identity to the sequence of SEQ ID NO: 16. e.g., at least 80% identity, at least 90% identity, at least 95% identity, or at least 98% identity. In some embodiments, a peroxisome localization signal can comprise a nucleic acid sequence encoding a polypeptide of SEQ ID NO: 5 or encoding a polypeptide having at least 80% identity to SEQ ID NO: 5. e.g., at least 80% identity, at least 90% identity, at least 95% identity, or at least 98% identity. In some embodiments, a peroxisome localization signal can comprise a nucleic acid sequence encoding a polypeptide of SEQ ID NO: 27 or encoding a polypeptide having at least 80% identity to SEQ ID NO: 27. e.g., at least 80% identity, at least 90% identity, at least 95% identity, or at least 98% identity. In some embodiments, a peroxisome localization signal can comprise the nucleic acid sequence of SEQ ID NO: 17 or a nucleic acid having at least 80% identity to the sequence of SEQ ID NO: 17. e.g., at least 80% identity, at least 90% identity, at least 95% identity, or at least 98% identity.

In any event, a localization signal which is a variant of a sequence described herein must retain at least 10% of the localization ability of the reference sequence from which is it derived, e.g. it must be able to direct localization of a cargo polypeptide to the desired target location at least 10% as effectively as the reference localization signal (as measured by absolute or relative concentration as described elsewhere herein), e.g. at least 10%, at least 20%, at least 30%, at least 50%, at least 70%, at least 80%, at least 90%, at least 95%, at least 100% effectively or more effectively.

In some embodiments, a localization signal has at least 70% identity with a reference localization signal sequence, e.g. a naturally-occurring localization signal sequence and/or a localization signal sequence described herein. In some embodiments, a localization signal has at least 80% identity with a reference localization signal sequence, e.g. a naturally-occurring localization signal sequence and/or a localization signal sequence described herein. In some embodiments, a localization signal has at least 90% identity with a reference localization signal sequence, e.g. a naturally-occurring localization signal sequence and/or a localization signal sequence described herein. Examples of localization signals and localization signal motifs are described in the art, e.g. in Bruce B D 2000 Trends Cell Biol 10:440-47; Sakamoto W et al 2008 The Arabidopsis Book 6:e110; Bruce B D 2001 Biochim Biophys Acta 1541:2-21; Lee D W et a12008 The Plant Cell 20:1603-22; and Lee D W et al 2008 The Plant Cell 20:1603-22; each of which is incorporated by reference herein in its entirety.

The at least two localization signal sequences of an EML tag as described herein can be overlapping, contiguous (e.g. not separated by an exon), and/or separated by a short linker or exon sequence, which does not exceed 300 bp in length, e.g. it is 300 bp or shorter, 250 bp or shorter, 200 bp or shorter, 150 bp or shorter, 120 bp or shorter, 100 bp or shorter, 75 bp or shorter, 50 bp or shorter, 40 bp or shorter, or 30 bp or shorter. In some embodiments, the short linker or exon sequence does not exceed 120 bp in length. In some embodiments, the short linker or exon sequence does not exceed 30 bp in length. In some embodiments, the linker or exon sequence can comprise glycine and/or serine residues. In some embodiments, the linker or exon sequence can comprise a sequence which is at least 10% glycine and/or serine residues, e.g. at least 10%, at least 20%, at least 30%, at least 40%, at least 50%, at least 60%, at least 70%, at least 80%, at least 90% or more glycine and/or serine residues. In some embodiments, the linker or exon sequence can consist of glycine and/or serine residues. As used herein, a sequence comprising at least one exon also comprises at least one intron and/or requires at least one splicing event to occur in order to generate the mature mRNA.

An engineered multiple localization tag, when operably linked to a second nucleic acid sequence encoding a cargo polypeptide, will cause the cargo polypeptide to accumulate at detectable levels in at least two subcellular locations in the same cell, e.g. a first organelle and second organelle and optionally, the cytoplasm. In some embodiments, the engineered multiple localization tag will cause the cargo polypeptide to accumulate at detectable levels in at least two subcellular locations other than the cytoplasm, e.g. a first organelle and a second organelle. In some embodiments, the engineered multiple localization tag will cause the cargo polypeptide to accumulate at detectable levels in at least three subcellular locations, e.g. a first organelle, a second organelle, a third organelle, and optionally, the cytoplasm.

Specific exemplary embodiments of engineered multiple localization tags described herein are referred to as “TriTags,” e.g. TriTag1, TriTag2, and TriTag3, which are described elsewhere herein.

Two general classes of engineered multiple localization tags are described herein. The first class utilizes alternate splicing events to generate multiple peptide sequences from a single EML tag nucleic acid sequence, where each splice variant demonstrates different localization characteristics. This first class is referred to herein as “alternate splice EML tags.” The second class of EML tags is referred to as “embedded EML tags” and comprises EML tags where the multiple localization signal sequences are overlapping and/or embedded one within another such that a single translated product having multiple localization targets is generated.

Splicing of a transcript occurs when a segment of a RNA transcript or pre-mRNA between a donor splice site and an acceptor splice site is removed from the RNA molecule and the remaining two segments are ligated, resulting in a shortened mRNA transcript and an excised segment which will not be translated. This process is widely used, particularly in eukaryotic cells, to remove introns and to generate variants encoding different isoforms of a given protein. By flanking at least one of the localization signal sequences with a set of splicing sequences or signals, e.g. a donor and an acceptor splice site, a population of transcripts will result, comprising at least two species: 1) full-length transcripts comprising the flanked localization signal sequence and 2) shorter variants comprising sequences in which the flanked localization signal sequence has been removed by the occurrence of a splicing event. Splicing can be catalyzed by enzymes (e.g. the spliceosome) or by the sequence itself.

As used herein, “a set of compatible splicing sequences” refers to a group of RNA sequences, comprising at least one acceptor splice site and at least one donor splice site that, when transcribed as part of the same RNA molecule in a cell can, at a detectable rate, cause the intervening sequence to be removed from the RNA molecule. For example, a set of compatible splicing sequences can cause at least 5%, at least 10%, at least 20%, at least 40%, at least 60%, at least 80%, or at least 90% of a population of transcripts to have the intervening sequence removed prior to translation. The reeingineering of naturally-occurring splice sites/sequences is described in, e.g. Orengo et al 2006, Nucleic Acids Research 34:22:e148; Younis et al 2010 Molec. Cell. Biol. 30(7):1718-1728; and Syed et al. 2012 Trends Plant Sci 17(10):6161-23; each of which is incorporated by reference herein in its entirety. Splice prediction software is known in the art, (e.g. the Fruit Fly Splice Predictor, Human Splicing Finder, RegRNA, Exonic Splicing Enhancers Finder, the MIT Splice Predictor, GeneSplicer, Splice Predictor (DK), ASPic, SplicePort, NetPlantGene server (Hebsgaard et al., 1996)., and ASSP (Wang and Marin 2006 Gene 366:219-227). Each of the foregoing references is incorporated by reference herein in its entirety.

Where an alternate splice EML tag comprises multiple sets of compatible splicing sequences, the splicing sequences of each set do not interact with members of other sets, e.g. a donor splice sequence of a first set and an acceptor splice sequence of a second set do not engage in a splicing event at a significant level, e.g. less than 5% of the transcripts should experience such a splicing event. Non-limiting examples of multiple sets of compatible splicing sequences that can be used together are provided herein. Whether a first set of compatible splicing sequences will interact with a second set of compatible splicing sequences can be predicted by methods known in the art, e.g. by splicing prediction algorithms that are freely available on the world wide web. Non-limiting examples of such algorithms can be found at http://www.interactive-biosoftware.com/alamut/doc/2.0/splicing.html; http://www.wyomingbioinformatics.org/˜achurban/; and http://www.cbs.dtu.dk/services/NetPGene/.

In some embodiments, an alternate splice EML tag as described herein can further comprise at least a set of compatible splicing sequences, wherein the set of compatible splicing sequences flanks at least one localization signal sequence, and at least one localization signal sequence is not flanked by the set of compatible splicing sequences. In some embodiments, the localization signal sequence not flanked by the set of compatible splicing sequences is the 3′-most localization signal sequence of the EML tag.

In some embodiments, a set of compatible splicing sequences can comprise multiple donor splice sites and/or acceptor splice sets. In some embodiments, the multiple donor or acceptor splice sites can be alternative splice sites, e.g. with one donor splice site and two acceptor splice sites, a set can generate at least two alternative splice products. In some embodiments, the alternative donor or acceptor splice sites can have varying rates of splicing frequency, e.g. one of the alternative donor or acceptor splice sites can be “strong” and the other can be “weak.” In some embodiments, a pair of alternative splice sites comprises a weak and a strong splice site. As used herein, a “strong” donor or acceptor sequence is one that participates in a splicing event at a frequency at least 10% greater than the frequency of the “weak” sequence, e.g. at least 10%, at least 20%, at least 30%, at least 40%, at least 50%, at least 75%, at least 100%, at least 200%, at least 300%, at least 500% or greater. In some embodiments, wherein a set of compatible splicing sequences comprises alternative splice sequences (e.g. alternative donors or alternative acceptors), the weak splice site can be located 5′ of the flanked localization signal and the strong splice site can be located 3′ of the flanked localization signal.

In some embodiments, an EML tag described herein can further comprise a set of compatible splicing sequences, wherein the set comprises two alternative splice donor sequences and one splice acceptor sequence, wherein the two alternative splice donor sequences flank a first localization signal sequence. In some embodiments, the acceptor splice site can be located 3′ of both donor splice sets of the set. In some embodiments, the entire set of splicing sequences can be located 5′ of a second localization signal. In some embodiments, the entire set of splicing sequences can be located 3′ of a second localization signal.

In some embodiments, an EML tag described herein can further comprise a set of compatible splicing sequences, wherein the set comprises two alternative acceptor splice sites and one donor splice site, wherein the two alternative acceptor splice sites flank a first localization signal sequence. In some embodiments, the donor splice site can be located 5′ of both acceptor splice sites of the set. In some embodiments, the entire set of splicing sequences can be located 3′ of a second localization signal. In some embodiments, the entire set of splicing sequences can be located 5′ of a second localization signal.

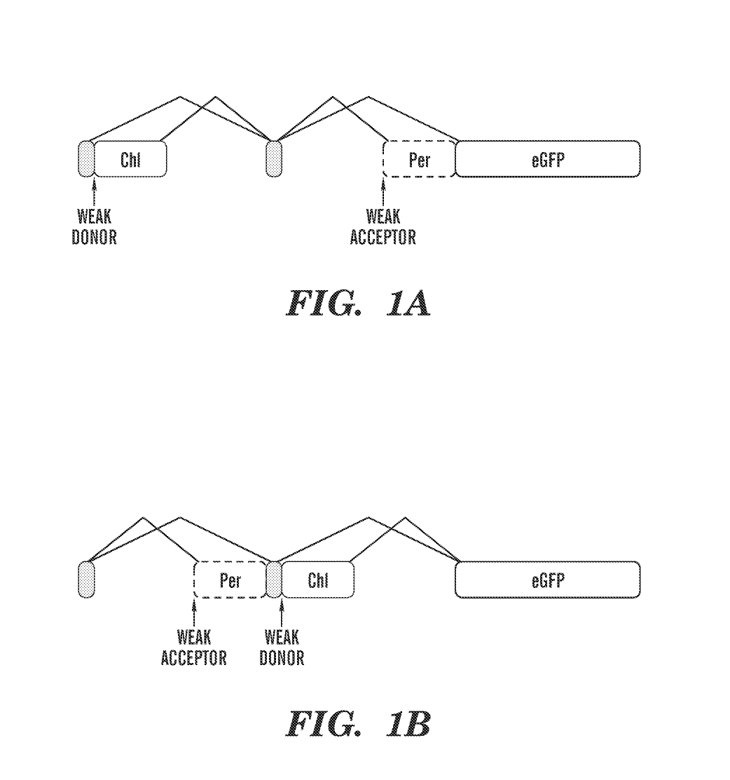

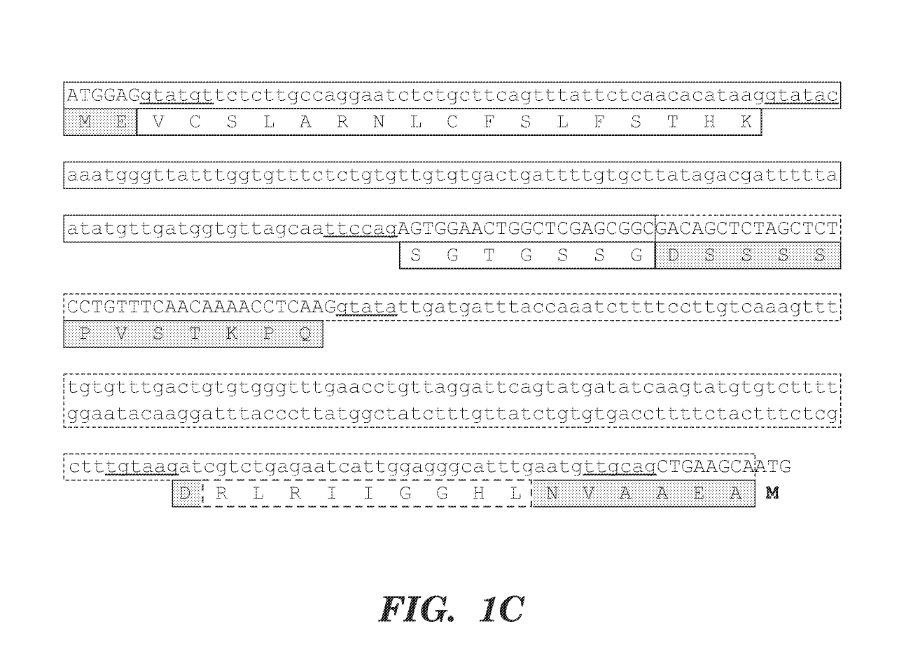

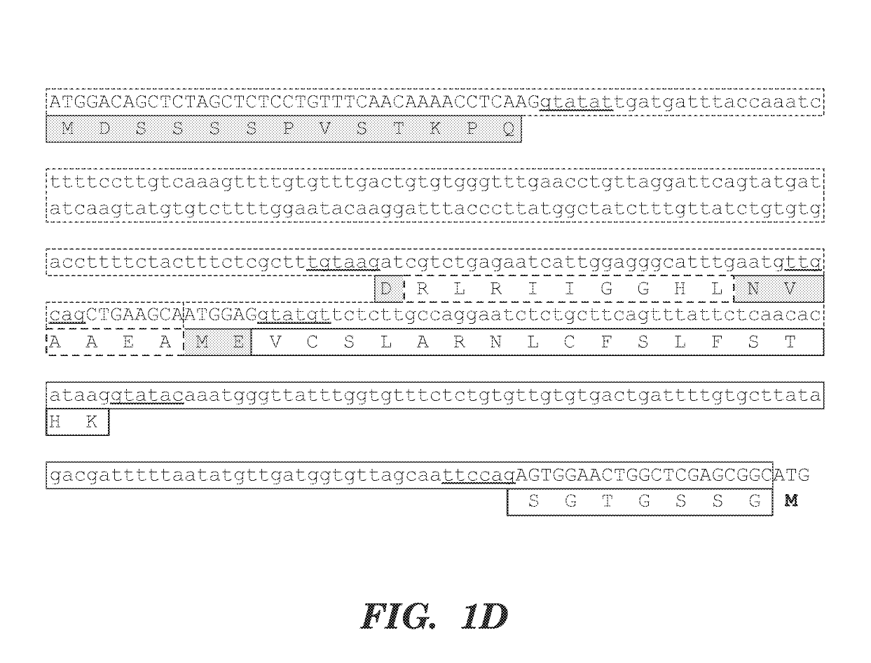

Exemplary sets of compatible splicing sites are described herein. By way of non-limiting example, a set of compatible splicing sites can comprise the sequences of SEQ ID NO:8 and SEQ ID NO: 10; SEQ ID NO: 9 and SEQ ID NO: 10; or the weak splice donor site of SEQ ID NO: 8, the strong splice donor site of SEQ ID NO: 9, and the splice acceptor site of SEQ ID NO: 10. By way of further non-limiting example, a second set of compatible splicing sites can comprise the sequences of SEQ ID NO:11 and SEQ ID NO: 13; SEQ ID NO: 12 and SEQ ID NO: 13; or the splice donor site of SEQ ID NO: 11, the weak splice acceptor site of SEQ ID NO: 12, and the strong splice acceptor site of SEQ ID NO: 13. FIGS. 1A-1D depict exemplary embodiments of alternate splice EML tags comprising sets of compatible splicing sites and depicting how the sets of splicing sequences can interact to generate splice variants.

Non-limiting examples of alternate splice EML tags can include tags having the nucleic acid sequences of SEQ ID NOs: 18 or 19, or nucleic acid sequences having at least 8% identity to SEQ ID NOs: 18 or 19, e.g. 80% or greater, 90% or greater, 95% or greater, or 98% or greater identity. Further non-limiting examples of alternate splice EML tags can include tags comprising the polypeptides of any of SEQ ID NOs: 3, 4, or 21-26 or polypeptides having at least 90% identity to any of SEQ ID NOs: 3, 4, or 21-26. Further non-limiting examples of alternate splice EML tags can include tags comprising a nucleic acid encoding any of the polypeptides of SEQ ID NOs: 3, 4, or 21-26 or nucleic acids encoding polypeptides having at least 90% identity to any of SEQ ID NOs: 3, 4, or 21-26. In some embodiments, an alternate splice EML tag can comprise a nucleic acid sequence which, when translated in a cell, will generate a population of variant polypeptides, wherein the population comprises a detectable level of at least two (e.g. two, three, or all) of the sequences selected from the group consisting of SEQ ID NOs: 3 and 21-23. In some embodiments, an alternate splice EML tag can comprise a nucleic acid sequence which, when translated in a cell, will generate a population of variant polypeptides, wherein the population comprises a detectable level of at least two (e.g. two, three, or all) of the sequences selected from the group consisting of SEQ ID NOs: 4 and 24-26. In any event, an EML tag which is a variant of a sequence described herein must retain at least 10% of the localization ability of the reference sequence from which is it derived, e.g. it must be able to direct localization of a cargo polypeptide to the desired target location(s) at least 10% as effectively as the reference localization signal (as measured by absolute or relative concentration as described elsewhere herein), e.g. at least 10%, at least 20%, at least 30%, at least 50%, at least 70%, at least 80%, at least 90%, at least 95%, at least 100% effectively or more effectively.

A second class of EML tags described herein comprises “embedded” EML tags. As the inventors have demonstrated herein, certain less conserved sequences of a first localization signal sequence can be replaced with a second localization signal sequence, embedding the second sequence within the first. The resulting EML tags can direct the polypeptides of which they are a part to the target organelles of both of the localization signal sequences.

The sequence of the second localization signal which is to be replaced can be identified, e.g. by aligning related localization signals to define poorly conserved regions. Where, for example, an alignment shows two identical or similar amino acids at corresponding positions, it is more likely that that site is important functionally. Where, conversely, alignment shows residues in corresponding positions to differ significantly in size, charge, hydrophobicity, etc., it is more likely that that site can tolerate variation in a functional polypeptide. Such alignments are readily created by one of ordinary skill in the art, e.g. using the default settings of the alignment tool of the BLASTP program, freely available on the world wide web at http://blast.ncbi.nlm.nih.gov/. Furthermore, homologs of any given polypeptide or nucleic acid sequence can be found using BLAST programs, e.g. by searching freely available databases of sequence for homologous sequences, or by querying those databases for annotations indicating a homolog (e.g. search strings that comprise a gene name or describe the activity of a gene). Such databases can be found, e.g. on the world wide web at http://ncbi.nlm.nih.gov/.

Poorly conserved regions of a localization signal that can permit embedding of another localization signal can be identified with, for example, SignalP software. See, e.g. Petersen et al Nature Methods 2011 8:785; which is incorporated by reference herein in its entirety.

As a non-limiting example, CTPb comprises a poorly conserved region from amino acid 37 to 46 of SEQ ID NO: 6. In some embodiments, an EML tag as described herein can comprise a first localization signal which has been substituted for the amino acids equivalent to residues 37 to 46 of SEQ ID NO: 6 in a second localization signal.

In some embodiments, an embedded EML tag as described herein can comprise a polypeptide having the sequence of SEQ ID NO:7 or a polypeptide having at least 80% identity, e.g. at least 80%, at least 90%, at least 95%, or at least 98% or greater identity, with the sequence of SEQ ID NO: 7. In some embodiments, an embedded EML tag as described herein can comprise a nucleic acid encoding a polypeptide having the sequence of SEQ ID NO:7 or a nucleic acid encoding a polypeptide having at least 80% identity, e.g. at least 80%, at least 90%, at least 95%, or at least 98% or greater identity, with the sequence of SEQ ID NO: 7. In some embodiments, an embedded EML tag as described herein can comprise a nucleic acid having the sequence of SEQ ID NO:20 or a nucleic acid having at least 80% identity, e.g. at least 80%, at least 90%, at least 95%, or at least 98% or greater identity, with the sequence of SEQ ID NO: 20. In any event, an EML tag which is a variant of a sequence described herein must retain at least 10% of the localization ability of the reference sequence from which is it derived, e.g. it must be able to direct localization of a cargo polypeptide to the desired target location(s) at least 10% as effectively as the reference localization signal (as measured by absolute or relative concentration as described elsewhere herein), e.g. at least 10%, at least 20%, at least 30%, at least 50%, at least 70%, at least 80%, at least 90%, at least 95%, at least 100% effectively or more effectively.

An EML tag, as described herein can comprise nucleic acid and/or polypeptide sequences comprising localization signals and/or splice sites. Non-limiting examples of such sequences are provided herein. In some embodiments, an EML tag can comprise a sequence provided herein. In some embodiments, an EML tag can comprise a functional variant of a sequence provided herein. In some embodiments, the functional variant can be a conservative substitution variant. A functional variant will result in localization to at least 2 different sub-cellular locations

In some embodiments, an EML tag, as described herein, can be suitable for expression in a plant or plant cell, e.g. it can comprise localization signals and splice sites that are functional in a plant cell. In some embodiments, an EML tag, as described herein, will not be functional in a cell other than a plant cell, e.g. a yeast or animal cell.

In one aspect, described herein is a vector comprising an EML tag as described herein. In one aspect, described herein is a cell or organism comprising an EML tag as described herein or comprising a vector comprising an EML tag as described herein. In some embodiments, the cell or organism can be a plant or a plant cell. In some embodiments, the cell or organism can be a photosynthetic cell or organism.

In some embodiments, the vector can further comprise a nucleic acid sequence encoding an operably linked polypeptide (i.e. a cargo polypeptide) or a cloning site suitable for the introduction of a nucleic acid sequence encoding an operably linked polypeptide (i.e. a cargo polypeptide). In some embodiments, the EML tag can be located entirely on one flank of the nucleic acid sequence encoding the cargo polypeptide or the cloning site. In some embodiments, the EML tag can be located 5′ of the nucleic acid sequence encoding the cargo polypeptide or the cloning site. In some embodiments, the EML tag can be located 3′ of the nucleic acid sequence encoding a cargo polypeptide or the cloning site.

In some embodiments, an expression vector can comprise an EML tag as described herein, e.g. for expression and post-translational targeting of a cargo polypeptide in a cell and/or organism of interest. As used herein, the term “expression vector” refers to a vector that has the ability to incorporate and express exogenous nucleotide fragments in a cell. A cloning or expression vector may comprise additional elements, for example, the expression vector may have two replication systems, thus allowing it to be maintained in two organisms, for example in plant cells for expression and in a prokaryotic host for cloning and amplification. The term vector may also be used to describe a recombinant virus, e.g., a virus modified to contain the coding sequence for a gene of interest. As used herein, a vector may be of viral or non-viral origin. Suitable vectors are discussed further herein below.

The expression vector can include 5′ and/or 3′ regulatory sequences (e.g. an EML tag as described herein) operably linked to a gene encoding a cargo polypeptide; a construct referred to herein as the “transgene.” The term “operably linked” as used herein refers to a functional linkage between a regulatory element and a second sequence, wherein the regulatory element influences the expression and/or processing of the second sequence. Generally, operably linked means that the nucleic acid sequences being linked are contiguous and, where necessary to join two protein coding regions, contiguous and in the same reading frame. The transgene can include in the 5′ to 3′ direction of transcription: a transcriptional and translational initiation region (i.e., a promoter or translation initiation region), a nucleotide sequence encoding a polypeptide, and a transcriptional and translational termination region (i.e., termination region) functional in the organism serving as a host. An EML tag as described herein can be included between the inititation region and the nucleotide sequence encoding a cargo polypeptide or between the nucleotide sequence encoding a cargo polypeptide and the termination region. The transcriptional initiation region (i.e., the promoter) may be native, analogous, foreign or heterologous to the host organism and/or to the nucleotide sequence encoding a cargo polypeptide. Additionally, the promoter may be the natural sequence associated with that cargo polypeptide's gene or alternatively a synthetic sequence. A single vector can comprise multiple transgenes. The additional transgenes can optionally further comprise an EML tag as described herein.

The expression vector can additionally contain selectable marker genes. Expression vectors can be provided with a plurality of restriction sites for insertion of the transgene and/or the nucleotide sequence encoding a cargo polypeptide to be under the transcriptional regulation of the regulatory regions already present in the vector.

Most genes have regions of DNA sequence that are known as promoters and which regulate gene expression. Promoter regions are typically found in the flanking DNA sequence upstream from the coding sequence in both prokaryotic and eukaryotic cells. A promoter sequence provides for regulation of transcription of the downstream gene sequence and typically includes from about 50 to about 2,000 nucleotide base pairs. Promoter sequences can also contain regulatory sequences such as enhancer sequences that can influence the level of gene expression. Some isolated promoter sequences can provide for gene expression of heterologous genes, that is, a gene different from the native or homologous gene. Promoter sequences are also known to be strong or weak or inducible. A strong promoter provides for a high level of gene expression, whereas a weak promoter provides for a very low level of gene expression. An inducible promoter is a promoter that provides for turning on and off of gene expression in response to an exogenously added agent or to an environmental or developmental stimulus. Promoters can also provide for tissue specific or developmental regulation. An isolated promoter sequence that is a strong promoter for heterologous genes can be advantageous because it provides for a sufficient level of gene expression to allow for easy detection and selection of transformed cells and provides for a high level of gene expression when desired.

A promoter comprised by some embodiments of the present technology can provide for expression of an EML tag and an operably linked cargo polypeptide from a nucleotide sequence encoding the EML tag and the cargo polypeptide. In some embodiments, the promoter can cause expression of a detectable level of the EML tag and the cargo polypeptide. In some embodiments, the promoter can cause expression of a level of the EML tag and the cargo polypeptide such that detectable levels of the cargo polypeptide can be found in the subcellular locations the EML tag is designed to target (e.g. in the choloroplast and the peroxisome if the EML tag comprises chloroplast and peroxisome localization signals).

Promoters can be functional in, e.g. plastids or plant cells. Examples of promoters that can be used in an expression vector as described herein include, but are not limited to, the CaMV 35S promoter (Odell et al., Nature, 313:810 (1985)), the CaMV 19S (Lawton et al., Plant Mol. Biol., 9:31F (1987)), nos (Ebert et al., Proc. Nat. Acad. Sci. (U.S.A.), 84:5745 (1987)), Adh (Walker et al., Proc. Nat. Acad. Sci. (U.S.A.), 84:6624 (1987)), sucrose synthase (Yang et al., Proc. Nat. Acad. Sci. (U.S.A.), 87:4144 (1990)), the octapine synthase (OCS) promoter, the figwort mosaic virus 35S promoter, α-tubulin, napin, actin (Wang et al., Mol. Cell. Biol., 12:3399 (1992)), cab (Sullivan et al., Mol. Gen. Genet., 215:431 (1989)), PEPCase promoter (Hudspeth et al., Plant Mol. Biol., 12:579 (1989)), the 7S-alpha′-conglycinin promoter (Beachy et al., EMBO J, 4:3047 (1985)), those associated with the R gene complex (Chandler et al., The Plant Cell, 1:1175 (1989)), the core promoter of the Rsyn7 promoter and other constitutive promoters disclosed in WO 99/43838 and U.S. Pat. No. 6,072,050; the core CaMV 35S promoter (Odell et al. (1985) Nature 313: 810-812); rice actin (McElroy et al. (1990) Plant Cell 2: 163-171); ubiquitin (Christensen et al. (1989) Plant Mol. Biol. 12: 619-632 and Christensen et al. (1992) Plant Mol. Biol. 18: 675-689); pEMU (Last et al. (1991) Theor. Appl. Genet. 81: 581-588); MAS (Velten et al. (1984) EMBO J. 3:2723-2730); ALS promoter (U.S. Pat. No. 5,659,026), and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611. The preceding references are incorporated by reference herein in their entireties.

Moreover, transcription enhancers or duplications of enhancers can be used to increase expression from a particular promoter. Examples of such enhancers include, but are not limited to, elements from the CaMV 35S promoter and octopine synthase genes (Last et al., U.S. Pat. No. 5,290,924). For example, it is contemplated that vectors for use in accordance with the present technology can be constructed to include the ocs enhancer element. This element was first identified as a palindromic enhancer from the octopine synthase (ocs) gene of Agrobacterium (Ellis et al., EMBO J., 6:3203 (1987)), and is present in at least 10 other promoters (Bouchez et al., EMBO J., 8:4197 (1989)); which are incorporated by reference herein in their entireties. It is proposed that the use of an enhancer element, such as the ocs element and particularly multiple copies of the element, will act to increase the level of transcription from adjacent promoters.

Where low level expression is desired, weak promoters will be used. Generally, the term “weak promoter” as used herein refers to a promoter that drives expression of a coding sequence at a low level. By low level expression at levels of about 1/1000 transcripts to about 1/100,000 transcripts to about 1/500,000 transcripts is intended. Alternatively, it is recognized that the term “weak promoters” also encompasses promoters that drive expression in only a few cells and not in others to give a total low level of expression. Where a promoter drives expression at unacceptably high levels, portions of the promoter sequence can be deleted or modified to decrease expression levels. Such weak constitutive promoters include, for example the core promoter of the Rsyn7 promoter (WO 99/43838 and U.S. Pat. No. 6,072,050), the core 35S CaMV promoter, and the like. Other weak constitutive promoters include, for example, those disclosed in U.S. Pat. Nos. 5,608,149; 5,608,144; 5,604,121; 5,569,597; 5,466,785; 5,399,680; 5,268,463; 5,608,142; and 6,177,611; herein incorporated by reference.

In some embodiments, a promoter that provides tissue specific expression or developmentally regulated gene expression in plants can be used. In some embodiments, the promoter comprised by an expression vector as described herein can be a tissue-specific promoter, examples of which are known in the art.

In some embodiments, the promoter can also be inducible so that gene expression can be turned on or off by an exogenously added agent. Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference. A further example of an inducible promoter is the light inducible promoter from the small subunit of Rubisco (Pellegrineschi et al., Biochem. Soc. Trans. 23(2):247-250 (1995); which is incorporated by reference herein in its entirety).

Transgenes can also include the EML tag and the nucleic acid encoding a cargo polypeptide along with a nucleic acid sequence that acts as a transcription termination signal and that allows for the polyadenylation of the resultant mRNA. Such transcription termination signals are placed 3′ or downstream of the coding region of interest. The termination region may be native with the transcriptional initiation region, may be native with the operably linked nucleic acid encoding the cargo polypeptide, may be native with the host organism, or may be derived from another source (i.e., foreign or heterologous to the promoter, the sequence of interest, the host organism, or any combination thereof). Preferred transcription termination signals contemplated include the transcription termination signal from the nopaline synthase gene of Agrobacterium tumefaciens (Bevan et al., Nucl. Acid Res., 11:369 (1983)), the terminator from the octopine synthase gene of Agrobacterium tumefaciens, and the 3′ end of genes encoding protease inhibitor I or II from potato or tomato, although other transcription termination signals known to those of skill in the art are also contemplated. Regulatory elements such as Adh intron 1 (Callis et al., Genes Develop., 1:1183 (1987)), sucrose synthase intron (Vasil et al., Plant Physiol., 91:5175 (1989)) or TMV omega element (Gallie et al., The Plant Cell, 1:301 (1989)) may further be included where desired. These 3′ nontranslated regulatory sequences can be obtained as described in An, Methods in Enzymology, 153:292 (1987) or are already present in plasmids available from commercial sources such as Clontech, (Palo Alto, Calif.). The 3′ nontranslated regulatory sequences can be operably linked to the 3′ terminus of a gene by standard methods. Other such regulatory elements useful in the practice of the invention are known to those of skill in the art. The preceding references are incorporated by reference herein in their entireties.

Selectable marker genes or reporter genes are also useful in the methods and compositions described herein. Such genes can impart a distinct phenotype to cells expressing the marker gene and thus allow such transformed cells to be distinguished from cells that do not have the marker. Selectable marker genes confer a trait that one can ‘select’ for by chemical means, i.e., through the use of a selective agent (e.g., a herbicide, antibiotic, or the like). Reporter genes, or screenable genes, confer a trait that one can identify through observation or testing, i.e., by ‘screening.’ Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional examples of suitable selectable marker genes include, but are not limited to, genes encoding resistance to chloramphenicol (Herrera Estrella et al. (1983) EMBO J. 2:987-992); methotrexate (Herrera Estrella et al. (1983) Nature 303:209-213; and Meijer et al. (1991) Plant Mol. Biol. 16:807-820); streptomycin (Jones et al. (1987) Mol. Gen. Genet. 210:86-91); spectinomycin (Bretagne-Sagnard et al. (1996) Transgenic Res. 5:131-137); bleomycin (Hille et al. (1990) Plant Mol. Biol. 7:171-176); sulfonamide (Guerineau et al. (1990) Plant Mol. Biol. 15:127-136); bromoxynil (Stalker et al. (1988) Science 242:419-423); glyphosate (Shaw et al. (1986) Science 233:478-481; and U.S. application Ser. Nos. 10/004,357; and 10/427,692); phosphinothricin (DeBlock et al. (1987) EMBO J. 6:2513-2518) and genes encoding DHFR or dalapon dehalogenase. See generally, Yarranton (1992) Curr. Opin. Biotech. 3: 506-511; Christopherson et al. (1992) Proc. Natl. Acad. Sci. USA 89: 6314-6318; Yao et al. (1992) Cell 71: 63-72; Reznikoff (1992) Mol. Microbiol. 6: 2419-2422; Barkley et al. (1980) in The Operon, pp. 177-220; Hu et al. (1987) Cell 48: 555-566; Brown et al. (1987) Cell 49: 603-612; Figge et al. (1988) Cell 52: 713-722; Deuschle et al. (1989) Proc. Natl. Acad. Sci. USA 86: 5400-5404; Fuerst et al. (1989) Proc. Natl. Acad. Sci. USA 86: 2549-2553; Deuschle et al. (1990) Science 248: 480-483; Gossen (1993) Ph.D. Thesis, University of Heidelberg; Reines et al. (1993) Proc. Natl. Acad. Sci. USA 90: 1917-1921; Labow et al. (1990) Mol. Cell. Biol. 10: 3343-3356; Zambretti et al. (1992) Proc. Natl. Acad. Sci. USA 89: 3952-3956; Baim et al. (1991) Proc. Natl. Acad. Sci. USA 88: 5072-5076; Wyborski et al. (1991) Nucleic Acids Res. 19: 4647-4653; Hillenand-Wissman (1989) Topics Mol. Struc. Biol. 10: 143-162; Degenkolb et al. (1991) Antimicrob. Agents Chemother. 35: 1591-1595; Kleinschnidt et al. (1988) Biochemistry 27: 1094-1104; Bonin (1993) Ph.D. Thesis, University of Heidelberg; Gossen et al. (1992) Proc. Natl. Acad. Sci. USA 89: 5547-5551; Oliva et al. (1992) Antimicrob. Agents Chemother. 36: 913-919; Hlavka et al. (1985) Handbook of Experimental Pharmacology, Vol. 78 (Springer-Verlag, Berlin); and Gill et al. (1988) Nature 334: 721-724; which are incorporated by reference herein in their entireties. Screenable markers that may be employed include, but are not limited to, a β-glucuronidase or uidA gene (GUS) which encodes an enzyme for which various chromogenic substrates are known; an R-locus gene, which encodes a product that regulates the production of anthocyanin pigments (red color) in plant tissues (Dellaporta et al., in Chromosome Structure and Function, pp. 263-282 (1988)); a β-lactamase gene (Sutcliffe, Proc. Nat. Acad. Sci. (U.S.A.), 75:3737 (1978)), which encodes an enzyme for which various chromogenic substrates are known (e.g., PADAC, a chromogenic cephalosporin); a xylE gene (Zukowsky et al., Proc. Nat. Acad. Sci. (U.S.A), 80:1101 (1983)) that encodes a catechol dioxygenase that can convert chromogenic catechols; an α-amylase gene (Ikuta et al., Biotech., 8:241 (1990)); a tyrosinase gene (Katz et al., J. Gen. Microbiol., 129:2703 (1983)) that encodes an enzyme capable of oxidizing tyrosine to DOPA and dopaquinone which in turn condenses to form the easily detectable compound melanin; a β-galactosidase gene, which encodes an enzyme for which there are chromogenic substrates; a luciferase (lux) gene (Ow et al., Science, 234:856 (1986)), which allows for bioluminescence detection; or even an aequorin gene (Prasher et al., Biochem. Biophys. Res. Comm, 126:1259 (1985)), which may be employed in calcium-sensitive bioluminescence detection, or a green fluorescent protein gene (Niedz et al., Plant Cell Reports, 14:403 (1995)). The preceding references are incorporated by reference herein in their entireties. The presence of the lux gene in transformed cells may be detected using, for example, X-ray film, scintillation counting, fluorescent spectrophotometry, low-light video cameras, photon-counting cameras, or multiwell luminometry. It is also envisioned that this system may be developed for populational screening for bioluminescence, such as on tissue culture plates, or even for whole plant screening.

Expression vectors can include additional DNA sequences that provide for easy selection, amplification, and transformation of the transgene in prokaryotic and eukaryotic cells. The additional DNA sequences can include origins of replication to provide for autonomous replication of the vector, selectable marker genes, preferably encoding antibiotic or herbicide resistance, unique multiple cloning sites providing for multiple sites to insert DNA sequences or genes encoded in the transgene, and sequences that enhance transformation of prokaryotic and/or eukaryotic cells.

Non-limiting examples of expression vectors suitable for use in the methods and compositions described herein include pBR322 and related plasmids, pACYC and related plasmids, transcription vectors, expression vectors, phagemids, yeast expression vectors, plant expression vectors, pDONR201 (Invitrogen), pBI121, pBIN20, pEarleyGate100 (ABRC), pEarleyGate102 (ABRC), pCAMBIA, pUC-derived vectors, pSK-derived vectors, pGEM-derived vectors, pSP-derived vectors, pBS-derived vectors, T-DNA, transposons, and artificial chromosomes.

Another vector that is useful for expression in both plant and prokaryotic cells is the binary Ti plasmid (as disclosed in Schilperoort et al., U.S. Pat. No. 4,940,838; which is incorporated by reference herein in its entirety) as exemplified by vector pGA582. This binary Ti plasmid vector has been previously characterized by An, cited supra. This binary Ti vector can be replicated in prokaryotic bacteria such as E. coli and Agrobacterium. The Agrobacterium plasmid vectors can also be used to transfer the transgene to plant cells. The binary Ti vectors preferably include the nopaline T DNA right and left borders to provide for efficient plant cell transformation, a selectable marker gene, unique multiple cloning sites in the T border regions, the colE1 replication of origin and a wide host range replicon. The binary Ti vectors carrying a transgene as described herein (e.g. comprising an EML tag and a nucleic acid sequence encoding a cargo polypeptide) can be used to transform both prokaryotic and eukaryotic cells, but is preferably used to transform plant cells. See, for example, Glassman et al., U.S. Pat. No. 5,258,300; which is incorporated by reference herein in its entirety.

In preparing the expression vector, the various nucleotide fragments may be manipulated so as to provide for the nucleotide sequences in the proper orientation and, as appropriate, in the proper reading frame. Toward this end, adapters or linkers may be employed to join the nucleotide fragments or other manipulations may be involved to provide for convenient restriction sites, removal of superfluous nucleotide sequences, removal of restriction sites, or the like. For this purpose, in vitro mutagenesis, primer repair, restriction, annealing, resubstitutions, e.g., transitions and transversions, may be involved.

The following discusses the introduction of an EML-tagged construct to a host organism, specifically exemplifying introduction to plants. It should be understood however, that any method suitable for the given host, whether it is plant, animal, fungus, or protist, can be used to introduce the EML-tagged construct.

After constructing or obtaining an expression vector comprising an EML tag and a nucleic acid sequence encoding a cargo polypeptide, the vector can then be introduced into a host organism, e.g. plant or plant cell. “Introducing” is intended to mean presenting the expression vector to the host organism (e.g. the plant) in such a manner that the sequence gains access to the interior of a cell. The methods of the various embodiments do not depend on a particular method for introducing a vector into a plant, only that the expression vector gains access to the interior of at least one cell of the plant. Methods for introducing an expression vector into plants are known in the art including, but not limited to, stable transformation methods, transient transformation methods, and virus-mediated methods. “Stable transformation” is intended to mean that the nucleotide construct introduced into a plant integrates into the genome of the plant and is capable of being inherited by the progeny thereof “Transient transformation” is intended to mean that a polynucleotide is introduced into the plant and does not integrate into the genome of the plant, or for example, that a polypeptide is directly introduced into a plant.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4: 320-334), electroporation (Riggs et al. (1986) Proc. Natl. Acad. Sci. USA 83: 5602-5606), Agrobacterium-mediated transformation (U.S. Pat. Nos. 5,563,055 and 5,981,840), direct gene transfer (Paszkowski et al. (1984) EMBO J. 3: 2717-2722), ballistic particle acceleration (see, for example, U.S. Pat. Nos. 4,945,050; 5,879,918; 5,886,244; 5,990,390; and 5,932,782; Tomes et al. (1995) in Plant Cell, Tissue, and Organ Culture: Fundamental Methods, ed. Gamborg and Phillips (Springer-Verlag, Berlin); and McCabe et al. (1988) Biotechnology 6: 923-926); Lea transformation (WO 00/28058); Type II embryogenic callus cells (W. J. Gordon-Kamm et al. Plant Cell, 2:603 (1990); M. E. Fromm et al. Bio/Technology, 8:833 (1990); D. A. Walters et al. Plant Molecular Biology, 18:189 (1992)); or electroporation of type I embryogenic calluses (D'Halluin et al. The Plant Cell, 4:1495 (1992); U.S. Pat. No. 5,384,253). For potato transformation see Tu et al. (1998) Plant Molecular Biology 37: 829-838 and Chong et al. (2000) Transgenic Research 9: 71-78. Transformation of plant cells by vortexing with DNA-coated tungsten whiskers (Coffee et al., U.S. Pat. No. 5,302,523) and transformation by exposure of cells to DNA-containing liposomes can also be used. Additional transformation procedures can be found in Weissinger et al. (1988) Aim. Rev. Genet. 22: 421-477; Sanford et al. (1987) Particulate Science and Technology 5: 27-37 (onion); Christou et al. (1988) Plant Physiol. 87: 671-674 (soybean); McCabe et al. (1988) Bio/Technology 6: 923-926 (soybean); Finer and McMullen (1991) In Vitro Cell Dev. Biol. 27P: 175-182 (soybean); Singh et al. (1998) Theor. Appl. Genet. 96: 319-324 (soybean); Datta et al. (1990) Biotechnology 8: 736-740 (rice); Klein et al. (1988) Proc. Natl. Acad. Sci. USA 85: 4305-4309 (maize); Klein et al. (1988) Biotechnology 6:559-563 (maize); U.S. Pat. Nos. 5,240,855; 5,322,783 and 5,324,646; Klein et al. (1988) Plant Physiol. 91: 440-444 (maize); Fromm et al. (1990) Biotechnology 8: 833-839 (maize); Hooykaas-Van Slogteren et al. (1984) Nature (London) 311: 763-764; U.S. Pat. No. 5,736,369 (cereals); Bytebier et al. (1987) Proc. Natl. Acad. Sci. USA 84: 5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9: 415-418 and Kaeppler et al. (1992) Theor. Appl. Genet. 84: 560-566 (whisker-mediated transformation); D'Halluin et al. (1992) Plant Cell 4: 1495-1505 (electroporation); Li et al. (1993) Plant Cell Reports 12: 250-255 and Christou and Ford (1995) Annals of Botany 75: 407-413 (rice); Osjoda et al. (1996) Nature Biotechnology 14: 745-750 (maize via Agrobacterium tumefaciens); all of which are herein incorporated by reference.

In some embodiments, the nucleotide sequence encoding an EML tag and an operably linked nucleic acid sequence encoding a cargo polypeptide can be provided to a plant using a variety of transient transformation methods. Such transient transformation methods include, but are not limited to, the introduction of the nucleotide sequence directly into the plant or the introduction of the transcript into the plant. Such methods include, for example, microinjection or particle bombardment. See, for example, Crossway et al. (1986) Mol Gen. Genet. 202: 179-185; Nomura et al. (1986) Plant Sci. 44: 53-58; Hepler et al. (1994) Proc. Natl. Acad. Sci. 91: 2176-2180 and Hush et al. (1994) The Journal of Cell Science 107: 775-784, all of which are herein incorporated by reference. Alternatively, the nucleotide sequence can be transiently transformed into the plant using techniques known in the art. Such techniques include the use of a viral vector system and the precipitation of the polynucleotide in a manner that precludes subsequent release of the DNA. Thus, transcription from the particle-bound DNA can occur, but the frequency with which it is released to become integrated into the genome is greatly reduced. Such methods include the use of particles coated with polyethylimine (PEI; Sigma #P3143).

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the nucleotide sequence encoding an EML tag and an operably linked polypeptide can be contained in a transfer cassette, comprised by an expression vector, flanked by two non-identical recombination sites. The transfer cassette can be introduced into a plant and stably incorporated into its genome at a target site which is flanked by two non-identical recombination sites that correspond to the sites of the transfer cassette. An appropriate recombinase can be provided and the transfer cassette is integrated at the target site. The nucleotide sequence encoding an EML tag and an operably linked polypeptide can thereby be integrated at a specific chromosomal position in the plant genome.

In some embodiments, the nucleotide sequence encoding an EML tag and an operably linked polypeptide can be provided to the plant by contacting the plant with a virus or viral nucleic acids. Generally, such methods involve incorporating the nucleotide construct of interest within a viral DNA or RNA molecule. It is recognized that the EML tag and operably linked polypeptide can be initially synthesized as part of a viral polyprotein, which later may be processed by proteolysis in vivo or in vitro to produce the final polypeptide comprising an EML tag. It is also recognized that such a viral polyprotein, comprising at least a portion of the amino acid sequence of an EML tag and an operably linked polypeptide as described herein, may have the desired activity. Such viral polyproteins and the nucleotide sequences that encode for them are encompassed by the various embodiments. Methods for providing plants with nucleotide constructs and producing the encoded proteins in the plants, which involve viral DNA or RNA molecules, are known in the art. See, for example, U.S. Pat. Nos. 5,889,191; 5,889,190; 5,866,785; 5,589,367; and 5,316,931; herein incorporated by reference.

Expression of a gene can be detected and quantitated in the transformed cells. Gene expression can be quantitated by RT-PCR analysis, a quantitative Western blot using antibodies specific for the EML tag and/or cargo polypeptide or by detecting the activity of the operably linked cargo polypeptide. The tissue and subcellular location of the operably linked cargo polypeptide can be determined by immunochemical staining methods using antibodies specific for the cargo polypeptide or subcellular fractionation and subsequent biochemical and/or immunological analyses. Transformed cells can also be selected by detecting the presence of a selectable marker gene or a reporter gene, for example, by detecting a selectable herbicide resistance marker. Transient expression of a transgene can be detected in the transgenic embryogenic calli using antibodies specific for the cloned cargo polypeptide, or by RT-PCR analyses. The skilled artisan will also recognize that different independent transformation events will result in different levels and patterns of expression of a transgene (Jones et al., EMBO J. 4:2411-2418 (1985); De Almeida et al., Mol. Gen. Genetics 218:78-86 (1989)). Thus, multiple events must be screened in order to obtain lines displaying the desired expression level and pattern. Such screening may be accomplished by northern analysis of mRNA expression, western analysis of protein expression, or phenotypic analysis.

Transformed embryogenic calli, meristematic tissue, embryos, leaf discs and the like can then be used to generate transgenic plants that exhibit stable inheritance of the trangene. Plant cell lines exhibiting satisfactory levels of expression and/or activity of an EML tag and an operably linked cargo polypeptide can be put through a plant regeneration protocol to obtain mature plants and seeds by methods well known in the art (for example, see, U.S. Pat. Nos. 5,990,390 and 5,489,520; and Laursen et al., Plant Mol. Biol., 24:51 (1994); which are incorporated by reference herein in their entireties). The plant regeneration protocol allows the development of somatic embryos and the subsequent growth of roots and shoots. To determine whether the desired trait is expressed in differentiated organs of the plant, and not solely in undifferentiated cell culture, regenerated plants can be assayed for the levels of transgene expression and/or activity in various portions of the plant relative to regenerated, non-transformed plants. If possible, the regenerated plants can be self pollinated. In addition, pollen obtained from the regenerated plants can be crossed to seed grown plants of agronomically important inbred lines. In some cases, pollen from plants of these inbred lines can be used to pollinate regenerated plants. The transgenic trait can be genetically characterized by evaluating the segregation of the trait in first and later generation progeny. The heritability and expression in plants of traits selected in tissue culture are of particular importance if the traits are to be commercially useful.

The transgenic plants produced herein are expected to be useful for a variety of commercial and research purposes. In some embodiments, the plants possess traits beneficial to agricultural use (e.g., improved biosynthetic or metabolic pathways). The transgenic plants may also be used in commercial breeding programs, or may be crossed or bred to plants of related crop species. Improvements encoded by the recombinant DNA may be transferred, e.g., from the originally transgenic cells of one species to cells of other species, e.g., by protoplast fusion.

In some embodiments, an EML tag as described herein is operably linked to a nucleic acid sequence encoding a cargo polypeptide comprising an enzyme of the 3-hydroxypropionate (3-HOP) pathway. Such enzymes, variants thereof, and methods of identifying them are described, e.g. in PCT Application No: PCT/US13/27620, filed Feb. 25, 2013 and which is incorporated by reference herein in its entirety. Non-limiting examples of enzymes of the 3-HOP pathway can include malonyl-CoA reductase (MCR); propionyl-CoA synthase (PCS); (S)-malyl-CoA/β-methylmalyl-CoA/(S)-citramalyl-CoA (MMC lyase); mesaconyl-C1-CoA hydratase (β-methmalyl-CoA-dehydratase); mesaconyl-CoA C1-C4 transferase; mesaconyl-C4-CoA hydratase; nicotinic cofactor-dependent glycolate dehydrogenase; pyruvate kinase; enolase; phosphoglycerate mutase; 3-phosphoglycerate kinase; malonyl-CoA reductase; and propionyl-CoA synthase.

In some embodiments, the technology described herein can relate to a nucleic acid molecule having the sequence of, or encoding the polypeptide having the sequence of, any of SEQ ID NOs: 28-87, or a variant of such a sequence. In some embodiments, the variant can have at least 80% (e.g. 80% or greater, 90% or greater, 95% or greater, or 98% or greater) identity with the sequence of one of SEQ ID NOs: 28-87. In some embodiments, the technology described herein relates to a vector comprising a nucleic acid molecule described in the present paragraph. In some embodiments, the technology described herein relates to an engineered cell or organism comprising a nucleic acid molecule or vector as described in the present paragraph. In any event, a nucleic acid molecule which is a variant of a sequence described herein must retain at least 10% of the localization ability of the reference sequence from which is it derived, e.g. it must be able to direct localization of the cargo polypeptide to the desired target location at least 10% as effectively as the reference sequence (as measured by absolute or relative concentration as described elsewhere herein), e.g. at least 10%, at least 20%, at least 30%, at least 50%, at least 70%, at least 80%, at least 90%, at least 95%, at least 100% effectively or more effectively.

In some embodiments, the cell or organism can be a photosynthetic organism (e.g. a plant or cyanobacterium). As used herein, “photosynthesis” refers to the process in green plants and certain other organisms by which carbohydrates are synthesized from carbon dioxide and water using light as an energy source. Most forms of photosynthesis release oxygen as a byproduct. As is well known in the art, the photosynthetic process includes several independent reactions, including reactions that are conducted in the presence of and utilizing light energy as well as reactions that can be conducted in the dark or without light energy, in which carbon dioxide and water are converted into organic compounds, e.g., carbohydrates and others, by bacteria, algae and plants in the presence of a pigment, e.g. chlorophyll. As used herein, the term “non-photospithetic” refers to a cell or organism which does not have a natural ability to perform photosynthesis.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

A “variant,” as referred to herein, is a polypeptide substantially homologous to a given native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein. As to amino acid sequences, one of skill will recognize that individual substitutions, deletions or additions to a nucleic acid, peptide, polypeptide, or protein sequence which alters a single amino acid or a small percentage, (i.e. 5% or fewer, e.g. 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or note, has more than 100% of the activity of the wildtype localization signal, e.g. 110%, 125%, 150%, 175%, 200%, 500%, 1000% or more. One method of identifying amino acid residues which can be substituted is to align, for example, homologs from one or more species. Alignment can provide guidance regarding not only residues likely to be necessary for function but also, conversely, those residues likely to tolerate change. Where, for example, an alignment shows two identical or similar amino acids at corresponding positions, it is more likely that that site is important functionally. Where, conversely, alignment shows residues in corresponding positions to differ significantly in size, charge, hydrophobicity, etc., it is more likely that that site can tolerate variation in a functional polypeptide. Similarly, alignment with a related polypeptide from the same species, which does not show the same activity, can also provide guidance with respect to regions or structures required for activity. Alignments are readily generated by one of skill in the art using freely available programs. The variant amino acid or DNA sequence can be at least 90%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web. The variant amino acid or DNA sequence can be at least 90%, at least 91%, at least 92%, at least 93%, at least 94%, at least 95%, at least 96%, at least 97%, at least 98%, at least 99%, or more, similar to the sequence from which it is derived (referred to herein as an “original” sequence). The degree of similarity (percent similarity) between an original and a mutant sequence can be determined, for example, by using a similarity matrix. Similarity matrices are well known in the art and a number of tools for comparing two sequences using similarity matrices are freely available online, e.g. BLASTp (available on the world wide web at http://blast.ncbi.nlm.nih.gov), with default parameters set.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity of a native or reference polypeptide is retained (e.g. the ability to localize a cargo polypeptide). Conservative substitution tables providing functionally similar amino acids are well known in the art. Such conservatively modified variants are in addition to and do not exclude polymorphic variants, interspecies homologs, and alleles consistent with the disclosure. Typically conservative substitutions for one another include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

In general, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. For example, in some embodiments of the present invention, an engineered EML tag comprises multiple localization signals that are each found in nature, but are not found in the same transcript in nature, or are not found in the same transcript as the splice sites comprised by the EML in nature, and/or are not operably linked to the cargo polypeptide in nature which is operably linked to the EML tag. As is common practice and is understood by those in the art, progeny and copies of an engineered polynucleotide are typically still referred to as “engineered” even though the actual manipulation was performed on a prior entity.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); and Current Protocols in Protein Science (CPPS) (John E. Coligan, et. al., ed., John Wiley and Sons, Inc.), which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. An engineered multiple localization tag comprising a nucleic acid sequence encoding at least two localization signal sequences;
   wherein each of the localization signal sequences will direct localization of a polypeptide encoded by an operably linked sequence to a different set of subcellular compartments.
2. The engineered multiple localization tag of paragraph 1, wherein the localization signal sequences are not separated by an exon.
3. The engineered multiple localization tag of paragraph 1, wherein the localization signal sequence are separated by an exon of no more than 300 bases.
4. The engineered multiple localization tag of paragraph 3, wherein the exon comprises glycine and serine residues.
5. The engineered multiple localization tag of any of paragraphs 1-4, further comprising a set of compatible splicing sequences;
   wherein the set comprises two alternative splice donor sequences and one splice acceptor sequence;
   wherein the two alternative splice donor sequences flank one localization signal sequence; and
   the splice acceptor sequence is located 3′ of both splice donor sequences of the set.
6. The engineered multiple localization tag of paragraph 5, wherein the set of splicing sequences is located 5′ of a second localization signal.
7. The engineered multiple localization tag of paragraph 5, wherein the set of splicing sequences is located 3′ of a second localization signal.
8. The engineered multiple localization tag of any of paragraphs 1-7, further comprising a set of compatible splicing sequences;
   wherein the set comprises two alternative splice acceptor sequences and one splice donor sequence;
   wherein the two alternative splice acceptor sequences flank a localization signal sequence; and the splice donor sequence is located 5′ of both splice acceptor sequences of the set.
9. The engineered multiple localization tag of paragraph 8, wherein the set of splicing sequences is located 3′ of a second localization signal.
10. The engineered multiple localization tag of paragraph 8, wherein the set of splicing sequences is located 5′ of a second localization signal.
11. The engineered multiple localization tag of any of paragraphs 5-10, wherein a pair of alternative splice sites comprises a weak and a strong splice site.
12. The engineered multiple localization tag of paragraph 11, wherein the weak splice site is located 5′ of the flanked localization signal and the strong splice site is located 3′ of the flanked localization signal.
13. The engineered multiple localization tag of any of paragraphs 11-12, wherein a set of compatible splicing sites comprises the weak splice donor site of SEQ ID NO: 8; the strong splice donor site of SEQ ID NO: 9, and the splice acceptor site of SEQ ID NO: 10.
14. The engineered multiple localization tag of any of paragraphs 11-12, wherein a set of compatible splicing sites comprises the splice donor site of SEQ ID NO: 11, the weak splice acceptor site of SEQ ID NO: 12; and the strong splice acceptor site of SEQ ID NO: 13.
15. The engineered multiple localization tag of any of paragraphs 1-14, wherein each of the localization signals is selected from the group consisting of:
   a chloroplast localization signal; a peroxisome localization signal; a mitochondrion localization signal; a secretory pathway localization signal; an endoplasmic reticulum localization signal; and a vacuole secretion localization signal.
16. The engineered multiple localization tag of paragraph 15, wherein the chloroplast localization signal comprises a nucleic acid sequence encoding CTPa (SEQ ID NO:1) or a polypeptide having at least 90% identity to CTPa.
17. The engineered multiple localization tag of paragraph 16, wherein the chloroplast localization signal comprises the nucleic acid sequence of SEQ ID NO:14 or a sequence having at least 90% identity to SEQ ID NO:14.
18. The engineered multiple localization tag of paragraph 15, wherein the chloroplast localization signal comprises a nucleic acid sequence encoding CTPb (SEQ ID NO:6) or a polypeptide having at least 90% identity to CTPb.
19. The engineered multiple localization tag of paragraph 18, wherein the chloroplast localization signal comprises the nucleic acid sequence of SEQ ID NO:15 or a sequence having at least 90% identity to SEQ ID NO:15.
20. The engineered multiple localization tag of paragraph 15, wherein the peroxisome localization signal comprises a nucleic acid sequence encoding PTS2 (SEQ ID NO:2) or a polypeptide having at least 90% identity to PTS2.
21. The engineered multiple localization tag of paragraph 20, wherein the peroxisome localization signal comprises the nucleic acid sequence of SEQ ID NO:16 or a polypeptide having at least 90% identity to SEQ ID NO:16.
22. The engineered multiple localization tag of paragraph 15, wherein the peroxisome localization signal comprises SEQ ID NO: 5.
23. The engineered multiple localization tag of paragraph 23, wherein the peroxisome localization signal comprises the nucleic acid sequence of SEQ ID NO:17 or a sequence having at least 90% identity to SEQ ID NO:17.
24. The engineered multiple localization tag of any of paragraphs 1-23, comprising the nucleic acid sequence encoding a polypeptide of any of SEQ ID NOs:3 and 21-23 or a polypeptide having at least 90% identity to any of SEQ ID NOs:3 and 21-23.
25. The engineered multiple localization tag of paragraph 24, comprising the nucleic acid sequence of SEQ ID NO:18 or a sequence having at least 90% identity to SEQ ID NO:18.
26. The engineered multiple localization tag of any of paragraphs 1-23, comprising the sequence of any of SEQ ID NOs:4 and 24-26 or a sequence having at least 90% identity to any of SEQ ID NOs:4 and 24-26.
27. The engineered multiple localization tag of paragraph 26, comprising the nucleic acid sequence of SEQ ID NO:19 or a sequence having at least 90% identity to SEQ ID NO:19.
28. The engineered multiple localization tag of any of paragraphs 1-23, wherein a first localization signal is comprised within a second localization signal.
29. The engineered multiple localization tag of paragraph 28, wherein the first localization signal is substituted for the amino acids equivalent to residues 37 to 46 of SEQ ID NO: 6.
30. The engineered multiple localization tag of paragraph 29, comprising the sequence of SEQ ID NO:7 or a sequence having at least 90% identity to SEQ ID NO:7.
31. The engineered multiple localization tag of paragraph 30, comprising the nucleic acid sequence of SEQ ID NO:20 or a sequence having at least 90% identity to SEQ ID NO:20.
32. A vector comprising the engineered multiple localization tag of any of paragraphs 1-31.
33. The vector of paragraph 32, wherein the entirety of the engineered multiple localization tag is located on one flank of a cloning site or an operably linked sequence encoding a peptide.
34. The vector of paragraph 33, wherein the engineered multiple localization tag is located 5′ of an operably linked sequence encoding a polypeptide.
35. An engineered cell or organism comprising the engineered multiple localization tag of any of paragraphs 1-31, or the vector of any of paragraphs 32-34.
36. A nucleic acid molecule having the sequence of, or encoding the polypeptide having the sequence of, any of SEQ ID NO: 28-87, or a sequence having at least 90% identity thereto.
37. A vector comprising the nucleic acid molecule of paragraph 36.
38. An engineered cell or organism comprising the nucleic acid molecule of paragraph 36 or the vector of paragraph 37.

### EXAMPLES

### Example 1

### Multicompartment Protein Targeting in Plants Via Engineered Alternative Splicing and Embedded Signals

Plant bioengineers require simple genetic devices for predictable localization of heterologous proteins to multiple subcellular locations.

Described in this example are novel hybrid signal sequences for multiple-compartment localization and the characterization of their function when fused to GFP in Nicotiana benthamiana leaf tissue. TriTag-1 and TriTag-2 use alternative splicing to generate differentially localized GFP isoforms, localizing it to the chloroplasts, peroxisomes and cytosol. TriTag-1 shows a bias for targeting the chloroplast envelope while TriTag-2 preferentially targets the peroxisomes. TriTag-3 embeds a conserved peroxisomal targeting signal within a chloroplast transit peptide, directing GFP to the chloroplasts and peroxisomes.

The signal sequences described herein can reduce the amount of cloning and the size of DNA constructs required to target a heterologous protein to multiple locations in, e.g. plant tissue. This work harnesses alternative splicing and signal embedding for engineering plants with multi-functional proteins from single genetic constructs.

List of abbreviations. PTS2, peroxisome targeting signal 2. TTL, Arabidopsis transthyretin-like S-allantoin synthase gene. CTP, chloroplast targeting peptide. PIMT2, Arabidopsis protein-L-isoaspartate methyltransferase gene. CTPa, chloroplast targeting peptide from PIMT2. rbcS1, Solanum tuberosum ribulose-1,5-biphosphate carboxylase (RuBisCO) small-subunit gene. CTPb, chloroplast targeting peptide from rbcS1. smGFP, soluble modified green fluorescent protein.

### Background

Plant cells harbor many distinct compartments that share some overlapping function, or are functionally associated in metabolic pathways and development. To permit complex metabolic engineering, plant engineers require tools to direct single transgenes to multiple compartments. For example, re-engineering photorespiration (Kebeish et al., 2007; Maier et al., 2012) and isoprenoid synthesis (Kumar et al., 2012; Sapir-Mir et al., 2008) will involve both the chloroplasts and peroxisomes. A number of synthetic N-terminal and C-terminal extensions are readily available to target heterologous proteins to desired subcellular compartments, such as the chloroplast, peroxisome, mitochondrion, endoplasmic reticulum or the nucleus. Issues around protein targeting have arisen in (1) studying protein function in a coordinated fashion (Hooks et al., 2012; Zhang & Hu, 2010), (2) improving holistic plant metabolic engineering efforts (Baudisch & Klosgen, 2012; Brandão & Silva-Filho, 2011; Severing et al., 2011) and (3) increasing yields attained by molecular farming and other protein factory applications (Hyunjong et al., 2006).

One approach to target proteins to more than one location involves cloning multiple genetic copies, each containing a different localization peptide. Each copy must be introduced by successive retransformation, or alternatively, by backcrossing single transforms (Que et al., 2010). These procedures are time-intensive and yield transformants with multiple spatially distinct copies of a protein expression cassette. Coordinate expression may not be ensured due to context-dependent regulatory effects and/or homology-based silencing (Dafny-Yelin & Tzfira, 2007). Although dual targeting to certain organelles may instead be achieved by adding a second localization peptide (Hyunjong et al., 2006), this approach is limited to the possible combinations that can be made from available N- and C-terminal extensions.

Herein is described a simple technique for targeting of transgenic proteins to multiple organelles, specifically the chloroplast, peroxisome, and cytosol. This combination of organelles is particularly interesting due to their close functional association in photorespiration, isoprenoid biosynthesis, β-oxidation and other metabolic processes (Baker et al., 2006; Peterhansel et al., 2010; Sapir-Mir et al., 2008).

### Results

Design for Multiple-Compartment Localization by Alternative Splicing: TriTag-1 and TriTag-2.

To construct TriTag-1 and TriTag-2, a chloroplast-targeting region (CTPa) was taken from protein-L-isoaspartate methyltransferase (PIMT2, At5g50240). PIMT2 is a ubiquitous repair protein, converting exposed isoaspartate residues to aspartate or asparagine residues in aging polypeptides (Dinkins et al., 2008; Lowenson & Clarke, 1992). Various mRNAs produced from PIMT2 are produced by alternative transcription initiation sites and alternative splicing events (Dinkins et al., 2008). The spliceforms produced from the 3′ transcription initiation site target the protein to the chloroplast when the targeting sequence is retained, and to the cytosol when it is not.

A peroxisome targeting sequence, PTS2, containing the RLx5HL nonapeptide (Lanyon-Hogg et al., 2010), was taken from the transthyretin-like S-allantoin synthase gene (TTL; At5g58220). This synthase catalyzes two steps in the allantoin biosynthesis pathway (Reumann et al., 2007). At least two spliceforms are produced from TTL from internal alternative acceptor junctions. The translated proteins are targeted to the peroxisome if they retain the internal PTS2 site and to the cytosol if the site is removed (Reumann et al., 2007).

Harnessing the sequences attained from the above genes, two novel 5′ protein tags (TriTag-1 and TriTag-2) that targeted GFP to chloroplast, peroxisome and/or cytosol using alternative splicing were designed (FIGS. 1A-1D). TriTag-1 contains the elements in this order: a short sequence of PIMT2 containing the start codon, two alternative donor sites flanking CTPa, a single acceptor site, a short exon that encodes glycine and serine residues, a single donor site, and two alternative acceptor sites flanking the PTS2 of the TTL gene (FIGS. 1A, 1C). In TriTag-2 the positions of the sequences taken from genes PIMT2 and TTL are reversed (FIGS. 1B, 1D).

Both tags are designed so that the two alternative splicing events occur independently of each other. As a result, mRNAs encoding cytoplasmic, peroxisomal, and cytoplasmically localized proteins are expected.

Design for Dual-Targeting by Signal Embedding: TriTag-3.

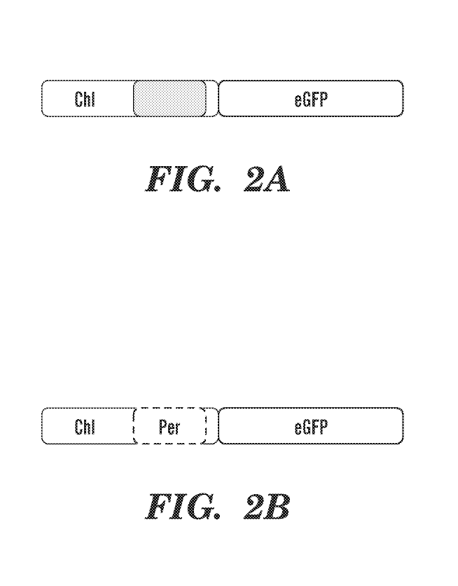

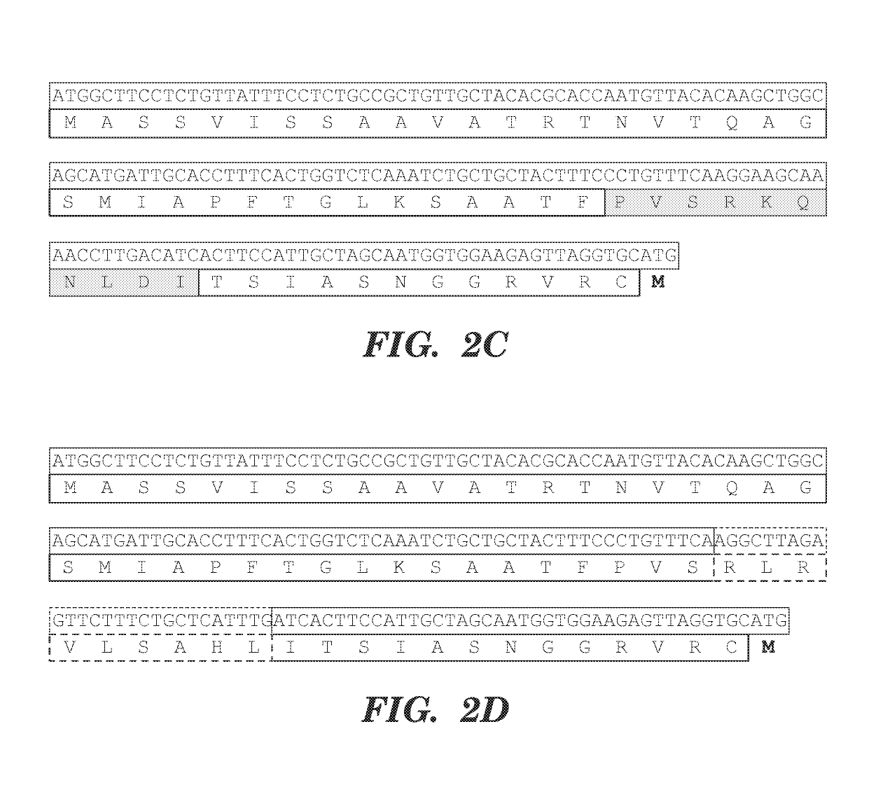

For targeting to two intracellular locations with a single N-terminal extension, a peroxisome targeting sequence was embedded within a chloroplast targeting sequence (TriTag-3, FIGS. 2B, 2D). The PTS2 RLx5HL nonapeptide was placed within the chloroplast targeting region from the ribulose-1,5-biphosphate carboxylase (RuBisCO) small-subunit rbcS1 (CTPb, FIG. 2a,c, GenBank: X69759.1) (Fritz et al., 1991), substituting for a poorly conserved segment in the CTP that is predicted to form an unfolded segment (determined by PROFbval on the ROSTLAB server (Schlessinger et al., 2006)). Specifically, the amino acids closest to the N-terminus of the protein are the most effective at differentiating between targeting to the chloroplast and the mitochondria.

Inspection of the A. thaliana chloroplast-targeted proteins revealed a decrease in conservation of CTPs toward the C-terminus (Bhushan et al., 2006; Sadler et al., 1989). Based on these findings, PTS2 was embedded at the 40th amino acid. The resulting targeting peptide, TriTag-3, retains a predicted structure similar to the native CTPb in terms of flexibility. It was determined that proteins containing the N-terminal TriTag-3 extension would be targeted to the peroxisomes and chloroplasts using TargetP (Emanuelsson et al., 2007) and PeroxisomeDB 2.0 (Schluter et al., 2009).

Subcellular Localization of GFP Controls in Transient Assays.

The targeting properties of the TriTag-GFP fusions were tested in Nicotiana benthamiana leaf epidermal cells using biolistic particle delivery (Bio-Rad Helios Gene Gun) for transient expression. Transient expression is useful for studying alternative splicing in vivo (Reddy et al., 2012; Stauffer et al., 2010). Expression was controlled by the constitutive promoter PENTCUP2 and the nopaline synthetase (NOS) termination signal (Coutu et al., 2007). Images were taken by confocal microscopy (Leica SP5 X MP, Buffalo Grove, Ill. 60089 United States) 48-96 hours after particle delivery (data not shown). The subcellular fluorescent localization patterns in transfected leaf tissue were compared to chlorophyll autofluorescence; untagged GFP localized to the cytosol and nucleus (data not shown; see also (Li et al., 2010)); GFP fused to the native chloroplast targeting peptide of the Solanum tuberosum potato RuBisCO protein rbcS1 (Kebeish et al., 2007) (data not shown); and the peroxisomal-targeted GFP in delivered via baculovirus (BacMam 2.0 CellLight Peroxisome-GFP, Cat No. C10604, Life Technologies, Carlsbad, Calif.; data not shown).

Subcellular Localization of TriTag-1 and TriTag-2 Fused GFP.

TriTag-1 and TriTag-2 showed localization to the cytoplasm plus nucleus, chloroplast, and peroxisome (data not shown). Transient expression of TriTag-1-GFP resulted in cytosolic and chloroplast localization, with the latter inferred by chlorophyll co-localization in the transfected cell. Additional punctate staining was observed that did not correspond to chloroplasts, but was similar to the staining observed with the peroxisomal-targeted BacMam vector (data not shown) and was attributed to peroxisomal targeting. Transiently expressed TriTag-2-GFP (data not shown) display cytosolic plus nuclear localization, as well as a bright punctate pattern indicating a high level of peroxisomal targeting and a lower signal in the chloroplasts. Overall, TriTag-1 localized GFP preferentially to the chloroplasts, while TriTag-2 localized this protein to the peroxisomes, with similar targeting to the cytoplasm plus nucleus.

Subcellular Localization of TriTag-3 Fused to GFP.

N. benthamiana epidermal leaf cells transiently expressing TriTag-3-GFP display chloroplast localization and punctate peroxisomal localization (data not shown). Essentially no GFP was observed in the cytosol. This observation indicates that the hybrid chloroplast/peroxisome targeting sequence is efficiently recognized by the corresponding localization systems, and also that the cytoplasmic plus nuclear localization observed with TriTags 1 and 2 is likely due to mRNAs spliced so that they lack both the peroxisomal and chloroplast targeting sequences.

### Discussion

Described herein are strategies for localizing a single transgenic protein to multiple cellular compartments, e.g. in plants. Variation in N-terminal targeting sequences was encoded by alternative splicing, which greatly economized on the amount of DNA transfected. In addition, dual targeting was achieved by an ambiguous N-terminal signal with elements of chloroplast and peroxisomal targeting sequences. Three different examples of short, N-terminal elements were designed for coordinate chloroplast, peroxisome and cytosol targeting, termed ‘TriTags”. TriTag-1 and TriTag-2 (FIGS. 1A-1D) were designed by combining DNAs encoding alternatively spliced mRNAs that direct the encoded proteins to either the chloroplast plus cytoplasm (Dinkins et al., 2008) or the peroxisome plus cytoplasm (Reumann et al., 2007). TriTag-3 (FIG. 2A-2D) does not rely on alternative splicing and consists of a chloroplast targeting sequence in which a naturally unstructured portion has been replaced with a peroxisomal targeting sequence (Silva-Filho, 2003).

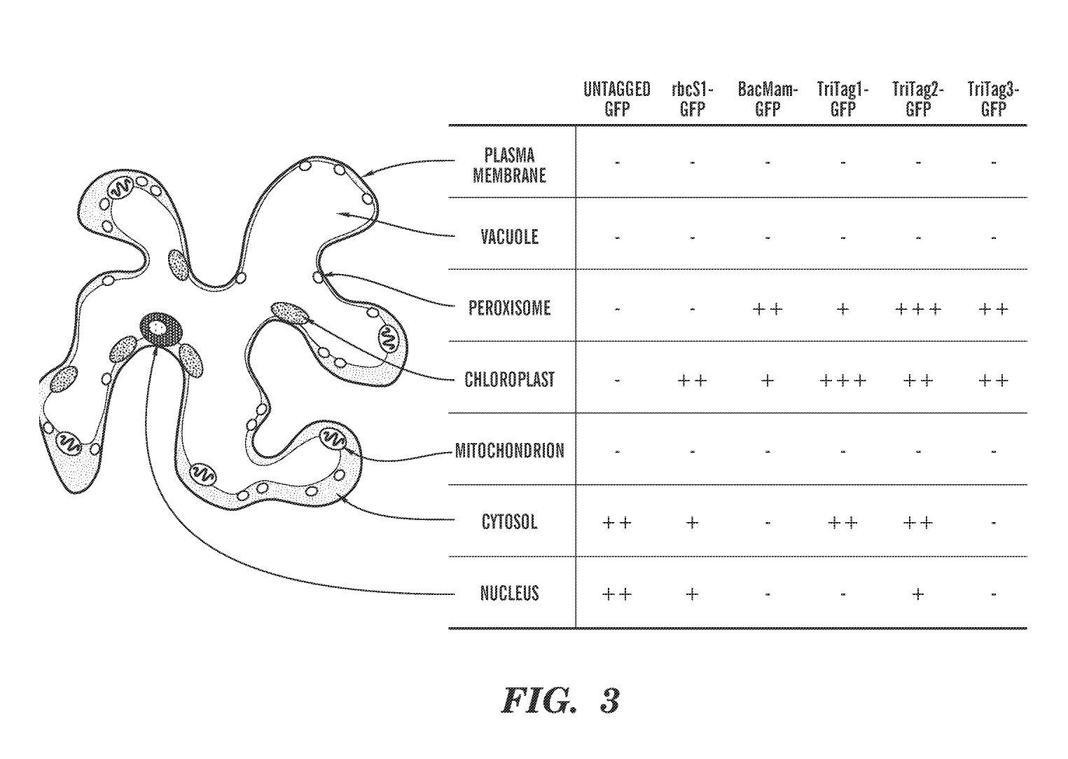

The TriTags function in vivo to target GFP in Nicotiana benthamiana leaf epidermal cells (FIG. 3). Confocal images of the TriTags were compared to controls of untagged GFP, a Rubisco-derived localization signal for the chloroplast, and a baculovirus system that targets to the peroxisome. Plasmid DNA was delivered into leaf cells by standard biolistic transfection. Untagged GFP was localized to the cytoplasm and nucleus, with some nuclear localization being expected because the nuclear pore has a large, aqueous channel that permits entry of molecules up to about 70 kD. TriTag-1 and TriTag-2 mediated GFP expression in the chloroplast, peroxisome, and cytoplasm (plus nucleus), with TriTag-1 showing a slight preference for the chloroplast over peroxisome and TriTag-2 showing the opposite behavior. TriTag-3 mediated strong localization to both the peroxisome and chloroplast, but not detectably to the cytoplasm. These behaviors suggest that the three alternatively spliced mRNA forms are all being produced (FIG. 3).

The re-engineering of photorespiration pathways (Kebeish et al., 2007) illustrates the potential utility of such multiple-targeting elements. Normally during photorespiration, glycolate is generated in the chloroplast and then transported into the cytoplasm and then into the peroxisome, where it is oxidized to glyoxylate in an O₂-dependent reaction. The reduction of oxygen, rather than NAD(P)+ as an oxidizing agent represents a waste of reducing equivalents and energy. Kebeish et al. engineered plants to express in the chloroplast an NAD+-dependent bacterial glycolate metabolizing pathway and found this enhanced the growth of light-limited Arabidopsis. In this situation, the added bacterial pathway competes with transport of glycolate from the chloroplast into the cytoplasm. Expression of the pathway in the cytoplasm and peroxisome could further enhance the amount of glycolate that is metabolized by this more efficient pathway.

The results described herein also indicate that alternative splicing systems can be engineered in a straightforward manner.

Plant metabolic engineering remains a formidable effort in terms of time and resources. The field requires simple and efficient technologies for transforming plants with multi-functional proteins. It is demonstrated herein that alternative splicing can be engineered to target a single transgene to multiple locations, in this instance, the chloroplast, cytosol, and peroxisome. In addition, it was demonstrated that a peroxisomal signal embedded within a chloroplast signal allows dual targeting of the transgene. These devices can reduce time and resources spent on plant metabolic engineering.

### Methods

Strains and Plasmids.

E. coli K12 strains (NEB Turbo, New England Biolabs) were used as plasmid hosts for cloning work on binary vectors for transient expression and/or stable genomic integration. Plasmids (Table 1), were constructed with traditional cloning methods (Sambrook & Russell, 2001), BglBricks (Anderson et al., 2010), BioBricks (Knight, 2003), or Gibson assembly (Gibson, 2011). E. coli K12 cells were grown in Luria-Bertani medium with appropriate antibiotics (100 μg/mL Kanamycin).

TriTag Synthesis and Cloning.

TriTag-1, TriTag-2 and TriTag-3 were synthesized (GeneBlocks, IDT, Coralville, Iowa), and cloned in-frame 5′ of the soluble modified GFP (smGFP) using Gibson assembly. This modified GFP contains three site-directed mutations that increase the protein's solubility and fluorescence intensity (Davis & Vierstra, 1998). Based on splice site prediction with NetPlantGene (Hebsgaard et al., 1996), the inventors predicted that the processed spliceforms of TriTag-1 and TriTag 2 encodes for GFP variants containing regions for chloroplast targeting, peroxisomal targeting or neither. Spliceforms other than those found using NetPlantGene would either incorporate a stop codon or lack organelle-targeting information, causing premature translation or sole targeting to the cytosol, respectively.

Plant Material.

All plants were incubated at 16-20° C. in a 16/8 hour light/dark cycle and watered twice weekly. Peat-based soil-free media (Metromix, SunGro Horticulture, Vancouver, Canada) was autoclaved 45 min before use. Leaves from 3-5 month old Nicotiana benthamiana seedling plants were collected for bombardment.

Biolistic Delivery.

DNA-gold particle complexes for biolistic delivery were prepared according to manufacturer's instructions for use with the Helios Gene Gun (Bio-Rad, Hercules, Calif.) as follows: Plasmid DNA (50 μg) containing the tagged GFP gene was pelleted onto 1 μm gold particles (6-8 mg) in a spermidine (100 μL, 0.05M) and CaCl2 (100 μL, 1.0 M) mixture and resuspended in a polyvinylpyrrolidone/EtOH solution (5.7 mg/mL). The resulting suspension was deposited onto the inside surface area of Tygon plastic tubing (o.d.=2 mm) and diced into cartridges facilitated by the Tubing Prep Station (Bio-Rad, Hercules, Calif.). Cartridges were stable up to 6 months dessicated at 4° C. The underside of Nicotiana benthamiana leaves were transformed biolistically using the Helios Gene Gun (Bio-Rad, Hercules, Calif.) at 150-250 psi He (Woods & Zito, 2008). The leaves were placed on wet filter paper in Petri dishes and stored on a bench-top under ambient lighting and room temperature for 48 hours before imaging analysis.

Target Control Proteins.

As expected, control proteins showed untagged smGFP was distributed extensively in the cytosol and nucleus (data not shown), but not the vacuole, which makes up the bulk of the plant cell volume. This localization pattern matches previous untagged GFP localization studies (Li et al., 2010). Cytosolic and chloroplast localization controls were determined by transient expression of GFP fused to the native chloroplast targeting peptide of the Solanum tuberosum potato RuBisCO protein rbcS1 (Kebeish et al., 2007) (data not shown).

BacMam Staining.

A solution of 24 μL BacMam peroxisomal dye (BacMam 2.0 CellLight Peroxisome-GFP, Cat. No. C10604, Life Technologies, Carlsbad, Calif.) in 2.5 mL 0.1% Triton X-100 was prepared along with similar solutions of the BacMam transduction control dye (Cat. No. B10383) and no-dye controls. Three-millimeter slices of N. benthamiana leaves were incubated in the solutions overnight and imaged by confocal microscopy. Although the BacMam 2.0 (Life Technologies) baculovirus peroxisomal GFP dye was designed for mammalian cells, its use in plant tissues has also been demonstrated (Takemoto et al., 2003). Images of N. benthamiana leaf tissue with transfected BacMam were representative of the distribution, size and shape of the peroxisomes (data not shown).

Prediction Software.

Splice junctions within the TriTag-1 and TriTag-2 sequences were predicted using the NetPlantGene server (Hebsgaard et al., 1996). Targeting to the chloroplast and peroxisome of the TriTag-1 and TriTag-2 splice variants and TriTag-3 were predicted using TargetP (Emanuelsson et al., 2007) and PeroxisomeDB 2.0 (Schluter et al., 2009). Peptide structures of CTPb and TriTag-3 were determined using PROFbval on the ROSTLAB server (Schlessinger et al., 2006).

Imaging and Processing.

Bombarded leaves were diced and placed on glass slides in 0.1% Triton-X100 and imaged by fluorescence confocal microscopy (excitation at 489 nm, detection at 500-569 for GFP and 630-700 for chlorophyll autofluorescence) using a 40× water-based objective (numerical aperture 1.10).

### REFERENCES

Anderson, J. C., Dueber, J. E., Leguia, M., Wu, G. C., Goler, J. A., Arkin, A. P. & Keasling, J. D. (2010). BglBricks: A flexible standard for biological part assembly. Journal of biological engineering 4, 1. Department of Bioengineering, University of California, Berkeley, Calif. 94720, USA.
Baker, A., Graham, I. a, Holdsworth, M., Smith, S. M. & Theodoulou, F. L. (2006). Chewing the fat: beta-oxidation in signalling and development. Trends in plant science 11, 124-32.
Baudisch, B. & Klosgen, R. B. (2012). Dual targeting of a processing peptidase into both endosymbiotic organelles mediated by a transport signal of unusual architecture. Molecular plant 5, 494-503.
Bhushan, S., Kuhn, C., Berglund, A.-K., Roth, C. & Glaser, E. (2006). The role of the N-terminal domain of chloroplast targeting peptides in organellar protein import and miss-sorting. FEBS letters 580, 3966-72.
Brandão, M. M. & Silva-Filho, M. C. (2011). Evolutionary history of Arabidopsis thaliana aminoacyl-tRNA synthetase dual-targeted proteins. Molecular biology and evolution 28, 79-85. Coutu, C.,
Brandle, J., Brown, D., Brown, K., Miki, B., Simmonds, J. & Hegedus, D. D. (2007). pORE: a modular binary vector series suited for both monocot and dicot plant transformation. Transgenic research 16, 771-781.
Dafny-Yelin, M. & Tzfira, T. (2007). Delivery of multiple transgenes to plant cells. Plant physiology 145, 1118-28.
Davis, S. J. & Vierstra, R. D. (1998). Soluble, highly fluorescent variants of green fluorescent protein (GFP) for use in higher plants. Plant molecular biology 36, 521-8.
Dinkins, R. D., Majee, S. M., Nayak, N. R., Martin, D., Xu, Q., Belcastro, M. P., Houtz, R. L., Beach, C. M. & Downie, A. B. (2008). Changing transcriptional initiation sites and alternative 5′- and 3′-splice site selection of the first intron deploys Arabidopsis protein isoaspartyl methyltransferase2 variants to different subcellular compartments. The Plant journal: for cell and molecular biology 55, 1-13.
Emanuelsson, O., Brunak, S., Von Heijne, G. & Nielsen, H. (2007). Locating proteins in the cell using TargetP, SignalP and related tools. Nature protocols 2, 953-71.
Fritz, C. C., Herget, T., Wolter, F. P., Schell, J. & Schreier, P. H. (1991). Reduced steady-state levels of rbcS mRNA in plants kept in the dark are due to differential degradation. Proceedings of the National Academy of Sciences of the United States of America 88, 4458-62.
Gibson, D. G. (2011). Enzymatic assembly of overlapping DNA fragments. Methods in enzymology 498, 349-61.
Hebsgaard, S. M., Korning, P. G., Tolstrup, N., Engelbrecht, J., Rouzé, P. & Brunak, S. (1996). Splice site prediction in Arabidopsis thaliana pre-mRNA by combining local and global sequence information. Nucleic acids research 24, 3439-52.
Hooks, K. B., Turner, J. E., Graham, I. a, Runions, J. & Hooks, M. a. (2012). GFP-tagging of Arabidopsis acyl-activating enzymes raises the issue of peroxisome-chloroplast import competition versus dual localization. Journal of plant physiology 169, 1631-8.
Hyunjong, B., Lee, D.-S. & Hwang, I. (2006). Dual targeting of xylanase to chloroplasts and peroxisomes as a means to increase protein accumulation in plant cells. Journal of experimental botany 57, 161-9.
Kebeish, R., Niessen, M., Thiruveedhi, K., Bari, R., Hirsch, H.-J. J., Rosenkranz, R., Stabler, N., Schönfeld, B., Kreuzaler, F. & Peterhänsel, C. (2007). Chloroplastic photorespiratory bypass increases photosynthesis and biomass production in Arabidopsis thaliana. Nature biotechnology 25, 593-9.
Knight, T. (2003). Idempotent Vector Design for Standard Assembly of Biobricks. MIT Artificial Intelligence Laboratory; MIT Synthetic Biology Working Group 1-11.
Kumar, S., Hahn, F. M., Baidoo, E., Kahlon, T. S., Wood, D. F., McMahan, C. M., Cornish, K., Keasling, J. D., Daniell, H. & Whalen, M. C. (2012). Remodeling the isoprenoid pathway in tobacco by expressing the cytoplasmic mevalonate pathway in chloroplasts. Metabolic engineering 14, 19-28.
Lanyon-Hogg, T., Warriner, S. L. & Baker, A. (2010). Getting a camel through the eye of a needle: the import of folded proteins by peroxisomes. Biology of the cell/under the auspices of the European Cell Biology Organization 102, 245-63.
Li, F., Liu, W., Tang, J., Chen, J., Tong, H., Hu, B., Li, C., Fang, J., Chen, M. & Chu, C. (2010). Rice DENSE AND ERECT PANICLE 2 is essential for determining panicle outgrowth and elongation. Cell research 20, 838-849.
Lowenson, J. D. & Clarke, S. (1992). Recognition of D-aspartyl residues in polypeptides by the erythrocyte L-isoaspartyl/D-aspartyl protein methyltransferase. Implications for the repair hypothesis. The Journal of biological chemistry 267, 5985-95.
Maier, A., Fahnenstich, H., Von Caemmerer, S., Engqvist, M. K. M., Weber, A. P. M., Flũgge, U.-I. & Maurino, V. G. (2012). Transgenic Introduction of a Glycolate Oxidative Cycle into A. thaliana Chloroplasts Leads to Growth Improvement. Frontiers in plant science 3, 38.
Peterhansel, C., Horst, I., Niessen, M., Blume, C., Kebeish, R., Kürkcüoglu, S. & Kreuzaler, F. (2010). Photorespiration. In The Arabidopsis book, p. e0130. American Society of Plant Biologists.
Que, Q., Chilton, M.-D. M., De Fontes, C. M., He, C., Nuccio, M., Zhu, T., Wu, Y., Chen, J. S. & Shi, L. (2010). Trait stacking in transgenic crops: challenges and opportunities. GM crops 1, 220-9.
Reddy, A. S. N., Rogers, M. F., Richardson, D. N., Hamilton, M. & Ben-Hur, A. (2012). Deciphering the plant splicing code: experimental and computational approaches for predicting alternative splicing and splicing regulatory elements. Frontiers in plant science 3, 18.
Reumann, S., Babujee, L., Ma, C., Wienkoop, S., Siemsen, T., Antonicelli, G. E., Rasche, N., Lüder, F., Weckwerth, W. & Jahn, O. (2007). Proteome analysis of Arabidopsis leaf peroxisomes reveals novel targeting peptides, metabolic pathways, and defense mechanisms. The Plant cell 19, 3170-3193.
Sadler, I., Chiang, A., Kurihara, T., Rothblatt, J., Way, J. & Silver, P. (1989). A yeast gene important for protein assembly into the endoplasmic reticulum and the nucleus has homology to DnaJ, an Escherichia coli heat shock protein. The Journal of cell biology 109, 2665-75.
Sambrook, J. & Russell, D. W. (2001). Molecular Cloning: A Laboratory Manual. Cold Spring Harbor, N.Y.: Cold Spring Harbor Laboratory Press, 3rd edn. (J. Sambrook & D. W. Russell, Eds.). Cold Spring Harbor, N.Y.: Cold Spring Harbor Laboratory Press.
Sapir-Mir, M., Mett, A., Belausov, E., Tal-Meshulam, S., Frydman, A., Gidoni, D. & Eyal, Y. (2008). Peroxisomal localization of Arabidopsis isopentenyl diphosphate isomerases suggests that part of the plant isoprenoid mevalonic acid pathway is compartmentalized to peroxisomes. Plant physiology 148, 1219-28.
Schlessinger, A., Yachdav, G. & Rost, B. (2006). PROFbval: predict flexible and rigid residues in proteins. Bioinformatics (Oxford, England) 22, 891-3.
Schluter, a., Real-Chicharro, A., Gabaldon, T., Sanchez-Jimenez, F. & Pujol, A. (2009). PeroxisomeDB 2.0: an integrative view of the global peroxisomal metabolome. Nucleic Acids Research 38, D800-D805.
Severing, E. I., Van Dijk, A. D. & Van Ham, R. C. (2011). Assessing the contribution of alternative splicing to proteome diversity in Arabidopsis thaliana using proteomics data. BMC plant biology 11, 82. Applied Bioinformatics, Plant Research International, PO Box 619, 6700 AP Wageningen, The Netherlands.
Stauffer, E., Westermann, A., Wagner, G. & Wachter, A. (2010). Polypyrimidine tract-binding protein homologues from Arabidopsis underlie regulatory circuits based on alternative splicing and downstream control. The Plant journal: for cell and molecular biology 64, 243-55.
Takemoto, D., Jones, D. A. & Hardham, A. R. (2003). GFP-tagging of cell components reveals the dynamics of subcellular re-organization in response to infection of Arabidopsis by oomycete pathogens. The Plant journal: for cell and molecular biology 33, 775-92.
Woods, G. & Zito, K. (2008). Preparation of gene gun bullets and biolistic transfection of neurons in slice culture. Journal of visualized experiments: JoVE 10-13.
Zhang, X. & Hu, J. (2010). The Arabidopsis chloroplast division protein DYNAMIN-RELATED PROTEIN5B also mediates peroxisome division. The Plant cell 22, 431-42.

TABLE 1
Plasmids constructed in this study

| | |
|---|---|
| pORE-GFP | pORE binary vector expressing untagged |
| | soluble modified GFP (smGFP) controlled |
| | by the pENTCUP2 promoter (Coutu et al., 2007). |
| pORE-rbcS1-GFP | pORE vector expressing A. thaliana |
| | codon-optimized GFP, fused to the |
| | Solanum tuberosum rbcS1 plastid localization tag |
| | and flanked by nuclear scaffold regions (RB7) |
| | (Kebeish et al., 2007). |
| pORE-TriTag-1-GFP | pORE vector expressing TriTag-1-fused GFP |
| | controlled by the pENTCUP2 promoter. |
| pORE-TriTag-2-GFP | pORE vector expressing TriTag-2-fused GFP |
| | controlled by the pENTCUP2 promoter. |
| pORE-TriTag-3-GFP | pORE vector expressing TriTag-3-fused GFP |
| | controlled by the pENTCUP2 promoter. |

### Example 2

### Multicompartment Targeting in Plants by Alternative Splicing and Embedded Signals to Decrease the Number of Clones Generated

Plant cells contain multiple membrane-bound compartments, including the cytoplasm, nucleus, mitochondrion, chloroplast, and peroxisome, as well as the extracellular space. Some of these compartments are defined by multiple membranes and can further be subdivided into inter-membrane spaces and innermost areas. Targeting of proteins to these different spaces is typically achieved using targeting sequences that are often found at the N-terminus of the protein. These targeting sequences are often proteolytically removed during the localization process.

In some embodiments, the technology described herein relates to targeting proteins to multiple compartments within plant cells. For example, in the course of metabolic engineering, it may be useful to introduce a foreign pathway into multiple compartments, essentially duplicating the pathway. In principle, this could be achieved by placing a specific targeting sequence upstream of a coding sequence, and creating a duplicate construct for each compartment to be targeted.

For example, if it is desirable to express an enzyme in the cytoplasm, chloroplast, and peroxisome, three separate DNA constructs could be generated: one with a gene including a coding sequence for the enzyme, a second construct with the gene preceded by a chloroplast targeting sequence, and a third construct with the gene preceded by a peroxisomal targeting sequence. In practice, such an approach is often not desirable, because it involves the duplication of the coding sequence for the enzyme as well as promoter and 3′ end regions. In situations where introduction of multiple proteins into multiple compartments is desired, such an approach is particularly undesirable because of limits on the size of plasmids that can easily be constructed, limits on the amounts of DNA that can be transferred into plants at one time, and potential recombination between repeated DNA elements that may be present in the plasmids.

One advantage of the technology described herein is that it avoids such duplication. According to the presently described technology, a given protein that is to be expressed through genetic engineering in multiple compartments of plant cells may be so expressed by the introduction of a short DNA element that encodes multiple targeting sequences for different subcellular compartment. These coding sequences may be separated by introns and an alternative splicing system, such that different spliced mRNAs will have a single one of several possible targeting sequences, or no targeting sequence such that the protein is localized to the cytoplasm. Alternatively, a single protein coding sequence can be encoded, such that the multiple targeting sequences are functionally available or a single targeting sequence can be recognized by multiple localization systems. The targeting elements can be placed at the 5′ end, 3′ end, or internal to the coding sequence to be targeted. Internally-placed tags can, e.g. be located within open-coil regions of the host protein, e.g. those regions that are exposed to the surrounding cytosolic or organellar solution.

Expression of Glycolate Dehydrogenase in Multiple Compartments of Arabidopsis Cells.

Photorespiration is a biochemical process of plants that is initiated by the reaction of Rubisco (Ribulose bisphosphate carboxylase/oxygenase) with oxygen instead of carbon dioxide. Specifically, oxygen reacts with ribulose 1,5-bisphosphate to produce 3-phosphoglycerate and 2-phosphoglycolate. The latter compound is not an essential part of metabolism and must be recycled or else the carbon and phosphorus it contains will be lost. The pathway is initiated with the dephosphorylation of phosphoglycolate to glycolate, conversion of glycolate to glyoxylate, and then a complex shuttling of glyoxylate and its metabolites through multiple cellular compartments before returning.

The natural phosphoglycolate recycling pathway is wasteful in that oxygen, rather than NAD or NADP is used as an electron acceptor. Kebeish et al. (Nature Biotech 25[5]:593-9) demonstrated that plants can grow faster if they are engineered to express a bacterial NAD-dependent glycolate dehydrogenase in the chloroplast. It is thought that in non-engineered plants, glycolate is transported from the chloroplast into the cytoplasm, and then from the cytoplasm into the peroxisome, where glycolate oxidase converts glycolate into glyoxylate. One implication of the results of Kebeish et al. is that glyoxylate, when artificially produced in the chloroplast, is likely transported into the peroxisome for further metabolism.

As described herein, in plants engineered to express NAD-dependent glycolate dehydrogenase, the conversion of glycolate to glyoxylate in the chloroplast occurs in competition with transport of glycolate out of the chloroplast. Thus, it is ideal to express glycolate dehydrogenase in the cytoplasm and the peroxisome in addition to the chloroplast.

Expression vectors for expression of E. coli glycolate simultaneously in the chloroplast, cytoplasm and peroxisome were constructed as follows. The multiple targeting sequence TriTag-1, TriTag-2, TriTag-3, shown in FIGS. 1-2 (SEQ ID 18, 19, 20 respectively) was fused to each of the three subunits of E. coli glycolate dehydrogenase (SEQ ID 28, 29, 30, respectively). Each of the resulting genes was placed downstream of the pENTCUP plant promoter and upstream of the nopaline synthetase terminator nosT 3′ end sequence that includes a polyadenylation site. The three constructs were placed together between nuclear scaffold attachment sequences in a single large plasmid that also contained a selectable marker conferring resistance to the herbicide BASTA™.

It should be noted that prior methods would make it necessary to use three copies of each of the three genes encoding subunits of glycolate dehydrogenase.

The BASTA™ resistant Arabidopsis seedlings are primarily plants that are heterozygous for the transfected DNA at one or more loci. Each seedling represents an independent transformation event and presumably an integration at a different chromosomal locus. Because integration at some loci is expected to be deleterious in the heterozygous or homozygous state, many independent Ti plants are self-crossed to obtain T2 lines, ¼ of which are homozygous for the transgene in those cases where the T1 plant contains a single transgene integrated at a non-essential site. Such single-locus homozygous T2 plants are most useful in illustrating the value of the technology described herein and in determining which specific lines have the greatest commercial potential.

The T2 lines are then self-crossed according to standard procedures to produce T3 plants. The T2 plants that are homozygous and have an insertion at a single locus have the following characteristics. First, all of their progeny are resistant to BASTA™ and contain the transgene as determined by Southern blot or PCR. Second, the T1 plant that gave rise to them produced a 1:3 ratio of plants lacking the transgene to plants containing the transgene.

The T3 plants are grown under controlled conditions and compared to each other and to non-engineered Arabidposis. A subset of the engineered plants grow more quickly than wild-type. The enhanced growth rate is particularly pronounced under short-day conditions. In addition, the plants engineered with the construct of the invention, in which glycolate dehydrogenase is expressed in the chloroplast, cytoplasm, and peroxisome, grow more quickly and accumulate more biomass than plants engineered to express glycolate dehydrogenase only in the chloroplast.

Confirmation of the localization of glycolate dehydrogenase to the chloroplast, cytoplasm, and peroxisome is achieved by immunofluorescence staining.

Expression of Glycolate Dehydrogenase in Multiple Compartments of Camelina Sativa Cells.

In a similar set of experiments, other plants such as Camelina sativa, sugar beets, wheat and rice are engineered to express glycolate dehydrogenase. Camelina is considered an excellent crop for production of biofuels, as its seeds are rich in vegetable oil, and it grows in northerly climates such as the Baltic regions, the northern United States and Canada, where growth of other biofuel crops such as sugar cane and corn is not feasible.

For example, Camelina sativa is transformed by the method of Kushvinov (U.S. Pat. No. 7,910,803) or Lu et al. (Plant Cell Rep (2008) 27:273-278). It is noteworthy that Arabidopsis and Camelina are very similar in their genome sequences, and expression plasmids that work in one organism are likely to work in the other.

### Example 3

### Molecular Techniques for Increasing Crop Yield Potential by Enhancing Carbon Fixation and Reducing Photorespiration in C3 Plants

The supply of major food crops is increasingly unable to keep up with rising global food demand. Methods for increasing crop yield potential have mainly focused on conventional breeding with more fit sub-species of crops and/or integration of heterologous proteins conferring abiotic stress resistances to crops. However, studies taking the evolutionary trajectories of C3 plants into account have suggested that substantial gains in yield potential can be obtained by increasing the efficiency of the molecular mechanisms involved in photosynthesis and decreasing photorespiration. This will require the engineering of a multitude of genes in plant cells, and efficient molecular techniques to localize them within the cell to optimize their functionality. Here, methods inspired by the field of synthetic biology are described to face these challenges. In particular, the polycistronic nature of gene expression in chloroplasts is used for plastid-localized multiple bacterial gene expression. Furthermore, the possibility of multiple-compartment targeting from one transgene is addressed by making use of the host's alternative splicing machinery. These techniques further standardize and simplify engineering of plant central metabolism, supporting future endeavors towards greater crop yields.

Introduction

The global population is expected to increase to 9.2 billion by 2050 (Clarke and Daniell 2011). All the while, the agricultural industry is confronted with limited available biotic (i.e. genetic) and abiotic (i.e. land, water, and nutrients) resources. Innovations in the field have mainly helped in addressing factors that contribute to a crop's inability to attain its maximum yield potential. For most mono-cultured cash crops, these yield gaps have been bridged by intensified agronomical practices and conventional crossbreeding. While such methods have been and will be beneficial to future world food security, further improvements would likely prove both knowledge- and labor-intensive for the agricultural workforce. With the vast constraints placed on our agricultural future, more drastic approaches to sustainable agriculture are required, such as engineering the inherent metabolism of crops (von Caemmerer, Quick and Furbank 2012)(Peterhansel, Niessen and Kebeish 2008). The yield ceiling of major crops can be lifted by reengineering C₃-plant carbon fixation and metabolism using methods inspired by synthetic biology (Ducat and Silver 2012).

Until 65 million years ago C₃ photosynthesis had evolved under CO₂ levels higher than those found in the current atmosphere (0.04%). After the Cretaceous-Paleocene extinction event, CO₂ levels fell at a higher rate than the evolutionary response in plants (Zachos, et al. 2001)(Pagani, et al. 2005). Relatively suddenly, the low specificity of RuBisCO—which catalyzes the carbon-fixing step in the Calvin cycle—for CO₂ compared to O₂ became an energetic constraint for plant growth: a significant amount of energy is required to salvage the carbons onto which the O₂ molecule is incorporated—with only 75% carbon-retention efficiency—in a process termed photorespiration.

While most plants responded to the decreased levels of CO₂ by producing vast amounts of RuBisCO (>30% of total plant protein), some plants evolved mechanisms to recover photorespired CO₂ (C₄ photosynthesis which makes up 3% of total plant species, and crassulacean acid metabolism). While C₄ photosynthesis is known to have evolved separately at least 66 times in the past, such an evolutionary trajectory could have only been feasible under high RuBisCO-oxygenase activity (i.e. high O₂, dry and hot climate) as many relatively complex structural changes were required (Sage, Sage and Kocacinar 2012). Taking into account the effect climate change can have on current C₃ cash crops, a metabolic engineering approach to bypass the RuBisCO carbon-fixing step while concurrently minimizing carbon loss by photorespiration was undertaken.

Augmenting C₃ Photosynthesis Using the 3-Hydroxypropionate Cycle.

Photosynthetic microbes, due to their higher growth rate and lack of multicellular constraints, were able to respond to the atmospheric changes in a more elaborate and expansive manner, having created a range of novel carbon fixing pathways such as the reductive citric acid or Arnon-Buchanan cycle (Buchanan and Arnon 1990), the reductive acetyl-CoA or Wood-Ljungdahl pathway (Ljungdahl, Irion and Wood 1965), the dicarboxylate/4-hydroxybutyrate cycle (Huber, et al. 2008), the 3-hydroxypropionate/4-hydroxybutyrate cycle (Berg, et al. 2007), and the 3-hydroxypropionate bicycle (3-HOP)(Zarzycki, Brecht and Müller 2009)(Zarzycki. 2011). With the exception of the 3-HOP pathway, these microbial pathways include oxygen-sensitive enzymes and are thus only functional under anaerobic conditions. Furthermore, the 3-HOP pathway does not use RuBisCO as the initial carbon-fixing step, thus increasing the catalytic rate of fixation without a competing oxygenase reaction.

The engineering of the 3-HOP pathway into C₃-plants was undertaken in the context of lifting yield ceilings of food and cash crops; reducing the abiotic resources required to feed an increasing population and, in the case of cash crops, alleviating competition for arable land. In addition to supplanting the RuBisCO carboxylase reaction, the 3-HOP pathway will be active in shunting photorespiration already occurring in C₃ plants using the citramalyl pathway (see, e.g. the right loop of FIG. 1 of Zarzycki et al. 2009); potentially increasing crop yields further (Zhu, Long and Ort 2010).

The green nonsulfur bacterium Chloroflexus aurantiacus lives commensally in hot springs, and fixes carbon with a unique bicyclic pathway that contains no oxygen-sensitive enzymes (Zarzycki, Brecht and Müller. 2009). It is believed to function primarily as a glycolate/glyoxylate salvage pathway, allowing Chloroflexus to use the glycolate that is excreted by its cyanobacterial neighbors (Zarzycki and Fuchs, 2011). Altogether the pathway is 19 reactions catalyzed by 13 enzymes (Zarzycki et al., 2009; Zarzycki and Fuchs, 2011). Briefly, acetyl-CoA carboxylase (ACC) fixes bicarbonate to acetyl-CoA at the expense of an ATP (reaction 1) releasing malonyl CoA as an intermediate. Malonyl-CoA is converted to 3-hydroxypropionate and then to propionyl-CoA at the cost of 3 NADPH reducing equivalents and 2 ATPs (reactions 2,3). Here the pathway branches. In the first cycle, propionyl-CoA carboxylase (PCC) fixes another bicarbonate, resulting in (S)-methylmalonyl-CoA. In Chloroflexus, an epimerase (reaction 5) converts this intermediate to the (R)-enantiomer, which is converted to succinyl-CoA by the methylmalonyl-CoA mutase (reaction 6). Coenzyme A is removed, and the resulting succinate is converted to malate by the TCA cycle, and then to malyl-CoA (reactions 7-9). Malyl-CoA is split to regenerate acetyl-CoA and a molecule of glyoxylate (reaction 10a). The first three steps of the cycle are repeated, and the glyoxylate is combined with a propionyl-CoA to form β-methylmalyl-CoA (reaction 10b), which is converted through a series of novel rearrangements, to regenerate acetyl-CoA and pyruvate (10c-13). For a single complete turn through the bicycle, a net three bicarbonate ions are fixed using 6 NADPH and 5 ATP.

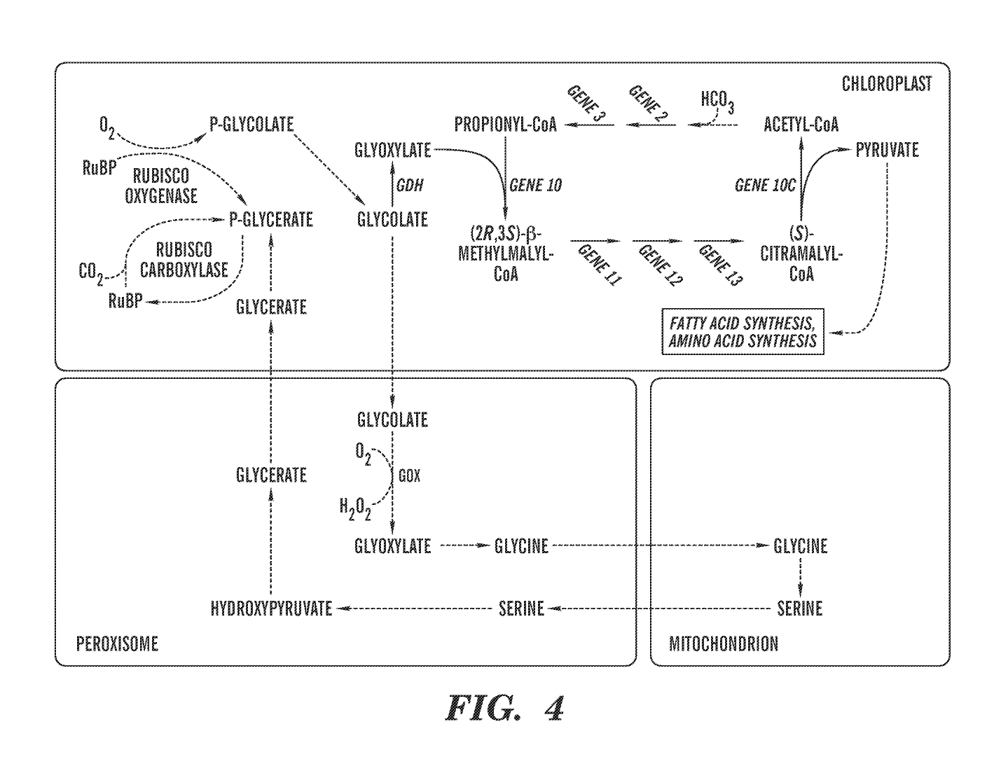

The engineering approach to augmenting C₃ photosynthesis using plastome integration of pathways 1 and 4, is further elucidated below herein. Introducing pathways 1 and 4 alone will constitute a carbon-fixing cycle. This cycle requires glyoxylate as a substrate, a molecule one enzymatic step away from glycolate, the product of the RuBisCO oxygenase reaction (FIG. 4). Introducing glycolate dehydrogenase (GDH) will constitute a full photorespiration bypass.

Shunting Photorespiration by Sub-Cellular Targeting of a Bacterial Glycolate Dehydrogenase.

Recently, Kebeish et al. demonstrated the inefficiency of photorespiration by expressing the three-enzyme glycolate pathway from E. coli into the C₃ model plant Arabidopsis thaliana chloroplasts (Kebeish, et al. 2007). This pathway essentially created a photorespiratory bypass, converting the product from the RuBisCO oxygenase reaction, phosphoglycolate, into phosphoglycerate, an intermediate of the Calvin cycle. Reducing the flux of photorespiratory metabolites through the peroxisomes and mitchondria resulted in a higher growth rate, higher soluble sugar content and a 3-fold increase in shoot and root biomass. Interestingly, enhanced photosynthesis and reduced photorespiration were similarly evident when expressing only the three subunits of the first enzyme in the glycolate pathway -GDH-, however at lower levels.

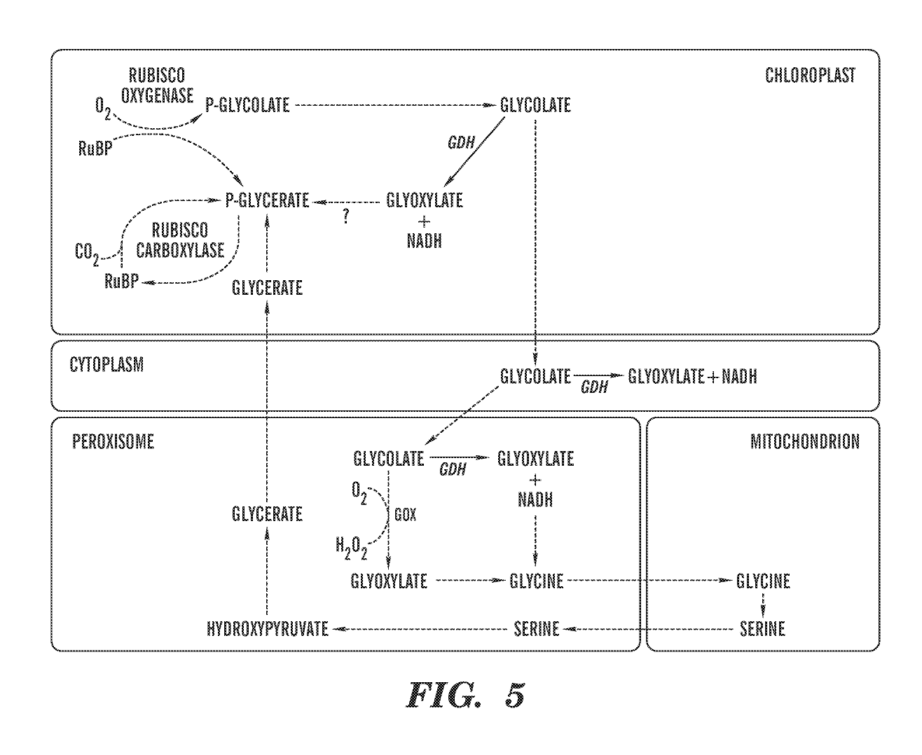

Kebeish and coworkers were able to reduce, but not eliminate photorespiratory glycolate flux to the peroxisomes. First, they sought to engineer transgenic A. thaliana with GDH localized solely to the chloroplasts by plastome integration. The E. coli GDH was added to the integration vector already containing genes of the 3-HOP cycle. By adding GDH, the first step in photorespiration, the conversion of glycolate to glyoxylate is performed within the chloroplast, making the product accessible to the heterogeneously expressed 3-HOP cycle (FIG. 4). Second, taking into account the native flux of glycolate from the chloroplast, through the cytoplasm to the peroxisome, GDH was localized to the chloroplast, peroxisomes and the cytoplasm, targeting the conversion of the “residual” glycolate in these compartments (FIG. 5). The approach using our novel TriTags, is further described in this work.

Plastome Integration for Augmenting Photosynthesis Using 3-HOP

The concept of a universal plastome integration vector has been described in several reviews (Lutz, et al. 2007)(Verma, 2007). Integration vectors designed for Nicotiana sp. had been used to stably transform strains of related nightshades tomato and potato, however with significantly lower efficiency (Sidorov, et al. 1999)(Ruf, et al. 2001). In order to increase the efficiency of transformation a universal integration vector was constructed using the A. thaliana plastome as a default reference.

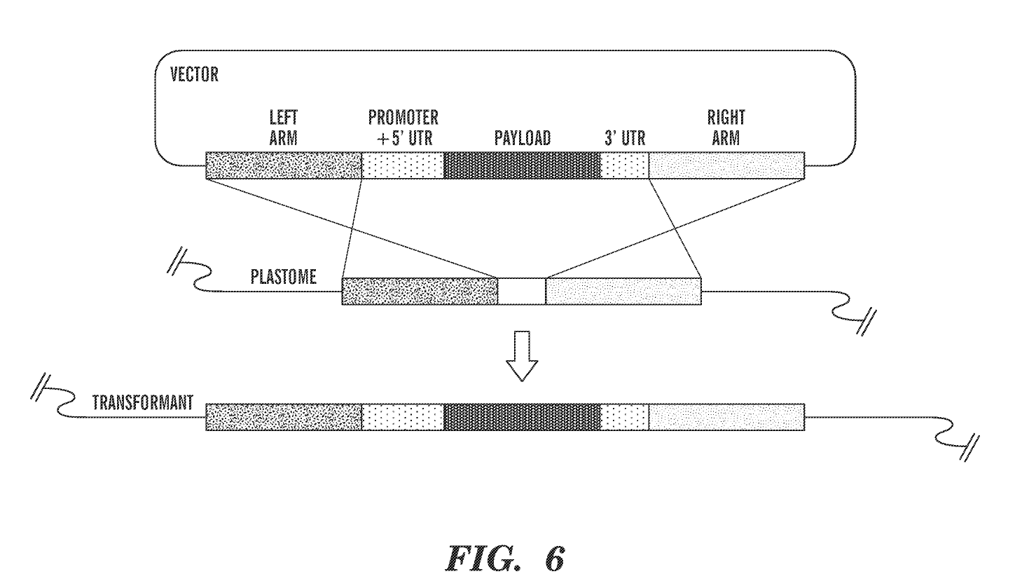

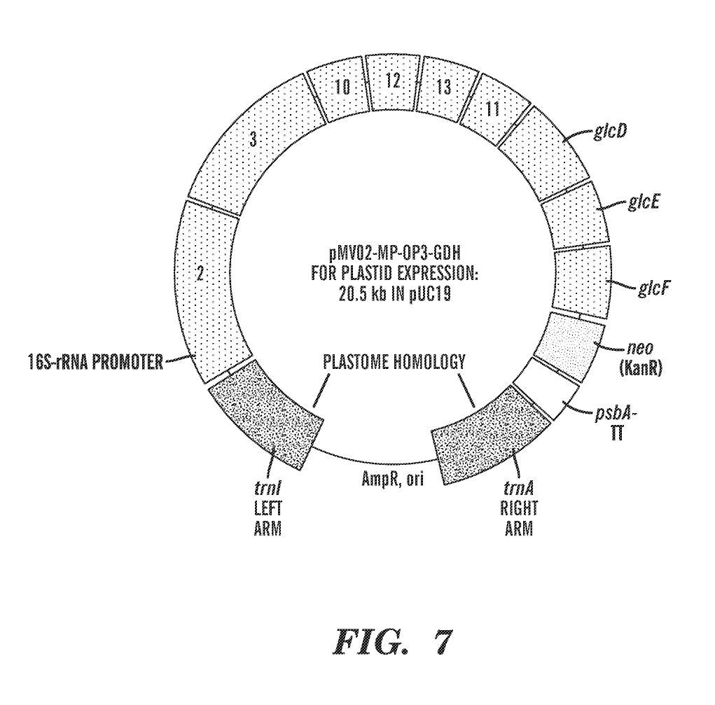

The integration vector (FIG. 7) consists of a multiple cloning site and functionally expressed kanamycin resistance cassette (payload) flanked by >800 nucleotide homology to isoleucine tRNA (trnI) and alanine tRNA (trnA) of the A. thaliana plastome, respectively. Homologous recombination will result in the integration of the payload into this transcriptionally-active neutral site within the plastome (FIG. 6). A BLAST comparison of the trnI and trnA region homology between higher plants is shown in Table 2.

TABLE 2
BLAST local alignment comparisons of trnI and trnA across
plastomes of various C₃ plant species.

| | trnI alignment | | trnA alignment | |
|---|---|---|---|---|
| | | Max | | Max |
| | | identity | Coverage | identity |
| Species | Coverage (%) | (%) | (%) | (%) |

| | | | | |
|---|---|---|---|---|
| Arabidopsis thaliana | 100 | 100 | 100 | 100 |
| Brassica napus | 98 | 99.22 | 94 | 99.11 |
| Theobroma cacao | 99 | 98.02 | 100 | 96.44 |
| Coffea arabica | 98 | 96.02 | 99 | 93.39 |
| Solanum tuberosum | 99 | 95.57 | 99 | 94.51 |
| Nicotiana tabacum | 99 | 95.32 | 89 | 95.62 |
| Soybean | 99 | 95.07 | 100 | 93.38 |

The functional expression cassette (i.e. payload) consists of the A. thaliana plastome 16S ribosomal RNA promoter (Prrn); a constitutively active promoter followed by a multiple cloning site used for inserting genes of interest in a polycistronic fashion, a kanamycin cassette and A. thaliana plastome photosystem B terminator (PsbA-TT) (Carrer, et al. 1993). The kanamycin cassette (neo) was strategically placed at the end of the polycistron in order to guarantee transcription of the entire upstream operon in obtained kanamycin transformants.

The universal integration vector pMV02 was constructed from six parts using Gibson assembly. The origins and techniques used to obtain each part are further described below herein. Regions trnI, trnA and psbA were acquired by gradient polymerase chain reaction (PCR) from plastid DNA obtained from A. thaliana using the DNeasy extraction kit (QIAGEN). For traditional cloning in E. coli the plasmid backbone containing an origin of replication and ampicillin resistance cassette was amplified by PCR from pUC19. The promoter, a plastid 16S rRNA promoter and MCS were constructed by assembly PCR from oligonucleotides (<20 nt) designed using the Gene2Oligo server. The kanamycin cassette was obtained by PCR amplification from the pORE family of plant integration vectors (Coutu, et al. 2007). The lactose promoter within the backbone of the pUC19 vector was subsequently excised to yield the vector (pMV02) used for 3-HOP operon insertion cloning and plastome integration in this project.

The vector is delivered into the cells of the leaf tissue by precipitation onto gold nano-particles and subsequent bombardment by a Biolistic® delivery system (BioRad). Due to the lack of accessibility to the PDS1000/He Biolistics delivery device, the Helios® Gene Gun was initially used to deliver the vector to chloroplasts of Nicotiana benthamiana leaf tissue. Mature leaves of Nicotiana species are regularly used for plastome transformation due to their high efficiency of DNA integration and ease of handling.

The second round of bombardments was performed using the PDS1000/He. Here, the target area and the size of a mature N. benthamiana leaf are of the same order of magnitude, resulting in a higher probability of stable plastid transformants. The protocol is given below herein.

When efficiency of transformation has been established, several points should be considered if further improvements are required. (1) The use of more efficient and effective plastid selectable markers. A spectinomycin resistant cassette (aadA) as opposed to the kanamycin cassette (nptII) used here could increase the transformation efficiency in N. tabacum leaves. Furthermore, 5′UTR and 3′UTR regions appear to play a larger role in determining selection efficiency than the class of antibiotic selection marker (Lutz, et al. 2007). As antibiotic and herbicide resistant markers are unfavorable in the current political agricultural milieu, one would be more inclined to search for marker-free based selection, such a photoautotrophy or metabolic complementation (Day and Goldschmidt-Clermont 2011). (2) Increasing the length of the homology regions for recombination into the trnI/trnA plastome sites. While this might seem intuitive, a trade-off exists between the length of homology on the one side and ease of cloning or decrease of transformation efficiency due to specificity on the other (Lutz, et al. 2007).

Sub-Cellular Targeting to Improve Shunting of Photorespiration

Central to the success of the field of plant engineering are the means by which engineers can control the sub-cellular location of expression and activity of heterologous enzymes or proteins. Generally, proteinaceous localization tags are sufficient for targeting to a single compartment and do not infer high demands on time and resources from the engineer. While single-compartment localization may be sufficient for fundamental functional characterization of subsets of plant genes, there is increasing evidence indicating that a multitude of genes involved in plant organellar protein synthesis and metabolic pathways are targeted to at least two or more compartments (Severing, van Dijk and van Ham 2011) (Brandão and Silva-Filho 2011) (Baudisch and Klosgen 2012).

Today's method of achieving targeting to multiple compartments involves the addition of multiple localization tags, which can be deleterious to the protein's function and greatly increased the amount of time and resources required as the number of targeted compartments increases. El Amrani, et al. 2004).

Herein are described three EMLs, termed TriTags, designed for the purpose of standardized multiple compartment localization of a transgene. Two elements are based on the plant cell's inherent capacity to create functional diversity from one gene: alternative splicing. The third element is based on the plant cell localization machinery's specificity: ambiguous protein tags. The targeting of fused green fluorescent protein (Aequeora victoria) to the cytoplasm, chloroplasts and peroxisomes in transiently transformed N. benthamiana is demonstrated. The technology is specifically contemplated for use in minimizing photorespiration in C₃ plants, as described elsewhere herein.

Alternative Splicing in Nature.

Alternative splicing is an event that occurs frequently in eukaryotic cells in which mRNA molecules are processed after having been transcribed from DNA (post-transcriptional modification, PTM). Overall, the processes result in the excision of particular regions of nucleic acid (introns) from the mRNA molecule. Splicing of mRNA is performed by an RNA and protein complex known as the spliceosome. The general process involves the recognition of the dinucleotide guanine and uracil (GU, donor site) at the 5′ end of an intron and adenine and guanine (AG, acceptor site) at the 3′ end by the spliceosome, followed by the excision of the intervening nucleotides and the reassembly of both ends (Severing, van Dijk and van Ham 2011).

TriTag-1 and TriTag-2 Design: Modularity in Alternative Splicing.

The first module of the sequence is described (Dinkins, et al. 2008) in the context of a variant of the protein-L-isoaspartate methyltransferase (PIMT2) gene of Arabidopsis thaliana and mechanisms involved in its sub-cellular localization. Alternative splicing events of the RNA product in vivo affords variants of the PIMT2 protein, which either localize to the cytoplasm or the chloroplasts. The second module of TriTag-1 is described in Reumann, et al. (2007). Therein an internally functional peroxisomal targeting signal (PTS2) is elucidated within a spliced version of a bifunctional transthyrtin-like protein of A. thaliana. Translocation of proteins to peroxisomes is mediated by this conserved sequence of amino acids (Arg-Leu-X₅-His-Leu (SEQ ID NO: 5)), generally found at the N-terminus of the protein expressed. Thus, this genetic module affords splice variants, which localize to either the peroxisome or the cytoplasm.

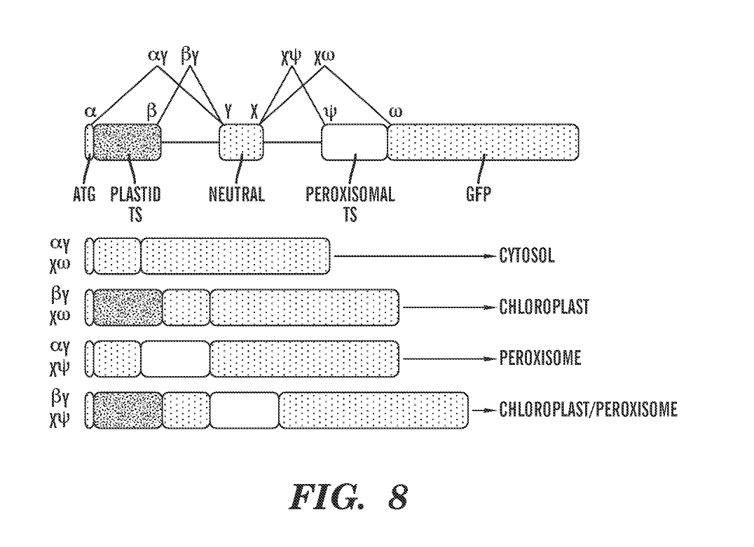

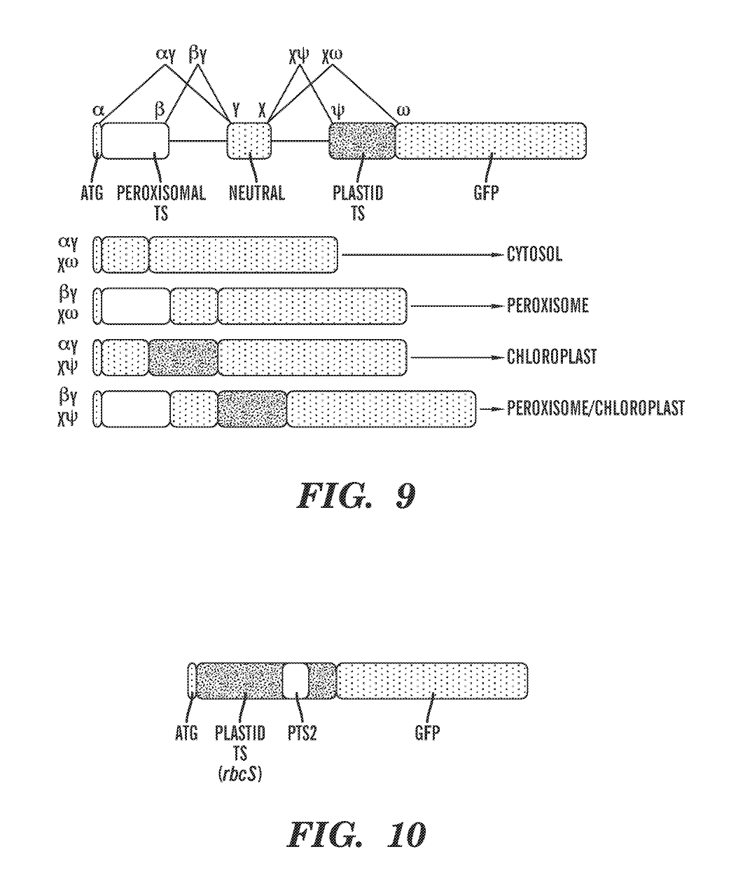

Combined, genetic modules 1 and 2 comprise the genetic element TriTag-1, which by means of alternative splicing, afford proteins of interest a transit peptide for localization to the chloroplast, peroxisome and cytoplasm (FIG. 8). TriTag-1 and TriTag-2 utilize alternative 5′ donor sites and alternative 3′ acceptor sites. TriTag-1 is composed of module 1 followed by module 2, in frame. This combination affords functional splice variants expressing transit peptides with PTS2 and/or CTP, or no defined targeting signal (resulting in cytoplasmic localization). Similar to TriTag-1, TriTag-2 combines modules 1 and 2, however TriTag-2 contains the modules in reverse arrangement; with module 2 at the 5′ end of the genetic element (FIG. 9).

TriTag-3 Design: Re-Thinking Specificity.

Translocation of proteins to chloroplasts is mediated by particular amino acid sequences (chloroplast transit peptides, cTP) consisting primarily of hydrophobic side-chains at the N-terminus and a preference for hydroxylated amino acids (serine, threonine, etc), as exemplified by the potato chloroplast targeting peptide region of the rbcS1 gene (gi21562). TriTag-3 is a synthetic designed nucleic acid expressing an ambiguous transit peptide. It was designed by superimposing the consensus PTS2 sequence over the potato RuBisCO chloroplast transit peptide (FIG. 10).

The N-terminus of this ambiguous transit peptide is sufficiently hydrophobic for chloroplast localization and the PTS2 signal amply recognized by its receptor, PEX7 resulting in peroxisomal localization. Here, equilibrium between fused protein levels in the peroxisome and chloroplast occurs as a result of the competition between organelles for the ambiguous signal. Furthermore, this push-and-pull mechanism will result in increased retention of the fused protein within the cytoplasm.

Subcellular Localization of TriTags in Transient Expression Assays.

To determine functionality of the TriTags, Nicotiana benthamiana epidermal cells were bombarded with the TriTag fused to GFP, the expression of which was controlled by the constitutively active promoter on the plasmid pENTCUP2. Images of transiently transformed cells were taken by confocal microscopy (Leica SP5 X MP, Buffalo Grove, Ill. 60089 United States) after 48 hours of incubation at RT.

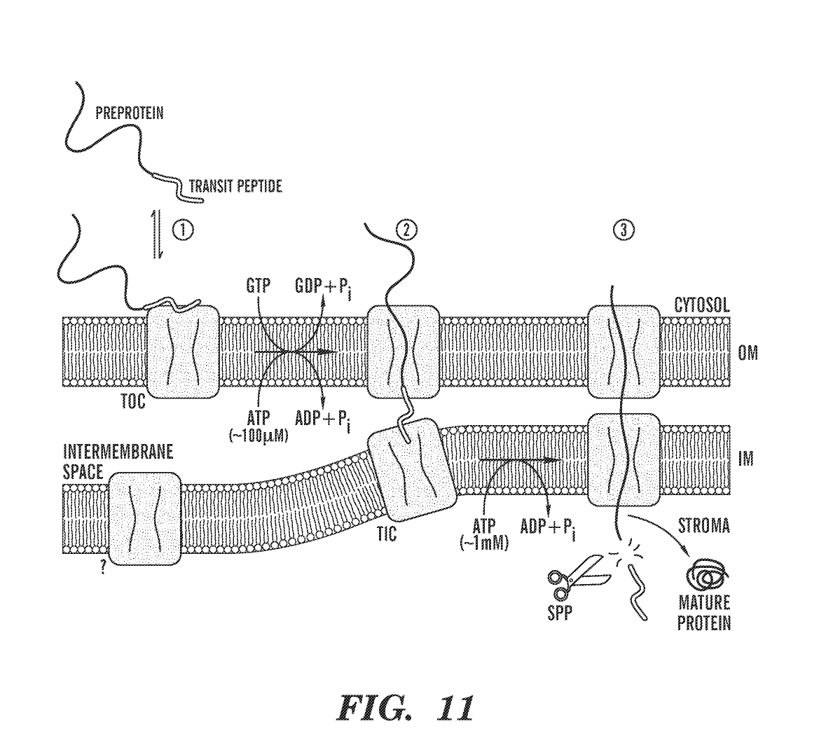

When expressed without a fusion, GFP is distributed exclusively in the cell's periphery and nucleus. This localization pattern is typical for free GFP (Li, et al. 2010). The peripheral pattern is due to the exclusion of the cytoplasm from the inside of the cell by the vacuole (data not shown). Cytoplasmic, nuclear and chloroplast localization patterns are observed with transient expression of GFP fused to the chloroplast targeting peptide of the potato RuBisCO protein (Kebeish, et al. 2007). The presence of GFP in the cell's periphery was unexpected; the chloroplast transit peptide from rbcS1 is one of the most specific known. However, one can imagine that, at high protein expression levels, equilibrium is established between the passive exclusion of GFP from the chloroplast and active import into the chloroplast. Furthermore, varying ATP/GTP levels within the cell influences the flux of active protein import (data not shown, see also the Tic-Toc chloroplast import machinery diagram in FIG. 11).

For transient expression of TriTag1-GFP, localization to the cytoplasm and the outer membrane of the chloroplast is observed. Furthermore, distinct punctate patterns of expression are observed (data not shown), in keeping with peroxisomal localization.

TriTag2-GFP is present in the cytoplasm of N. benthamiana. In addition, a similar punctate pattern of localization is observed (data not shown). However, exclusion from the chloroplast is evident (data not shown).

Overall, the localization patterns of the alternative splicing-based tags (TriTag1 and TriTag2) show a punctate pattern of expression in addition to the cytoplasmic distribution. The alternative splicing modules on which TriTag1 and TriTag2 are based were inspired from genes in A. thaliana. Subcellular localization will also be determined in A. thaliana epidermal leaf cells stably transformed with TriTag1-GFP and TriTag2-GFP.

In N. benthamiana epidermal leaf cells transiently expressed with TriTag3-GFP, chloroplast localization is observed along with a punctate pattern resembling peroxisomal localization (data not shown). There is a distinct difference in localization between TriTag3 and the control cTP-GFP (data not shown), with relatively low levels of GFP in the cytoplasm (e.g., the lack of visible cell periphery or cytosolic expression). Without wishing to be bound by theory, while cTP-GFP GFP is distributed between the chloroplast (active import) and free GFP in the cytoplasm (passive), it is possible that the added PTS2 actively shuttles—what would have been—free GFP to the peroxisomes, changing the distribution pattern compared to the rbcS1 transit peptide from which TriTag3 was designed (FIG. 10).

TABLE 3
Overview of subcellular localization found using TriTag technology.

| | Location | | |
|---|---|---|---|
| Construct | Cytoplasm | Chloroplast | Peroxisome |
| Untagged GFP | Yes | No | No |
| cTP-GFP | Yes | Yes | No |
| TriTag1-GFP | Yes | Outer membrane | Yes* |
| TriTag2-GFP | Yes | No | Yes* |
| TriTag3-GFP | Low | Yes | Yes* |

*Requires correlation experiments for confirmation

For all TriTags tested, additional punctate localization patterns are observed which distinctly differ from an untagged GFP pattern (Table 3).

Minimizing Photorespiration by TriTagging E. coli Glycolate Dehydrogenase

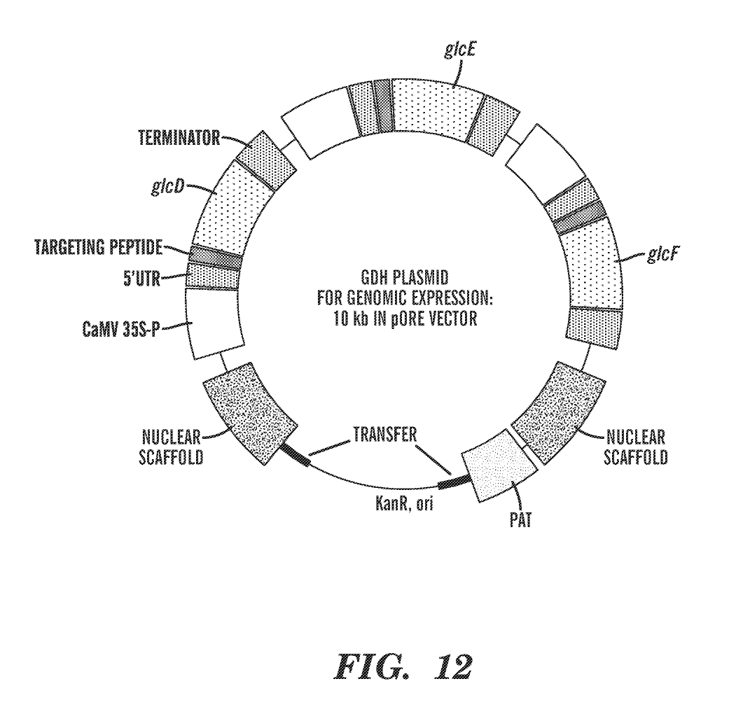

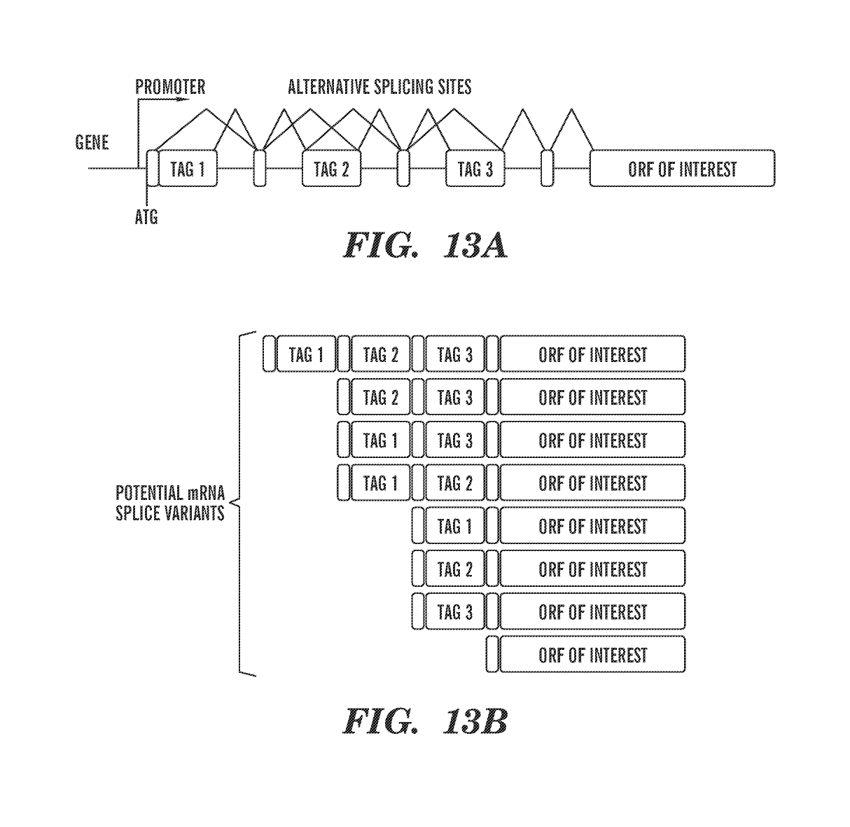

An economically relevant application for the TriTag system is its use in multiple-compartment shunting of photorespiration. Like many central metabolic pathways in plants, the reactions involved in photorespiration take place in more than one compartment, specifically the chloroplasts, cytoplasm, peroxisomes and mitochondria (FIG. 12). Kebeish et al. have succeeded in shunting photorespiration by implementing the bacterial glycerate pathway, converting glycolate into the Calvin cycle-ready phosphoglycerate (Kebeish, et al. 2007). This shunt had resulted in increased biomass yield of A. thaliana, specifically in the roots and overall rosette diameter.

Glycolate, the wasteful product of the oxygenase reaction catalyzed by the enzyme RuBisCO is shuttled via the cytosol to the peroxisomes where, by means of many energy-requiring reactions within various compartments the carbons are regenerated into the more reduced glycerate-3-P as substrate for RuBisCO. The cycle is generally considered futile as a carbon, which is fixed in the chloroplasts, is subsequently released in the mitochondria. This energetically wasteful reaction is termed photorespiration.

Interestingly, it has been shown that the first conversion step of the glycerate pathway, namely the conversion of glycolate to glyoxylate by glycolate dehydrogenase (gclDEF, GDH) is responsible for >60% of the increase in biomass yield, suggesting that A. thaliana chloroplasts can natively oxidize glycolate, however at an insufficient rate to increase photosynthesis efficiency (Peterhansel, Niessen and Kebeish 2008). Kebeish et al. achieved increased biomass yield by targeting solely E. coli GDH to the chloroplast. Assuming that at any particular moment the pool of glycolate within the plant cell is distributed between the chloroplasts, cytosol and peroxisomes, targeting GDH to all three compartments will (1) prevent the relatively energetically wasteful hydrogen peroxide-forming oxidation of glycolate to glyoxylate in the peroxisome, (2) produce an extra reducing equivalent (NADH) from glycolate in the cytosol and (3) boost glyoxylate formation in the chloroplast. Overall, with the increase in reducing equivalents, avoidance of peroxide-forming reactions, decreased requirement for metabolite shuttling between compartment and a boost in glyoxylate formation in the chloroplast, an increase in biomass yield is expected (see also FIG. 5).

Binary plasmids for the Agrobacterium-mediated genomic transformation of A. thaliana were constructed containing the E. coli glcD, glcE and glcF genes codon-optimized for A. thaliana expression and used to floral dip A. thaliana Col-0. Four different plasmids were constructed, each with a different set of targeting peptides attached to the GDH subunits (FIG. 12; pORE-cTP-GDH, pORE-TriTag1-GDH, pORE-TriTag2-GDH and pORE-TriTag3-GDH). Currently, transformants are being screened for resistance to glufosinate (Finale, Bayer). Stable genomic integration can be confirmed by PCR and transformants characterized for their biomass accumulation rates and rates of photorespiration.

Discussion

Today, the field of crop engineering is saturated with standards created more than a decade ago, which have not had the ability to further evolve in the industrial settings they proliferated in. Synthetic biology provides us with a new engineering perspective and concepts, which will prove useful in bio-energy, pharmaceuticals and increasing yields in planta to better suit the needs of an increasing global population.

The design and construction of a universal plastome integration vector naturally follows from the broad-host range perspective of synthetic biology. With the knowledge that the chloroplast transcription/translation machinery functions akin to its bacterial counterpart, and a universal vector for integration, engineers can now achieve multiple gene expression within a plant cell's compartment by the construction of relatively inexpensive and little-effort polycistronic bacterial operons. Furthermore, the increasingly vast array of standard genetic parts (promoters, ribosome binding sites, terminators) found in bacterial genetic databases such as the PartsRegistry (partsregistry.org) have now essentially been made available to plant genetic engineers.

A powerful tool within the synthetic biology movement is the use of abstraction to simplify complexity for the bioengineer by omitting certain details. Standardization of biological parts used further supports this level of abstraction. Provided herein is a simplified abstraction model for the use of standardized localization tags based on alternative splicing, and proof that these same synthetic biology principles are at play in the system.

The methods and compositions described herein allow for a set of parts that, when arranged appropriately, will yield localization tags for any subset of desired compartments within the plant cell by alternative splicing.

Methods

Strains and Plasmids.

E. coli K12 strains (NEB Turbo, New England Biolabs) were used as plasmid hosts for cloning work on plastome integration vectors and binary vectors for transient expression and/or stable genomic integration. Strains and plasmids are listed in Table 4. Plasmids were constructed with traditional cloning methods (Sambrook J. and Russell D. W. 2001), BglBricks (Anderson, et al. 2010), BioBricks (Knight, T 2003), or Gibson assembly (Gibson 2011) using genes codon-optimized for either A. thaliana (all binary vectors) or E. coli (plastome integration vectors) (Genscript, Piscataway, N.J.).

TABLE 4
Plasmids used in this study

| Plasmid | Description | SEQ ID NO: |
|---|---|---|
| pMV02 | Universal plastome integration vector. | 88 |
| pMV02-OP3 | Universal plastome integration vector | 89 |
| | expressing 3-HOP sub-pathway 4. | |
| pMV02-MP | Universal plastome integration vector | 90 |
| | expressing 3-HOP sub-pathway 1. | |
| pMV02-GDH | Universal plastome integration vector | 91 |
| | expressing E. coli glycolate dehydrogenase | |
| | (glcDEF). | |
| pMV02-GFP | Universal plastome integration vector | 92 |
| | expressing smGFP. | |
| pMV02-MP-OP3 | Universal plastome integration vector | 93 |
| | expressing 3-HOP sub-pathways 1 and 4. | |
| pMV02-MP-GDH | Universal plastome integration vector | 94 |
| | expressing 3-HOP sub-pathway 1 and E. coli | |
| | glcDEF. | |
| pMV02-OP3-GDH | Universal plastome integration vector | 95 |
| | expressing 3-HOP sub-pathway 4 and E. coli | |
| | glcDEF. | |
| pMV02-MP-OP3-GDH | Universal plastome integration vector | 96 |
| | expressing 3-HOP sub-pathways 1, 4 and E. coli | |
| | glcDEF. | |
| pORE-GFP | pORE family of binary vectors expressing | 97 |
| | untagged GFP controlled by the pENTCUP2 | |
| | promoter. | |
| pORE-rbcS1-GFP | pORE family of binary vectors expressing A. thaliana | 98 |
| | codon optimized GFP, fused to the | |
| | potato rbcS1 plastid localization tag and | |
| | flanked by nuclear scaffold regions (RB7). | |
| pORE-TriTag1-GFP | pORE family of binary vectors expressing | 99 |
| | TriTag1-fused GFP controlled by the | |
| | pENTCUP2 promoter. | |
| pORE-TriTag2-GFP | pORE family of binary vectors expressing | 100 |
| | TriTag2-fused GFP controlled by the | |
| | pENTCUP2 promoter. | |
| pORE-TriTag3-GFP | pORE family of binary vectors expressing | 101 |
| | TriTag3-fused GFP controlled by the | |
| | pENTCUP2 promoter. | |
| pORE-cTP-GDH | pORE family of binary vectors expressing E. coli | 102 |
| | glcD, glcE, and glcF codon optimized for | |
| | A. thaliana. Each subunit is N-terminally | |
| | fused in-frame to the potato rbcS1 plastid | |
| | localization tag and is moderated by its own | |
| | CaMV35S promoter, 5′UTR and NOS | |
| | terminator. The expression cassette is flanked | |
| | by nuclear scaffold regions (RB7); minimizing | |
| | silencing. | |
| pORE-TriTag1-GDH | pORE family of binary vectors expressing E. coli | 103 |
| | glcD, glcE, and glcF codon optimized for | |
| | A. thaliana. Each subunit is N-terminally | |
| | fused in-frame to TriTag1 and is moderated | |
| | by its own CaMV35S promoter, 5′UTR and | |
| | NOS terminator. The expression cassette is | |
| | flanked by nuclear scaffold regions (RB7); | |
| | minimizing silencing. | |
| pORE-TriTag2-GDH | pORE family of binary vectors expressing E. coli | 104 |
| | glcD, glcE, and glcF codon optimized for | |
| | A. thaliana. Each subunit is N-terminally | |
| | fused in-frame to TriTag2 and is moderated | |
| | by its own CaMV35S promoter, 5′UTR and | |
| | NOS terminator. The expression cassette is | |
| | flanked by nuclear scaffold regions (RB7); | |
| | minimizing silencing. | |
| pORE-TriTag3-GDH | pORE family of binary vectors expressing E. coli | 105 |
| | glcD, glcE, and glcF codon optimized for | |
| | A. thaliana. Each subunit is N-terminally | |
| | fused in-frame to TriTag3 and is moderated | |
| | by its own CaMV35S promoter, 5′UTR and | |
| | NOS terminator. The expression cassette is | |
| | flanked by nuclear scaffold regions (RB7); | |
| | minimizing silencing. | |

Media.

E. coli K12 cells were grown in Luria-Bertani medium with appropriate antibiotics.

Universal plastome integration vector construction and cloning.

pMV02 was constructed by Gibson assembly of the following 6 parts. trnI (1) and trnA (2) regions of homology were acquired by A. thaliana plastomic PCR. (3) A. thaliana 16S rRNA promoter and MCS were synthesized by assembly PCR from oligos designed using the Gene2Oligo server (http://berry.engin.umich.edu/gene2oligo/). (4) The nptII kanamycin resistance cassette was obtained from the pORE family of vectors by PCR(Coutu, et al. 2007). (5) The A. thaliana chloroplast photosystem II protein D terminator region was acquired by PCR from extracted plastomic DNA of A. thaliana leaves (DNeasy Plant Mini Kit, QIAGEN). (6) The pUC19 backbone with its origin of replication and ampicillin resistance cassette was obtained by PCR. The 6 parts had a 20 basepairs of overlap at each end in order to facilitate proper annealing during the assembly reaction. The resulting plasmid, pMV02, was confirmed by sequencing (GeneWiz, Cambridge, Mass. USA). The Chloroflexus aurantiacus 3-HOP sub-pathways 1, 4 (codon optimized for E. coli expression, GenScript, Piscatawny, N.J. USA) and E. coli glcDEF (PCR from E. coli genomic DNA) were cloned into the MCS using EcoRI and SalI sites, resulting in the configurations noted in Table 4.

TriTag Synthesis and Cloning.

TriTag1-3 were synthesized by IDT (gBlocks, Coralville, Iowa) and were fused in-frame to the GFP ORF in pORE-GFP by Gibson assembly, resulting in pORE-TriTag1-GFP, pORE-TriTag2-GFP and pORE-TriTag3-GFP. In-frame insertions into pORE-GDH by Gibson assembly resulted in plasmids pORE-TriTag1-GDH, pORE-TriTag2-GDH and pORE-TriTag3-GDH (Table 4).

Glycolate Dehydrogenase Synthesis and Cloning.

E. coli GDH subunits glcD, glcE and glcF were codon optimized for A. thaliana expression and placed under control of the CaMV35S promoter, 5′UTR from Tobacco Etch Virus and the nopaline synthase (NOS) terminator. BioBrick combinatorial cloning was used to assemble the subunits together. RB7 nuclear scaffold regions were used to flank the 3-subunit expression cassette, thus minimizing gene silencing of the region (Halweg, Thompson and Spiker 2005). The RB7-glcD-glcE-glcF-RB7 component was inserted into a pORE glufosinate-resistant binary vector for floral dipping (Coutu, et al. 2007).

Plant Material.

All plants were incubated at RT in a 16/8 h light/dark cycle and watered biweekly. Peat-based potting soil was autoclaved before use. Nicotiana benthamiana seedlings were 4 to 6 weeks old. Leaves from 6 to 8 week old plants were collected for bombardment. Flowering Arabidopsis thaliana ecotype Columbia-0 plants were used for the Agrobacterium-mediated transformation procedures.

Biolistic Methods.

Transient GFP-fusion tag assays were conducted by precipitating 50 μg plasmid DNA onto 8 mg of 1 μm gold particles. N. benthamiana leaves were transformed biolistically using the Helios Gene Gun (Bio-Rad) at 150-250 psi. The leaves were placed on wet filter paper in Petri dishes and stored on a bench-top under ambient lighting and RT for 48 hours before analysis. Bombarded leaves were diced and placed on glass slides in ddH₂O+Triton-X100 and imaged by fluorescence confocal microscopy (excitation at 489 nm, emission at 500-569 for GFP and 630-700 for chlorophyll).

Agrobacterium-Mediated Transformation of A. thaliana.

Flowering A. thaliana (Columbia ecotype, Col-0) were transformed by the floral dip method (Clough and Bent 1998). A binary vector taken from the pORE family of vectors (Coutu, et al. 2007) containing the cloned-in expression cassettes of interest was electroporated into Agrobacterium tumefaciens GV3101 bearing the helper plasmid pMP90. Plants containing the trangenes were allowed to self-pollinate and subject to rounds of selection on either glufosinate (PAT resistance marker) or kanamycin (nptII resistance marker).

### REFERENCES

Anderson, J Christopher, et al. “BglBricks: A flexible standard for biological part assembly.” Journal of biological engineering 4, no. 1 (January 2010): 1.
Baudisch, Bianca, and Ralf Bernd Klösgen. “Dual targeting of a processing peptidase into both endosymbiotic organelles mediated by a transport signal of unusual architecture.” Molecular plant 5, no. 2 (March 2012): 494-503.
Berg, Ivan A, Daniel Kockelkorn, Wolfgang Buckel, and Georg Fuchs. “A 3-hydroxypropionate/4-hydroxybutyrate autotrophic carbon dioxide assimilation pathway in Archaea.” Science (New York, N.Y.) 318, no. 5857 (December 2007): 1782-6.
Brandão, Marcelo M, and Marcio C Silva-Filho. “Evolutionary history of Arabidopsis thaliana aminoacyl-tRNA synthetase dual-targeted proteins.” Molecular biology and evolution 28, no. 1 (January 2011): 79-85.
Buchanan, B B, and D I Arnon. “A reverse KREBS cycle in photosynthesis: consensus at last.” Photosynthesis research 24 (January 1990): 47-53.
Carrer, H, T N Hockenberry, Z Svab, and P Maliga. “Kanamycin resistance as a selectable marker for plastid transformation in tobacco.” Molecular & general genetics: MGG 241, no. 1-2 (October 1993): 49-56.
Clarke, Jihong Liu, and Henry Daniell. “Plastid biotechnology for crop production: present status and future perspectives.” Plant molecular biology 76, no. 3-5 (July 2011): 211-20.
Clough, S J, and A F Bent. “Floral dip: a simplified method for Agrobacterium-mediated transformation of Arabidopsis thaliana.” The Plant journal: for cell and molecular biology 16, no. 6 (December 1998): 735-43.
Coutu, Catherine, et al. “pORE: a modular binary vector series suited for both monocot and dicot plant transformation.” Transgenic research 16, no. 6 (December 2007): 771-81.
Day, Anil, and Michel Goldschmidt-Clermont. “The chloroplast transformation toolbox: selectable markers and marker removal.” Plant biotechnology journal 9, no. 5 (June 2011): 540-53.
De Cosa, B, W Moar, S Lee, and M Miller . . . “Overexpression of the Bt cry2Aa2 operon in chloroplasts leads to formation of insecticidal crystals.” Nature Biotechnology, January 2001.
Dinkins, Randy D, et al. “Changing transcriptional initiation sites and alternative 5′- and 3′-splice site selection of the first intron deploys Arabidopsis protein isoaspartyl methyltransferase2 variants to different subcellular compartments.” The Plant journal: for cell and molecular biology 55, no. 1 (July 2008): 1-13.
Ducat, Daniel C, and Pamela A Silver. “Improving carbon fixation pathways.” Current opinion in chemical biology, May 2012.
El Amrani, Abdelhak, et al. “Coordinate expression and independent subcellular targeting of multiple proteins from a single transgene.” Plant Physiology 135, no. 1 (May 2004): 16-24.
Flannery, M L, et al. “Plastid genome characterisation in Brassica and Brassicaceae using a new set of nine SSRs.” TAG Theoretical and applied genetics Theoretische and angewandte Genetik 113, no. 7 (November 2006): 1221-31.
Gibson, Daniel G. “Enzymatic assembly of overlapping DNA fragments.” Methods in enzymology 498 (January 2011): 349-61.
Halweg, Christopher, William F Thompson, and Steven Spiker. “The rb7 matrix attachment region increases the likelihood and magnitude of transgene expression in tobacco cells: a flow cytometric study.” The Plant cell 17, no. 2 (February 2005): 418-29.
Hickey, Scott F, et al. “Transgene regulation in plants by alternative splicing of a suicide exon.” Nucleic acids research 40, no. 10 (May 2012): 4701-10.
Horstmann, Verena, Claudia M Huether, Wolfgang Jost, Ralf Reski, and Eva L Decker. “Quantitative promoter analysis in Physcomitrella patens: a set of plant vectors activating gene expression within three orders of magnitude.” BMC Biotechnology 4 (July 2004): 13.
Huber, Harald, et al. “A dicarboxylate/4-hydroxybutyrate autotrophic carbon assimilation cycle in the hyperthermophilic Archaeum Ignicoccus hospitalis.” Proceedings of the National Academy of Sciences of the United States of America 105, no. 22 (June 2008): 7851-6.
Kebeish, Rashad, et al. “Chloroplastic photorespiratory bypass increases photosynthesis and biomass production in Arabidopsis thaliana.” Nature biotechnology 25, no. 5 (May 2007): 593-9.
Knight, T. “Idempotent Vector Design for Standard Assembly of Biobricks.” MIT Artificial Intelligence Laboratory; MIT Synthetic Biology Working Group, August 2003: 1-11.
Li, Feng, et al. “Rice DENSE AND ERECT PANICLE 2 is essential for determining panicle outgrowth and elongation.” Cell research 20, no. 7 (July 2010): 838-49.
Ljungdahl, L, E Irion, and H G Wood. “Total synthesis of acetate from CO2. I. Co-methylcobyric acid and CO-(methyl)-5-methoxybenzimidazolylcobamide as intermediates with Clostridium thermoaceticum.” Biochemistry 4, no. 12 (December 1965): 2771-80.
Lutz, Kerry Ann, Arun Kumar Azhagiri, Tarinee Tungsuchat-Huang, and Pal Maliga. “A guide to choosing vectors for transformation of the plastid genome of higher plants.” Plant Physiology 145, no. 4 (December 2007): 1201-10.
Pagani, Mark, James C Zachos, Katherine H Freeman, Brett Tipple, and Stephen Bohaty. “Marked decline in atmospheric carbon dioxide concentrations during the Paleogene.” Science (New York, N.Y.) 309, no. 5734 (July 2005): 600-3.
Peterhansel, Christoph, Markus Niessen, and Rashad M Kebeish. “Metabolic engineering towards the enhancement of photosynthesis.” Photochemistry and photobiology 84, no. 6 (January 2008): 1317-23.
Reumann, Sigrun, et al. “Proteome analysis of Arabidopsis leaf peroxisomes reveals novel targeting peptides, metabolic pathways, and defense mechanisms.” The Plant cell 19, no. 10 (October 2007): 3170-93.
Ruf, S, M Hermann, I J Berger, H Carrer, and R Bock. “Stable genetic transformation of tomato plastids and expression of a foreign protein in fruit.” Nature Biotechnology 19, no. 9 (September 2001): 870-5.
Sage, Rowan F, Tammy L Sage, and Ferit Kocacinar. “Photorespiration and the evolution of C4 photosynthesis.” Annual review of plant biology 63 (June 2012): 19-47.
Sambrook J., and Russell D. W. “Molecular Cloning: A Laboratory Manual 3rd ed.” Cold Spring Harbor, N.Y.: Cold Spring Harbor Laboratory Press., 2001.
Severing, Edouard I, Aalt D J van Dijk, and Roeland C H J van Ham. “Assessing the contribution of alternative splicing to proteome diversity in Arabidopsis thaliana using proteomics data.” BMC plant biology 11, no. 1 (January 2011): 82.
Sidorov, V, D Kasten, S Pang, P Hajdukiewicz, J Staub, and N Nehra. “Technical Advance: Stable chloroplast transformation in potato: use of green fluorescent protein as a plastid marker.” The Plant journal: for cell and molecular biology 19, no. 2 (July 1999): 209-216.
Verma . . . , D. “Chloroplast vector systems for biotechnology applications.” Plant Physiology, January 2007.
von Caemmerer, Susanne, W Paul Quick, and Robert T Furbank. “The development of C₄ rice: current progress and future challenges.” Science (New York, N.Y.) 336, no. 6089 (June 2012): 1671-2.
Zachos, J, M Pagani, L Sloan, E Thomas, and K Billups. “Trends, rhythms, and aberrations in global climate 65 Ma to present.” Science (New York, N.Y.) 292, no. 5517 (April 2001): 686-93.
Zarzycki, J, V Brecht, and M Müller . . . “Identifying the missing steps of the autotrophic 3-hydroxypropionate CO2 fixation cycle in Chloroflexus aurantiacus.” Proceedings of the . . . , January 2009.
Zarzycki . . . , J. “Coassimilation of Organic Substrates via the Autotrophic 3-Hydroxypropionate Bi-Cycle in Chloroflexus aurantiacus.” Applied and environmental microbiology, January 2011.
Zhu, Xin-Guang, Stephen P Long, and Donald R Ort. “Improving photosynthetic efficiency for greater yield.” Annual review of plant biology 61 (January 2010): 235-61.

### Example 4

### Triple Destination Transit Elements for Efficient Targeting of Heterologous Proteins and Uses Thereof

Embodiments of the present invention relate to the use of genetic elements which when combined with any gene of interest will afford element-tagged polypeptides with the ability to localize to various targeted subcellular locations within eukaryotic cells. Specifically, this technology can be beneficial for the targeting of enzymes involved in, but not limited to, plant central metabolism, including bypassing photorespiration (1), high levels of protein accumulation within eukaryotic cells (2) and defined targeting of proteins required for cellular regulation and stress tolerance (3).

Described herein are TriTag-1, TriTag-2 and TriTag-3.

The nucleic acid sequence for TriTag-1 as fused to glycolate dehydrogenase is shown underlined in SEQ ID NO: 28 below:

SEQ ID NO: 28
atggaggtatgttctcttgccaggaatctctgcttcagtttattctcaac
acataaggtatacaaatgggttatttggtgtttctctgtgttgtgtgact
gattttgtgcttatagacgatttttaatatgttgatggtgttagcaattc
cagagtggaactggctcgagcggcgacagctctagctctcctgtttcaac
aaaacctcaggtatgatttaccaaatcttttccttgtcaaagttttgtgt
ttgactgtgtgggtttgaacctgttaggattcagtatgatatcaagtatg
tgtcttttggaatacaaggatttaccatatggctatctttgttatctgtg
tgaccttttctactttctcgctttgtaagatcgtctgagaatcattggag
ggcatttgaatgttgcagctgaagcaATGTCTATTCTTTATGAAGAGAGA
CTCGATGGAGCTTTACCAGATGTTGATAGAACCTCAGTGCTCATGGCATT
AAGGGAACATGTTCCTGGACTTGAAATTCTTCACACAGATGAAGAGATTA
TCCCATATGAATGTGATGGTTTGTCTGCTTACAGAACTAGGCCTCTTTTG
GTTGTGCTCCCAAAGCAGATGGAACAGGTTACAGCTATTCTTGCAGTGTG
CCATAGATTGAGGGTTCCTGTTGTGACAAGAGGAGCTGGTACCGGACTTT
CAGGAGGTGCACTCCCATTAGAAAAGGGTGTTCTCTTAGTGATGGCTAGG
TTCAAAGAGATATTGGATATTAATCCTGTGGGAAGAAGGGCTAGAGTTCA
ACCAGGTGTGAGGAATCTCGCAATTAGTCAGGCTGTTGCACCTCACAACC
TTTATTACGCTCCTGATCCATCTTCACAAATCGCATGTTCTATAGGTGGT
AATGTGGCTGAAAACGCAGGAGGTGTTCATTGCCTTAAGTACGGATTGAC
TGTGCACAACCTTTTGAAAATCGAAGTTCAGACTCTTGATGGAGAGGCTC
TTACATTGGGTAGTGATGCATTGGATTCTCCTGGTTTTGATCTCTTAGCT
CTCTTCACAGGTTCTGAAGGAATGTTAGGTGTTACTACAGAGGTTACCGT
TAAACTTTTGCCAAAACCTCCAGTTGCTAGAGTGCTCTTAGCATCTTTTG
ATTCAGTGGAAAAAGCTGGACTTGCAGTTGGAGATATAATTGCTAACGGA
ATTATTCCTGGAGGTCTCGAAATGATGGATAACTTATCTATAAGAGCTGC
TGAAGATTTCATTCATGCTGGATATCCAGTTGATGCTGAGGCAATACTTT
TGTGTGAACTTGATGGTGTTGAGTCAGATGTGCAAGAAGATTGCGAGAGA
GTTAATGATATTCTCTTAAAGGCTGGAGCAACTGATGTGAGGTTGGCTCA
GGATGAAGCAGAGAGAGTTAGGTTTTGGGCTGGAAGAAAAAACGCTTTCC
CTGCTGTTGGTAGGATCTCACCAGATTATTACTGTATGGATGGTACAATA
CCTAGAAGGGCTCTCCCAGGAGTTTTAGAGGGTATTGCAAGACTTAGTCA
ACAGTACGATTTGAGGGTTGCTAATGTGTTTCATGCAGGAGATGGAAACA
TGCACCCTCTCATCTTATTTGATGCTAATGAGCCAGGAGAGTTCGCTAGA
GCAGAAGAGCTTGGAGGAAAGATTCTTGAACTTTGTGTTGAAGTGGGAGG
TAGTATCTCTGGTGAACATGGTATTGGAAGAGAGAAAATCAATCAAATGT
GCGCTCAGTTCAACTCTGATGAAATCACCACTTTTCATGCTGTTAAGGCT
GCATTCGATCCTGATGGACTTTTGAATCCTGGAAAGAATATACCAACATT
GCACAGATGCGCTGAGTTCGGAGCAATGCACGTTCACCACGGACACCTTC
CTTTTCCTGAGTTGGAGAGATTCTGA

The first module of the sequence was first described in Dinkins et al. (2008) in the context of a variant of the PROTEIN—L-ISOASPARTATE METHYLTRANSFERASE (PIMT2) gene of Arabidopsis thaliana and mechanisms involved in its subcellular localization. Alternative splicing of the RNA product in vivo affords variants of the PIMT2 protein, which localizes either to the cytoplasm or the chloroplasts. The second module of TriTag-1 is first described in Reumann et al. (2007), which describes an internally functional peroxisomal targeting signal 2 (PTS2) signal elucidated within a spliced version of a bifunctional transthyrtin-like protein of A. thaliana. Thus, this module creates splice variants which localize to either the peroxisome or the cytoplasm.

Combined, modules 1 and 2 comprise a genetic element (TriTag-1), which by means of alternative splicing, will tag proteins of interest with a transit peptide for localization to the chloroplast and/or peroxisome and/or cytoplasm. This is an advance over prior methods, which would target significant amounts of the cargo polypeptide to only one subcellular location, typically to whichever location was indicated by the N-terminal-most localization signal. The embodiments of the technology described herein permit one gene to traffic significant quantities of cargo polypeptide to multiple subcellular locations, something not possible with the mere combination of two separate localization signals in one gene.

Similar to TriTag-1, TriTag-2 combines modules 1 and 2, however TriTag-2 contains the modules in reverse arrangement; with module 2 at the 5′ end of the genetic element. TriTag-2 is shown underlines in SEQ ID NO: 33 below:

SEQ ID NO: 33
atggacagctctagctctcctgtttcaacaaaacctcaaggtatattgat
gatttaccaaatcttttccttgtcaaagttttgtgtttgactgtgtgggt
ttgaacctgttaggattcagtatgatatcaagtatgtgtcttttggaata
caaggatttacccttatggctatctttgttatctgtgtgaccttttctac
tttctcgctttgtaagatcgtctgagaatcattggagggcatttgaatgt
tgcagctgaagcaatggaggtatgttctcttgccaggaatctctgcttca
gtttattctcaacacataaggtatacaaatgggttatttggtgtttctct
gtgttgtgtgactgattttgtgcttatagacgatttttaatatgttgatg
gtgttagcaattccagagtggaactggctcgagcggcATGTCTATTCTTT
ATGAAGAGAGACTCGATGGAGCTTTACCAGATGTTGATAGAACCTCAGTG
CTCATGGCATTAAGGGAACATGTTCCTGGACTTGAAATTCTTCACACAGA
TGAAGAGATTATCCCATATGAATGTGATGGTTTGTCTGCTTACAGAACTA
GGCCTCTTTTGGTTGTGCTCCCAAAGCAGATGGAACAGGTTACAGCTATT
CTTGCAGTGTGCCATAGATTGAGGGTTCCTGTTGTGACAAGAGGAGCTGG
TACCGGACTTTCAGGAGGTGCACTCCCATTAGAAAAGGGTGTTCTCTTAG
TGATGGCTAGGTTCAAAGAGATATTGGATATTAATCCTGTGGGAAGAAGG
GCTAGAGTTCAACCAGGTGTGAGGAATCTCGCAATTAGTCAGGCTGTTGC
ACCTCACAACCTTTATTACGCTCCTGATCCATCTTCACAAATCGCATGTT
CTATAGGTGGTAATGTGGCTGAAAACGCAGGAGGTGTTCATTGCCTTAAG
TACGGATTGACTGTGCACAACCTTTTGAAAATCGAAGTTCAGACTCTTGA
TGGAGAGGCTCTTACATTGGGTAGTGATGCATTGGATTCTCCTGGTTTTG
ATCTCTTAGCTCTCTTCACAGGTTCTGAAGGAATGTTAGGTGTTACTACA
GAGGTTACCGTTAAACTTTTGCCAAAACCTCCAGTTGCTAGAGTGCTCTT
AGCATCTTTTGATTCAGTGGAAAAAGCTGGACTTGCAGTTGGAGATATAA
TTGCTAACGGAATTATTCCTGGAGGTCTCGAAATGATGGATAACTTATCT
ATAAGAGCTGCTGAAGATTTCATTCATGCTGGATATCCAGTTGATGCTGA
GGCAATACTTTTGTGTGAACTTGATGGTGTTGAGTCAGATGTGCAAGAAG
ATTGCGAGAGAGTTAATGATATTCTCTTAAAGGCTGGAGCAACTGATGTG
AGGTTGGCTCAGGATGAAGCAGAGAGAGTTAGGTTTTGGGCTGGAAGAAA
AAACGCTTTCCCTGCTGTTGGTAGGATCTCACCAGATTATTACTGTATGG
ATGGTACAATACCTAGAAGGGCTCTCCCAGGAGTTTTAGAGGGTATTGCA
AGACTTAGTCAACAGTACGATTTGAGGGTTGCTAATGTGTTTCATGCAGG
AGATGGAAACATGCACCCTCTCATCTTATTTGATGCTAATGAGCCAGGAG
AGTTCGCTAGAGCAGAAGAGCTTGGAGGAAAGATTCTTGAACTTTGTGTT
GAAGTGGGAGGTAGTATCTCTGGTGAACATGGTATTGGAAGAGAGAAAAT
CAATCAAATGTGCGCTCAGTTCAACTCTGATGAAATCACCACTTTTCATG
CTGTTAAGGCTGCATTCGATCCTGATGGACTTTTGAATCCTGGAAAGAAT
ATACCAACATTGCACAGATGCGCTGAGTTCGGAGCAATGCACGTTCACCA
CGGACACCTTCCTTTTCCTGAGTTGGAGAGATTCTGA

It is known in the art that alternative splicing is an event that occurs frequently in all eukaryotic cells in which mRNA molecules are processed after having been transcribed from DNA (post-transcriptional modification, PTM). Overall, the processes result in the excision of particular regions of nucleic acid (introns) from the mRNA molecule.

It is important to note that while the mechanism of alternative splicing is generally understood, predicting alternative splicing events remains challenging and most of the understood systems have been investigated empirically. This includes the modules found in TriTag-1 and TriTag-2. Keeping this in mind, splice variants that may be afforded by TriTag-1 and TriTag-2 or other constructs prepared on the basis of this disclosure are not limited to those studied and described in Dinkins et al. (2008) and Reumann et al. (2007). For any given set of alternative splice signals, however, it is a straightforward matter to determine which products are formed, and hence, which localization signal will be attached to a given polypeptide via alternative splicing of a single RNA transcript.

It is known in the art that translocation of proteins to peroxisomes is mediated by a conserved sequence of amino acids. One such sequence is the peroxisomal targeting signal 2 (PTS2), generally found at the N-terminus of the protein expressed. The consensus sequence is as follows: Arg-Leu-X₅-His-Leu (SEQ ID NO: 5). As shown in Reumann et al (2007), the nucleic acid sequence, from which module 2 is obtained, is predicted to contain at least one functional alternative 3′ acceptor site, yielding at least two splice variants. One variant results in the translation of a peptide containing a functional PTS2, with the other variant lacking this signal. Module 2 can be necessary and sufficient for functional splicing and affording splice variants for either/both peroxisome targeting or cytoplasmic localization (i.e. containing no transit peptide).

It is known in the art that translocation of proteins to chloroplasts is mediated by particular amino acid sequences (chloroplast transit peptides, CTP) consisting primarily of hydrophobic side-chains at the N-terminus and a preference for hydroxylated amino acids (serine, threonine, etc), as exemplified by potato rubisco CTP.

As shown empirically by Dinkins et al. (2008), a splice variant of a nucleic acid sequence, from which module 1 is obtained, permits the localization of a GFP-tagged PIMT2 protein to the chloroplasts. Module 1 can be necessary and sufficient for functional splicing and affording splice variants for either/both of chloroplast targeting or cytoplasmic localization (i.e. containing no transit peptide).

It is known in the art that amino acids may be substituted by other amino acids having a similar hydropathic index or score and still result in a protein with similar biological activity, i.e., still obtain a biological functionally equivalent protein. In making such changes, the substitution of amino acids whose hydropathic indices are within +2 is preferred, those which are within +1 are particularly preferred, and those within +0.5 are even more particularly preferred.

It is also understood in the art that the substitution of like amino acids can be made effectively on the basis of hydrophilicity. U.S. Pat. No. 4,554,101 states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0.+−.1); glutamate (+3.0.+−.1); serine (+0.3); asparagine (+0.2); glutamine (+0.2); glycine (0); threonine (−0.4); proline (−0.5.+−.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent protein. In such changes, the substitution of amino acids whose hydrophilicity values are within +2 is preferred, those which are within +1 are particularly preferred, and those within +0.5 are even more particularly preferred. Exemplary substitutions which take these and various of the foregoing characteristics into consideration are well known to those of skill in the art and include: arginine and lysine; glutamate and aspartate; serine and threonine; glutamine and asparagine; and valine, leucine and isoleucine

TriTag-1 is composed of module 1 followed by module 2, in frame. This combination will afford functional splice variants expressing transit peptides with either/and PTS2 or/and CTP or/and no defined targeting signal (cytoplasmic localization). The predicted polypeptides are shown in FIG. 8. The following examples should be taken as such and should not limit the scope of the claimed invention. Splice variant BC-XZ will express a fused protein of interest with a CTP directing it towards the chloroplast. Splice variant AC-XY will express a fused protein of interest with a PTS2 directing it to the peroxisome. Splice variant AC-XZ will express a fused protein of interest without a transit peptide, which will localize in the cytoplasm. Splice variant BC-XY will express a fused protein of interest with a CTP along with PTS2; i.e. an ambiguous signal.

TriTag-2 is composed of module 2 followed by module 1, in frame. This combination will afford functional splice variants expressing transit peptides with either/and PTS2 or/and CTS or/and no defined targeting signal (cytoplasmic localization). The predicted polypeptides are shown in FIG. 9. The following examples should be taken as such and should not limit the scope of the claimed invention. Splice variant BC-XY will express a fused protein of interest with a CTP directing it towards the chloroplast. Splice variant AC-XZ will express a fused protein of interest with a PTS2 directing it to the peroxisome. Splice variant AC-XZ will express a fused protein of interest without a transit peptide, which will localize in the cytoplasm. Splice variant BC-XY will express a fused protein of interest with a CTP along with PTS2; i.e. an ambiguous signal.

TriTag-3 is a synthetic designed nucleic acid expressing an ambiguous transit peptide. It was designed by superimposing the consensus PTS2 sequence over the potato rubisco chloroplast transit peptide. The N-terminus of this ambiguous transit peptide will be sufficiently hydrophobic for chloroplast localization and the PTS2 signal amply recognized by its receptor, PEX7 resulting in peroxisomal localization. Without wishing to be bound by theory, there can be equilibrium between fused protein levels in the peroxisome and chloroplast as a result of the competition between organelles for the ambiguous signal. Furthermore, this push-and-pull mechanism can result in increased retention of the fused protein within the cytoplasm.

Further provided herein are methods for producing food, feed, or an industrial product comprising a plant containing a TriTag construct or a part of such a plant and preparing the food, feed, fiber, or industrial product from the plant or part thereof, wherein the food or feed is grain, meal, oil, starch, flour, or protein and the industrial product is biofuel, fiber, industrial chemicals, a pharmaceutical, or nutraceutical.

SEQ ID NO: 28 shows the nucleotide sequence of a DNA molecule encoding E. coli GDH subunit glcD codon optimized for A. thaliana expression with underlined the N-terminal triple-targeter sequence #1. SEQ ID NO: 29 shows the nucleotide sequence of a DNA molecule encoding E. coli GDH subunit glcE codon optimized for A. thaliana expression with underlined N-terminal triple-targeter sequence #1:

SEQ ID NO: 29
atggaggtatgttctcttgccaggaatctctgcttcagtttattctcaac
acataaggtatacaaatgggttatttggtgtttctctgtgttgtgtgact
gattttgtgcttatagacgatttttaatatgttgatggtgttagcaattc
cagagtggaactggctcgagcggcgacagctctagctctcctgtttcaac
aaaacctcaaggtatattgatgatttaccaaatcttttccttgtcaaagt
tttgtgtttgactgtgtgggtttgaacctgttaggattcagtatgatatc
aagtatgtgtcttttggaatacaaggatttaccatatggctatctttgtt
atctgtgtgaccttttctactttctcgctttgtaagatcgtctgagaatc
attggagggcatttgaatgttgcagctgaagcaATGCTCAGAGAATGCGA
TTATTCTCAGGCTCTTTTGGAGCAAGTGAATCAGGCAATTTCAGATAAGA
CTCCTCTTGTTATCCAAGGTTCTAACTCAAAGGCTTTTCTTGGTAGACCA
GTGACTGGACAGACACTTGATGTTAGATGTCATAGGGGTATCGTGAACTA
CGATCCTACTGAATTGGTTATAACAGCTAGAGTGGGAACCCCACTTGTTA
CTATTGAAGCTGCATTGGAGTCTGCTGGTCAAATGCTCCCATGTGAGCCT
CCACACTACGGAGAAGAGGCAACTTGGGGTGGTATGGTTGCTTGCGGACT
TGCAGGTCCTAGAAGGCCATGGAGTGGTTCTGTTAGAGATTTTGTGTTGG
GAACAAGGATTATCACCGGAGCTGGAAAGCATCTCAGATTCGGAGGTGAA
GTTATGAAAAATGTGGCAGGTTATGATCTCTCAAGGTTAATGGTTGGAAG
TTACGGTTGTCTTGGAGTGTTGACAGAAATTTCTATGAAGGTTCTTCCTA
GACCAAGGGCTTCACTTAGTTTGAGAAGGGAAATATCTTTGCAAGAGGCT
ATGTCAGAAATTGCAGAGTGGCAACTCCAGCCTTTACCAATTAGTGGATT
GTGCTATTTTGATAACGCTCTCTGGATCAGATTAGAAGGAGGAGAGGGTT
CAGTGAAAGCTGCAAGGGAACTCTTAGGAGGTGAAGAGGTTGCTGGACAG
TTCTGGCAACAGCTTAGAGAGCAACAGTTGCCTTTCTTTTCTCTTCCAGG
TACATTGTGGAGGATAAGTCTTCCTTCTGATGCTCCAATGATGGATCTCC
CTGGAGAACAATTAATCGATTGGGGAGGTGCTCTTAGATGGTTGAAGTCA
ACAGCAGAGGATAATCAGATCCATAGAATAGCTAGGAACGCAGGAGGTCA
CGCTACCAGATTTTCAGCAGGAGATGGAGGTTTCGCTCCTCTCAGTGCAC
CACTTTTTAGATACCACCAACAGTTGAAGCAGCAGTTAGATCCTTGTGGT
GTGTTCAATCCTGGAAGAATGTACGCTGAGTTGTGAATGCTCAGAGAATG
CGATTATTCTCAGGCTCTTTTGGAGCAAGTGAATCAGGCAATTTCAGATA
AGACTCCTCTTGTTATCCAAGGTTCTAACTCAAAGGCTTTTCTTGGTAGA
CCAGTGACTGGACAGACACTTGATGTTAGATGTCATAGGGGTATCGTGAA
CTACGATCCTACTGAATTGGTTATAACAGCTAGAGTGGGAACCCCACTTG
TTACTATTGAAGCTGCATTGGAGTCTGCTGGTCAAATGCTCCCATGTGAG
CCTCCACACTACGGAGAAGAGGCAACTTGGGGTGGTATGGTTGCTTGCGG
ACTTGCAGGTCCTAGAAGGCCATGGAGTGGTTCTGTTAGAGATTTTGTGT
TGGGAACAAGGATTATCACCGGAGCTGGAAAGCATCTCAGATTCGGAGGT
GAAGTTATGAAAAATGTGGCAGGTTATGATCTCTCAAGGTTAATGGTTGG
AAGTTACGGTTGTCTTGGAGTGTTGACAGAAATTTCTATGAAGGTTCTTC
CTAGACCAAGGGCTTCACTTAGTTTGAGAAGGGAAATATCTTTGCAAGAG
GCTATGTCAGAAATTGCAGAGTGGCAACTCCAGCCTTTACCAATTAGTGG
ATTGTGCTATTTTGATAACGCTCTCTGGATCAGATTAGAAGGAGGAGAGG
GTTCAGTGAAAGCTGCAAGGGAACTCTTAGGAGGTGAAGAGGTTGCTGGA
CAGTTCTGGCAACAGCTTAGAGAGCAACAGTTGCCTTTCTTTTCTCTTCC
AGGTACATTGTGGAGGATAAGTCTTCCTTCTGATGCTCCAATGATGGATC
TCCCTGGAGAACAATTAATCGATTGGGGAGGTGCTCTTAGATGGTTGAAG
TCAACAGCAGAGGATAATCAGATCCATAGAATAGCTAGGAACGCAGGAGG
TCACGCTACCAGATTTTCAGCAGGAGATGGAGGTTTCGCTCCTCTCAGTG
CACCACTTTTTAGATACCACCAACAGTTGAAGCAGCAGTTAGATCCTTGT
GGTGTGTTCAATCCTGGAAGAATGTACGCTGAGTTGTGA

SEQ ID NO: 30 shows the nucleotide sequence of a DNA molecule encoding E. coli GDH subunit glcF codon optimized for A. thaliana expression with underlined N-terminal triple-targeter sequence #1.

SEQ ID NO: 30
atggaggtatgttctcttgccaggaatctctgcttcagtttattctcaac
acataaggtatacaaatgggttatttggtgtttctctgtgttgtgtgact
gattttgtgcttatagacgatttttaatatgttgatggtgttagcaattc
cagagtggaactggctcgagcggcgacagctctagctctcctgtttcaac
aaaacctcaaggtatgatttaccaaatcttttccttgtcaaagttttgtg
tttgactgtgtgggtttgaacctgttaggattcagtatgatatcaagtat
gtgtcttttggaatacaaggatttaccatatggctatctttgttatctgt
gtgaccttttctactttctcgctttgtaagatcgtctgagaatcattgga
gggcatttgaatgttgcagctgaagcaATGCAAACTCAGCTTACAGAAGA
GATGAGACAAAATGCTAGGGCACTCGAAGCTGATTCTATCTTAAGAGCAT
GTGTTCATTGCGGATTCTGTACCGCTACTTGCCCTACTTATCAACTTTTG
GGAGATGAGCTTGATGGACCAAGAGGTAGAATATACCTCATTAAGCAAGT
TTTAGAAGGAAACGAGGTGACCTTGAAAACTCAGGAACATCTTGATAGAT
GCTTGACATGTAGGAATTGCGAGACTACATGTCCATCAGGAGTTAGGTAT
CACAACCTCTTAGATATCGGTAGAGATATAGTTGAACAGAAGGTGAAAAG
ACCTCTTCCAGAAAGAATACTCAGGGAGGGATTAAGACAAGTTGTGCCTA
GGCCAGCTGTGTTTAGAGCATTGACTCAAGTTGGTCTTGTGTTGAGGCCT
TTCCTTCCAGAACAGGTTAGAGCAAAGTTGCCTGCTGAAACAGTGAAGGC
TAAACCAAGACCTCCACTTAGGCATAAAAGAAGGGTTCTCATGTTAGAGG
GATGTGCTCAGCCTACTTTGTCTCCAAATACAAACGCTGCAACCGCTAGA
GTTCTTGATAGGTTGGGTATTTCAGTGATGCCTGCAAATGAGGCTGGATG
TTGCGGTGCTGTTGATTACCACCTCAACGCACAAGAGAAGGGATTAGCTA
GAGCAAGGAATAACATAGATGCTTGGTGGCCAGCAATTGAAGCTGGTGCA
GAGGCTATCCTTCAAACTGCTTCAGGATGCGGTGCATTTGTTAAGGAATA
TGGACAGATGCTTAAAAATGATGCATTGTACGCTGATAAGGCAAGACAAG
TGAGTGAACTTGCTGTTGATTTGGTGGAGCTTTTGAGAGAAGAGCCTCTT
GAAAAACTTGCTATAAGAGGAGATAAGAAATTGGCATTTCATTGTCCATG
CACACTTCAACACGCTCAGAAGTTGAACGGAGAAGTTGAGAAAGTGCTCT
TAAGACTCGGTTTCACATTAACCGATGTTCCTGATAGTCATCTCTGTTGC
GGATCTGCTGGTACTTATGCATTAACACACCCTGATCTTGCTAGACAGTT
GAGGGATAATAAGATGAACGCTCTCGAAAGTGGAAAACCTGAGATGATTG
TTACCGCTAATATCGGTTGTCAAACTCATTTGGCATCTGCTGGTAGGACC
TCTGTGAGGCACTGGATTGAGATCGTGGAACAGGCTCTTGAGAAGGAGTG
A

SEQ ID NO: 31 shows the nucleotide sequence of a DNA molecule encoding E. coli GDH subunit glcF codon optimized for A. thaliana expression with underlined N-terminal triple-targeter sequence #1 and underlined C-terminal myc epitope tag.

SEQ ID NO: 31
atggaggtatgttctcttgccaggaatctctgcttcagtttattctcaac
acataaggtatacaaatgggttatttggtgtttctctgtgttgtgtgact
gattttgtgcttatagacgatttttaatatgttgatggtgttagcaattc
cagagtggaactggctcgagcggcgacagctctagctctcctgtttcaac
aaaacctcaaggtatattgatgatttaccaaatcttttccttgtcaaagt
tttgtgtttgactgtgtgggtttgaacctgttaggattcagtatgatatc
aagtatgtgtcttttggaatacaaggatttaccatatggctatctttgtt
atctgtgtgaccttttctactttctcgctttgtaagatcgtctgagaatc
attggagggcatttgaatgttgcagctgaagcaATGCAAACTCAGCTTAC
AGAAGAGATGAGACAAAATGCTAGGGCACTCGAAGCTGATTCTATCTTAA
GAGCATGTGTTCATTGCGGATTCTGTACCGCTACTTGCCCTACTTATCAA
CTTTTGGGAGATGAGCTTGATGGACCAAGAGGTAGAATATACCTCATTAA
GCAAGTTTTAGAAGGAAACGAGGTGACCTTGAAAACTCAGGAACATCTTG
ATAGATGCTTGACATGTAGGAATTGCGAGACTACATGTCCATCAGGAGTT
AGGTATCACAACCTCTTAGATATCGGTAGAGATATAGTTGAACAGAAGGT
GAAAAGACCTCTTCCAGAAAGAATACTCAGGGAGGGATTAAGACAAGTTG
TGCCTAGGCCAGCTGTGTTTAGAGCATTGACTCAAGTTGGTCTTGTGTTG
AGGCCTTTCCTTCCAGAACAGGTTAGAGCAAAGTTGCCTGCTGAAACAGT
GAAGGCTAAACCAAGACCTCCACTTAGGCATAAAAGAAGGGTTCTCATGT
TAGAGGGATGTGCTCAGCCTACTTTGTCTCCAAATACAAACGCTGCAACC
GCTAGAGTTCTTGATAGGTTGGGTATTTCAGTGATGCCTGCAAATGAGGC
TGGATGTTGCGGTGCTGTTGATTACCACCTCAACGCACAAGAGAAGGGAT
TAGCTAGAGCAAGGAATAACATAGATGCTTGGTGGCCAGCAATTGAAGCT
GGTGCAGAGGCTATCCTTCAAACTGCTTCAGGATGCGGTGCATTTGTTAA
GGAATATGGACAGATGCTTAAAAATGATGCATTGTACGCTGATAAGGCAA
GACAAGTGAGTGAACTTGCTGTTGATTTGGTGGAGCTTTTGAGAGAAGAG
CCTCTTGAAAAACTTGCTATAAGAGGAGATAAGAAATTGGCATTTCATTG
TCCATGCACACTTCAACACGCTCAGAAGTTGAACGGAGAAGTTGAGAAAG
TGCTCTTAAGACTCGGTTTCACATTAACCGATGTTCCTGATAGTCATCTC
TGTTGCGGATCTGCTGGTACTTATGCATTAACACACCCTGATCTTGCTAG
ACAGTTGAGGGATAATAAGATGAACGCTCTCGAAAGTGGAAAACCTGAGA
TGATTGTTACCGCTAATATCGGTTGTCAAACTCATTTGGCATCTGCTGGT
AGGACCTCTGTGAGGCACTGGATTGAGATCGTGGAACAGGCTCTTGAGAA
GGAGgaacaaaaactcatctcagaagaggatcttTGA

SEQ ID NO:32 shows the nucleotide sequence of a DNA molecule encoding green fluorescent protein (GFP) codon optimized for A. thaliana expression with underlined N-terminal triple-targeter sequence #1.

SEQ ID NO: 32
atggaggtatgttctcttgccaggaatctctgcttcagtttattctcaac
acataaggtatacaaatgggttatttggtgtttctctgtgttgtgtgact
gattttgtgcttatagacgatttttaatatgttgatggtgttagcaattc
cagagtggaactggctcgagcggcgacagctctagctctcctgtttcaac
aaaacctcaaggtatgatttaccaaatcttttccttgtcaaagttttgtg
tttgactgtgtgggtttgaacctgttaggattcagtatgatatcaagtat
gtgtcttttggaatacaaggatttaccatatggctatctttgttatctgt
gtgaccttttctactttctcgctttgtaagatcgtctgagaatcattgga
gggcatttgaatgttgcagctgaagcaATGGCGAGTAAAGGAGAAGAACT
TTTCACTGGAGTTGTCCCAATTCTTGTTGAATTAGATGGTGATGTTAATG
GGCACAAATTTTCTGTCAGTGGAGAGGGTGAAGGTGATGCAACATACGGA
AAACTTACCCTTAAATTTATTTGCACTACTGGAAAACTACCTGTTCCTTG
GCCAACACTTGTCACTACTTTCTCTTATGGTGTTCAATGCTTTTCAAGAT
ACCCAGATCATATGAAGCGGCACGACTTCTTCAAGAGCGCCATGCCTGAG
GGATACGTGCAGGAGAGGACCATCTCTTTCAAGGACGACGGGAACTACAA
GACACGTGCTGAAGTCAAGTTTGAGGGAGACACCCTCGTCAACAGGATCG
AGCTTAAGGGAATTGATTTCAAGGAGGACGGAAACATCCTCGGCCACAAG
TTGGAATACAACTACAACTCCCACAACGTATACATCACGGCAGACAAACA
AAAGAATGGAATCAAAGCTAACTTCAAAATTAGACACAACATTGAAGATG
GAAGCGTTCAACTAGCAGACCATTATCAACAAAATACTCCTATTGGCGAT
GGCCCTGTCCTTTTACCAGACAACCATTACCTGTCCACACAATCTGCCCT
TTCGAAAGATCCCAACGAAAAGAGAGACCACATGGTCCTTCTTGAGTTTG
TAACAGCTGCTGGGATTACACATGGCATGGATGAACTATACAAATAA

SEQ ID NO: 33 shows the nucleotide sequence of a DNA molecule encoding E. coli GDH subunit glcD codon optimized for A. thaliana expression with underlined N-terminal triple-targeter sequence #2. SEQ ID NO: 34 shows the nucleotide sequence of a DNA molecule encoding E. coli GDH subunit glcE codon optimized for A. thaliana expression with underlined N-terminal triple-targeter sequence #2.

SEQ ID NO: 34
atggacagctctagctctcctgtttcaacaaaacctcaaggtatattgat
gatttaccaaatcttttccttgtcaaagttttgtgtttgactgtgtgggt
ttgaacctgttaggattcagtatgatatcaagtatgtgtcttttggaata
caaggatttacccttatggctatctttgttatctgtgtgaccttttctac
tttctcgctttgtaagatcgtctgagaatcattggagggcatttgaatgt
tgcagctgaagcaatggaggtatgttctcttgccaggaatctctgcttca
gtttattctcaacacataaggtatacaaatgggttatttggtgtttctct
gtgttgtgtgactgattttgtgcttatagacgatttttaatatgttgatg
gtgttagcaattccagagtggaactggctcgagcggcATGCTCAGAGAAT
GCGATTATTCTCAGGCTCTTTTGGAGCAAGTGAATCAGGCAATTTCAGAT
AAGACTCCTCTTGTTATCCAAGGTTCTAACTCAAAGGCTTTTCTTGGTAG
ACCAGTGACTGGACAGACACTTGATGTTAGATGTCATAGGGGTATCGTGA
ACTACGATCCTACTGAATTGGTTATAACAGCTAGAGTGGGAACCCCACTT
GTTACTATTGAAGCTGCATTGGAGTCTGCTGGTCAAATGCTCCCATGTGA
GCCTCCACACTACGGAGAAGAGGCAACTTGGGGTGGTATGGTTGCTTGCG
GACTTGCAGGTCCTAGAAGGCCATGGAGTGGTTCTGTTAGAGATTTTGTG
TTGGGAACAAGGATTATCACCGGAGCTGGAAAGCATCTCAGATTCGGAGG
TGAAGTTATGAAAAATGTGGCAGGTTATGATCTCTCAAGGTTAATGGTTG
GAAGTTACGGTTGTCTTGGAGTGTTGACAGAAATTTCTATGAAGGTTCTT
CCTAGACCAAGGGCTTCACTTAGTTTGAGAAGGGAAATATCTTTGCAAGA
GGCTATGTCAGAAATTGCAGAGTGGCAACTCCAGCCTTTACCAATTAGTG
GATTGTGCTATTTTGATAACGCTCTCTGGATCAGATTAGAAGGAGGAGAG
GGTTCAGTGAAAGCTGCAAGGGAACTCTTAGGAGGTGAAGAGGTTGCTGG
ACAGTTCTGGCAACAGCTTAGAGAGCAACAGTTGCCTTTCTTTTCTCTTC
CAGGTACATTGTGGAGGATAAGTCTTCCTTCTGATGCTCCAATGATGGAT
CTCCCTGGAGAACAATTAATCGATTGGGGAGGTGCTCTTAGATGGTTGAA
GTCAACAGCAGAGGATAATCAGATCCATAGAATAGCTAGGAACGCAGGAG
GTCACGCTACCAGATTTTCAGCAGGAGATGGAGGTTTCGCTCCTCTCAGT
GCACCACTTTTTAGATACCACCAACAGTTGAAGCAGCAGTTAGATCCTTG
TGGTGTGTTCAATCCTGGAAGAATGTACGCTGAGTTGTGAATGCTCAGAG
AATGCGATTATTCTCAGGCTCTTTTGGAGCAAGTGAATCAGGCAATTTCA
GATAAGACTCCTCTTGTTATCCAAGGTTCTAACTCAAAGGCTTTTCTTGG
TAGACCAGTGACTGGACAGACACTTGATGTTAGATGTCATAGGGGTATCG
TGAACTACGATCCTACTGAATTGGTTATAACAGCTAGAGTGGGAACCCCA
CTTGTTACTATTGAAGCTGCATTGGAGTCTGCTGGTCAAATGCTCCCATG
TGAGCCTCCACACTACGGAGAAGAGGCAACTTGGGGTGGTATGGTTGCTT
GCGGACTTGCAGGTCCTAGAAGGCCATGGAGTGGTTCTGTTAGAGATTTT
GTGTTGGGAACAAGGATTATCACCGGAGCTGGAAAGCATCTCAGATTCGG
AGGTGAAGTTATGAAAAATGTGGCAGGTTATGATCTCTCAAGGTTAATGG
TTGGAAGTTACGGTTGTCTTGGAGTGTTGACAGAAATTTCTATGAAGGTT
CTTCCTAGACCAAGGGCTTCACTTAGTTTGAGAAGGGAAATATCTTTGCA
AGAGGCTATGTCAGAAATTGCAGAGTGGCAACTCCAGCCTTTACCAATTA
GTGGATTGTGCTATTTTGATAACGCTCTCTGGATCAGATTAGAAGGAGGA
GAGGGTTCAGTGAAAGCTGCAAGGGAACTCTTAGGAGGTGAAGAGGTTGC
TGGACAGTTCTGGCAACAGCTTAGAGAGCAACAGTTGCCTTTCTTTTCTC
TTCCAGGTACATTGTGGAGGATAAGTCTTCCTTCTGATGCTCCAATGATG
GATCTCCCTGGAGAACAATTAATCGATTGGGGAGGTGCTCTTAGATGGTT
GAAGTCAACAGCAGAGGATAATCAGATCCATAGAATAGCTAGGAACGCAG
GAGGTCACGCTACCAGATTTTCAGCAGGAGATGGAGGTTTCGCTCCTCTC
AGTGCACCACTTTTTAGATACCACCAACAGTTGAAGCAGCAGTTAGATCC
TTGTGGTGTGTTCAATCCTGGAAGAATGTACGCTGAGTTGTGA

SEQ ID NO: 35 shows the nucleotide sequence of a DNA molecule encoding E. coli GDH subunit glcF codon optimized for A. thaliana expression with underlined N-terminal triple-targeter sequence #2.

SEQ ID NO: 35
atggacagctctagctctcctgtttcaacaaaacctcaaggtatattgat
gatttaccaaatcttttccttgtcaaagttttgtgtttgactgtgtgggt
ttgaacctgttaggattcagtatgatatcaagtatgtgtcttttggaata
caaggatttacccttatggctatctttgttatctgtgtgaccttttctac
tttctcgctttgtaagatcgtctgagaatcattggagggcatttgaatgt
tgcagctgaagcaatggaggtatgttctcttgccaggaatctctgcttca
gtttattctcaacacataaggtatacaaatgggttatttggtgtttctct
gtgttgtgtgactgattttgtgcttatagacgatttttaatatgttgatg
gtgttagcaattccagagtggaactggctcgagcggcATGCAAACTCAGC
TTACAGAAGAGATGAGACAAAATGCTAGGGCACTCGAAGCTGATTCTATC
TTAAGAGCATGTGTTCATTGCGGATTCTGTACCGCTACTTGCCCTACTTA
TCAACTTTTGGGAGATGAGCTTGATGGACCAAGAGGTAGAATATACCTCA
TTAAGCAAGTTTTAGAAGGAAACGAGGTGACCTTGAAAACTCAGGAACAT
CTTGATAGATGCTTGACATGTAGGAATTGCGAGACTACATGTCCATCAGG
AGTTAGGTATCACAACCTCTTAGATATCGGTAGAGATATAGTTGAACAGA
AGGTGAAAAGACCTCTTCCAGAAAGAATACTCAGGGAGGGATTAAGACAA
GTTGTGCCTAGGCCAGCTGTGTTTAGAGCATTGACTCAAGTTGGTCTTGT
GTTGAGGCCTTTCCTTCCAGAACAGGTTAGAGCAAAGTTGCCTGCTGAAA
CAGTGAAGGCTAAACCAAGACCTCCACTTAGGCATAAAAGAAGGGTTCTC
ATGTTAGAGGGATGTGCTCAGCCTACTTTGTCTCCAAATACAAACGCTGC
AACCGCTAGAGTTCTTGATAGGTTGGGTATTTCAGTGATGCCTGCAAATG
AGGCTGGATGTTGCGGTGCTGTTGATTACCACCTCAACGCACAAGAGAAG
GGATTAGCTAGAGCAAGGAATAACATAGATGCTTGGTGGCCAGCAATTGA
AGCTGGTGCAGAGGCTATCCTTCAAACTGCTTCAGGATGCGGTGCATTTG
TTAAGGAATATGGACAGATGCTTAAAAATGATGCATTGTACGCTGATAAG
GCAAGACAAGTGAGTGAACTTGCTGTTGATTTGGTGGAGCTTTTGAGAGA
AGAGCCTCTTGAAAAACTTGCTATAAGAGGAGATAAGAAATTGGCATTTC
ATTGTCCATGCACACTTCAACACGCTCAGAAGTTGAACGGAGAAGTTGAG
AAAGTGCTCTTAAGACTCGGTTTCACATTAACCGATGTTCCTGATAGTCA
TCTCTGTTGCGGATCTGCTGGTACTTATGCATTAACACACCCTGATCTTG
CTAGACAGTTGAGGGATAATAAGATGAACGCTCTCGAAAGTGGAAAACCT
GAGATGATTGTTACCGCTAATATCGGTTGTCAAACTCATTTGGCATCTGC
TGGTAGGACCTCTGTGAGGCACTGGATTGAGATCGTGGAACAGGCTCTTG
AGAAGGAGTGA

SEQ ID NO: 36 shows the nucleotide sequence of a DNA molecule encoding E. coli GDH subunit glcF codon optimized for A. thaliana expression with underlined N-terminal triple-targeter sequence #2 and underlined C-terminal myc epitope tag.

SEQ ID NO: 36
atggacagctctagctctcctgtttcaacaaaacctcaaggtatattgat
gatttaccaaatcttttccttgtcaaagttttgtgtttgactgtgtgggt
ttgaacctgttaggattcagtatgatatcaagtatgtgtcttttggaata
caaggatttacccttatggctatctttgttatctgtgtgaccttttctac
tttctcgctttgtaagatcgtctgagaatcattggagggcatttgaatgt
tgcagctgaagcaatggaggtatgttctcttgccaggaatctctgcttca
gtttattctcaacacataaggtatacaaatgggttatttggtgtttctct
gtgttgtgtgactgattttgtgcttatagacgatttttaatatgttgatg
gtgttagcaattccagaggtgaactggctcgagcggcATGCAAACTCAGC
TTACAGAAGAGATGAGACAAAATGCTAGGGCACTCGAAGCTGATTCTATC
TTAAGAGCATGTGTTCATTGCGGATTCTGTACCGCTACTTGCCCTACTTA
TCAACTTTTGGGAGATGAGCTTGATGGACCAAGAGGTAGAATATACCTCA
TTAAGCAAGTTTTAGAAGGAAACGAGGTGACCTTGAAAACTCAGGAACAT
CTTGATAGATGCTTGACATGTAGGAATTGCGAGACTACATGTCCATCAGG
AGTTAGGTATCACAACCTCTTAGATATCGGTAGAGATATAGTTGAACAGA
AGGTGAAAAGACCTCTTCCAGAAAGAATACTCAGGGAGGGATTAAGACAA
GTTGTGCCTAGGCCAGCTGTGTTTAGAGCATTGACTCAAGTTGGTCTTGT
GTTGAGGCCTTTCCTTCCAGAACAGGTTAGAGCAAAGTTGCCTGCTGAAA
CAGTGAAGGCTAAACCAAGACCTCCACTTAGGCATAAAAGAAGGGTTCTC
ATGTTAGAGGGATGTGCTCAGCCTACTTTGTCTCCAAATACAAACGCTGC
AACCGCTAGAGTTCTTGATAGGTTGGGTATTTCAGTGATGCCTGCAAATG
AGGCTGGATGTTGCGGTGCTGTTGATTACCACCTCAACGCACAAGAGAAG
GGATTAGCTAGAGCAAGGAATAACATAGATGCTTGGTGGCCAGCAATTGA
AGCTGGTGCAGAGGCTATCCTTCAAACTGCTTCAGGATGCGGTGCATTTG
TTAAGGAATATGGACAGATGCTTAAAAATGATGCATTGTACGCTGATAAG
GCAAGACAAGTGAGTGAACTTGCTGTTGATTTGGTGGAGCTTTTGAGAGA
AGAGCCTCTTGAAAAACTTGCTATAAGAGGAGATAAGAAATTGGCATTTC
ATTGTCCATGCACACTTCAACACGCTCAGAAGTTGAACGGAGAAGTTGAG
AAAGTGCTCTTAAGACTCGGTTTCACATTAACCGATGTTCCTGATAGTCA
TCTCTGTTGCGGATCTGCTGGTACTTATGCATTAACACACCCTGATCTTG
CTAGACAGTTGAGGGATAATAAGATGAACGCTCTCGAAAGTGGAAAACCT
GAGATGATTGTTACCGCTAATATCGGTTGTCAAACTCATTTGGCATCTGC
TGGTAGGACCTCTGTGAGGCACTGGATTGAGATCGTGGAACAGGCTCTTG
AGAAGGAGgaacaaaaactcatctcagaagaggatcttTGA

SEQ ID NO: 37 shows the nucleotide sequence of a DNA molecule encoding green fluorescent protein (GFP) codon optimized for A. thaliana expression with underlined N-terminal triple-targeter sequence #2.

SEQ ID NO: 37
atggacagctctagctctcctgtttcaacaaaacctcaaggtatattgat
gatttaccaaatcttttccttgtcaaagttttgtgtttgactgtgtgggt
ttgaacctgttaggattcagtatgatatcaagtatgtgtcttttggaata
caaggatttacccttatggctatctttgttatctgtgtgaccttttctac
tttctcgctttgtaagatcgtctgagaatcattggagggcatttgaatgt
tgcagctgaagcaatggaggtatgttctcttgccaggaatctctgcttca
gtttattctcaacacataaggtatacaaatgggttatttggtgtttctct
gtgttgtgtgactgattttgtgcttatagacgatttttaatatgttgatg
gtgttagcaattccagagtggaactggctcgagcggcATGGCGAGTAAAG
GAGAAGAACTTTTCACTGGAGTTGTCCCAATTCTTGTTGAATTAGATGGT
GATGTTAATGGGCACAAATTTTCTGTCAGTGGAGAGGGTGAAGGTGATGC
AACATACGGAAAACTTACCCTTAAATTTATTTGCACTACTGGAAAACTAC
CTGTTCCTTGGCCAACACTTGTCACTACTTTCTCTTATGGTGTTCAATGC
TTTTCAAGATACCCAGATCATATGAAGCGGCACGACTTCTTCAAGAGCGC
CATGCCTGAGGGATACGTGCAGGAGAGGACCATCTCTTTCAAGGACGACG
GGAACTACAAGACACGTGCTGAAGTCAAGTTTGAGGGAGACACCCTCGTC
AACAGGATCGAGCTTAAGGGAATTGATTTCAAGGAGGACGGAAACATCCT
CGGCCACAAGTTGGAATACAACTACAACTCCCACAACGTATACATCACGG
CAGACAAACAAAAGAATGGAATCAAAGCTAACTTCAAAATTAGACACAAC
ATTGAAGATGGAAGCGTTCAACTAGCAGACCATTATCAACAAAATACTCC
TATTGGCGATGGCCCTGTCCTTTTACCAGACAACCATTACCTGTCCACAC
AATCTGCCCTTTCGAAAGATCCCAACGAAAAGAGAGACCACATGGTCCTT
CTTGAGTTTGTAACAGCTGCTGGGATTACACATGGCATGGATGAACTATA
CAAATAA

SEQ ID NO: 38 shows the nucleotide sequence of a DNA molecule encoding E. coli GDH subunit glcD codon optimized for A. thaliana expression with underlined N-terminal triple-targeter sequence #3.

SEQ ID NO: 38
atggcttcctctgttatttcctctgccgctgttgctacacgcaccaatgt
tacacaagctggcagcatgattgcacctttcactggtctcaaatctgctg
ctactaccctgtttcaaggcttagagttctttctgctcatttgatcactt
ccattgctagcaatggtggaagagttaggtgcATGTCTATTCTTTATGAA
GAGAGACTCGATGGAGCTTTACCAGATGTTGATAGAACCTCAGTGCTCAT
GGCATTAAGGGAACATGTTCCTGGACTTGAAATTCTTCACACAGATGAAG
AGATTATCCCATATGAATGTGATGGTTTGTCTGCTTACAGAACTAGGCCT
CTTTTGGTTGTGCTCCCAAAGCAGATGGAACAGGTTACAGCTATTCTTGC
AGTGTGCCATAGATTGAGGGTTCCTGTTGTGACAAGAGGAGCTGGTACCG
GACTTTCAGGAGGTGCACTCCCATTAGAAAAGGGTGTTCTCTTAGTGATG
GCTAGGTTCAAAGAGATATTGGATATTAATCCTGTGGGAAGAAGGGCTAG
AGTTCAACCAGGTGTGAGGAATCTCGCAATTAGTCAGGCTGTTGCACCTC
ACAACCTTTATTACGCTCCTGATCCATCTTCACAAATCGCATGTTCTATA
GGTGGTAATGTGGCTGAAAACGCAGGAGGTGTTCATTGCCTTAAGTACGG
ATTGACTGTGCACAACCTTTTGAAAATCGAAGTTCAGACTCTTGATGGAG
AGGCTCTTACATTGGGTAGTGATGCATTGGATTCTCCTGGTTTTGATCTC
TTAGCTCTCTTCACAGGTTCTGAAGGAATGTTAGGTGTTACTACAGAGGT
TACCGTTAAACTTTTGCCAAAACCTCCAGTTGCTAGAGTGCTCTTAGCAT
CTTTTGATTCAGTGGAAAAAGCTGGACTTGCAGTTGGAGATATAATTGCT
AACGGAATTATTCCTGGAGGTCTCGAAATGATGGATAACTTATCTATAAG
AGCTGCTGAAGATTTCATTCATGCTGGATATCCAGTTGATGCTGAGGCAA
TACTTTTGTGTGAACTTGATGGTGTTGAGTCAGATGTGCAAGAAGATTGC
GAGAGAGTTAATGATATTCTCTTAAAGGCTGGAGCAACTGATGTGAGGTT
GGCTCAGGATGAAGCAGAGAGAGTTAGGTTTTGGGCTGGAAGAAAAAACG
CTTTCCCTGCTGTTGGTAGGATCTCACCAGATTATTACTGTATGGATGGT
ACAATACCTAGAAGGGCTCTCCCAGGAGTTTTAGAGGGTATTGCAAGACT
TAGTCAACAGTACGATTTGAGGGTTGCTAATGTGTTTCATGCAGGAGATG
GAAACATGCACCCTCTCATCTTATTTGATGCTAATGAGCCAGGAGAGTTC
GCTAGAGCAGAAGAGCTTGGAGGAAAGATTCTTGAACTTTGTGTTGAAGT
GGGAGGTAGTATCTCTGGTGAACATGGTATTGGAAGAGAGAAAATCAATC
AAATGTGCGCTCAGTTCAACTCTGATGAAATCACCACTTTTCATGCTGTT
AAGGCTGCATTCGATCCTGATGGACTTTTGAATCCTGGAAAGAATATACC
AACATTGCACAGATGCGCTGAGTTCGGAGCAATGCACGTTCACCACGGAC
ACCTTCCTTTTCCTGAGTTGGAGAGATTCTGA

SEQ ID NO: 39 shows the nucleotide sequence of a DNA molecule encoding E. coli GDH subunit glcE codon optimized for A. thaliana expression with underlined N-terminal triple-targeter sequence #3.

SEQ ID NO: 39
atggcttcctctgttatttcctctgccgctgttgctacacgcaccaatgt
tacacaagctggcagcatgattgcacctttcactggtctcaaatctgctg
ctactaccctgtttcaaggcttagagttctttctgctcatttgatcactt
ccattgctagcaatggtggaagagttaggtgcATGCTCAGAGAATGCGAT
TATTCTCAGGCTCTTTTGGAGCAAGTGAATCAGGCAATTTCAGATAAGAC
TCCTCTTGTTATCCAAGGTTCTAACTCAAAGGCTTTTCTTGGTAGACCAG
TGACTGGACAGACACTTGATGTTAGATGTCATAGGGGTATCGTGAACTAC
GATCCTACTGAATTGGTTATAACAGCTAGAGTGGGAACCCCACTTGTTAC
TATTGAAGCTGCATTGGAGTCTGCTGGTCAAATGCTCCCATGTGAGCCTC
CACACTACGGAGAAGAGGCAACTTGGGGTGGTATGGTTGCTTGCGGACTT
GCAGGTCCTAGAAGGCCATGGAGTGGTTCTGTTAGAGATTTTGTGTTGGG
AACAAGGATTATCACCGGAGCTGGAAAGCATCTCAGATTCGGAGGTGAAG
TTATGAAAAATGTGGCAGGTTATGATCTCTCAAGGTTAATGGTTGGAAGT
TACGGTTGTCTTGGAGTGTTGACAGAAATTTCTATGAAGGTTCTTCCTAG
ACCAAGGGCTTCACTTAGTTTGAGAAGGGAAATATCTTTGCAAGAGGCTA
TGTCAGAAATTGCAGAGTGGCAACTCCAGCCTTTACCAATTAGTGGATTG
TGCTATTTTGATAACGCTCTCTGGATCAGATTAGAAGGAGGAGAGGGTTC
AGTGAAAGCTGCAAGGGAACTCTTAGGAGGTGAAGAGGTTGCTGGACAGT
TCTGGCAACAGCTTAGAGAGCAACAGTTGCCTTTCTTTTCTCTTCCAGGT
ACATTGTGGAGGATAAGTCTTCCTTCTGATGCTCCAATGATGGATCTCCC
TGGAGAACAATTAATCGATTGGGGAGGTGCTCTTAGATGGTTGAAGTCAA
CAGCAGAGGATAATCAGATCCATAGAATAGCTAGGAACGCAGGAGGTCAC
GCTACCAGATTTTCAGCAGGAGATGGAGGTTTCGCTCCTCTCAGTGCACC
ACTTTTTAGATACCACCAACAGTTGAAGCAGCAGTTAGATCCTTGTGGTG
TGTTCAATCCTGGAAGAATGTACGCTGAGTTGTGAATGCTCAGAGAATGC
GATTATTCTCAGGCTCTTTTGGAGCAAGTGAATCAGGCAATTTCAGATAA
GACTCCTCTTGTTATCCAAGGTTCTAACTCAAAGGCTTTTCTTGGTAGAC
CAGTGACTGGACAGACACTTGATGTTAGATGTCATAGGGGTATCGTGAAC
TACGATCCTACTGAATTGGTTATAACAGCTAGAGTGGGAACCCCACTTGT
TACTATTGAAGCTGCATTGGAGTCTGCTGGTCAAATGCTCCCATGTGAGC
CTCCACACTACGGAGAAGAGGCAACTTGGGGTGGTATGGTTGCTTGCGGA
CTTGCAGGTCCTAGAAGGCCATGGAGTGGTTCTGTTAGAGATTTTGTGTT
GGGAACAAGGATTATCACCGGAGCTGGAAAGCATCTCAGATTCGGAGGTG
AAGTTATGAAAAATGTGGCAGGTTATGATCTCTCAAGGTTAATGGTTGGA
AGTTACGGTTGTCTTGGAGTGTTGACAGAAATTTCTATGAAGGTTCTTCC
TAGACCAAGGGCTTCACTTAGTTTGAGAAGGGAAATATCTTTGCAAGAGG
CTATGTCAGAAATTGCAGAGTGGCAACTCCAGCCTTTACCAATTAGTGGA
TTGTGCTATTTTGATAACGCTCTCTGGATCAGATTAGAAGGAGGAGAGGG
TTCAGTGAAAGCTGCAAGGGAACTCTTAGGAGGTGAAGAGGTTGCTGGAC
AGTTCTGGCAACAGCTTAGAGAGCAACAGTTGCCTTTCTTTTCTCTTCCA
GGTACATTGTGGAGGATAAGTCTTCCTTCTGATGCTCCAATGATGGATCT
CCCTGGAGAACAATTAATCGATTGGGGAGGTGCTCTTAGATGGTTGAAGT
CAACAGCAGAGGATAATCAGATCCATAGAATAGCTAGGAACGCAGGAGGT
CACGCTACCAGATTTTCAGCAGGAGATGGAGGTTTCGCTCCTCTCAGTGC
ACCACTTTTTAGATACCACCAACAGTTGAAGCAGCAGTTAGATCCTTGTG
GTGTGTTCAATCCTGGAAGAATGTACGCTGAGTTGTGA

SEQ ID NO: 40 shows the nucleotide sequence of a DNA molecule encoding E. coli GDH subunit glcF codon optimized for A. thaliana expression with underlined N-terminal triple-targeter sequence #3.

SEQ ID NO: 40
atggcttcctctgttatttcctctgccgctgttgctacacgcaccaatgt
tacacaagctggcagcatgattgcacctttcactggtctcaaatctgctg
ctactaccctgtttcaaggcttagagttctttctgctcatttgatcactt
ccattgctagcaatggtggaagagttaggtgcATGCAAACTCAGCTTACA
GAAGAGATGAGACAAAATGCTAGGGCACTCGAAGCTGATTCTATCTTAAG
AGCATGTGTTCATTGCGGATTCTGTACCGCTACTTGCCCTACTTATCAAC
TTTTGGGAGATGAGCTTGATGGACCAAGAGGTAGAATATACCTCATTAAG
CAAGTTTTAGAAGGAAACGAGGTGACCTTGAAAACTCAGGAACATCTTGA
TAGATGCTTGACATGTAGGAATTGCGAGACTACATGTCCATCAGGAGTTA
GGTATCACAACCTCTTAGATATCGGTAGAGATATAGTTGAACAGAAGGTG
AAAAGACCTCTTCCAGAAAGAATACTCAGGGAGGGATTAAGACAAGTTGT
GCCTAGGCCAGCTGTGTTTAGAGCATTGACTCAAGTTGGTCTTGTGTTGA
GGCCTTTCCTTCCAGAACAGGTTAGAGCAAAGTTGCCTGCTGAAACAGTG
AAGGCTAAACCAAGACCTCCACTTAGGCATAAAAGAAGGGTTCTCATGTT
AGAGGGATGTGCTCAGCCTACTTTGTCTCCAAATACAAACGCTGCAACCG
CTAGAGTTCTTGATAGGTTGGGTATTTCAGTGATGCCTGCAAATGAGGCT
GGATGTTGCGGTGCTGTTGATTACCACCTCAACGCACAAGAGAAGGGATT
AGCTAGAGCAAGGAATAACATAGATGCTTGGTGGCCAGCAATTGAAGCTG
GTGCAGAGGCTATCCTTCAAACTGCTTCAGGATGCGGTGCATTTGTTAAG
GAATATGGACAGATGCTTAAAAATGATGCATTGTACGCTGATAAGGCAAG
ACAAGTGAGTGAACTTGCTGTTGATTTGGTGGAGCTTTTGAGAGAAGAGC
CTCTTGAAAAACTTGCTATAAGAGGAGATAAGAAATTGGCATTTCATTGT
CCATGCACACTTCAACACGCTCAGAAGTTGAACGGAGAAGTTGAGAAAGT
GCTCTTAAGACTCGGTTTCACATTAACCGATGTTCCTGATAGTCATCTCT
GTTGCGGATCTGCTGGTACTTATGCATTAACACACCCTGATCTTGCTAGA
CAGTTGAGGGATAATAAGATGAACGCTCTCGAAAGTGGAAAACCTGAGAT
GATTGTTACCGCTAATATCGGTTGTCAAACTCATTTGGCATCTGCTGGTA
GGACCTCTGTGAGGCACTGGATTGAGATCGTGGAACAGGCTCTTGAGAAG
GAGTGA

SEQ ID NO: 41 shows the nucleotide sequence of a DNA molecule encoding E. coli GDH subunit glcF codon optimized for A. thaliana expression with underlined N-terminal triple-targeter sequence #3 and underlined C-terminal myc epitope tag.

SEQ ID NO: 41
atggcttcctctgttatttcctctgccgctgttgctacacgcaccaatgt
tacacaagctggcagcatgattgcacctttcactggtctcaaatctgctg
ctactaccctgtttcaaggcttagagttctttctgctcatttgatcactt
ccattgctagcaatggtggaagagttaggtgcATGCAAACTCAGCTTACA
GAAGAGATGAGACAAAATGCTAGGGCACTCGAAGCTGATTCTATCTTAAG
AGCATGTGTTCATTGCGGATTCTGTACCGCTACTTGCCCTACTTATCAAC
TTTTGGGAGATGAGCTTGATGGACCAAGAGGTAGAATATACCTCATTAAG
CAAGTTTTAGAAGGAAACGAGGTGACCTTGAAAACTCAGGAACATCTTGA
TAGATGCTTGACATGTAGGAATTGCGAGACTACATGTCCATCAGGAGTTA
GGTATCACAACCTCTTAGATATCGGTAGAGATATAGTTGAACAGAAGGTG
AAAAGACCTCTTCCAGAAAGAATACTCAGGGAGGGATTAAGACAAGTTGT
GCCTAGGCCAGCTGTGTTTAGAGCATTGACTCAAGTTGGTCTTGTGTTGA
GGCCTTTCCTTCCAGAACAGGTTAGAGCAAAGTTGCCTGCTGAAACAGTG
AAGGCTAAACCAAGACCTCCACTTAGGCATAAAAGAAGGGTTCTCATGTT
AGAGGGATGTGCTCAGCCTACTTTGTCTCCAAATACAAACGCTGCAACCG
CTAGAGTTCTTGATAGGTTGGGTATTTCAGTGATGCCTGCAAATGAGGCT
GGATGTTGCGGTGCTGTTGATTACCACCTCAACGCACAAGAGAAGGGATT
AGCTAGAGCAAGGAATAACATAGATGCTTGGTGGCCAGCAATTGAAGCTG
GTGCAGAGGCTATCCTTCAAACTGCTTCAGGATGCGGTGCATTTGTTAAG
GAATATGGACAGATGCTTAAAAATGATGCATTGTACGCTGATAAGGCAAG
ACAAGTGAGTGAACTTGCTGTTGATTTGGTGGAGCTTTTGAGAGAAGAGC
CTCTTGAAAAACTTGCTATAAGAGGAGATAAGAAATTGGCATTTCATTGT
CCATGCACACTTCAACACGCTCAGAAGTTGAACGGAGAAGTTGAGAAAGT
GCTCTTAAGACTCGGTTTCACATTAACCGATGTTCCTGATAGTCATCTCT
GTTGCGGATCTGCTGGTACTTATGCATTAACACACCCTGATCTTGCTAGA
CAGTTGAGGGATAATAAGATGAACGCTCTCGAAAGTGGAAAACCTGAGAT
GATTGTTACCGCTAATATCGGTTGTCAAACTCATTTGGCATCTGCTGGTA
GGACCTCTGTGAGGCACTGGATTGAGATCGTGGAACAGGCTCTTGAGAAG
GAGgaacaaaaactcatctcagaagaggatcttTGA

SEQ ID NO: 42 shows the nucleotide sequence of a DNA molecule encoding green fluorescent protein (GFP) codon optimized for A. thaliana expression with underlined N-terminal triple-targeter sequence #3.

SEQ ID NO: 42
atggcttcctctgttatttcctctgccgctgttgctacacgcaccaatgt
tacacaagctggcagcatgattgcacctttcactggtctcaaatctgctg
ctactaccctgtttcaaggcttagagttctttctgctcatttgatcactt
ccattgctagcaatggtggaagagttaggtgcATGGCGAGTAAAGGAGAA
GAACTTTTCACTGGAGTTGTCCCAATTCTTGTTGAATTAGATGGTGATGT
TAATGGGCACAAATTTTCTGTCAGTGGAGAGGGTGAAGGTGATGCAACAT
ACGGAAAACTTACCCTTAAATTTATTTGCACTACTGGAAAACTACCTGTT
CCTTGGCCAACACTTGTCACTACTTTCTCTTATGGTGTTCAATGCTTTTC
AAGATACCCAGATCATATGAAGCGGCACGACTTCTTCAAGAGCGCCATGC
CTGAGGGATACGTGCAGGAGAGGACCATCTCTTTCAAGGACGACGGGAAC
TACAAGACACGTGCTGAAGTCAAGTTTGAGGGAGACACCCTCGTCAACAG
GATCGAGCTTAAGGGAATTGATTTCAAGGAGGACGGAAACATCCTCGGCC
ACAAGTTGGAATACAACTACAACTCCCACAACGTATACATCACGGCAGAC
AAACAAAAGAATGGAATCAAAGCTAACTTCAAAATTAGACACAACATTGA
AGATGGAAGCGTTCAACTAGCAGACCATTATCAACAAAATACTCCTATTG
GCGATGGCCCTGTCCTTTTACCAGACAACCATTACCTGTCCACACAATCT
GCCCTTTCGAAAGATCCCAACGAAAAGAGAGACCACATGGTCCTTCTTGA
GTTTGTAACAGCTGCTGGGATTACACATGGCATGGATGAACTATACAAAT
AA

SEQ ID NO: 43 shows the amino acid sequence of splice variant AC-XZ of triple-targeter #1 fused to the amino acid sequence of E. coli GDH subunit #1. SEQ ID NO: 44 shows the amino acid sequence of splice variant AC-XZ of triple-targeter #1 fused to the amino acid sequence of E. coli GDH subunit #2. SEQ ID NO: 45 shows the amino acid sequence of splice variant AC-XZ of triple-targeter #1 fused to the amino acid sequence of E. coli GDH subunit #3. SEQ ID NO: 46 shows the amino acid sequence of splice variant AC-XZ of triple-targeter #1 fused to the amino acid sequence of E. coli GDH subunit #3 with myc-epitope tag. SEQ ID NO: 47 shows the amino acid sequence of splice variant AC-XZ of triple-targeter #1 fused to the amino acid sequence of Green Fluorescent Protein (GFP). SEQ ID NO: 48 shows the amino acid sequence of splice variant BC-XZ of triple-targeter #1 fused to the amino acid sequence of E. coli GDH subunit #1. SEQ ID NO: 49 shows the amino acid sequence of splice variant BC-XZ of triple-targeter #1 fused to the amino acid sequence of E. coli GDH subunit #2. SEQ ID NO: 50 shows the amino acid sequence of splice variant BC-XZ of triple-targeter #1 fused to the amino acid sequence of E. coli GDH subunit #3. SEQ ID NO: 51 shows the amino acid sequence of splice variant BC-XZ of triple-targeter #1 fused to the amino acid sequence of E. coli GDH subunit #3 with myc epitope tag. SEQ ID NO: 52 shows the amino acid sequence of splice variant BC-XZ of triple-targeter #1 fused to the amino acid sequence of GFP. SEQ ID NO: 53 shows the amino acid sequence of splice variant AC-XY of triple-targeter #1 fused to the amino acid sequence of E. coli GDH subunit #1. SEQ ID NO: 54 shows the amino acid sequence of splice variant AC-XY of triple-targeter #1 fused to the amino acid sequence of E. coli GDH subunit #2. SEQ ID NO: 55 shows the amino acid sequence of splice variant AC-XY of triple-targeter #1 fused to the amino acid sequence of E. coli GDH subunit #3. SEQ ID NO: 56 shows the amino acid sequence of splice variant AC-XY of triple-targeter #1 fused to the amino acid sequence of E. coli GDH subunit #3 with myc epitope tag. SEQ ID NO: 57 shows the amino acid sequence of splice variant AC-XY of triple-targeter #1 fused to the amino acid sequence of GFP. SEQ ID NO: 58 shows the amino acid sequence of splice variant BC-XY of triple-targeter #1 fused to the amino acid sequence of E. coli GDH subunit #1. SEQ ID NO: 59 shows the amino acid sequence of splice variant BC-XY of triple-targeter #1 fused to the amino acid sequence of E. coli GDH subunit #2. SEQ ID NO: 60 shows the amino acid sequence of splice variant BC-XY of triple-targeter #1 fused to the amino acid sequence of E. coli GDH subunit #3. SEQ ID NO: 61 shows the amino acid sequence of splice variant BC-XY of triple-targeter #1 fused to the amino acid sequence of E. coli GDH subunit #3 with myc epitope tag. SEQ ID NO: 62 shows the amino acid sequence of splice variant BC-XY of triple-targeter #1 fused to the amino acid sequence of GFP. SEQ ID NO: 63 shows the amino acid sequence of splice variant AC-XZ of triple-targeter #2 fused to the amino acid sequence of E. coli GDH subunit #1. SEQ ID NO: 64 shows the amino acid sequence of splice variant AC-XZ of triple-targeter #2 fused to the amino acid sequence of E. coli GDH subunit #2. SEQ ID NO: 65 shows the amino acid sequence of splice variant AC-XZ of triple-targeter #2 fused to the amino acid sequence of E. coli GDH subunit #3. SEQ ID NO: 66 shows the amino acid sequence of splice variant AC-XZ of triple-targeter #2 fused to the amino acid sequence of E. coli GDH subunit #3 with myc epitope tag. SEQ ID NO: 67 shows the amino acid sequence of splice variant AC-XZ of triple-targeter #2 fused to the amino acid sequence of GFP. SEQ ID NO: 68 shows the amino acid sequence of splice variant BC-XZ of triple-targeter #2 fused to the amino acid sequence of E. coli GDH subunit #1. SEQ ID NO: 69 shows the amino acid sequence of splice variant BC-XZ of triple-targeter #2 fused to the amino acid sequence of E. coli GDH subunit #2. SEQ ID NO: 70 shows the amino acid sequence of splice variant BC-XZ of triple-targeter #2 fused to the amino acid sequence of E. coli GDH subunit #3. SEQ ID NO: 71 shows the amino acid sequence of splice variant BC-XZ of triple-targeter #2 fused to the amino acid sequence of E. coli GDH subunit #3 with myc epitope tag. SEQ ID NO: 72 shows the amino acid sequence of splice variant BC-XZ of triple-targeter #2 fused to the amino acid sequence of GFP. SEQ ID NO: 73 shows the amino acid sequence of splice variant AC-XY of triple-targeter #2 fused to the amino acid sequence of E. coli GDH subunit #1. SEQ ID NO: 74 shows the amino acid sequence of splice variant AC-XY of triple-targeter #2 fused to the amino acid sequence of E. coli GDH subunit #2. SEQ ID NO: 75 shows the amino acid sequence of splice variant AC-XY of triple-targeter #2 fused to the amino acid sequence of E. coli GDH subunit #3. SEQ ID NO: 76 shows the amino acid sequence of splice variant AC-XY of triple-targeter #2 fused to the amino acid sequence of E. coli GDH subunit #3 with myc epitope tag. SEQ ID NO: 77 shows the amino acid sequence of splice variant AC-XY of triple-targeter #2 fused to the amino acid sequence of GFP. SEQ ID NO: 78 shows the amino acid sequence of splice variant BC-XY of triple-targeter #2 fused to the amino acid sequence of E. coli GDH subunit #1. SEQ ID NO: 79 shows the amino acid sequence of splice variant BC-XY of triple-targeter #2 fused to the amino acid sequence of E. coli GDH subunit #2. SEQ ID NO: 80 shows the amino acid sequence of splice variant BC-XY of triple-targeter #2 fused to the amino acid sequence of E. coli GDH subunit #3. SEQ ID NO: 81 shows the amino acid sequence of splice variant BC-XY of triple-targeter #2 fused to the amino acid sequence of E. coli GDH subunit #3 with myc epitope tag. SEQ ID NO: 82 shows the amino acid sequence of splice variant BC-XY of triple-targeter #2 fused to the amino acid sequence of GFP. SEQ ID NO: 83 shows the amino acid sequence of triple-targeter #3 fused to the amino acid sequence of E. coli GDH subunit #1. SEQ ID NO: 84 shows the amino acid sequence of triple-targeter #3 fused to the amino acid sequence of E. coli GDH subunit #2. SEQ ID NO: 85 shows the amino acid sequence of triple-targeter #3 fused to the amino acid sequence of E. coli GDH subunit #3. SEQ ID NO: 86 shows the amino acid sequence of triple-targeter #3 fused to the amino acid sequence of E. coli GDH subunit #3 with myc epitope tag. SEQ ID NO: 87 shows the amino acid sequence of triple-targeter #3 fused to the amino acid sequence of GFP.

TABLE 2
Localization Tag Sequences

| SEQ | | |
|---|---|---|
| ID | | |
| NO: | Description | Sequence |
| 1 | CTPa | VCSLARNLCFSLFSTHKSGTGSSG |
| | polypeptide | |
| 2 | PTS2 | RLRIIGGHL |
| | polypeptide | |
| 3 | TriTag1 | MEVCSLARNLCFSLFSTHKSGTGSSGDSSSSGVSTKPQDRLRIIGGHLNV |
| | polypeptide; | AAEA |
| | variant 1 | |
| 4 | TriTag2 | MDSSSSPVSTKPQDRLRIIGGHLNVAAEAMEVCSLARNLCF |
| | polypeptide | SLFSTHKSGTGSSG |
| 5 | RLX₅HL | RLXXXXXHL; wherein X is any amino acid |
| | nonapeptide | |
| 6 | CTPb | MASSVISSAAVATRTNVTQAGSMIAPFTGLKSAATFPVSRKQNLDITSIAS |
| | polypeptide | NGGRVRC |
| 7 | TriTag3 | MASSVISSAAVATRTNVTQAGSMIAPFTGLKSAATFPVSRLRVLSAHLITS |
| | polypeptide | IASNGGRVRC |
| 8 | Weak Splice | GTATGT |
| | Donor Site; | |
| | Set 1 | |
| 9 | Strong Splice | GTATAC |
| | Donor Site; | |
| | Set 1 | |
| 10 | Splice | TTCCAG |
| | Acceptor Site; | |
| | Set 1 | |
| 11 | Splice Donor | GTATAT |
| | Site; Set 2 | |
| 12 | Weak Splice | TGTAAG |
| | Acceptor Site; | |
| | Set 2 | |
| 13 | Strong Splice | TTGCAG |
| | Acceptor Site; | |
| | Set 2 | |
| 14 | CTPa nucleic | GTATGTTCTCTTGCCAGGAATCTCTGCTTCAGTTTATTCTCAACACAT |
| | acid | AAGAGTGGAACTGGCTCGAGCGGC |
| 15 | CTPb nucleic | ATGGCTTCCTCTGTTATTTCCTCTGCCGCTGTTGCTACACGCACCAAT |
| | acid | GTTACACAAGCTGGCAGCATGATTGCACCTTTCACTGGTCTCAAATCT |
| | | GCTGCTACTTTCCCTGTTTCAAGGAAGCAAAACCTTGACATCACTTCC |
| | | ATTGCTAGCAATGGTGGAAGAGTTAGGTGCATG |
| 16 | PTS2 nucleic | CGTCTGAGAATCATTGGAGGGCATTTG |
| | acid | |
| 17 | Nonapeptide | AGGCTTAGAGTTCTTTCTGCTCATTTG |
| | nucleic acid | |
| 18 | TriTag1 | ATGGAGGTATGTTCTCTTGCCAGGAATCTCTGCTTCAGTTTATTCTCA |
| | nucleic acid | ACACATAAGGTATACAAATGGGTTATTTGGTGTTTCTCTGTGTTGTGT |
| | | GACTGATTTTGTGCTTATAGACGATTTTTAATATGTTGATGGTGTTAG |
| | | CAATTCCAGAGTGGAACTGGCTCGAGCGGCGACAGCTCTAGCTCTCC |
| | | TGTTTCAACAAAACCTCAAGGTATATTGATGATTTACCAAATCTTTTC |
| | | CTTGTCAAAGTTTTGTGTTTGACTGTGTGGGTTTGAACCTGTTAGGAT |
| | | TCAGTATGATATCAAGTATGTGTCTTTTGGAATACAAGGATTTACCCT |
| | | TATGGCTATCTTTGTTATCTGTGTGACCTTTTCTACTTTCTCGCTTTGT |
| | | AAGATCGTCTGAGAATCATTGGAGGGCATTTGAATGTTGCAGCTGAA |
| | | GCAATG |
| 19 | TriTag2 | ATGGACAGCTCTAGCTCTCCTGTTTCAACAAAACCTCAAGGTATATTG |
| | nucleic acid | ATGATTTACCAAATCTTTTCCTTGTCAAAGTTTTGTGTTTGACTGTGTG |
| | | GGTTTGAACCTGTTAGGATTCAGTATGATATCAAGTATGTGTCTTTTG |
| | | GAATACAAGGATTTACCCTTATGGCTATCTTTGTTATCTGTGTGACCT |
| | | TTTCTACTTTCTCGCTTTGTAAGATCGTCTGAGAATCATTGGAGGGCA |
| | | TTTGAATGTTGCAGCTGAAGCAATGGAGGTATGTTCTCTTGCCAGGA |
| | | ATCTCTGCTTCAGTTTATTCTCAACACATAAGGTATACAAATGGGTTA |
| | | TTTGGTGTTTCTCTGTGTTGTGTGACTGATTTTGTGCTTATAGACGATT |
| | | TTTAATATGTTGATGGTGTTAGCAATTCCAGAGTGGAACTGGCTCGAG |
| | | CGGCATG |
| 20 | TriTag3 | ATGGCTTCCTCTGTTATTTCCTCTGCCGCTGTTGCTACACGCACCAAT |
| | nucleic acid | GTTACACAAGCTGGCAGCATGATTGCACCTTTCACTGGTCTCAAATCT |
| | | GCTGCTACTTTCCCTGTTTCAAGGCTTAGA |
| | | GTTCTTTCTGCTCATTTGATCACTTCCATTGCTAGCAATGGTGGAAGA |
| | | GTTAGGTGCATG |
| 21 | TriTag1 | MESGTGSSGDSSSSGVSTKPQDRLRIIGGHLNVAAEA |
| | polypeptide; | |
| | splice variant | |
| | 2 | |
| 22 | TriTag1 | MEVCSLARNLCFSLFSTHKSGTGSSGDSSSSGVSTKPQAEA |
| | polypeptide; | |
| | splice variant | |
| | 3 | |
| 23 | TriTag1 | MESGTGSSGDSSSSGVSTKPQAEA |
| | polypeptide; | |
| | splice variant | |
| | 4 | |
| 24 | TriTag2 | MDSSSSPVSTKPQAEAMEVCSLARNLCFSLFSTHKSGTGSSG |
| | polypeptide; | |
| | splice variant | |
| | 2 | |
| 25 | TriTag2 | MDSSSSPVSTKPQDRLRIIGGHLNVAAEAMESGTGSSG |
| | polypeptide; | |
| | splice variant | |
| | 3 | |
| 26 | TriTag2 | MDSSSSPVSTKPQAEAMESGTGSSG |
| | polypeptide; | |
| | splice variant | |
| | 4 | |
| 27 | Nonapeptide | RLRVLSAHL |

1. An engineered multiple localization tag comprising a nucleic acid sequence encoding at least two localization signal sequences;
wherein each of the localization signal sequences will direct localization of a polypeptide encoded by an operably linked sequence to a different set of subcellular compartments.

2. The engineered multiple localization tag of claim 1, wherein the localization signal sequences are not separated by an exon.

3. The engineered multiple localization tag of claim 1, wherein the localization signal sequence are separated by an exon of no more than 300 bases.

4. The engineered multiple localization tag of claim 3, wherein the exon comprises glycine and serine residues.

5. The engineered multiple localization tag of claim 1, further comprising a set of compatible splicing sequences;
wherein the set comprises two alternative splice donor sequences and one splice acceptor sequence;
wherein the two alternative splice donor sequences flank one localization signal sequence; and
the splice acceptor sequence is located 3′ of both splice donor sequences of the set.

6. (canceled)

7. (canceled)

8. The engineered multiple localization tag of claim 1, further comprising a set of compatible splicing sequences;
wherein the set comprises two alternative splice acceptor sequences and one splice donor sequence;
wherein the two alternative splice acceptor sequences flank a localization signal sequence; and
the splice donor sequence is located 5′ of both splice acceptor sequences of the set.

9. (canceled)

10. (canceled)

11. The engineered multiple localization tag of claim 5 or 8, wherein a pair of alternative splice sites comprises a weak and a strong splice site.

12. (canceled)

13. (canceled)

14. (canceled)

15. The engineered multiple localization tag of claim 1, wherein each of the localization signals is selected from the group consisting of:
a chloroplast localization signal; a peroxisome localization signal; a mitochondrion localization signal; a secretory pathway localization signal; an endoplasmic reticulum localization signal; and a vacuole secretion localization signal.

16. The engineered multiple localization tag of claim 15, wherein the chloroplast localization signal comprises a nucleic acid sequence selected from the group consisting of: a nucleic acid sequence encoding CTPa (SEQ ID NO:1) or a polypeptide having at least 90% identity to CTPa; a nucleic acid sequence of SEQ ID NO:14 or a sequence having at least 90% identity to SEQ ID NO:14; a nucleic acid sequence encoding CTPb (SEQ ID NO:6) or a polypeptide having at least 90% identity to CTPb; the nucleic acid sequence of SEQ ID NO:15 or a sequence having at least 90% identity to SEQ ID NO:15.

17. (canceled)

18. (canceled)

19. (canceled)

20. The engineered multiple localization tag of claim 15, wherein the peroxisome localization signal comprises a nucleic acid sequence selected from the group consisting of: a nucleic acid sequence encoding PTS2 (SEQ ID NO:2) or a polypeptide having at least 90% identity to PTS2; the nucleic acid sequence of SEQ ID NO:16 or a polypeptide having at least 90% identity to SEQ ID NO:16; the nucleic acid sequence of SEQ ID NO: 5; and the nucleic acid sequence of SEQ ID NO:17 or a sequence having at least 90% identity to SEQ ID NO:17.

21. (canceled)

22. (canceled)

23. (canceled)

24. The engineered multiple localization tag of claim 1, comprising the nucleic acid sequence encoding a polypeptide of any of SEQ ID NOs:3 and 21-23 or a polypeptide having at least 90% identity to any of SEQ ID NOs:3 and 21-23.

25. The engineered multiple localization tag of claim 24, comprising the nucleic acid sequence of SEQ ID NO:18 or a sequence having at least 90% identity to SEQ ID NO:18.

26. The engineered multiple localization tag of claim 1, comprising the sequence of any of SEQ ID NOs:4 and 24-26 or a sequence having at least 90% identity to any of SEQ ID NOs:4 and 24-26.

27. The engineered multiple localization tag of claim 26, comprising the nucleic acid sequence of SEQ ID NO:19 or a sequence having at least 90% identity to SEQ ID NO:19.

28. The engineered multiple localization tag of claim 1, wherein a first localization signal is comprised within a second localization signal.

29. The engineered multiple localization tag of claim 28, wherein the first localization signal is substituted for the amino acids equivalent to residues 37 to 46 of SEQ ID NO: 6.

30. The engineered multiple localization tag of claim 29,
comprising a nucleic acid sequence selected from the group consisting of: a nucleic acid sequence encoding the sequence of SEQ ID NO:7 or encoding a sequence having at least 90% identity to SEQ ID NO:7; and the nucleic acid sequence of SEQ ID NO:20 or a sequence having at least 90% identity to SEQ ID NO:20.

31. (canceled)

32. A vector comprising the engineered multiple localization tag of claim 1.

33. (canceled)

34. (canceled)

35. An engineered cell or organism comprising the engineered multiple localization tag of claim 1.

36. A nucleic acid molecule having the sequence of, or encoding the polypeptide having the sequence of, any of SEQ ID NO: 28-87, or a sequence having at least 90% identity thereto.

37. A vector comprising the nucleic acid molecule of claim 36.

38. An engineered cell or organism comprising the nucleic acid molecule of claim 36.
